SEQUENCE LISTING (431 sequences)

SEQ ID NO: 1 - RNA, 45 nt, Unknown
  Description of Unknown GluR2 domain sequence
guggaauaguauaacaauaugcuaaauguuguuauaguaucccac

SEQ ID NO: 2 - DNA, 45 bp, Artificial Sequence
  Description of Artificial Sequence Synthetic oligonucleotide
gaaagtactttgatgaatacatccacggctaatgaattcctttac

SEQ ID NO: 3 - DNA, 45 bp, Artificial Sequence
  Description of Artificial Sequence Synthetic oligonucleotide
gaaagtcctttcaagaatacatccacggctatactattcctttac

SEQ ID NO: 4 - DNA, 45 bp, Artificial Sequence
  Description of Artificial Sequence Synthetic oligonucleotide
gaaagtcctttgatgcatacatccacggctaatgaattcctttac

SEQ ID NO: 5 - DNA, 45 bp, Artificial Sequence
  Description of Artificial Sequence Synthetic oligonucleotide
gaaagtcctttgatgcatacatccacggctatactattcctttac

SEQ ID NO: 6 - DNA, 45 bp, Artificial Sequence
  Description of Artificial Sequence Synthetic oligonucleotide
gaaagtcctttgaggcatacatccacggctatactattcctttac

SEQ ID NO: 7 - DNA, 45 bp, Artificial Sequence
  Description of Artificial Sequence Synthetic oligonucleotide
gaaagtcctttgaggaatacatccacggctatactattcctttac

SEQ ID NO: 8 - DNA, 45 bp, Artificial Sequence
  Description of Artificial Sequence Synthetic oligonucleotide
gaaagtcctttgaagcatacatccacggctatactattcctttac

SEQ ID NO: 9 - DNA, 45 bp, Artificial Sequence
  Description of Artificial Sequence Synthetic oligonucleotide
gaaagtactttcacgaatacatccacggctatactattcctttac

SEQ ID NO: 10 - DNA, 45 bp, Artificial Sequence
  Description of Artificial Sequence Synthetic oligonucleotide
gaaagtcctttcacgcatacatccacggctatactattcctttac

SEQ ID NO: 11 - DNA, 45 bp, Artificial Sequence
  Description of Artificial Sequence Synthetic oligonucleotide
gaaagtcctttcaagcatacatccacggctatactattcctttac

SEQ ID NO: 12 - RNA, 101 nt, Artificial Sequence
  Description of Artificial Sequence Synthetic polynucleotide
gccacaugaggguccuuggccuuugaaaguccuuucaugaauacagccacggcuaauugacauggcuuacuagucacugucguacauuggccacucccagu

SEQ ID NO: 13 - RNA, 100 nt, Artificial Sequence
  Description of Artificial Sequence Synthetic polynucleotide
gccacauggagguccuuggccuuugaaaguccuuucaugaauacaaccacggcgucgcuauuccuuuacuagucacugucguacauuggccacucccagu

SEQ ID NO: 14 - RNA, 100 nt, Artificial Sequence
  Description of Artificial Sequence Synthetic polynucleotide
gccacacagaccuccucuacgguugaaaguccuuucaugaauacucuccacggcaaugaaugaauugacuagucacugucguacauuggccacucccagu

SEQ ID NO: 15 - RNA, 100 nt, Artificial Sequence
  Description of Artificial Sequence Synthetic polynucleotide
gccacacagaccuccuuggcaaagcuaaguccuuucaugaauaauuccacggcugauccggggcuuuacuagucacugucguacauuggccacucccagu

SEQ ID NO: 16 - RNA, 100 nt, Artificial Sequence
  Description of Artificial Sequence Synthetic polynucleotide
gccacacagaccucgaucuacuuugaaaguccuuucauggcuacauccacggcucaagaauguacagacuagucacugucguacauuggccacucccagu

SEQ ID NO: 17 - RNA, 100 nt, Artificial Sequence
  Description of Artificial Sequence Synthetic polynucleotide
gccacacagaccuccuaaagaguugaaaguccuuucauguauacauccacgacuccugaccaguguuacuagucacugucguacauuggccacucccagu

SEQ ID NO: 18 - RNA, 99 nt, Artificial Sequence
  Description of Artificial Sequence Synthetic oligonucleotide
gccacacagaccuccuuguaagcagaaagucuuucaugauuagccccacggcuaaugaauucaucagauagucacugucguacauuggccacucccagu

SEQ ID NO: 19 - RNA, 100 nt, Artificial Sequence
  Description of Artificial Sequence Synthetic polynucleotide
gccacacgaggauccuuggccuuugaaaguccucucaugaauacacgcacggcuauugaauuccucccaaagucacugucguacauuggccacucccagu

SEQ ID NO: 20 - RNA, 100 nt, Artificial Sequence
  Description of Artificial Sequence Synthetic polynucleotide
gccacacagaccuccuugaaugcagaaaguccuuucaugcuuacauccacggcuaauuaauucgacgaauagucacugucguacauuggccacucccagu

SEQ ID NO: 21 - RNA, 100 nt, Artificial Sequence
  Description of Artificial Sequence Synthetic polynucleotide
gccacacagaccuccuuguuaagggaaaguccuuucccgaauacauccacggcuaauucucgacuuuacuagucacugucguacauuggccacucccagu

SEQ ID NO: 22 - RNA, 98 nt, Artificial Sequence
  Description of Artificial Sequence Synthetic oligonucleotide
gccacacagaccuccugaaugauugaaaguccuuucaugaauacauccacggcaugaucugaguuacuagucacugucguacauuggccacucccagu

SEQ ID NO: 23 - RNA, 100 nt, Artificial Sequence
  Description of Artificial Sequence Synthetic polynucleotide
gccacacagaccuccuuguaaucagaaaguccuuuaaugaauaccuccacggcuucucaauuccucauucggucacugucguacauuggccacucccagu

SEQ ID NO: 24 - RNA, 101 nt, Artificial Sequence
  Description of Artificial Sequence Synthetic polynucleotide
gccacacagaccuccuugauacacgaaaguccuuuaaugaacgacauccacggcuaaagaauucauuguauagucacugucguacauuggccacucccagu

SEQ ID NO: 25 - RNA, 100 nt, Artificial Sequence
  Description of Artificial Sequence Synthetic polynucleotide
gccacacagaccaggaucgccuuugaaaguccuucccugaauacauccacggcuacgacaguccuuuacuagucacugucguacauuggccacucccagu

SEQ ID NO: 26 - RNA, 100 nt, Artificial Sequence
  Description of Artificial Sequence Synthetic polynucleotide
gccacacagaccucauucgacuuugaaaguccuuucaggaauaacuccacggcacggucauuccuuuacuagucacugucguacauuggccacucccagu

SEQ ID NO: 27 - RNA, 100 nt, Artificial Sequence
  Description of Artificial Sequence Synthetic polynucleotide
gccacacagaccuccuugauagcagaaagucaauuuaugaauacauccacggcuauuuauguccuuuacuagucacugucguacauuggccacucccagu

SEQ ID NO: 28 - RNA, 99 nt, Artificial Sequence
  Description of Artificial Sequence Synthetic oligonucleotide
gccacacagaccuccucuuuuguugaaaguagguucaugacacauccacggcuacguuucuccuuuacuagucacugucguacauuggccacucccagu

SEQ ID NO: 29 - RNA, 98 nt, Artificial Sequence
  Description of Artificial Sequence Synthetic oligonucleotide
gccacacagaccuccuauucgguugaaaguguuucaucauacauccacggcuaacgaauucagaucauagucacugucguacauuggccacucccagu

SEQ ID NO: 30 - RNA, 100 nt, Artificial Sequence
  Description of Artificial Sequence Synthetic polynucleotide
gccacacagaccuccuauauucuugaaaguccuugcaugaaugcauucacggcuaucaggcuccuuuacuagucacugucguacauuggccacucccagu

SEQ ID NO: 31 - RNA, 100 nt, Artificial Sequence
  Description of Artificial Sequence Synthetic polynucleotide
gccacacagaccucauccugcuuugaaaguccuuucaugaaucucuccacggcuaagaaauauuaagacuagucacugucguacauuggccacucccagu

SEQ ID NO: 32 - RNA, 99 nt, Artificial Sequence
  Description of Artificial Sequence Synthetic oligonucleotide
gccacacaggaaaacuuggccuuugaaaguccuuguaugaaccaaccacggcuacuaagauccuuuacuagucacugucguacauuggccacucccagu

SEQ ID NO: 33 - RNA, 101 nt, Artificial Sequence
  Description of Artificial Sequence Synthetic polynucleotide
gccacacagaccuccuuguaugacgaaaguccuuucaugaauacaauccacggcuaaugacgcguauuacuagucacugucguacauuggccacucccagu

SEQ ID NO: 34 - RNA, 100 nt, Artificial Sequence
  Description of Artificial Sequence Synthetic polynucleotide
gccacacaguauggcuuggccuuugaaaguccuuugaugaauacauccacggcucuugaauuccuuuacuguggucugucguacauuggccacucccagu

SEQ ID NO: 35 - RNA, 100 nt, Artificial Sequence
  Description of Artificial Sequence Synthetic polynucleotide
gccacacagaccuccuaauaaguugaaagucuuuucaugaauacauucacggcuaaugacgcgaauuacuagucacugucguacauuggccacucccagu

SEQ ID NO: 36 - RNA, 100 nt, Artificial Sequence
  Description of Artificial Sequence Synthetic polynucleotide
gccacaugaggauccuuggccuuugaaaguccuuucaugcauaguuccacggcuaaagaaugugcgaacuagucacugucguacauuggccacucccagu

SEQ ID NO: 37 - RNA, 100 nt, Artificial Sequence
  Description of Artificial Sequence Synthetic polynucleotide
gccacacagaccggugacgccuuugaaaguacuuucauuaauacauccacggcuaaugaauuccugucaaagucacugucguacauuggccacucccagu

SEQ ID NO: 38 - RNA, 100 nt, Artificial Sequence
  Description of Artificial Sequence Synthetic polynucleotide
gccacacagaccuccuaguagguugaaaguccuuucauuaauaacuccacggcuaaugaucugaguuacuagucacugucguacauuggccacucccagu

SEQ ID NO: 39 - RNA, 100 nt, Artificial Sequence
  Description of Artificial Sequence Synthetic polynucleotide
gccacacagaccaaagaugccuuugaaaguccuuucaggaauacauccacggcuaaugaauuccuuuacugaggucugucguacauuggccacucccagu

SEQ ID NO: 40 - RNA, 100 nt, Artificial Sequence
  Description of Artificial Sequence Synthetic polynucleotide
gccacacaggaaaacuuggccuuugaaaguccuuucauuaauacauccacggcuaauccgcggcuuuacuagucacugucguacauuggccacucccagu

SEQ ID NO: 41 - RNA, 101 nt, Artificial Sequence
  Description of Artificial Sequence Synthetic polynucleotide
gccacacagagggaacuggccuuugaaaguucuuucaugaauacaucgcacggcuacaaagguccuuuacuagucacugucguacauuggccacucccagu

SEQ ID NO: 42 - RNA, 100 nt, Artificial Sequence
  Description of Artificial Sequence Synthetic polynucleotide
gccacacaagggcgcuuggccuuugaaaguccuuucauaaauacauccacggcuaaucuuaggcuuuacuagucacugucguacauuggccacucccagu

SEQ ID NO: 43 - RNA, 98 nt, Artificial Sequence
  Description of Artificial Sequence Synthetic oligonucleotide
gccacacagaccuccuugaauggggaaaguaaauucaugaauacauccacggccggaauuccucagguagucacugucguacauuggccacucccagu

SEQ ID NO: 44 - RNA, 100 nt, Artificial Sequence
  Description of Artificial Sequence Synthetic polynucleotide
gccacauagggauccuuggccuuugaaaguccuuucaggaauacauccacggcuaaucugcggcuuuacuagucacugucguacauuggccacucccagu

SEQ ID NO: 45 - RNA, 100 nt, Artificial Sequence
  Description of Artificial Sequence Synthetic polynucleotide
gccacacagaagaggauggccuuugaaaguccuuucauuaauacauccacggcuaaucuucgacuuuacuagucacugucguacauuggccacucccagu

SEQ ID NO: 46 - RNA, 99 nt, Artificial Sequence
  Description of Artificial Sequence Synthetic oligonucleotide
gccacacagaagcauauggccuuugaaaguccuuucaugaauacauccacggcuaacaauuccuuuuggaaucacugucguacauuggccacucccagu

SEQ ID NO: 47 - RNA, 100 nt, Artificial Sequence
  Description of Artificial Sequence Synthetic polynucleotide
gccacacagaccuccuuguagggggaaaguccuuucauaaagacauccacggcuaaugaauucgcgguguagucacugucguacauuggccacucccagu

SEQ ID NO: 48 - RNA, 100 nt, Artificial Sequence
  Description of Artificial Sequence Synthetic polynucleotide
gccacacagaccucgcgcaacuuugaaaguccuuucaugcauagauccacggcuacauaauuccuuuuggagucacugucguacauuggccacucccagu

SEQ ID NO: 49 - RNA, 99 nt, Artificial Sequence
  Description of Artificial Sequence Synthetic oligonucleotide
gccacacagaccuccuuggcagccuuaagucguuucaugaauagauccacggcuaaaaauuccugauucggucacugucguacauuggccacucccagu

SEQ ID NO: 50 - RNA, 98 nt, Artificial Sequence
  Description of Artificial Sequence Synthetic oligonucleotide
gccacacagaccuccuugaagauggaaaguccuuucaugaagacauccacggcuaucaaagcuuuacuagucacugucguacauuggccacucccagu

SEQ ID NO: 51 - RNA, 100 nt, Artificial Sequence
  Description of Artificial Sequence Synthetic polynucleotide
gccacacagaccuccucucauguugaaagucgguucaugacuacauccacggcuaaugaauucaaccgauagucacugucguacauuggccacucccagu

SEQ ID NO: 52 - RNA, 100 nt, Artificial Sequence
  Description of Artificial Sequence Synthetic polynucleotide
gccacacagaccucggacaacuuugaaaguccuuucaugaauacaaccacggcucaacaauucgaaccguagucacugucguacauuggccacucccagu

SEQ ID NO: 53 - RNA, 100 nt, Artificial Sequence
  Description of Artificial Sequence Synthetic polynucleotide
gccacacagaccgggucagccuuugaaaguccguucaugaauacauccacggcuauagaauucgccccguagucacugucguacauuggccacucccagu

SEQ ID NO: 54 - RNA, 100 nt, Artificial Sequence
  Description of Artificial Sequence Synthetic polynucleotide
gccacacagaccuccuugauggaggaaaguagcaucauguuuacauccacggcuaugccuguccuuuacuagucacugucguacauuggccacucccagu

SEQ ID NO: 55 - RNA, 99 nt, Artificial Sequence
  Description of Artificial Sequence Synthetic oligonucleotide
gccacacagaccuccuuggcauacuuaaguccuuucaucauacauccacggcuaauuuugggcuuuacuagucacugucguacauuggccacucccagu

SEQ ID NO: 56 - RNA, 100 nt, Artificial Sequence
  Description of Artificial Sequence Synthetic polynucleotide
gccacacagaccuccuauuagguugaaaguacuuucaugaauacauccacggcuaauuuaaaacuuuacuagucacugucguacauuggccacucccagu

SEQ ID NO: 57 - RNA, 100 nt, Artificial Sequence
  Description of Artificial Sequence Synthetic polynucleotide
gccacacagaaagaacuggccuuugaaaguccuuucaugaauacacccacgacuaaugaauuccuaccugagucacugucguacauuggccacucccagu

SEQ ID NO: 58 - RNA, 99 nt, Artificial Sequence
  Description of Artificial Sequence Synthetic oligonucleotide
gccacaugggaguccuuggccuuugaaaguccuuuaaugaauacauccacggaaaugauauagcuuacuagucacugucguacauuggccacucccagu

SEQ ID NO: 59 - RNA, 100 nt, Artificial Sequence
  Description of Artificial Sequence Synthetic polynucleotide
gccacacagaccucagacagcuuugaaaguccuaucaugaauagauccacggcuaaugacgguaguuacuagucacugucguacauuggccacucccagu

SEQ ID NO: 60 - RNA, 100 nt, Artificial Sequence
  Description of Artificial Sequence Synthetic polynucleotide
gccacauagcgguccuuggccuuugaaaguccuuucaugaauacaaccacggcuaauuuagcgcuuuacuagucacugucguacauuggccacucccagu

SEQ ID NO: 61 - RNA, 100 nt, Artificial Sequence
  Description of Artificial Sequence Synthetic polynucleotide
gccacacagaccuccuugaagggcgaaagucguuucaugaauacauccacggcuaaagaauuccucguuaagucacugucguacauuggccacucccagu

SEQ ID NO: 62 - RNA, 97 nt, Artificial Sequence
  Description of Artificial Sequence Synthetic oligonucleotide
gccacacagaccucgacuuacuuugaaagucccucaugaauacauccacggaaugaauuccuaguagagucacugucguacauuggccacucccagu

SEQ ID NO: 63 - RNA, 100 nt, Artificial Sequence
  Description of Artificial Sequence Synthetic polynucleotide
gccacacagaccuccuuggcagaaauaaguccuuggaugaauacauccacggcuaaugaauuccucguugagucacugucguacauuggccacucccagu

SEQ ID NO: 64 - RNA, 100 nt, Artificial Sequence
  Description of Artificial Sequence Synthetic polynucleotide
gccacacagaccuccuuguugauggaaaguccguucauaaauacauccacgacccaugaauuccugggucggucacugucguacauuggccacucccagu

SEQ ID NO: 65 - RNA, 99 nt, Artificial Sequence
  Description of Artificial Sequence Synthetic oligonucleotide
gccacacagaccuccuuguagguagaaaguccgagcaggaauacauccacggcaaugaauuccuaguucggucacugucguacauuggccacucccagu

SEQ ID NO: 66 - RNA, 100 nt, Artificial Sequence
  Description of Artificial Sequence Synthetic polynucleotide
gccacacagaaaaacuuggccuuugaaaguccuuuuaugccuacuuccacggcuacgucaguccuuuacuagucacugucguacauuggccacucccagu

SEQ ID NO: 67 - RNA, 99 nt, Artificial Sequence
  Description of Artificial Sequence Synthetic oligonucleotide
gccacaugggaguccuuggccuuugaaaguccuuucaugauacauccacggcuaauccuuggcuuuacuagucacugucguacauuggccacucccagu

SEQ ID NO: 68 - RNA, 98 nt, Artificial Sequence
  Description of Artificial Sequence Synthetic oligonucleotide
gccacacagaccuccuacacgguugaaaguccauucaugaacauccacggcuaauuucaagcuuuacuagucacugucguacauuggccacucccagu

SEQ ID NO: 69 - RNA, 100 nt, Artificial Sequence
  Description of Artificial Sequence Synthetic polynucleotide
gccacacagaccuccugaauacuugaaaguccugucaugaauacauccacgacuaccgucauccuuuacuagucacugucguacauuggccacucccagu

SEQ ID NO: 70 - RNA, 100 nt, Artificial Sequence
  Description of Artificial Sequence Synthetic polynucleotide
gccacacagaccuccuugauggcggaaaguccuuucaugaauacauccacggcuaaagaauucgauccauagucacugucguacauuggccacucccagu

SEQ ID NO: 71 - RNA, 101 nt, Artificial Sequence
  Description of Artificial Sequence Synthetic polynucleotide
gccacacagaccuccuuggcgcgauuaaguccccuucaugaauacauccacggguacugaauuccuuuuguugucacugucguacauuggccacucccagu

SEQ ID NO: 72 - RNA, 100 nt, Artificial Sequence
  Description of Artificial Sequence Synthetic polynucleotide
gccacacagaccuccuuggcagcgauaaguccguucaugaaaagauccacggcuaauccacgacuuuacuagucacugucguacauuggccacucccagu

SEQ ID NO: 73 - RNA, 99 nt, Artificial Sequence
  Description of Artificial Sequence Synthetic oligonucleotide
gccacacagaccuccucuuagauugaaaguguucaugaauagaucacacggcuaaugaauucacacuguagucacugucguacauuggccacucccagu

SEQ ID NO: 74 - RNA, 100 nt, Artificial Sequence
  Description of Artificial Sequence Synthetic polynucleotide
gccacacaagauagcuuggccuuugaaaguccuuggauuaaugcauccacggcuaauucuuagcuuuacuagucacugucguacauuggccacucccagu

SEQ ID NO: 75 - RNA, 97 nt, Artificial Sequence
  Description of Artificial Sequence Synthetic oligonucleotide
gccacacagaggaacuuggccuuugaaagucccuucaauacauccacgacuauaguuguccuuuacuagucacugucguacauuggccacucccagu

SEQ ID NO: 76 - RNA, 100 nt, Artificial Sequence
  Description of Artificial Sequence Synthetic polynucleotide
gccacacaauagaacuuggccuuugaaaguccuuucaugaauaaauccacggcuaauaacaggcuuuacuagucacugucguacauuggccacucccagu

SEQ ID NO: 77 - RNA, 100 nt, Artificial Sequence
  Description of Artificial Sequence Synthetic polynucleotide
gccacacagaccucgaacgacuuugaaaguccuuucaugaauagauccacggcuaauuauaagcuuuacuagucacugucguacauuggccacucccagu

SEQ ID NO: 78 - RNA, 100 nt, Artificial Sequence
  Description of Artificial Sequence Synthetic polynucleotide
gccacacagaccucgaacgacuuugaaaguccuuucaugaauagauccacggcuaauuauaagcuuuacaccacacugucgucgaauggccacucccagu

SEQ ID NO: 79 - RNA, 100 nt, Artificial Sequence
  Description of Artificial Sequence Synthetic polynucleotide
gccacacagaccuccuuggcgaucacaaguccuuucaugaauaaauccacgguuaaugaccaguguuacuagucacugucguacauuggccacucccagu

SEQ ID NO: 80 - RNA, 100 nt, Artificial Sequence
  Description of Artificial Sequence Synthetic polynucleotide
gccacacagaccuccugauuaauugaaaguccuuucaagaauacacacacggguaaugaauucgcauuguagucacugucguacauuggccacucccagu

SEQ ID NO: 81 - RNA, 99 nt, Artificial Sequence
  Description of Artificial Sequence Synthetic oligonucleotide
gccacacagaccagguaugccuuugaaaguaguucaugaauacaaccacggcuacacaauuccuuucuuggucacugucguacauuggccacucccagu

SEQ ID NO: 82 - RNA, 100 nt, Artificial Sequence
  Description of Artificial Sequence Synthetic polynucleotide
gccacacagaauaagcuggccuuugaaaguccuuucaugaauacacccacgacuaaugaauuccugcaauggucacugucguacauuggccacucccagu

SEQ ID NO: 83 - RNA, 100 nt, Artificial Sequence
  Description of Artificial Sequence Synthetic polynucleotide
gccacacagaccuccuuggcggcgccaaguccuuucaugaauacaucaacggcuaaugaucgaaauuacuagucacugucguacauuggccacucccagu

SEQ ID NO: 84 - RNA, 99 nt, Artificial Sequence
  Description of Artificial Sequence Synthetic oligonucleotide
gccacacagaccuccuuggccucccuccguccucucauaaauacauccacggccaugaauggagacacuagucacugucguacauuggccacucccagu

SEQ ID NO: 85 - RNA, 99 nt, Artificial Sequence
  Description of Artificial Sequence Synthetic oligonucleotide
gccacacagaccuccuuguugaaggaaaguccuucgugaauacauccacggcuaaucuggggcuuuacuagucacugucguacauuggccacucccagu

SEQ ID NO: 86 - RNA, 100 nt, Artificial Sequence
  Description of Artificial Sequence Synthetic polynucleotide
gccacacagaccuccuuguaggaggaaaguccuuucaugaauacaugcacggcuaaugaucgguguuacuagucacugucguacauuggccacucccagu

SEQ ID NO: 87 - RNA, 100 nt, Artificial Sequence
  Description of Artificial Sequence Synthetic polynucleotide
gccacacagaccuccuuggcggaagcaaguccugccaugcauacauucacggcuaaugaauucgauggauagucacugucguacauuggccacucccagu

SEQ ID NO: 88 - RNA, 100 nt, Artificial Sequence
  Description of Artificial Sequence Synthetic polynucleotide
gccacacagaccuccuauaaacuugaaaguccauucaugaauagucccacggcuauugaauucacgucguagucacugucguacauuggccacucccagu

SEQ ID NO: 89 - RNA, 101 nt, Artificial Sequence
  Description of Artificial Sequence Synthetic polynucleotide
gccacacagaaagggcuggccuuugaaaguccuuucaugaauacauccacggcuaaaugaauucgccacguagucacugucguacauuggccacucccagu

SEQ ID NO: 90 - RNA, 102 nt, Artificial Sequence
  Description of Artificial Sequence Synthetic polynucleotide
gccacacagaccuccuuggcggucucaagucagcuuucaugauuacacccacggcuacggaauucggcugauagucacugucguacauuggccacucccagu

SEQ ID NO: 91 - RNA, 100 nt, Artificial Sequence
  Description of Artificial Sequence Synthetic polynucleotide
gccacacagaccuccuccaauguugaaaguacuuucaugaauacauccacggcgacugaauuccugucccagucacugucguacauuggccacucccagu

SEQ ID NO: 92 - RNA, 100 nt, Artificial Sequence
  Description of Artificial Sequence Synthetic polynucleotide
gccacacagaccuccuuguauuacgaaagugcuuugaugaauacauccacugcuaaugaauucagcccguagucacugucguacauuggccacucccagu

SEQ ID NO: 93 - RNA, 100 nt, Artificial Sequence
  Description of Artificial Sequence Synthetic polynucleotide
gccacacagaccauagacgccuuugaaagugcuuucaugaauacauccacggcuacugaauuccuuucuuggucacugucguacauuggccacucccagu

SEQ ID NO: 94 - RNA, 100 nt, Artificial Sequence
  Description of Artificial Sequence Synthetic polynucleotide
gccacacagaccuccuuggcggcaucaaguaauuucaugaauacauccacggcuacgacgguccuuuacuagucacugucguacauuggccacucccagu

SEQ ID NO: 95 - RNA, 100 nt, Artificial Sequence
  Description of Artificial Sequence Synthetic polynucleotide
gccacacagaccggaaaugccuuugaaagucccgccaugaauaauuccacggcuauggacgaugguuacuagucacugucguacauuggccacucccagu

SEQ ID NO: 96 - RNA, 100 nt, Artificial Sequence
  Description of Artificial Sequence Synthetic polynucleotide
gccacacagaccuccuuggcgaugcuaaguagcuucaugaauaccuccacguauaaugaauucagaccauagucacugucguacauuggccacucccagu

SEQ ID NO: 97 - RNA, 100 nt, Artificial Sequence
  Description of Artificial Sequence Synthetic polynucleotide
gccacacaggauagcuuggccuuugaaaguccuuucaugaauacuuccacggcuaaugaccaauguuacuagucacugucguacauuggccacucccagu

SEQ ID NO: 98 - RNA, 100 nt, Artificial Sequence
  Description of Artificial Sequence Synthetic polynucleotide
gccacacagaccuccuuggcggcgccaagugauuugaugaauacauccacggcuaguuuccaacuuuacuagucacugucguacauuggccacucccagu

SEQ ID NO: 99 - RNA, 100 nt, Artificial Sequence
  Description of Artificial Sequence Synthetic polynucleotide
gccacacagaccuccuuggcacgguuaaguccuucgaugaauacguccacggcuuuugaaugcaucgacuagucacugucguacauuggccacucccagu

SEQ ID NO: 100 - RNA, 100 nt, Artificial Sequence
  Description of Artificial Sequence Synthetic polynucleotide
gccacacagaccuccuuguaaacagaaaguccuuuggugaauacguccacggcuaaugaauuccucuuuuggucacugucguacauuggccacucccagu

SEQ ID NO: 101 - RNA, 100 nt, Artificial Sequence
  Description of Artificial Sequence Synthetic polynucleotide
gccacacagaccuccuugauaguggaaaguccuuucaugaauacauccacggguaaucgccgacuuuacuagucacugucguacauuggccacucccagu

SEQ ID NO: 102 - RNA, 100 nt, Artificial Sequence
  Description of Artificial Sequence Synthetic polynucleotide
gccacacagaccucaagauacuuugaaaguccuuucaugcauacauucacggcuaaugaauuccuuuacugauuucugucguacauuggccacucccagu

SEQ ID NO: 103 - RNA, 99 nt, Artificial Sequence
  Description of Artificial Sequence Synthetic oligonucleotide
gccacacagaccuccucaugaguugaaaguccuuucaugaauacauccacggacuugaauuccuggaauggucacugucguacauuggccacucccagu

SEQ ID NO: 104 - RNA, 100 nt, Artificial Sequence
  Description of Artificial Sequence Synthetic polynucleotide
gccacaugggcauccuuggccuuugaaaguccuuucauguauacauccacggcaguuacauuccuuuacuagucacugucguacauuggccacucccagu

SEQ ID NO: 105 - RNA, 100 nt, Artificial Sequence
  Description of Artificial Sequence Synthetic polynucleotide
gccacacaguagggcuuggccuuugaaaguccuuucaugaauacauucacggcuaaugaauaaaggcacuagucacugucguacauuggccacucccagu

SEQ ID NO: 106 - RNA, 100 nt, Artificial Sequence
  Description of Artificial Sequence Synthetic polynucleotide
gccacacagaccuccuuggcgacaacaaguccucucaugaauaauuccacggcuaccguucuccuuuacuagucacugucguacauuggccacucccagu

SEQ ID NO: 107 - RNA, 100 nt, Artificial Sequence
  Description of Artificial Sequence Synthetic polynucleotide
gccacauggggguccuuggccuuugaaaguccuuucaugaauauauccacggcuaaugacgauuauuacuagucacugucguacauuggccacucccagu

SEQ ID NO: 108 - RNA, 99 nt, Artificial Sequence
  Description of Artificial Sequence Synthetic oligonucleotide
gccacacagaggcgaauggccuuugaaaguccuuucaugaauacauccacggaaaugaauggcauaacuagucacugucguacauuggccacucccagu

SEQ ID NO: 109 - RNA, 101 nt, Artificial Sequence
  Description of Artificial Sequence Synthetic polynucleotide
gccacacagaccuccuuguagugcgaaaguccuuucaugaauaagauccacggcuacguaacuccuuuacuagucacugucguacauuggccacucccagu

SEQ ID NO: 110 - RNA, 100 nt, Artificial Sequence
  Description of Artificial Sequence Synthetic polynucleotide
gccacacagaccuccuaauaaauugaaaguccuuucaagaauacauccacggcuaaugaauuccuuuuuguuucacugucguacauuggccacucccagu

SEQ ID NO: 111 - RNA, 99 nt, Artificial Sequence
  Description of Artificial Sequence Synthetic oligonucleotide
gccacacagaaguagcuggccuuugaaaguccugcaugaauacauccacggcuaaagaauggcagaacuagucacugucguacauuggccacucccagu

SEQ ID NO: 112 - RNA, 102 nt, Artificial Sequence
  Description of Artificial Sequence Synthetic polynucleotide
gccacacagaccuccuugaauagggaaaguccauauucaugaauacccccacggcuaaugaauggaccgacuagucacugucguacauuggccacucccagu

SEQ ID NO: 113 - RNA, 101 nt, Artificial Sequence
  Description of Artificial Sequence Synthetic polynucleotide
gccacacagaccaaugcugccuuugaaaguccuuugcaucauuacauccacggcuaauaggcggcuuuacuagucacugucguacauuggccacucccagu

SEQ ID NO: 114 - RNA, 100 nt, Artificial Sequence
  Description of Artificial Sequence Synthetic polynucleotide
gccacacagaccuccucucaucuugaaagucgacuuucaugaauacauccacggaaugaauuccuuuuguaaucacugucguacauuggccacucccagu

SEQ ID NO: 115 - RNA, 101 nt, Artificial Sequence
  Description of Artificial Sequence Synthetic polynucleotide
gccacacagaccuccucucgacuugaaagucccuuucaugaauuucuccacggcuacgacuguccuuuacuagucacugucguacauuggccacucccagu

SEQ ID NO: 116 - RNA, 100 nt, Artificial Sequence
  Description of Artificial Sequence Synthetic polynucleotide
gccacacagaggcagauggccuuugaaaguccugucauuaauacagccacggcaugcuuauuccuuuacuagucacugucguacauuggccacucccagu

SEQ ID NO: 117 - RNA, 101 nt, Artificial Sequence
  Description of Artificial Sequence Synthetic polynucleotide
gccacacagaccuccuuguauaucgaaaguccuuuaaugaauagaauccacggcuaauccuuggcuuuacuagucacugucguacauuggccacucccagu

SEQ ID NO: 118 - RNA, 100 nt, Artificial Sequence
  Description of Artificial Sequence Synthetic polynucleotide
gccacacaagagggcuuggccuuugaaaguccuuuguuuaauacauccacggcuuaugaauuccuuucuuuuucacugucguacauuggccacucccagu

SEQ ID NO: 119 - RNA, 100 nt, Artificial Sequence
  Description of Artificial Sequence Synthetic polynucleotide
gccacacagaccucgcaucacuuugaaaguacuuucauuaauacauccacggcuacgucgcuccuuuacuagucacugucguacauuggccacucccagu

SEQ ID NO: 120 - RNA, 99 nt, Artificial Sequence
  Description of Artificial Sequence Synthetic oligonucleotide
gccacacagaccuccuuggcaaguccaaguccuuucaucaauacauccacggccaugacgcggguuacuagucacugucguacauuggccacucccagu

SEQ ID NO: 121 - RNA, 100 nt, Artificial Sequence
  Description of Artificial Sequence Synthetic polynucleotide
gccacacagaccuccucacuaguugaaagugauuccuugaauaccuccacggcuaaugaauuccuuuacuagucacugucguacauuggccacucccagu

SEQ ID NO: 122 - RNA, 100 nt, Artificial Sequence
  Description of Artificial Sequence Synthetic polynucleotide
gccacacagaccagaagugccuuugaaaguccuuucaugaauacaaccacggcuaauuugguacuuuacuagucacugucguacauuggccacucccagu

SEQ ID NO: 123 - RNA, 100 nt, Artificial Sequence
  Description of Artificial Sequence Synthetic polynucleotide
gccacacaggggagcuuggccuuugaaaguccguucaugaauucauccacggcuaucaacguccuuuacuagucacugucguacauuggccacucccagu

SEQ ID NO: 124 - RNA, 101 nt, Artificial Sequence
  Description of Artificial Sequence Synthetic polynucleotide
gccacacagaccuccuaauagguugaaaguccuuucguaaauacauccacggcuaauagaugaggcuuacuagucacugucguacauuggccacucccagu

SEQ ID NO: 125 - RNA, 100 nt, Artificial Sequence
  Description of Artificial Sequence Synthetic polynucleotide
gccacacagaccucgacacacuuugaaagucguaacaugaauacccccacggcuauggaauucaugccauagucacugucguacauuggccacucccagu

SEQ ID NO: 126 - RNA, 100 nt, Artificial Sequence
  Description of Artificial Sequence Synthetic polynucleotide
gccacacagaccuccuuggcgcgcauaaguccuuucaugaauaccuccacggcuaaucgcccgcuuuacuagucacugucguacauuggccacucccagu

SEQ ID NO: 127 - RNA, 101 nt, Artificial Sequence
  Description of Artificial Sequence Synthetic polynucleotide
gccacacagaccuccuugauggcggaaagucccuucaugaauacuauccacggcucuacaauuccuuuacuggagucugucguacauuggccacucccagu

SEQ ID NO: 128 - RNA, 100 nt, Artificial Sequence
  Description of Artificial Sequence Synthetic polynucleotide
gccacacagaccuccuauagaguugaaaguccuuucaugaauacaucuacggcuaaucgccgacuuuacuagucacugucguacauuggccacucccagu

SEQ ID NO: 129 - RNA, 100 nt, Artificial Sequence
  Description of Artificial Sequence Synthetic polynucleotide
gccacacagaccuccucaugaauugaaaguccuuucaugaauacagacacggcuaaugaauuccuuuuguuuucacugucguacauuggccacucccagu

SEQ ID NO: 130 - RNA, 100 nt, Artificial Sequence
  Description of Artificial Sequence Synthetic polynucleotide
gccacacgggcauccuuggccuuugaaaguccuuucaugaauacauccacggcuaacgaccgggcuuacuagucacugucguacauuggccacucccagu

SEQ ID NO: 131 - RNA, 100 nt, Artificial Sequence
  Description of Artificial Sequence Synthetic polynucleotide
gccacacagaccuccuugauuacagaaaguccuuucaugaauacagacacggcuaaugaauuccucaaauggucacugucguacauuggccacucccagu

SEQ ID NO: 132 - RNA, 100 nt, Artificial Sequence
  Description of Artificial Sequence Synthetic polynucleotide
gccacacagaccuccuauagacuugaaagucguuugaugaauacauccacggcuaaugaccgguauuacuagucacugucguacauuggccacucccagu

SEQ ID NO: 133 - RNA, 100 nt, Artificial Sequence
  Description of Artificial Sequence Synthetic polynucleotide
gccacacagaccuccucaaaacuugaaaguccuuagaugaauacauccacggguaaugaauuccuuuacuagucacugucguacauuggccacucccagu

SEQ ID NO: 134 - RNA, 99 nt, Artificial Sequence
  Description of Artificial Sequence Synthetic oligonucleotide
gccacacagaggcgcuuggccuuugaaaguccuuucugaauccauccacggcuaaucuacggcuuuacuagucacugucguacauuggccacucccagu

SEQ ID NO: 135 - RNA, 99 nt, Artificial Sequence
  Description of Artificial Sequence Synthetic oligonucleotide
gccacacagaccaagcucgccuuugaaaguccaucaugaauagauccacggcuaacuaauuccucuuguagucacugucguacauuggccacucccagu

SEQ ID NO: 136 - RNA, 99 nt, Artificial Sequence
  Description of Artificial Sequence Synthetic oligonucleotide
gccacacagaggcgaauggccuuugaaaguaguuuaaugaauacauccacggcuacgaauucgggguauagucacugucguacauuggccacucccagu

SEQ ID NO: 137 - RNA, 99 nt, Artificial Sequence
  Description of Artificial Sequence Synthetic oligonucleotide
gccacacagaagaaucuggccuuugaaaguccuuucuguuauacauccacgauaaugaauuccucguucggucacugucguacauuggccacucccagu

SEQ ID NO: 138 - RNA, 100 nt, Artificial Sequence
  Description of Artificial Sequence Synthetic polynucleotide
gccacacagaccuccuacaagguugaaaguccuuacaugaauucauccacguguauugaauuccuuuugaggucacugucguacauuggccacucccagu

SEQ ID NO: 139 - RNA, 100 nt, Artificial Sequence
  Description of Artificial Sequence Synthetic polynucleotide
gccacacagaccuccuuggcgacgccaaguccuuucaugaauacauccacggauaauucuuagcuuuacuagucacugucguacauuggccacucccagu

SEQ ID NO: 140 - RNA, 100 nt, Artificial Sequence
  Description of Artificial Sequence Synthetic polynucleotide
gccacacagaccuccucauugauugaaaguaauuucaugaauacauccacggcuacuacuguccuuuacuagucacugucguacauuggccacucccagu

SEQ ID NO: 141 - RNA, 99 nt, Artificial Sequence
  Description of Artificial Sequence Synthetic oligonucleotide
gccacacagaccuccucucagauugaaaguacuuucaugaaccuuccacggcuacgaaauucgguuuguagucacugucguacauuggccacucccagu

SEQ ID NO: 142 - RNA, 99 nt, Artificial Sequence
  Description of Artificial Sequence Synthetic oligonucleotide
gccacacagaccuccuugauacgcgaaaguaauuucaugaauacauccacggcuacgaauuccugguugagucacugucguacauuggccacucccagu

SEQ ID NO: 143 - RNA, 100 nt, Artificial Sequence
  Description of Artificial Sequence Synthetic polynucleotide
gccacacagaccuccuuguuaucggaaaguccuuucaugaauacaaccacggcuaauacccggcuuuacuagucacugucguacauuggccacucccagu

SEQ ID NO: 144 - RNA, 100 nt, Artificial Sequence
  Description of Artificial Sequence Synthetic polynucleotide
gccacacagaccucgacuaacuuugaaaguccuuucaugaauagcuccacggcuacuaguguccuuuacuagucacugucguacauuggccacucccagu

SEQ ID NO: 145 - RNA, 100 nt, Artificial Sequence
  Description of Artificial Sequence Synthetic polynucleotide
gccacacagaccuccuguuauguugaaaguaguuucaugaauacagccacgacuaaugaaugcgacgacuagucacugucguacauuggccacucccagu

SEQ ID NO: 146 - RNA, 100 nt, Artificial Sequence
  Description of Artificial Sequence Synthetic polynucleotide
gccacacagaccuccuguuaucuugaaagucguuucaugaauacaaccacggcuaaugaauuccuaguagagucacugucguacauuggccacucccagu

SEQ ID NO: 147 - RNA, 99 nt, Artificial Sequence
  Description of Artificial Sequence Synthetic oligonucleotide
gccacacaauaggacuuggccuuugaaaguccuuucuugaauacauccacggcaaugacgauacuuacuagucacugucguacauuggccacucccagu

SEQ ID NO: 148 - RNA, 98 nt, Artificial Sequence
  Description of Artificial Sequence Synthetic oligonucleotide
gccacacagaccauagccgccuuugaaaguacuuucaugacacauccacggaaaugaauuccucauucggucacugucguacauuggccacucccagu

SEQ ID NO: 149 - RNA, 100 nt, Artificial Sequence
  Description of Artificial Sequence Synthetic polynucleotide
gccacacagaaaggacuggccuuugaaaguccuuucauuaauacaaacacggcuacgacaguccuuuacuagucacugucguacauuggccacucccagu

SEQ ID NO: 150 - RNA, 97 nt, Artificial Sequence
  Description of Artificial Sequence Synthetic oligonucleotide
gccacacagaccggacaagccuuugaaaguaacuucauaucauccacggcuacguuucuccuuuacuagucacugucguacauuggccacucccagu

SEQ ID NO: 151 - RNA, 100 nt, Artificial Sequence
  Description of Artificial Sequence Synthetic polynucleotide
gccacacagaccuccuccagaguugaaaguccuuuuaugaauacauccacggcuaauuccuagcuuuacuagucacugucguacauuggccacucccagu

SEQ ID NO: 152 - RNA, 100 nt, Artificial Sequence
  Description of Artificial Sequence Synthetic polynucleotide
gccacacagaccucgaccgacuuugaaaguccuuucaugccuacauccacggcuaaucccgaacuuuacuagucacugucguacauuggccacucccagu

SEQ ID NO: 153 - RNA, 99 nt, Artificial Sequence
  Description of Artificial Sequence Synthetic oligonucleotide
gccacacagaccuccuuggcaggcacaagucgauucauguuacauccacggcuacgucuacgcuuuacuagucacugucguacauuggccacucccagu

SEQ ID NO: 154 - RNA, 100 nt, Artificial Sequence
  Description of Artificial Sequence Synthetic polynucleotide
gccacacagaccuccuaucuaguugaaagucgagccaugacuacuuccacggcuacugaauuccugccguggucacugucguacauuggccacucccagu

SEQ ID NO: 155 - RNA, 100 nt, Artificial Sequence
  Description of Artificial Sequence Synthetic polynucleotide
gccacacagaccucaccccacuuugaaaguccuuucauuaauacuuccacggcuaaugaauuccuuuuugugucacugucguacauuggccacucccagu

SEQ ID NO: 156 - RNA, 100 nt, Artificial Sequence
  Description of Artificial Sequence Synthetic polynucleotide
gccacacagaccucgaacgacuuugaaaguccuuucaugaauaguuccacggcuaccaauguccuuuacuagucacugucguacauuggccacucccagu

SEQ ID NO: 157 - RNA, 100 nt, Artificial Sequence
  Description of Artificial Sequence Synthetic polynucleotide
gccacacagaccucagcaagcuuugaaaguccuuucaugaauacauccacggccggcuaauucgagcuguagucacugucguacauuggccacucccagu

SEQ ID NO: 158 - RNA, 100 nt, Artificial Sequence
  Description of Artificial Sequence Synthetic polynucleotide
gccacacagaccuccuacaaaauugaaaguaaaaucaugaauaccaccacggcuuaugaauuccuguuucggucacugucguacauuggccacucccagu

SEQ ID NO: 159 - RNA, 101 nt, Artificial Sequence
  Description of Artificial Sequence Synthetic polynucleotide
gccacacagagggaaauggccuuugaaaguccuuucaugaauacauccacgugcuaccgaauucauucuguagucacugucguacauuggccacucccagu

SEQ ID NO: 160 - RNA, 100 nt, Artificial Sequence
  Description of Artificial Sequence Synthetic polynucleotide
gccacacagaaagaaauggccuuugaaaguccuuucaugaauagauccacggcuaaugaucgguauuacuagucacugucguacauuggccacucccagu

SEQ ID NO: 161 - RNA, 100 nt, Artificial Sequence
  Description of Artificial Sequence Synthetic polynucleotide
gccacacagaccuccuugaugguggaaaguccuuaccugaauacauccacggcuaaugaucugaguuacuagucacugucguacauuggccacucccagu

SEQ ID NO: 162 - RNA, 100 nt, Artificial Sequence
  Description of Artificial Sequence Synthetic polynucleotide
gccacacagaccuccuaaugaauugaaaguccuuucauguauacauccacgguuaaucaccgacuuuacuagucacugucguacauuggccacucccagu

SEQ ID NO: 163 - RNA, 100 nt, Artificial Sequence
  Description of Artificial Sequence Synthetic polynucleotide
gccacacagaccuccuuggcggugccaagucccgucaugaauacauccacggcuaaugaauucgggggauagucacugucguacauuggccacucccagu

SEQ ID NO: 164 - RNA, 99 nt, Artificial Sequence
  Description of Artificial Sequence Synthetic oligonucleotide
gccacacagaccucaauuaacuuugaaaguacuuucaugauuacauccacgauaaugaauucacaacguagucacugucguacauuggccacucccagu

SEQ ID NO: 165 - RNA, 101 nt, Artificial Sequence
  Description of Artificial Sequence Synthetic polynucleotide
gccacacagaccuccuaucagguugaaaguccauucaugaauauauccacgauaagcggaauuccuuuacuagucacugucguacauuggccacucccagu

SEQ ID NO: 166 - RNA, 100 nt, Artificial Sequence
  Description of Artificial Sequence Synthetic polynucleotide
gccacacagaccuccuguuauauugaaagugacgacaugaauacauccacggcuaaugaauuccuuuugaaaucacugucguacauuggccacucccagu

SEQ ID NO: 167 - RNA, 100 nt, Artificial Sequence
  Description of Artificial Sequence Synthetic polynucleotide
gccacacagaccuccuagaagauugaaaguccguucaugaauacauccacgguuaaugaauucgauggauagucacugucguacauuggccacucccagu

SEQ ID NO: 168 - RNA, 100 nt, Artificial Sequence
  Description of Artificial Sequence Synthetic polynucleotide
gccacacagaccuccuuguuagcagaaaguaucuucauaaauagauccacggcuaaugaauuccuuuacuagaaucugucguacauuggccacucccagu

SEQ ID NO: 169 - RNA, 100 nt, Artificial Sequence
  Description of Artificial Sequence Synthetic polynucleotide
gccacacagaccuccuacauucuugaaaguaguuucgugaauacauccacggcuauucaccuccuuuacuagucacugucguacauuggccacucccagu

SEQ ID NO: 170 - RNA, 98 nt, Artificial Sequence
  Description of Artificial Sequence Synthetic oligonucleotide
gccacacagaccuccuugauggcggaaaguccuuucauggcacauccacggcuaacaauuccuaccguggucacugucguacauuggccacucccagu

SEQ ID NO: 171 - RNA, 100 nt, Artificial Sequence
  Description of Artificial Sequence Synthetic polynucleotide
gccacacaaguaggcuuggccuuugaaaguccuuucccugaauaccccacggcuaauuuagagcuuuacuagucacugucguacauuggccacucccagu

SEQ ID NO: 172 - RNA, 100 nt, Artificial Sequence
  Description of Artificial Sequence Synthetic polynucleotide
gccacacagaccagagcugccuuugaaaguccuuuguugaauagauccacggcuaaugaauuccuuuugguaucacugucguacauuggccacucccagu

SEQ ID NO: 173 - RNA, 100 nt, Artificial Sequence
  Description of Artificial Sequence Synthetic polynucleotide
gccacauguauauccuuggccuuugaaaguccauucaugaauacauccacggcuacgucgguccuuuacuagucacugucguacauuggccacucccagu

SEQ ID NO: 174 - RNA, 99 nt, Artificial Sequence
  Description of Artificial Sequence Synthetic oligonucleotide
gccacacagaccggaguugccuuugaaaguccuuucaugaauacacacacggcaaugaauuccuuucucguucacugucguacauuggccacucccagu

SEQ ID NO: 175 - RNA, 100 nt, Artificial Sequence
  Description of Artificial Sequence Synthetic polynucleotide
gccacacagaccuccuauugacuugaaaguccauucaugaauacauccacgguuuaugaauggcaugacuagucacugucguacauuggccacucccagu

SEQ ID NO: 176 - RNA, 100 nt, Artificial Sequence
  Description of Artificial Sequence Synthetic polynucleotide
gccacacagaccuccuuguauguggaaaguccuuugcugaauacauccacguguaaucucuaacuuuacuagucacugucguacauuggccacucccagu

SEQ ID NO: 177 - RNA, 100 nt, Artificial Sequence
  Description of Artificial Sequence Synthetic polynucleotide
gccacacagaccuccuuggccuggacuuguccuuucacuaauacaugcacggcuaauacuauacuuuacuagucacugucguacauuggccacucccagu

SEQ ID NO: 178 - RNA, 100 nt, Artificial Sequence
  Description of Artificial Sequence Synthetic polynucleotide
gccacacagaauagaauggccuuugaaaguccuuucauuaauaccuccacagcuaacgauagagcuuacuagucacugucguacauuggccacucccagu

SEQ ID NO: 179 - RNA, 100 nt, Artificial Sequence
  Description of Artificial Sequence Synthetic polynucleotide
gccacacaggggcacuuggccuuugaaagucacuuucauaauacauacacggcucucgaauuccucucuuggucacugucguacauuggccacucccagu

SEQ ID NO: 180 - RNA, 100 nt, Artificial Sequence
  Description of Artificial Sequence Synthetic polynucleotide
gccacacagaccuccucaaugauugaaagucgguucaugaauagauccacggcuaaugaccgagauuacuagucacugucguacauuggccacucccagu

SEQ ID NO: 181 - RNA, 100 nt, Artificial Sequence
  Description of Artificial Sequence Synthetic polynucleotide
gccacacagaccaagcgcgccuuugaaaguccauucauuaauaacuccacggcuaauuucuuacuuuacuagucacugucguacauuggccacucccagu

SEQ ID NO: 182 - RNA, 100 nt, Artificial Sequence
  Description of Artificial Sequence Synthetic polynucleotide
gccacacagaccuccuaucagauugaaagugcuuucaugaauacauccacagcuaaugaauucgcagcguagucacugucguacauuggccacucccagu

SEQ ID NO: 183 - RNA, 98 nt, Artificial Sequence
  Description of Artificial Sequence Synthetic oligonucleotide
gccacacagaccucagccagcuuugaaaguacuuucaugaauacauccacggagugaauucgacgcauagucacugucguacauuggccacucccagu

SEQ ID NO: 184 - RNA, 100 nt, Artificial Sequence
  Description of Artificial Sequence Synthetic polynucleotide
gccacacagaccuccuaucauauugaaaguccuuucaugcauacacccacggcauguuacgagcuuuacuagucacugucguacauuggccacucccagu

SEQ ID NO: 185 - RNA, 101 nt, Artificial Sequence
  Description of Artificial Sequence Synthetic polynucleotide
gccacacagaccuccuuggcaaggucaaguccuuucaugaauaccuuccacggcucuagaauuccuacuggagucacugucguacauuggccacucccagu

SEQ ID NO: 186 - RNA, 99 nt, Artificial Sequence
  Description of Artificial Sequence Synthetic oligonucleotide
gccacacagaccaaggucgccuuugaaaguguuucaagaauaccuccacggcuacguagauccuuuacuagucacugucguacauuggccacucccagu

SEQ ID NO: 187 - RNA, 100 nt, Artificial Sequence
  Description of Artificial Sequence Synthetic polynucleotide
gccacacaaagacacuuggccuuugaaagugcuugcaugaauacauccacggccaaugaccgauguuacuagucacugucguacauuggccacucccagu

SEQ ID NO: 188 - RNA, 100 nt, Artificial Sequence
  Description of Artificial Sequence Synthetic polynucleotide
gccacacagaccuccuuggcaggcucaaguccuuucaugaauacccccacggcuaaugaauuccuuuacuuuugucugucguacauuggccacucccagu

SEQ ID NO: 189 - RNA, 100 nt, Artificial Sequence
  Description of Artificial Sequence Synthetic polynucleotide
gccacacagaccuccucaagaauugaaagucuuuucaugacgacauccacggcuaaugaauuccucgguaagucacugucguacauuggccacucccagu

SEQ ID NO: 190 - RNA, 100 nt, Artificial Sequence
  Description of Artificial Sequence Synthetic polynucleotide
gccacacagaaacagauggccuuugaaaguccuuucaucacuacauccacggcuaauugugcgcuuuacuagucacugucguacauuggccacucccagu

SEQ ID NO: 191 - RNA, 98 nt, Artificial Sequence
  Description of Artificial Sequence Synthetic oligonucleotide
gccacacagaccuccuuguaaaaggaaaguccuuucaugaauacauccacggguugaauuccuuuacuggggucugucguacauuggccacucccagu

SEQ ID NO: 192 - RNA, 99 nt, Artificial Sequence
  Description of Artificial Sequence Synthetic oligonucleotide
gccacacagaccuccuuggccugaacaugucuuucaugaauaccuccacggcuaaugaaugcagucacuagucacugucguacauuggccacucccagu

SEQ ID NO: 193 - RNA, 100 nt, Artificial Sequence
  Description of Artificial Sequence Synthetic polynucleotide
gccacacagaccuccuugaagacagaaaguucuuucaugaauacauccacggcuaauuacguacuuuacuagucacugucguacauuggccacucccagu

SEQ ID NO: 194 - RNA, 100 nt, Artificial Sequence
  Description of Artificial Sequence Synthetic polynucleotide
gccacacaagaagacuuggccuuugaaagucauuucaugcauacauucacggcuaaugaauuccuuuuuuguucacugucguacauuggccacucccagu

SEQ ID NO: 195 - RNA, 100 nt, Artificial Sequence
  Description of Artificial Sequence Synthetic polynucleotide
gccacacaagaagacuuggccuuugaaagucauuucaugcauacauucacggcuaaugaauuccuuuuuuguucacugucgucgaauggccacucccagu

SEQ ID NO: 196 - RNA, 100 nt, Artificial Sequence
  Description of Artificial Sequence Synthetic polynucleotide
gccacacagaccuccuaacagguugaaaguccuuucaucuuuacuuccacggcuaccguaguccuuuacuagucacugucguacauuggccacucccagu

SEQ ID NO: 197 - RNA, 100 nt, Artificial Sequence
  Description of Artificial Sequence Synthetic polynucleotide
gccacacagaccuccuuggcaauaauaaguccuuucaugaauacauccacggcuaauaaauaggacgacuagucacugucguacauuggccacucccagu

SEQ ID NO: 198 - RNA, 103 nt, Artificial Sequence
  Description of Artificial Sequence Synthetic polynucleotide
gccacacagagagauauggccuuugaaagucccuauuucaugauuacguccacggcuaauccaggacuuuacuagucacugucguacauuggccacucccagu

SEQ ID NO: 199 - RNA, 100 nt, Artificial Sequence
  Description of Artificial Sequence Synthetic polynucleotide
gccacacagaccucagccuacuuugaaaguccauucaugaauacauccacggcuuaugaauucacagcguagucacugucguacauuggccacucccagu

SEQ ID NO: 200 - RNA, 102 nt, Artificial Sequence
  Description of Artificial Sequence Synthetic polynucleotide
gccacacagaccuccuauuuacuugaaaguccugucaugaauacauccacggcgguaauccggggcuuuacuagucacugucguacauuggccacucccagu

SEQ ID NO: 201 - RNA, 100 nt, Artificial Sequence
  Description of Artificial Sequence Synthetic polynucleotide
gccacacagaccuccuccauucuugaaaguccuuuuaugaauagauccacggcaaaugaauuccuuuuagugucacugucguacauuggccacucccagu

SEQ ID NO: 202 - RNA, 101 nt, Artificial Sequence
  Description of Artificial Sequence Synthetic polynucleotide
gccacaugaaaguccuuggccuuugaaaguccuuugaugaauacauccacggcuacgcgacacagauuacuagucacugucguacauuggccacucccagu

SEQ ID NO: 203 - RNA, 99 nt, Artificial Sequence
  Description of Artificial Sequence Synthetic oligonucleotide
gccacacagaccuccuaacagauugaaaguuauucaugaauacauccacggcuaaugaauuccuuuugaaaucacugucguacauuggccacucccagu

SEQ ID NO: 204 - RNA, 97 nt, Artificial Sequence
  Description of Artificial Sequence Synthetic oligonucleotide
gccacacagaccuccucucaaguugaaaguccuuucaugaauauauccacggcuuaauuccuaaugaagucacugucguacauuggccacucccagu

SEQ ID NO: 205 - RNA, 100 nt, Artificial Sequence
  Description of Artificial Sequence Synthetic polynucleotide
gccacacagaccuccuugaauauggaaagucccuucaugaauauauccacggcuaaugaauucggcuuauagucacugucguacauuggccacucccagu

SEQ ID NO: 206 - RNA, 102 nt, Artificial Sequence
  Description of Artificial Sequence Synthetic polynucleotide
gccacaugguuauccuuggccuuugaaaguccuggugcuugaaucaauccacggcuacgacaguccuuuacuagucacugucguacauuggccacucccagu

SEQ ID NO: 207 - RNA, 97 nt, Artificial Sequence
  Description of Artificial Sequence Synthetic oligonucleotide
gccacacagaccuccuuggcauuacuaaguccuuucaugaucacauccacggcuaaaugcaucgacuagucacugucguacauuggccacucccagu

SEQ ID NO: 208 - RNA, 100 nt, Artificial Sequence
  Description of Artificial Sequence Synthetic polynucleotide
gccacacaaagggacuuggccuuugaaaguccuuacaugaauucauccacggcuaauucuuggcuuuacuagucacugucguacauuggccacucccagu

SEQ ID NO: 209 - RNA, 101 nt, Artificial Sequence
  Description of Artificial Sequence Synthetic polynucleotide
gccacacauauauccuuggccuuugaaaguccugucaugaguaggauccacggcuucauaauuccucagagagucacugucguacauuggccacucccagu

SEQ ID NO: 210 - RNA, 100 nt, Artificial Sequence
  Description of Artificial Sequence Synthetic polynucleotide
gccacacagaccuccucauaaguugaaaguccuuucaaaaauagccccacggcuauugaauucggcgcguagucacugucguacauuggccacucccagu

SEQ ID NO: 211 - RNA, 100 nt, Artificial Sequence
  Description of Artificial Sequence Synthetic polynucleotide
gccacacagaccuccuuggccuguucaugucuuuucaugaauacauccacggguaaugaucgggcuuacuagucacugucguacauuggccacucccagu

SEQ ID NO: 212 - RNA, 100 nt, Artificial Sequence
  Description of Artificial Sequence Synthetic polynucleotide
gccacaugguaguccuuggccuuugaaaguccuuucaugaauacauucacggcucaugaauuccuuuuuugaucacugucguacauuggccacucccagu

SEQ ID NO: 213 - RNA, 100 nt, Artificial Sequence
  Description of Artificial Sequence Synthetic polynucleotide
gccacacagaccuccugacagauugaaagugaguucaugauaccauccacggcuuucgaauuccugcaauagucacugucguacauuggccacucccagu

SEQ ID NO: 214 - RNA, 99 nt, Artificial Sequence
  Description of Artificial Sequence Synthetic oligonucleotide
gccacacagaccuccuguuuuauugaaaguccuugcauggauacuccacggcuacgauaguccuuuacuagucacugucguacauuggccacucccagu

SEQ ID NO: 215 - RNA, 100 nt, Artificial Sequence
  Description of Artificial Sequence Synthetic polynucleotide
gccacacaggagcacuuggccuuugaaaguccguucaugaauacauccacggcuauugaauucgacauguagucacugucguacauuggccacucccagu

SEQ ID NO: 216 - RNA, 99 nt, Artificial Sequence
  Description of Artificial Sequence Synthetic oligonucleotide
gccacacagaccagguaugccuuugaaaguccuuucaugaauacauccacgguuaaaaauucgguguguagucacugucguacauuggccacucccagu

SEQ ID NO: 217 - RNA, 100 nt, Artificial Sequence
  Description of Artificial Sequence Synthetic polynucleotide
gccacacagaccuccuccaagguugaaaguccuaucaugaauacauccacggcuaauuuacggcuuuacuagucacugucguacauuggccacucccagu

SEQ ID NO: 218 - RNA, 100 nt, Artificial Sequence
  Description of Artificial Sequence Synthetic polynucleotide
gccacacaagaaggcuuggccuuugaaaguccuuuguugaauacauccacggcuaaugaauucggcgcguagucacugucguacauuggccacucccagu

SEQ ID NO: 219 - RNA, 100 nt, Artificial Sequence
  Description of Artificial Sequence Synthetic polynucleotide
gccacacagaccuccuuguagacggaaagugguuucauucauagauccacggcuacgacgcuccuuuacuagucacugucguacauuggccacucccagu

SEQ ID NO: 220 - RNA, 100 nt, Artificial Sequence
  Description of Artificial Sequence Synthetic polynucleotide
gccacacaaauaaacuuggccuuugaaaguccuuucaugaauauauccacggcuaaugaaugcgaugacuagucacugucguacauuggccacucccagu

SEQ ID NO: 221 - RNA, 100 nt, Artificial Sequence
  Description of Artificial Sequence Synthetic polynucleotide
gccacacagaccuccuuguaaguagaaaguccuuucaugaauaccaccacggccaaugacacgacuuacuagucacugucguacauuggccacucccagu

SEQ ID NO: 222 - RNA, 100 nt, Artificial Sequence
  Description of Artificial Sequence Synthetic polynucleotide
gccacacagaccuccuagaaucuugaaaguccuuucaugaauauauccacggcuaauuaauucgagucauagucacugucguacauuggccacucccagu

SEQ ID NO: 223 - RNA, 100 nt, Artificial Sequence
  Description of Artificial Sequence Synthetic polynucleotide
gccacacagaccuccuuggcgggguuaaguccuuucaugaauacacccacggcuaauccuuggcuuuacuagucacugucguacauuggccacucccagu

SEQ ID NO: 224 - RNA, 100 nt, Artificial Sequence
  Description of Artificial Sequence Synthetic polynucleotide
gccacacagaccuccuguaauguugaaaguccuuucaugaauacaucuacggcuaacgaauucgggcuauagucacugucguacauuggccacucccagu

SEQ ID NO: 225 - RNA, 100 nt, Artificial Sequence
  Description of Artificial Sequence Synthetic polynucleotide
gccacacagaccuccucucagguugaaaguccuuucauggauacauccacggcuuccgaugcauauuacuagucacugucguacauuggccacucccagu

SEQ ID NO: 226 - RNA, 100 nt, Artificial Sequence
  Description of Artificial Sequence Synthetic polynucleotide
gccacacagaccuccugaaauguugaaaguccuuucaugaauacauccacggguaauuauacgcuuuacuagucacugucguacauuggccacucccagu

SEQ ID NO: 227 - RNA, 101 nt, Artificial Sequence
  Description of Artificial Sequence Synthetic polynucleotide
gccacacagaccuccuuguagaccgaaaguccuuucaugaauaucauccauagcuaauugcacacuuuacuagucacugucguacauuggccacucccagu

SEQ ID NO: 228 - RNA, 104 nt, Artificial Sequence
  Description of Artificial Sequence Synthetic polynucleotide
gccacacagaccuccucuauacuugaaaguccuuucaugaauagauccacgguuaccuugugaauuccuuuacuagucacugucguacauuggccacucccagu

SEQ ID NO: 229 - RNA, 100 nt, Artificial Sequence
  Description of Artificial Sequence Synthetic polynucleotide
gccacacagaccucgcaaugcuuugaaaguccuuucaugaauaaauccacggcuaaugaaugagacgacuagucacugucguacauuggccacucccagu

SEQ ID NO: 230 - RNA, 99 nt, Artificial Sequence
  Description of Artificial Sequence Synthetic oligonucleotide
gccacacagaccuccuaucaaguugaaaguccuuucagcauacauccacggcuaaugaauuccuuuuguuuucacugucguacauuggccacucccagu

SEQ ID NO: 231 - RNA, 100 nt, Artificial Sequence
  Description of Artificial Sequence Synthetic polynucleotide
gccacacagaccuccuuggcggagccaaguccuuucaugaauacaaccacggcgacggaccaggguuacuagucacugucguacauuggccacucccagu

SEQ ID NO: 232 - RNA, 100 nt, Artificial Sequence
  Description of Artificial Sequence Synthetic polynucleotide
gccacacagaccaaaagcgccuuugaaaguccuuucauggaucgauccacggcuucugaccaugauuacuagucacugucguacauuggccacucccagu

SEQ ID NO: 233 - RNA, 100 nt, Artificial Sequence
  Description of Artificial Sequence Synthetic polynucleotide
gccacacagaccuccugauuaauugaaaguccuuucaugaauacauccacggcuauauaauucaguauguagucacugucguacauuggccacucccagu

SEQ ID NO: 234 - RNA, 100 nt, Artificial Sequence
  Description of Artificial Sequence Synthetic polynucleotide
gccacacagaccuccucuuauauugaaaguccuuucaugaauauaucgacggcuacgacaauccuuuacuagucacugucguacauuggccacucccagu

SEQ ID NO: 235 - RNA, 100 nt, Artificial Sequence
  Description of Artificial Sequence Synthetic polynucleotide
gccacacaaggacacuuggccuuugaaaguccuuucaugaauacauucacgacuuuugaauucgaaguauagucacugucguacauuggccacucccagu

SEQ ID NO: 236 - RNA, 100 nt, Artificial Sequence
  Description of Artificial Sequence Synthetic polynucleotide
gccacacagaccuccuugaaucaagaaagucgguucaugaauacucccacggcuaauuagcgacuuuacuagucacugucguacauuggccacucccagu

SEQ ID NO: 237 - RNA, 100 nt, Artificial Sequence
  Description of Artificial Sequence Synthetic polynucleotide
gccacacagaccuccuguuauguugaaaguccuuucaugaauacaucgacggcuaaugaauuccuccuguggucacugucguacauuggccacucccagu

SEQ ID NO: 238 - RNA, 100 nt, Artificial Sequence
  Description of Artificial Sequence Synthetic polynucleotide
gccacacagaccuccuuguagggggaaaguccuuuccggcaugcauccacggcuagugaauuccugguauggucacugucguacauuggccacucccagu

SEQ ID NO: 239 - RNA, 101 nt, Artificial Sequence
  Description of Artificial Sequence Synthetic polynucleotide
gccacacagaccuccuuguaguaggaaagucguuuccaugaauacauccacggcuaaugaauuccuuuuucguucacugucguacauuggccacucccagu

SEQ ID NO: 240 - RNA, 100 nt, Artificial Sequence
  Description of Artificial Sequence Synthetic polynucleotide
gccacacagaccuccuugaaaauggaaaguccugucauuaauacauccacggcuaauuugggacuuuacuagucacugucguacauuggccacucccagu

SEQ ID NO: 241 - RNA, 99 nt, Artificial Sequence
  Description of Artificial Sequence Synthetic oligonucleotide
gccacacagaccuccucacgaauugaaaguccuucuugaauacauccacggcuacagaauuccuacaauggucacugucguacauuggccacucccagu

SEQ ID NO: 242 - RNA, 100 nt, Artificial Sequence
  Description of Artificial Sequence Synthetic polynucleotide
gccacacagaccuccuuggcgaugcuaaguagguucaugaauacguccacggcaauggaauucgggucauagucacugucguacauuggccacucccagu

SEQ ID NO: 243 - RNA, 100 nt, Artificial Sequence
  Description of Artificial Sequence Synthetic polynucleotide
gccacacaagggcgcuuggccuuugaaaguccuugcaugaauacacccacggcuacgacuguccuuuacuagucacugucguacauuggccacucccagu

SEQ ID NO: 244 - RNA, 100 nt, Artificial Sequence
  Description of Artificial Sequence Synthetic polynucleotide
gccacacaggaaagcuuggccuuugaaaguccuuucaugaauaccgccacggcuaauuugagacuuuacuagucacugucguacauuggccacucccagu

SEQ ID NO: 245 - RNA, 100 nt, Artificial Sequence
  Description of Artificial Sequence Synthetic polynucleotide
gccacacagaccuccucauuuguugaaaguccucucaugaauacauccacggcuaaucauuggcuuuacuagucacugucguacauuggccacucccagu

SEQ ID NO: 246 - RNA, 100 nt, Artificial Sequence
  Description of Artificial Sequence Synthetic polynucleotide
gccacacagaccucaagcagcuuugaaaguccuuucaugaauaccuccacggcuaauccaaggcuuuacuagucacugucguacauuggccacucccagu

SEQ ID NO: 247 - RNA, 100 nt, Artificial Sequence
  Description of Artificial Sequence Synthetic polynucleotide
gccacacagaccuccuuguaacacgaaaguccuuuuaugaauauauccacggcuauauaauuccuaaaauggucacugucguacauuggccacucccagu

SEQ ID NO: 248 - RNA, 100 nt, Artificial Sequence
  Description of Artificial Sequence Synthetic polynucleotide
gccacauggaaguccuuggccuuugaaaguccuuucaugaaucgauccacggcuaaucuacggcuuuacuagucacugucguacauuggccacucccagu

SEQ ID NO: 249 - RNA, 100 nt, Artificial Sequence
  Description of Artificial Sequence Synthetic polynucleotide
gccacacagaccgagcgugccuuugaaaguccuuucaugaauacaaccacggcuaaugaauucgacacguagucacugucguacauuggccacucccagu

SEQ ID NO: 250 - RNA, 100 nt, Artificial Sequence
  Description of Artificial Sequence Synthetic polynucleotide
gccacacagagauaaauggccuuugaaagugguuucaugaauacauccacggcuaaugaauucaaacgauagucacugucguacauuggccacucccagu

SEQ ID NO: 251 - RNA, 100 nt, Artificial Sequence
  Description of Artificial Sequence Synthetic polynucleotide
gccacacagaccuccuaauuuguugaaagucacuucaugaauaccuccacggcuaauuaauuccuaggauggucacugucguacauuggccacucccagu

SEQ ID NO: 252 - RNA, 100 nt, Artificial Sequence
  Description of Artificial Sequence Synthetic polynucleotide
gccacacagaccuccuugaaagcagaaaguccuuugaugaauacauccacggguaauaaaugugcgaacuagucacugucguacauuggccacucccagu

SEQ ID NO: 253 - RNA, 98 nt, Artificial Sequence
  Description of Artificial Sequence Synthetic oligonucleotide
gccacacagaccuccucucgaauugaaaguccuuucaugaauacgccacggcucggaauuccugauuuggucacugucguacauuggccacucccagu

SEQ ID NO: 254 - RNA, 99 nt, Artificial Sequence
  Description of Artificial Sequence Synthetic oligonucleotide
gccacacagaccuccuguuuacuugaaaguccucucaugaauacauccacgguaauucaaggcuuuacuagucacugucguacauuggccacucccagu

SEQ ID NO: 255 - RNA, 100 nt, Artificial Sequence
  Description of Artificial Sequence Synthetic polynucleotide
gccacacaaaaaggcuuggccuuugaaaguccugucauaaauacauccacggcuaauuaauucgggucauagucacugucguacauuggccacucccagu

SEQ ID NO: 256 - RNA, 100 nt, Artificial Sequence
  Description of Artificial Sequence Synthetic polynucleotide
gccacacagaccuccucauauguugaaaguccuuucuugaauaccauccacggcuaaaaauuccucucguggucacugucguacauuggccacucccagu

SEQ ID NO: 257 - RNA, 100 nt, Artificial Sequence
  Description of Artificial Sequence Synthetic polynucleotide
gccacacagaccuccuuggcggugcuaaguccuuucaucaauacauccacggcuaaugaauagacaaacuagucacugucguacauuggccacucccagu

SEQ ID NO: 258 - RNA, 100 nt, Artificial Sequence
  Description of Artificial Sequence Synthetic polynucleotide
gccacacagaccuccuuggcgaaaacaaguccuuucaugaauauuuccacggcuaaugaauucgccggauagucacugucguacauuggccacucccagu

SEQ ID NO: 259 - RNA, 100 nt, Artificial Sequence
  Description of Artificial Sequence Synthetic polynucleotide
gccacacagaccuccucgagaguugaaaguccucucaugaauagauccacggcuaaugaaugugggcacuagucacugucguacauuggccacucccagu

SEQ ID NO: 260 - RNA, 100 nt, Artificial Sequence
  Description of Artificial Sequence Synthetic polynucleotide
gccacacagagacaacuggccuuugaaaguccuuucauaaauacauccacgguuaaugaaugugggcacuagucacugucguacauuggccacucccagu

SEQ ID NO: 261 - RNA, 100 nt, Artificial Sequence
  Description of Artificial Sequence Synthetic polynucleotide
gccacacagaccuccuugaugacagaaaguccuuucaugaaugcagccacggcuaaugacgcuuguuacuagucacugucguacauuggccacucccagu

SEQ ID NO: 262 - RNA, 100 nt, Artificial Sequence
  Description of Artificial Sequence Synthetic polynucleotide
gccacauagggauccuuggccuuugaaaguccuuucaugaauacauccacggcgaaugaauuccuuuuguuaucacugucguacauuggccacucccagu

SEQ ID NO: 263 - RNA, 101 nt, Artificial Sequence
  Description of Artificial Sequence Synthetic polynucleotide
gccacacagaccuccuauugaauugaaaguccuuucaugaauaggauccacggauacugaauucgccccguagucacugucguacauuggccacucccagu

SEQ ID NO: 264 - RNA, 100 nt, Artificial Sequence
  Description of Artificial Sequence Synthetic polynucleotide
gccacacagaccucagccuacuuugaaaguccuuucaucaauaccaccacggcuaaugacaguacuuacuagucacugucguacauuggccacucccagu

SEQ ID NO: 265 - RNA, 102 nt, Artificial Sequence
  Description of Artificial Sequence Synthetic polynucleotide
gccacacagaccuccuuggcgacgauaaguccuuucaugaauacagccacggcuaucaugaauauaggcacuagucacugucguacauuggccacucccagu

SEQ ID NO: 266 - RNA, 100 nt, Artificial Sequence
  Description of Artificial Sequence Synthetic polynucleotide
gccacacagaguagcuuggccuuugaaaguccuuucaugaauagagccacggcuaauuucgcacuuuacuagucacugucguacauuggccacucccagu

SEQ ID NO: 267 - RNA, 100 nt, Artificial Sequence
  Description of Artificial Sequence Synthetic polynucleotide
gccacacagaccuccuuguagggggaaaguccuugaaugaauacauccacggcuaaugaauuccuuuacuguguucugucguacauuggccacucccagu

SEQ ID NO: 268 - RNA, 99 nt, Artificial Sequence
  Description of Artificial Sequence Synthetic oligonucleotide
gccacacagaccuccuuguauggggaaagucccuugaugacacauccacggcuaaugacgcggauuacuagucacugucguacauuggccacucccagu

SEQ ID NO: 269 - RNA, 101 nt, Artificial Sequence
  Description of Artificial Sequence Synthetic polynucleotide
gccacacagaccuccuacugacuugaaagucaguuucaugaauacauccacggcuaucuagcuccuuuacuagucacugucguacauuggccacucccagu

SEQ ID NO: 270 - RNA, 99 nt, Artificial Sequence
  Description of Artificial Sequence Synthetic oligonucleotide
gccacacagaccuccuuguugauggaaaguccuuucaugaauaccccacggcuaauacagggcuuuacuagucacugucguacauuggccacucccagu

SEQ ID NO: 271 - RNA, 100 nt, Artificial Sequence
  Description of Artificial Sequence Synthetic polynucleotide
gccacacagaccuccuuguugaaggaaaguccuuucaugaauacuuccacggcuaaugaauuccuuuuuuguucacugucguacauuggccacucccagu

SEQ ID NO: 272 - RNA, 100 nt, Artificial Sequence
  Description of Artificial Sequence Synthetic polynucleotide
gccacacagaccucgauccgcuuugaaaguccuuucauguauacauccacggcuaaugaauuccucauucggucacugucguacauuggccacucccagu

SEQ ID NO: 273 - RNA, 100 nt, Artificial Sequence
  Description of Artificial Sequence Synthetic polynucleotide
gccacacagaccuccuuguaggacgaaaguacuuucaugaauacauccauagaugcugaauuccuuuacuagaaucugucguacauuggccacucccagu

SEQ ID NO: 274 - RNA, 100 nt, Artificial Sequence
  Description of Artificial Sequence Synthetic polynucleotide
gccacacagaccucgcaauacuuugaaaguccuuucaugaauacagccacggcaaauuccuggcuuuacuagucacugucguacauuggccacucccagu

SEQ ID NO: 275 - RNA, 100 nt, Artificial Sequence
  Description of Artificial Sequence Synthetic polynucleotide
gccacauagggguccuuggccuuugaaagucuuuucaugaauucauccacggcuacgcuaguccuuuacuagucacugucguacauuggccacucccagu

SEQ ID NO: 276 - RNA, 100 nt, Artificial Sequence
  Description of Artificial Sequence Synthetic polynucleotide
gccacacagaccagguaugccuuugaaagucaauucaugaauacuuccacgacuauaaaacuccuuuacuagucacugucguacauuggccacucccagu

SEQ ID NO: 277 - RNA, 100 nt, Artificial Sequence
  Description of Artificial Sequence Synthetic polynucleotide
gccacacagaccuccuugauaggcgaaaguccuuucaugaauaacuccacggcuaucauacuccuuuacuagucacugucguacauuggccacucccagu

SEQ ID NO: 278 - RNA, 100 nt, Artificial Sequence
  Description of Artificial Sequence Synthetic polynucleotide
gccacacagaccuccuuguaugaggaaaguccuuuccagaauacauccacggcuaaugaauuccuuuuagugucacugucguacauuggccacucccagu

SEQ ID NO: 279 - RNA, 100 nt, Artificial Sequence
  Description of Artificial Sequence Synthetic polynucleotide
gccacacagaccuccugacuaauugaaaguccuuucaugaauacacgcacggcucgugacacggauuacuagucacugucguacauuggccacucccagu

SEQ ID NO: 280 - RNA, 100 nt, Artificial Sequence
  Description of Artificial Sequence Synthetic polynucleotide
gccacacagaccuccugauagauugaaaguccuuucaugccuacauccacggcuaauaugcggcuuuacuagucacugucguacauuggccacucccagu

SEQ ID NO: 281 - RNA, 101 nt, Artificial Sequence
  Description of Artificial Sequence Synthetic polynucleotide
gccacacagaccuccuuguaagcggaaagucccuucaugaauaagauccacggcuuuaaaauuccucauucggucacugucguacauuggccacucccagu

SEQ ID NO: 282 - RNA, 97 nt, Artificial Sequence
  Description of Artificial Sequence Synthetic oligonucleotide
gccacacagaccggguacgccuuugaaaguaucaugaauacauccacggcuaacgaauuccuuuuguuuucacugucguacauuggccacucccagu

SEQ ID NO: 283 - RNA, 100 nt, Artificial Sequence
  Description of Artificial Sequence Synthetic polynucleotide
gccacacagaccuccuaaugaguugaaaguucuuucaugaauacauccacgacuaaugaauuccuaucccggucacugucguacauuggccacucccagu

SEQ ID NO: 284 - RNA, 100 nt, Artificial Sequence
  Description of Artificial Sequence Synthetic polynucleotide
gccacacagacgaggaaccccuuugaaaguccuuucaugaauacauccacggcuaaacuucuccuuuacuagucacugucguacauuggccacucccagu

SEQ ID NO: 285 - RNA, 100 nt, Artificial Sequence
  Description of Artificial Sequence Synthetic polynucleotide
gccacacagaccuccuaacagguugaaaguccuuucaugaauacauccacggcuaauuugcuacuuuacuagucacugucguacauuggccacucccagu

SEQ ID NO: 286 - RNA, 100 nt, Artificial Sequence
  Description of Artificial Sequence Synthetic polynucleotide
gccacacagaccuccuaaaauguugaaagugcuuucaggaauacauccacggcuaaugaauuccuuuuuguuucacugucguacauuggccacucccagu

SEQ ID NO: 287 - RNA, 100 nt, Artificial Sequence
  Description of Artificial Sequence Synthetic polynucleotide
gccacacagaccgaacaugccuuugaaaguccuuugaugcauacauccacggcuaaugaauucgauauguagucacugucguacauuggccacucccagu

SEQ ID NO: 288 - RNA, 100 nt, Artificial Sequence
  Description of Artificial Sequence Synthetic polynucleotide
gccacacagaccauuagcgccuuugaaaguccuuugaugcauacauccacggcuauuuagguccuuuacuagucacugucguacauuggccacucccagu

SEQ ID NO: 289 - RNA, 100 nt, Artificial Sequence
  Description of Artificial Sequence Synthetic polynucleotide
gccacacaaaaaaacuuggccuuugaaaguccuuugaugcauacauccacggcuacaaguguccuuuacuagucacugucguacauuggccacucccagu

SEQ ID NO: 290 - RNA, 100 nt, Artificial Sequence
  Description of Artificial Sequence Synthetic polynucleotide
gccacacagaccuccugauaacuugaaaguccuuugaugcauacauccacggcuaaucuggggcuuuacuagucacugucguacauuggccacucccagu

SEQ ID NO: 291 - RNA, 100 nt, Artificial Sequence
  Description of Artificial Sequence Synthetic polynucleotide
gccacacaguauuacuuggccuuugaaaguccuuugaugcauacauccacggcuaauuagcagcuuuacuagucacugucguacauuggccacucccagu

SEQ ID NO: 292 - RNA, 100 nt, Artificial Sequence
  Description of Artificial Sequence Synthetic polynucleotide
gccacauacaaguccuuggccuuugaaaguccuuugaugcauacauccacggcuaauuuaguacuuuacuagucacugucguacauuggccacucccagu

SEQ ID NO: 293 - RNA, 100 nt, Artificial Sequence
  Description of Artificial Sequence Synthetic polynucleotide
gccacaacucccucgaacgacuuugaaaguccuuucaugaauagauccacggcuaauuauaagcuuuacaccacacugucgucgaauggccacucccagu

SEQ ID NO: 294 - RNA, 100 nt, Artificial Sequence
  Description of Artificial Sequence Synthetic polynucleotide
gccacaacucccucgaacgacuuugaaaguccuuucaugaauagauccacggcuaauuauaagcuuuacuagucacugucguacauuggccacucccagu

SEQ ID NO: 295 - RNA, 100 nt, Artificial Sequence
  Description of Artificial Sequence Synthetic polynucleotide
gccacaacucccuccuuggcaaguccaaguccuuucaugaauagauccacggcuaugccuguccuuuacaccacacugucgucgaauggccacucccagu

SEQ ID NO: 296 - RNA, 100 nt, Artificial Sequence
  Description of Artificial Sequence Synthetic polynucleotide
gccacacagaccuccuuggcaaguccaaguccuuucaugaauagauccacggcuaugccuguccuuuacuagucacugucguacauuggccacucccagu

SEQ ID NO: 297 - RNA, 100 nt, Artificial Sequence
  Description of Artificial Sequence Synthetic polynucleotide
gccacacagaccuccuuggcaaguccaaguccuuucaugaauagauccacggcuaugccuguccuuuacaccacacugucgucgaauggccacucccagu

SEQ ID NO: 298 - RNA, 100 nt, Artificial Sequence
  Description of Artificial Sequence Synthetic polynucleotide
gccacaacucccuccuuggcaaguccaaguccuuucaugaauagauccacggcuaugccuguccuuuacuagucacugucguacauuggccacucccagu

SEQ ID NO: 299 - RNA, 99 nt, Artificial Sequence
  Description of Artificial Sequence Synthetic oligonucleotide
gccacaacucccuccuugaaagcagaaaguccuuucgugaauacauccacggcacugaaugguuugacaccacacugucgucgaauggccacucccagu

SEQ ID NO: 300 - RNA, 99 nt, Artificial Sequence
  Description of Artificial Sequence Synthetic oligonucleotide
gccacacagaccuccuugaaagcagaaaguccuuucgugaauacauccacggcacugaaugguuugacuagucacugucguacauuggccacucccagu

SEQ ID NO: 301 - RNA, 99 nt, Artificial Sequence
  Description of Artificial Sequence Synthetic oligonucleotide
gccacacagaccuccuugaaagcagaaaguccuuucgugaauacauccacggcacugaaugguuugacaccacacugucgucgaauggccacucccagu

SEQ ID NO: 302 - RNA, 99 nt, Artificial Sequence
  Description of Artificial Sequence Synthetic oligonucleotide
gccacaacucccuccuugaaagcagaaaguccuuucgugaauacauccacggcacugaaugguuugacuagucacugucguacauuggccacucccagu

SEQ ID NO: 303 - RNA, 100 nt, Artificial Sequence
  Description of Artificial Sequence Synthetic polynucleotide
gccacaacucccucgcgcaacuuugaaaguccuuucaugcauagauccacggcuacauaauuccuuuuggagucacugucgucgaauggccacucccagu

SEQ ID NO: 304 - RNA, 100 nt, Artificial Sequence
  Description of Artificial Sequence Synthetic polynucleotide
gccacacagaccucgcgcaacuuugaaaguccuuucaugcauagauccacggcuacauaauuccuuuuggagucacugucgucgaauggccacucccagu

SEQ ID NO: 305 - RNA, 100 nt, Artificial Sequence
  Description of Artificial Sequence Synthetic polynucleotide
gccacaacucccucgcgcaacuuugaaaguccuuucaugcauagauccacggcuacauaauuccuuuuggagucacugucguacauuggccacucccagu

SEQ ID NO: 306 - RNA, 100 nt, Artificial Sequence
  Description of Artificial Sequence Synthetic polynucleotide
gccacaacucccucagccuacuuugaaaguccuuucaucaauaccaccacggcuaaugacaguacuuacaccacacugucgucgaauggccacucccagu

SEQ ID NO: 307 - RNA, 100 nt, Artificial Sequence
  Description of Artificial Sequence Synthetic polynucleotide
gccacacagaccucagccuacuuugaaaguccuuucaucaauaccaccacggcuaaugacaguacuuacaccacacugucgucgaauggccacucccagu

SEQ ID NO: 308 - RNA, 100 nt, Artificial Sequence
  Description of Artificial Sequence Synthetic polynucleotide
gccacaacucccucagccuacuuugaaaguccuuucaucaauaccaccacggcuaaugacaguacuuacuagucacugucguacauuggccacucccagu

SEQ ID NO: 309 - RNA, 100 nt, Artificial Sequence
  Description of Artificial Sequence Synthetic polynucleotide
gccacaacucccucggacaacuuugaaaguccuuucaugaauacaaccacggcucaacaauucgaaccgaccacacugucgucgaauggccacucccagu

SEQ ID NO: 310 - RNA, 100 nt, Artificial Sequence
  Description of Artificial Sequence Synthetic polynucleotide
gccacacagaccucggacaacuuugaaaguccuuucaugaauacaaccacggcucaacaauucgaaccgaccacacugucgucgaauggccacucccagu

SEQ ID NO: 311 - RNA, 100 nt, Artificial Sequence
  Description of Artificial Sequence Synthetic polynucleotide
gccacaacucccucggacaacuuugaaaguccuuucaugaauacaaccacggcucaacaauucgaaccguagucacugucguacauuggccacucccagu

SEQ ID NO: 312 - RNA, 100 nt, Artificial Sequence
  Description of Artificial Sequence Synthetic polynucleotide
gccacaacucccuccuugauggcggaaaguccuuucaugaauacauccacggcuaaagaauucgauccaaccacacugucgucgaauggccacucccagu

SEQ ID NO: 313 - RNA, 100 nt, Artificial Sequence
  Description of Artificial Sequence Synthetic polynucleotide
gccacacagaccuccuugauggcggaaaguccuuucaugaauacauccacggcuaaagaauucgauccaaccacacugucgucgaauggccacucccagu

SEQ ID NO: 314 - RNA, 100 nt, Artificial Sequence
  Description of Artificial Sequence Synthetic polynucleotide
gccacaacucccuccuugauggcggaaaguccuuucaugaauacauccacggcuaaagaauucgauccauagucacugucguacauuggccacucccagu

SEQ ID NO: 315 - RNA, 100 nt, Artificial Sequence
  Description of Artificial Sequence Synthetic polynucleotide
gccacaacucccucgacuaacuuugaaaguccuuucaugaauagcuccacggcuacuaguguccuuuacaccacacugucgucgaauggccacucccagu

SEQ ID NO: 316 - RNA, 100 nt, Artificial Sequence
  Description of Artificial Sequence Synthetic polynucleotide
gccacacagaccucgacuaacuuugaaaguccuuucaugaauagcuccacggcuacuaguguccuuuacaccacacugucgucgaauggccacucccagu

SEQ ID NO: 317 - RNA, 100 nt, Artificial Sequence
  Description of Artificial Sequence Synthetic polynucleotide
gccacaacucccucgacuaacuuugaaaguccuuucaugaauagcuccacggcuacuaguguccuuuacuagucacugucguacauuggccacucccagu

SEQ ID NO: 318 - RNA, 100 nt, Artificial Sequence
  Description of Artificial Sequence Synthetic polynucleotide
gccacaacucccuccuauagaguugaaaguccuuucaugaauacaucuacggcuaaucgccgacuuuacaccacacugucgucgaauggccacucccagu

SEQ ID NO: 319 - RNA, 97 nt, Artificial Sequence
  Description of Artificial Sequence Synthetic oligonucleotide
gccacaacucccuccucucaaguugaaaguccuuucaugaauauauccacggcuuaauuccuaaugaacacacugucgucgaauggccacucccagu

SEQ ID NO: 320 - RNA, 100 nt, Artificial Sequence
  Description of Artificial Sequence Synthetic polynucleotide
gccacaacucccucgcaucacuuugaaaguacuuucauuaauacauccacggcuacgucgcuccuuuacaccacacugucgucgaauggccacucccagu

SEQ ID NO: 321 - RNA, 99 nt, Artificial Sequence
  Description of Artificial Sequence Synthetic oligonucleotide
gccacaacucccucaaaacacuuugaaaguccuuucaugaaugucuccacggcaaugaauuccugagaugcacacugucgucgaauggccacucccagu

SEQ ID NO: 322 - RNA, 99 nt, Artificial Sequence
  Description of Artificial Sequence Synthetic oligonucleotide
gccacacagaccucaaaacacuuugaaaguccuuucaugaaugucuccacggcaaugaauuccugagauggucacugucguacauuggccacucccagu

SEQ ID NO: 323 - RNA, 99 nt, Artificial Sequence
  Description of Artificial Sequence Synthetic oligonucleotide
gccacaacucccuccuugauaaucgaaaguccuuucaugaauacauccacgauaaugaauaguaaaacaccacacugucgucgaauggccacucccagu

SEQ ID NO: 324 - RNA, 99 nt, Artificial Sequence
  Description of Artificial Sequence Synthetic oligonucleotide
gccacacagaccuccuugauaaucgaaaguccuuucaugaauacauccacgauaaugaauaguaaaacuagucacugucguacauuggccacucccagu

SEQ ID NO: 325 - RNA, 98 nt, Artificial Sequence
  Description of Artificial Sequence Synthetic oligonucleotide
gccacaacucccucacacugcuuugaaaguccuuucaggaauaccuccacggcuagaauuccuuuacaccacacugucgucgaauggccacucccagu

SEQ ID NO: 326 - RNA, 98 nt, Artificial Sequence
  Description of Artificial Sequence Synthetic oligonucleotide
gccacacagaccucacacugcuuugaaaguccuuucaggaauaccuccacggcuagaauuccuuuacuagucacugucguacauuggccacucccagu

SEQ ID NO: 327 - RNA, 100 nt, Artificial Sequence
  Description of Artificial Sequence Synthetic polynucleotide
gccacaacucccuccuugauaguggaaaguccuuucaugauuacauccacggcuaguaaaacacuuuacaccacacugucgucgaauggccacucccagu

SEQ ID NO: 328 - RNA, 100 nt, Artificial Sequence
  Description of Artificial Sequence Synthetic polynucleotide
gccacacagaccuccuugauaguggaaaguccuuucaugauuacauccacggcuaguaaaacacuuuacuagucacugucguacauuggccacucccagu

SEQ ID NO: 329 - RNA, 100 nt, Artificial Sequence
  Description of Artificial Sequence Synthetic polynucleotide
gccacaacucccuccuaguagguugaaaguccuuucauuaauaacuccacggcuaaugaucugaguuacaccacacugucgucgaauggccacucccagu

SEQ ID NO: 330 - RNA, 100 nt, Artificial Sequence
  Description of Artificial Sequence Synthetic polynucleotide
gccacaacucccuccuuggcaacgcuaaguccuuucaugaauacauccacggcucaugaauuccuuuugaagucacugucgucgaauggccacucccagu

SEQ ID NO: 331 - RNA, 100 nt, Artificial Sequence
  Description of Artificial Sequence Synthetic polynucleotide
gccacacagaccuccuuggcaacgcuaaguccuuucaugaauacauccacggcucaugaauuccuuuugaagucacugucguacauuggccacucccagu

SEQ ID NO: 332 - RNA, 100 nt, Artificial Sequence
  Description of Artificial Sequence Synthetic polynucleotide
gccacaacucccuccuuggcaacgcuaaguccuuucaugaauacauccacggcucaugaauucgaccugaccacacugucgucgaauggccacucccagu

SEQ ID NO: 333 - RNA, 100 nt, Artificial Sequence
  Description of Artificial Sequence Synthetic polynucleotide
gccacacagaccuccuuggcaacgcuaaguccuuucaugaauacauccacggcucaugaauucgaccuguagucacugucguacauuggccacucccagu

SEQ ID NO: 334 - RNA, 98 nt, Artificial Sequence
  Description of Artificial Sequence Synthetic oligonucleotide
gccacaacucccuccuacauuauugaaaguccuuucaugaauccauccacggcaugaauuccuauuuuacacacugucgucgaauggccacucccagu

SEQ ID NO: 335 - RNA, 98 nt, Artificial Sequence
  Description of Artificial Sequence Synthetic oligonucleotide
gccacacagaccuccuacauuauugaaaguccuuucaugaauccauccacggcaugaauuccuauuuuagucacugucguacauuggccacucccagu

SEQ ID NO: 336 - RNA, 100 nt, Artificial Sequence
  Description of Artificial Sequence Synthetic polynucleotide
gccacaacucccgaagccgccuuugaaaguccuuucaugaauagauccacggcuaccgcccuccuuuacaccacacugucgucgaauggccacucccagu

SEQ ID NO: 337 - RNA, 100 nt, Artificial Sequence
  Description of Artificial Sequence Synthetic polynucleotide
gccacacagaccgaagccgccuuugaaaguccuuucaugaauagauccacggcuaccgcccuccuuuacuagucacugucguacauuggccacucccagu

SEQ ID NO: 338 - RNA, 98 nt, Artificial Sequence
  Description of Artificial Sequence Synthetic oligonucleotide
gccacaacucccucgacacgcuuugaaaguccuuucaugaauacauccacggcuugaauucggaaugaccacacugucgucgaauggccacucccagu

SEQ ID NO: 339 - RNA, 98 nt, Artificial Sequence
  Description of Artificial Sequence Synthetic oligonucleotide
gccacacagaccucgacacgcuuugaaaguccuuucaugaauacauccacggcuugaauucggaauguagucacugucguacauuggccacucccagu

SEQ ID NO: 340 - RNA, 100 nt, Artificial Sequence
  Description of Artificial Sequence Synthetic polynucleotide
gccacaacucccuccuuggcgggguuaaguccuuucaugaauacacccacggcuaauccuuggcuuuacaccacacugucgucgaauggccacucccagu

SEQ ID NO: 341 - RNA, 101 nt, Artificial Sequence
  Description of Artificial Sequence Synthetic polynucleotide
gccacaacucccuccuuguaggaggaaaguccuuucauuaauacauccacggcuaauggaauuccuccucuacacacugucgucgaauggccacucccagu

SEQ ID NO: 342 - RNA, 101 nt, Artificial Sequence
  Description of Artificial Sequence Synthetic polynucleotide
gccacacagaccuccuuguaggaggaaaguccuuucauuaauacauccacggcuaauggaauuccuccucuagucacugucguacauuggccacucccagu

SEQ ID NO: 343 - RNA, 100 nt, Artificial Sequence
  Description of Artificial Sequence Synthetic polynucleotide
gccacaacucccuccuuguaacgcgaaaguccuuucaugaauacauccacggcucaugacgaagauuacaccacacugucgucgaauggccacucccagu

SEQ ID NO: 344 - RNA, 100 nt, Artificial Sequence
  Description of Artificial Sequence Synthetic polynucleotide
gccacacagaccuccuuguaacgcgaaaguccuuucaugaauacauccacggcucaugacgaagauuacuagucacugucguacauuggccacucccagu

SEQ ID NO: 345 - RNA, 100 nt, Artificial Sequence
  Description of Artificial Sequence Synthetic polynucleotide
gccacaacucccuccuuggcgccaguaaguccuuucaugaaugaauccacggcuacgacaggacuuuacaccacacugucgucgaauggccacucccagu

SEQ ID NO: 346 - RNA, 100 nt, Artificial Sequence
  Description of Artificial Sequence Synthetic polynucleotide
gccacacagaccuccuuggcgccaguaaguccuuucaugaaugaauccacggcuacgacaggacuuuacuagucacugucguacauuggccacucccagu

SEQ ID NO: 347 - RNA, 100 nt, Artificial Sequence
  Description of Artificial Sequence Synthetic polynucleotide
gccacaacucccuccuaucaacuugaaaguccuuucaugcauacauccacggcuaaugacgcagauuacaccacacugucgucgaauggccacucccagu

SEQ ID NO: 348 - RNA, 100 nt, Artificial Sequence
  Description of Artificial Sequence Synthetic polynucleotide
gccacacagaccuccuaucaacuugaaaguccuuucaugcauacauccacggcuaaugacgcagauuacuagucacugucguacauuggccacucccagu

SEQ ID NO: 349 - RNA, 98 nt, Artificial Sequence
  Description of Artificial Sequence Synthetic oligonucleotide
gccacaacucccucagccagcuuugaaaguacuuucaugaauacauccacggagugaauucgacgcaaccacacugucgucgaauggccacucccagu

SEQ ID NO: 350 - RNA, 100 nt, Artificial Sequence
  Description of Artificial Sequence Synthetic polynucleotide
gccacaacucccaggaucgccuuugaaaguccuucccugaauacauccacggcuacgacaguccuuuacaccacacugucgucgaauggccacucccagu

SEQ ID NO: 351 - RNA, 100 nt, Artificial Sequence
  Description of Artificial Sequence Synthetic polynucleotide
gccacaacucccuccucuuuacuugaaaguccuuucaugaauagauccacggcuaaugaauuccuuuacaccacacugucgucgaauggccacucccagu

SEQ ID NO: 352 - RNA, 100 nt, Artificial Sequence
  Description of Artificial Sequence Synthetic polynucleotide
gccacacagaccuccucuuuacuugaaaguccuuucaugaauagauccacggcuaaugaauuccuuuacuagucacugucguacauuggccacucccagu

SEQ ID NO: 353 - RNA, 100 nt, Artificial Sequence
  Description of Artificial Sequence Synthetic polynucleotide
gccacaacucccuccucucgaguugaaaguccuuucaugaauacauccacggcuacggauagagcuuacaccacacugucgucgaauggccacucccagu

SEQ ID NO: 354 - RNA, 100 nt, Artificial Sequence
  Description of Artificial Sequence Synthetic polynucleotide
gccacacagaccuccucucgaguugaaaguccuuucaugaauacauccacggcuacggauagagcuuacuagucacugucguacauuggccacucccagu

SEQ ID NO: 355 - RNA, 100 nt, Artificial Sequence
  Description of Artificial Sequence Synthetic polynucleotide
gccacaacucccuccucucaucuugaaagucgacuuucaugaauacauccacggaaugaauuccuuuuguaaucacugucgucgaauggccacucccagu

SEQ ID NO: 356 - RNA, 100 nt, Artificial Sequence
  Description of Artificial Sequence Synthetic polynucleotide
gccacaacucccuccuugauaggcgaaaguccuuucaugaauaacuccacggcuaucauacuccuuuacaccacacugucgucgaauggccacucccagu

SEQ ID NO: 357 - RNA, 99 nt, Artificial Sequence
  Description of Artificial Sequence Synthetic oligonucleotide
gccacaacucccuccucuaaaguugaaaguccuuucaugaauacauccacgauaauaacuggcuuuacaccacacugucgucgaauggccacucccagu

SEQ ID NO: 358 - RNA, 99 nt, Artificial Sequence
  Description of Artificial Sequence Synthetic oligonucleotide
gccacacagaccuccucuaaaguugaaaguccuuucaugaauacauccacgauaauaacuggcuuuacuagucacugucguacauuggccacucccagu

SEQ ID NO: 359 - RNA, 99 nt, Artificial Sequence
  Description of Artificial Sequence Synthetic oligonucleotide
gccacaacucccuccugauauguugaaaguccuuuccugaauacauccacggcuacgacaagaauuacaccacacugucgucgaauggccacucccagu

SEQ ID NO: 360 - RNA, 99 nt, Artificial Sequence
  Description of Artificial Sequence Synthetic oligonucleotide
gccacacagaccuccugauauguugaaaguccuuuccugaauacauccacggcuacgacaagaauuacuagucacugucguacauuggccacucccagu

SEQ ID NO: 361 - RNA, 101 nt, Artificial Sequence
  Description of Artificial Sequence Synthetic polynucleotide
gccacaacucccuccuacaaacuugaaaguccuuucaugaauacaaccacggcuaauugacggagauuacaccacacugucgucgaauggccacucccagu

SEQ ID NO: 362 - RNA, 101 nt, Artificial Sequence
  Description of Artificial Sequence Synthetic polynucleotide
gccacacagaccuccuacaaacuugaaaguccuuucaugaauacaaccacggcuaauugacggagauuacuagucacugucguacauuggccacucccagu

SEQ ID NO: 363 - RNA, 100 nt, Artificial Sequence
  Description of Artificial Sequence Synthetic polynucleotide
gccacaacucccuccuaaaaaguugaaaguccuuucaugaauacauccacggguaaugaauuccuuuacaccacacugucgucgaauggccacucccagu

SEQ ID NO: 364 - RNA, 100 nt, Artificial Sequence
  Description of Artificial Sequence Synthetic polynucleotide
gccacacagaccuccuaaaaaguugaaaguccuuucaugaauacauccacggguaaugaauuccuuuacuagucacugucguacauuggccacucccagu

SEQ ID NO: 365 - RNA, 98 nt, Artificial Sequence
  Description of Artificial Sequence Synthetic oligonucleotide
gccacaacucccuccugaaugauugaaaguccuuucaugaauacauccacggcaugaucugaguuacaccacacugucgucgaauggccacucccagu

SEQ ID NO: 366 - RNA, 99 nt, Artificial Sequence
  Description of Artificial Sequence Synthetic oligonucleotide
gccacaacucccuccucaugaguugaaaguccuuucaugaauacauccacggacuugaauuccuggaaugcacacugucgucgaauggccacucccagu

SEQ ID NO: 367 - RNA, 100 nt, Artificial Sequence
  Description of Artificial Sequence Synthetic polynucleotide
gccacaacucccuccuuguaaggagaaaguccuuucaugaauacauccacggcggcugaauucgcgcugaccacacugucgucgaauggccacucccagu

SEQ ID NO: 368 - RNA, 100 nt, Artificial Sequence
  Description of Artificial Sequence Synthetic polynucleotide
gccacacagaccuccuuguaaggagaaaguccuuucaugaauacauccacggcggcugaauucgcgcuguagucacugucguacauuggccacucccagu

SEQ ID NO: 369 - RNA, 99 nt, Artificial Sequence
  Description of Artificial Sequence Synthetic oligonucleotide
gccacaacucccuccucauugauugaaaguccuuucaugaauaccccacggcggcgauauuccuuuacaccacacugucgucgaauggccacucccagu

SEQ ID NO: 370 - RNA, 99 nt, Artificial Sequence
  Description of Artificial Sequence Synthetic oligonucleotide
gccacacagaccuccucauugauugaaaguccuuucaugaauaccccacggcggcgauauuccuuuacuagucacugucguacauuggccacucccagu

SEQ ID NO: 371 - RNA, 100 nt, Artificial Sequence
  Description of Artificial Sequence Synthetic polynucleotide
gccacaacucccuccuugauaaucgaaaguccuuucaugaauacauccacggcucuugacgauaguuacaccacacugucgucgaauggccacucccagu

SEQ ID NO: 372 - RNA, 100 nt, Artificial Sequence
  Description of Artificial Sequence Synthetic polynucleotide
gccacacagaccuccuugauaaucgaaaguccuuucaugaauacauccacggcucuugacgauaguuacuagucacugucguacauuggccacucccagu

SEQ ID NO: 373 - RNA, 101 nt, Artificial Sequence
  Description of Artificial Sequence Synthetic polynucleotide
gccacaacucccuccuugauggcggaaagucccuucaugaauacuauccacggcucuacaauuccuuuacuggagucugucgucgaauggccacucccagu

SEQ ID NO: 374 - RNA, 100 nt, Artificial Sequence
  Description of Artificial Sequence Synthetic polynucleotide
gccacaacucccuccuccaagauugaaaguccuuucaagaauacauccacggcgguuacauuccuuuacaccacacugucgucgaauggccacucccagu

SEQ ID NO: 375 - RNA, 100 nt, Artificial Sequence
  Description of Artificial Sequence Synthetic polynucleotide
gccacacagaccuccuccaagauugaaaguccuuucaagaauacauccacggcgguuacauuccuuuacuagucacugucguacauuggccacucccagu

SEQ ID NO: 376 - RNA, 100 nt, Artificial Sequence
  Description of Artificial Sequence Synthetic polynucleotide
gccacaacucccuccugacauauugaaaguccuuucaucaauacauccacggcgugcauauuccuuuacaccacacugucgucgaauggccacucccagu

SEQ ID NO: 377 - RNA, 100 nt, Artificial Sequence
  Description of Artificial Sequence Synthetic polynucleotide
gccacacagaccuccugacauauugaaaguccuuucaucaauacauccacggcgugcauauuccuuuacuagucacugucguacauuggccacucccagu

SEQ ID NO: 378 - RNA, 100 nt, Artificial Sequence
  Description of Artificial Sequence Synthetic polynucleotide
gccacaacucccuccugauauguugaaaguccuuuccugaauacauucacggcuauucaucuccuuuacaccacacugucgucgaauggccacucccagu

SEQ ID NO: 379 - RNA, 100 nt, Artificial Sequence
  Description of Artificial Sequence Synthetic polynucleotide
gccacacagaccuccugauauguugaaaguccuuuccugaauacauucacggcuauucaucuccuuuacuagucacugucguacauuggccacucccagu

SEQ ID NO: 380 - RNA, 100 nt, Artificial Sequence
  Description of Artificial Sequence Synthetic polynucleotide
gccacaacucccuccucuauuauugaaaguccuuucauuaauacauccacggcuaauuguuggcuuuacaccacacugucgucgaauggccacucccagu

SEQ ID NO: 381 - RNA, 100 nt, Artificial Sequence
  Description of Artificial Sequence Synthetic polynucleotide
gccacacagaccuccucuauuauugaaaguccuuucauuaauacauccacggcuaauuguuggcuuuacuagucacugucguacauuggccacucccagu

SEQ ID NO: 382 - RNA, 100 nt, Artificial Sequence
  Description of Artificial Sequence Synthetic polynucleotide
gccacaacucccuccuaacaucuugaaaguccuuucaugaauacauccacggcuuuggaauucgguccgaccacacugucgucgaauggccacucccagu

SEQ ID NO: 383 - RNA, 100 nt, Artificial Sequence
  Description of Artificial Sequence Synthetic polynucleotide
gccacacagaccuccuaacaucuugaaaguccuuucaugaauacauccacggcuuuggaauucgguccguagucacugucguacauuggccacucccagu

SEQ ID NO: 384 - RNA, 100 nt, Artificial Sequence
  Description of Artificial Sequence Synthetic polynucleotide
gccacaacucccuccucucagguugaaaguccuuucauggauacauccacggcuuccgaugcauauuacaccacacugucgucgaauggccacucccagu

SEQ ID NO: 385 - DNA, 32 bp, Homo sapiens
taggctttctggctttttaccggaaagcccct

SEQ ID NO: 386 - RNA, 32 nt, Homo sapiens
uaggcuuucuggcuuuuuaccggaaagccccu

SEQ ID NO: 387 - DNA, 31 bp, Mus sp.
caggttttctgacttcggtcggaaaacccct

SEQ ID NO: 388 - DNA, 11 bp, Artificial Sequence
  Description of Artificial Sequence Synthetic oligonucleotide
aatttttggag

SEQ ID NO: 389 - RNA, 11 nt, Artificial Sequence
  Description of Artificial Sequence Synthetic oligonucleotide
aauuuuuggag

SEQ ID NO: 390 - DNA, 249 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 391 - DNA, 341 bp, Artificial Sequence
  Description of Artificial Sequence Synthetic polynucleotide
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 392 - DNA, 263 bp, Artificial Sequence
  Description of Artificial Sequence Synthetic polynucleotide
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 393 - DNA, 257 bp, Artificial Sequence
  Description of Artificial Sequence Synthetic polynucleotide
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 394 - DNA, 393 bp, Artificial Sequence
  Description of Artificial Sequence Synthetic polynucleotide
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 395 - DNA, 698 bp, Artificial Sequence
  Description of Artificial Sequence Synthetic polynucleotide
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 396 - RNA, 31 nt, Mus sp.
cagguuuucugacuucggucggaaaaccccu

SEQ ID NO: 397 - RNA, 10 nt, Artificial Sequence
  Description of Artificial Sequence Synthetic oligonucleotide
ugguguaaag

SEQ ID NO: 398 - RNA, 10 nt, Artificial Sequence
  Description of Artificial Sequence Synthetic oligonucleotide
gacgaauagu

SEQ ID NO: 399 - RNA, 10 nt, Artificial Sequence
  Description of Artificial Sequence Synthetic oligonucleotide
aggagggagu

SEQ ID NO: 400 - RNA, 10 nt, Artificial Sequence
  Description of Artificial Sequence Synthetic oligonucleotide
cagagggaac

SEQ ID NO: 401 - RNA, 10 nt, Artificial Sequence
  Description of Artificial Sequence Synthetic oligonucleotide
cagaagagga

SEQ ID NO: 402 - RNA, 10 nt, Artificial Sequence
  Description of Artificial Sequence Synthetic oligonucleotide
cagaagcaua

SEQ ID NO: 403 - RNA, 10 nt, Artificial Sequence
  Description of Artificial Sequence Synthetic oligonucleotide
gcgguguagu

SEQ ID NO: 404 - RNA, 10 nt, Artificial Sequence
  Description of Artificial Sequence Synthetic oligonucleotide
cagaaagaac

SEQ ID NO: 405 - RNA, 10 nt, Artificial Sequence
  Description of Artificial Sequence Synthetic oligonucleotide
cagaauaagc

SEQ ID NO: 406 - RNA, 10 nt, Artificial Sequence
  Description of Artificial Sequence Synthetic oligonucleotide
acgucguagu

SEQ ID NO: 407 - RNA, 10 nt, Artificial Sequence
  Description of Artificial Sequence Synthetic oligonucleotide
cagaaagggc

SEQ ID NO: 408 - RNA, 10 nt, Artificial Sequence
  Description of Artificial Sequence Synthetic oligonucleotide
cagaggcgaa

SEQ ID NO: 409 - RNA, 10 nt, Artificial Sequence
  Description of Artificial Sequence Synthetic oligonucleotide
cagaaguagc

SEQ ID NO: 410 - RNA, 10 nt, Artificial Sequence
  Description of Artificial Sequence Synthetic oligonucleotide
cagaggcaga

SEQ ID NO: 411 - RNA, 10 nt, Artificial Sequence
  Description of Artificial Sequence Synthetic oligonucleotide
cagaagaauc

SEQ ID NO: 412 - RNA, 10 nt, Artificial Sequence
  Description of Artificial Sequence Synthetic oligonucleotide
cagaaaggac

SEQ ID NO: 413 - RNA, 10 nt, Artificial Sequence
  Description of Artificial Sequence Synthetic oligonucleotide
auucuguagu

SEQ ID NO: 414 - RNA, 10 nt, Artificial Sequence
  Description of Artificial Sequence Synthetic oligonucleotide
cagagggaaa

SEQ ID NO: 415 - RNA, 10 nt, Artificial Sequence
  Description of Artificial Sequence Synthetic oligonucleotide
cagaaagaaa

SEQ ID NO: 416 - RNA, 10 nt, Artificial Sequence
  Description of Artificial Sequence Synthetic oligonucleotide
cagaauagaa

SEQ ID NO: 417 - RNA, 10 nt, Artificial Sequence
  Description of Artificial Sequence Synthetic oligonucleotide
gaauucauua

SEQ ID NO: 418 - RNA, 10 nt, Artificial Sequence
  Description of Artificial Sequence Synthetic oligonucleotide
auguuacgag

SEQ ID NO: 419 - RNA, 10 nt, Artificial Sequence
  Description of Artificial Sequence Synthetic oligonucleotide
cagaaacaga

SEQ ID NO: 420 - RNA, 10 nt, Artificial Sequence
  Description of Artificial Sequence Synthetic oligonucleotide
cagagagaua

SEQ ID NO: 421 - RNA, 10 nt, Artificial Sequence
  Description of Artificial Sequence Synthetic oligonucleotide
aaggaauuca

SEQ ID NO: 422 - RNA, 10 nt, Artificial Sequence
  Description of Artificial Sequence Synthetic oligonucleotide
gacauguagu

SEQ ID NO: 423 - RNA, 10 nt, Artificial Sequence
  Description of Artificial Sequence Synthetic oligonucleotide
gguguguagu

SEQ ID NO: 424 - RNA, 10 nt, Artificial Sequence
  Description of Artificial Sequence Synthetic oligonucleotide
aguauguagu

SEQ ID NO: 425 - RNA, 10 nt, Artificial Sequence
  Description of Artificial Sequence Synthetic oligonucleotide
cagagauaaa

SEQ ID NO: 426 - RNA, 10 nt, Artificial Sequence
  Description of Artificial Sequence Synthetic oligonucleotide
gauauguagu

SEQ ID NO: 427 - RNA, 10 nt, Artificial Sequence
  Description of Artificial Sequence Synthetic oligonucleotide
gaccuguagu

SEQ ID NO: 428 - RNA, 10 nt, Artificial Sequence
  Description of Artificial Sequence Synthetic oligonucleotide
ggaauguagu

SEQ ID NO: 429 - RNA, 10 nt, Artificial Sequence
  Description of Artificial Sequence Synthetic oligonucleotide
gguccguagu

SEQ ID NO: 430 - DNA, 105 bp, Unknown
  Description of Unknown target sequence
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 431 - DNA, 105 bp, Artificial Sequence
  Description of Artificial Sequence Synthetic polynucleotide
  modified_base (105)..(105): a, c, t, g, unknown or other
agcagccacaactccgggaacccctttgaaagtcctttcccgaatacatccacggctaaacttctcctttacaccacactgtcgtcgattaggccactcccagtn

(19) United States
(12) Patent Application Publication
(10) Pub. No.: US 2024/0271139 A1
(43) Pub. Date: Aug. 15, 2024

(54) Engineered Guide RNAs and Polynucleotides

(71) Applicant: Shape Therapeutics Inc., Seattle, WA (US)

(72) Inventors: Richard Thomas SULLIVAN, Seattle, WA (US); Brian John BOOTH, West Roxbury, MA (US); Adrian BRIGGS, Seattle, WA (US); Yiannis SAVVA, Seattle, WA (US)

(21) Appl. No.: 18/568,895

(22) PCT Filed: Jun. 28, 2022
(86) PCT No.: PCT/US2022/035320

§ 371 Date: Dec. 11, 2023

Related U.S. Application Data
(60) Provisional application No. 63/345,059, filed on May 24, 2022.
(60) Provisional application No. 63/303,680, filed on Jan. 27, 2022.
(60) Provisional application No. 63/277,701, filed on Nov. 10, 2021.
(60) Provisional application No. 63/216,178, filed on Jun. 29, 2021.

(51) Int. Cl. C12N 15/113; C12N 15/86

(52) U.S. Cl. CPC C12N 15/113; C12N 15/86; C12N 2310/20; C12N 2750/14143

(57) ABSTRACT

Disclosed herein are engineered guide RNAs and compositions comprising the same for treatment of diseases or conditions in a subject. Also disclosed herein are methods of treating diseases or conditions in a subject by administering engineered guide RNAs or pharmaceutical compositions described herein.

### CROSS REFERENCE

This application claims priority under 35 U.S.C. § 119 from Provisional Application Ser. No. 63/216,178, filed Jun. 29, 2021, Provisional Application Ser. No. 63/277,701, filed Nov. 10, 2021, Provisional Application Ser. No. 63/303,680, filed Jan. 27, 2022, and Provisional Application Ser. No. 63/345,059, filed May 24, 2022, the disclosures of which are incorporated herein by reference in their entirety.

### BACKGROUND

Compositions that mediate RNA editing can be viable therapies for genetic diseases. However, highly efficacious compositions that can maximize on-target RNA editing while minimizing off-target RNA editing are needed. Moreover, compositions that are capable of facilitating RNA editing are also needed.

### SUMMARY

Disclosed herein are engineered guide RNAs and compositions comprising an engineered guide RNA, wherein: (a) the engineered guide RNA, upon hybridization to a sequence of a target SNCA RNA, forms a guide-target RNA scaffold with the sequence of the target SNCA RNA; (b) formation of the guide-target RNA scaffold substantially forms one or more structural features selected from the group consisting of: a bulge, an internal loop, and a hairpin; (c) the structural feature is not present within the engineered guide RNA prior to the hybridization of the engineered guide RNA to the SNCA target RNA; and (d) upon hybridization of the engineered guide RNA to the sequence of the target SNCA RNA, the engineered guide RNA facilitates RNA editing of one or more target adenosines in the sequence of the target SNCA RNA by an RNA editing entity. In some embodiments, the sequence of the target SNCA RNA is within the 3′ untranslated region (UTR). In some embodiments, the sequence of the target SNCA RNA is within the 5′ untranslated region (UTR). In some embodiments, the sequence of the target SNCA RNA in the 5′ UTR is a Kozak sequence. In some embodiments, the sequence of the target SNCA RNA in the 5′ UTR is an internal ribosomal entry site (IRES). In some embodiments, the sequence of the target SNCA RNA in the 5′ UTR is an iron response element (IRE). In some embodiments, the sequence of the target SNCA RNA comprises a translation initiation site. In some embodiments, the translation initiation site is the SNCA Codon 1 translation initiation site of Exon 2. In some embodiments, the translation initiation site is the SNCA Codon 1 translation initiation site of Exon 2 corresponding to position 226 of the SNCA transcript variant 1 of accession number NM_000345.4. In some embodiments, the one or more structural features comprises: a first 6/6 symmetric internal loop at a position selected from the group consisting of: 32, 30, 28, 26, and 24, relative to the target adenosine at position 0. In some embodiments, the first 6/6 symmetric internal loop is at position 32, relative to the target adenosine at position 0. In some embodiments, the one or more structural features further comprises at least one structural feature selected from the group consisting of: a 6/6 symmetric internal loop at position −6 relative to position 0, an A/C mismatch at position 0, a U/C mismatch at position 13 relative to position 0, an A/C mismatch at position 15 relative to position 0, and any combination thereof. In some embodiments, the one or more structural features comprise further a 6/6 symmetric internal loop at position −6 relative to position 0, an A/C mismatch at position 0, a U/C mismatch at position 13 relative to position 0, and an A/C mismatch at position 15 relative to position 0. In some embodiments, the engineered guide RNA comprises at least about: 80%, 85%, 90%, 92%, 95%, 97%, or 99% sequence identity to SEQ ID NO: 350. In some embodiments, the engineered guide RNA comprises SEQ ID NO: 350. In some embodiments, the first 6/6 symmetric internal loop is at position 30, relative to the target adenosine at position 0. In some embodiments, the one or more structural features further comprises at least one structural feature selected from the group consisting of: a 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof. In some embodiments, the one or more structural features further comprise a 6/6 symmetric internal loop at position −18 relative to position 0, a 3/3 symmetric bulge at position −6 relative to position 0, an A/C mismatch at position 0, a G/G mismatch at position 6 relative to position 0, and a U/C mismatch at position 10 relative to position 0. In some embodiments, the engineered guide RNA comprises at least about: 80%, 85%, 90%, 92%, 95%, 97%, or 99% sequence identity to SEQ ID NO: 303. In some embodiments, the engineered guide RNA comprises SEQ ID NO: 303. In some embodiments, the first 6/6 symmetric internal loop is at position 28, relative to the target adenosine at position 0. In some embodiments, the one or more structural features further comprises at least one structural feature selected from the group consisting of: a 6/6 symmetric internal loop at position −8 relative to position 0, an A/C mismatch at position 0, a G/U Wobble at position 2 relative to position 0, and any combination thereof. In some embodiments, the one or more structural features further comprise a 6/6 symmetric internal loop at position −8 relative to position 0, an A/C mismatch at position 0, and a G/U Wobble at position 2 relative to position 0. In some embodiments, the engineered guide RNA comprises at least about: 80%, 85%, 90%, 92%, 95%, 97%, or 99% sequence identity to SEQ ID NO: 318. In some embodiments, the engineered guide RNA comprises SEQ ID NO: 318. In some embodiments, the one or more structural features further comprises at least one structural feature selected from the group consisting of: a 6/6 symmetric internal loop at position −10 relative to position 0, a 2/2 symmetric bulge at position −6 relative to position 0, an A/C mismatch at position 0, and any combination thereof. In some embodiments, the one or more structural features further comprise a 6/6 symmetric internal loop at position −10 relative to position 0, a 2/2 symmetric bulge at position −6 relative to position 0, and an A/C mismatch at position 0. In some embodiments, the engineered guide RNA comprises at least about: 80%, 85%, 90%, 92%, 95%, 97%, or 99% sequence identity to SEQ ID NO: 353. In some embodiments, the engineered guide RNA comprises SEQ ID NO: 353. In some embodiments, the one or more structural features further comprises at least one structural feature selected from the group consisting of: a 6/6 symmetric internal loop at position −10 relative to position 0, a 0/1 asymmetric bulge at position −6 relative to position 0, an A/C mismatch at position 0, a A/A mismatch at position 4 relative to position 0, and any combination thereof. In some embodiments, the one or more structural features further comprise a 6/6 symmetric internal loop at position −10 relative to position 0, a 0/1 asymmetric bulge at position −6 relative to position 0, an A/C mismatch at position 0, and a A/A mismatch at position 4 relative to position 0. In some embodiments, the engineered guide RNA comprises at least about: 80%, 85%, 90%, 92%, 95%, 97%, or 99% sequence identity to SEQ ID NO: 361. In some embodiments, the engineered guide RNA comprises SEQ ID NO: 361. In some embodiments, the one or more structural features further comprises at least one structural feature selected from the group consisting of: a 6/6 symmetric internal loop at position −10 relative to position 0, a 2/0 asymmetric bulge at position −4 relative to position 0, an A/C mismatch at position 0, and any combination thereof. In some embodiments, the one or more structural features further comprise a 6/6 symmetric internal loop at position −10 relative to position 0, a 2/0 asymmetric bulge at position −4 relative to position 0, and an A/C mismatch at position 0. In some embodiments, the engineered guide RNA comprises at least about: 80%, 85%, 90%, 92%, 95%, 97%, or 99% sequence identity to SEQ ID NO: 365. In some embodiments, the engineered guide RNA comprises SEQ ID NO: 365. In some embodiments, the first 6/6 symmetric internal loop is at position 26, relative to the target adenosine at position 0. In some embodiments, the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof. In some embodiments, the one or more structural features further comprise a 6/6 symmetric internal loop at position −6 relative to position 0, an A/C mismatch at position 0, and a 2/2 symmetric bulge at position 5 relative to position 0. In some embodiments, the engineered guide RNA comprises at least about: 80%, 85%, 90%, 92%, 95%, 97%, or 99% sequence identity to SEQ ID NO: 356. In some embodiments, the engineered guide RNA comprises SEQ ID NO: 356. In some embodiments, the one or more structural features further comprises at least one structural feature selected from the group consisting of: a 6/6 symmetric internal loop at position −14 relative to position 0, a 3/3 symmetric bulge at position −4 relative to position 0, an A/C mismatch at position 0, and any combination thereof. In some embodiments, the one or more structural features further comprise a 6/6 symmetric internal loop at position −14 relative to position 0, a 3/3 symmetric bulge at position −4 relative to position 0, and an A/C mismatch at position 0. In some embodiments, the engineered guide RNA comprises at least about: 80%, 85%, 90%, 92%, 95%, 97%, or 99% sequence identity to SEQ ID NO: 367. In some embodiments, the engineered guide RNA comprises SEQ ID NO: 367. In some embodiments, the first 6/6 symmetric internal loop is at position 24, relative to the target adenosine at position 0. In some embodiments, the one or more structural features comprises at least a first 6/6 symmetric internal loop and at least a second 6/6 symmetric loop. In some embodiments, the one or more structural features comprises the bulge, and wherein the bulge is a symmetric bulge. In some embodiments, the one or more structural features comprises the bulge, and wherein the bulge is an asymmetric bulge. In some embodiments, the one or more structural features comprises the internal loop, and wherein the internal loop is a symmetric internal loop. In some embodiments, the one or more structural features comprises the internal loop, and wherein the internal loop is an asymmetric internal loop. In some embodiments, the guide-target RNA scaffold comprises a Wobble base pair. In some embodiments, the one or more structural features comprises the hairpin, and wherein the hairpin is a recruitment hairpin or a non-recruitment hairpin. In some embodiments, upon hybridization of the engineered guide RNA to the sequence of the target SNCA RNA, the engineered guide RNA facilitates RNA editing of one or more adenosines in the sequence of the target SNCA RNA by an RNA editing entity. In some embodiments, the RNA editing entity comprises ADAR1, ADAR2, ADAR3, or any combination thereof. In some embodiments, the engineered guide RNA comprises at least 80%, at least 85%, at least 90%, at least 95%, at least 99%, or 100% sequence identity to any one of SEQ ID NO: 2-SEQ ID NO: 11. In some embodiments, the engineered guide RNA is encoded by an engineered polynucleotide. In some embodiments, the engineered polynucleotide is comprised in or on a vector. In some embodiments, the vector is a viral vector, and wherein the engineered polynucleotide is encapsidated in the viral vector. In some embodiments, the viral vector is an adeno-associated viral (AAV) vector, a derivative thereof. In some embodiments, the viral vector is an adeno-associated viral (AAV) and wherein the AAV vector is AAV1, AAV2, AAV3, AAV4, AAV5, AAV6, AAV7, AAV8, AAV9, AAV10, AAV11, or a derivative, a chimera, or a variant of any of these. In some embodiments, the AAV vector is a recombinant AAV (rAAV) vector, a hybrid AAV vector, a chimeric AAV vector, a self-complementary AAV (scAAV) vector, or any combination thereof. In some embodiments, the engineered guide RNA has at least 80%, 85%, 90%, 92%, 95%, 97%, or 99% sequence identity to any one of SEQ ID NO: 12-SEQ ID NO: 384. In some embodiments, the engineered guide RNA has a sequence of any one of SEQ ID NO: 12-SEQ ID NO 384.

Also disclosed herein are pharmaceutical compositions comprising: (a) an engineered guide RNA as described herein or a composition comprising an engineered guide RNA as described herein; and (b) a pharmaceutically acceptable: excipient, carrier, or diluent.

Also disclosed herein are methods of treating a disease or a condition in a subject in need thereof, the method comprising administering to the subject a therapeutically effective amount of an engineered guide RNA as described herein, a composition comprising an engineered guide RNA as described herein, or a pharmaceutical composition comprising an engineered guide RNA as described herein. In some embodiments, the disease or condition comprises a synucleinopathy. In some embodiments, the synucleinopathy comprises Parkinson's disease. In some embodiments, the subject is a human or a non-human animal. In some embodiments, the pharmaceutical composition or the composition is in unit dose form. In some embodiments, the administering is sufficient to treat one or more symptoms of the disease of condition. In some embodiments, the disease or condition is a synucleinopathy. In some embodiments, the one of more symptoms treated comprises muscle tone rigidity, bradykinesia, resting tremor, or any combination thereof. In some embodiments, the administering is sufficient to reduce aggregation of alpha-synuclein protein, relative to: (a) a level of aggregation prior to the administering; (b) a level of accumulated aggregation in the subject in the absence of the administering; or (c) both.

Also disclosed herein are methods of treating Parkinson's disease in a subject in need thereof, the method comprising administering to the subject an engineered guide RNA as described herein or a composition comprising an engineered guide RNA as described herein, in an amount sufficient to treat the Parkinson's disease in the subject. In some embodiments, the administering is sufficient to treat one or more symptoms of the Parkinson's disease in the subject, relative to prior to the administering. In some embodiments, the one of more symptoms treated comprises muscle tone rigidity, bradykinesia, resting tremor, or any combination thereof. In some embodiments, the subject after the administering displays an increased Unified Parkinson's Disease Rating Scale (UPDRS) score, relative to a UPDRS score prior to the administering.

Also disclosed herein are methods of editing an SNCA RNA, the method comprising contacting the SNCA RNA with an engineered guide RNA as described herein or a composition comprising an engineered guide RNA as described herein, and an RNA editing entity, thereby editing the SNCA RNA. In some embodiments, the editing comprises editing one or more adenosines within the 3′ untranslated region (UTR) of the SNCA RNA. In some embodiments, the editing comprises editing one or more adenosines within the 5′ untranslated region (UTR) of the SNCA RNA. In some embodiments, the editing comprises editing one or more adenosines of a transcription initiation site (TIS) of the SNCA RNA. In some embodiments, the translation initiation site is the SNCA Codon 1 translation initiation site of Exon 2, the Codon 5 translation initiate site of Exon 2, or both. In some embodiments, the SNCA RNA comprises a pre-mRNA transcript of SNCA. In some embodiments, at least 40%, at least 50%, at least 60%, at least 70%, at least 80%, at least 85%, at least 90%, at least 95%, or at least 99% of the pre-mRNA transcripts of SNCA have at least one edit. In some embodiments, the editing of SNCA RNA facilitates a protein knockdown. In some embodiments, the protein knockdown comprises a reduction of at least 10%, relative to an amount of protein present prior to the contacting. In some embodiments, the protein knockdown comprises a reduction of from about 10% to about 25%, relative to an amount of protein present prior to the contacting. In some embodiments, the protein knockdown comprises a reduction of at least 50%, relative to an amount of protein present prior to the contacting. In some embodiments, the protein knockdown comprises a knockdown of alpha-synuclein. In some embodiments, the knockdown is measured in an in vitro assay. In some embodiments, the knockdown is measured in an in vivo assay. In some embodiments, the knockdown is measured in a human subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1 shows a graph of expression of SNCA as a percentage of wildtype after hardwired A to G mutations were introduced into cells at the Codon 1 TIS and the Codon 5 TIS.

FIG. 2 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side)

FIG. 3 is a plot showing, on the x-axis, the sequence similarity of the SNCA TIS-targeting engineered guide RNAs of the present disclosure to a canonical guide RNA design and, on the y-axis, the edited fraction by an ADAR2 enzyme. These data highlight the diverse sequence space represented by the SNCA TIS-targeting engineered guide RNAs of the present disclosure, which have a range of different structural features that drive sequence diversity and which exhibit high on-target editing efficiency.

FIG. 4 shows schematics of the transcription initiation sites (TIS) in SNCA. The top schematic shows an overarching diagram of the 5′ region+TIS and the bottom schematic shows a more detailed diagram of the different TISs.

FIG. 5 shows ELISA assessment of α-synuclein protein levels in SH-SY5Y A>G hardwired mutant cell lines.*p<0.05, ***p<0.001, n=3-4 biological replicates (except for primary neurons, n=1). Data represented as Mean SD. Statistical Test: One-way ANOVA with Tukey's multiple comparisons test.

FIGS. 6A-6B show immunoblot assessment of α-synuclein protein levels in SH-SY5Y A>G hardwired mutant cell lines. FIG. 6A shows representative immunoblot using α-synuclein specific antibody and beta-actin antibody as protein loading control. FIG. 6B shows quantitative densitometric analysis of immunoblot α-synuclein protein levels normalized to protein loading control. **p<0.01, ***p<0.001, n=3-4 biological replicates. Data represented as Mean±SD. Statistical Test: One-way ANOVA with Tukey's multiple comparisons test.

FIGS. 7A-7B show quantitative PCR assessment of SNCA mRNA transcript levels in SH-SY5Y A>G hardwired mutant cell lines. SNCA mRNA transcript levels were measured by quantitative PCR using TaqMan assays specific for either SNCA exon 2-3 junction (FIG. 7A) or SNCA exon 3-4 junction (FIG. 7B). **p<0.01, n=2-6 biological replicates. Data represented as Mean±SD. Statistical Test: One-way ANOVA with Tukey's multiple comparisons test.

FIG. 8 shows biological replicates of in-cell testing of 48 gRNAs selected through high throughput screening.

FIG. 9 shows in cell editing of target SNCA exon 1 TIS by a control guide (top) and a guide RNA of the current disclosure (SEQ ID NO: 382—bottom) via ADAR1 (left) or ADAR1+ADAR2 (right).

FIG. 10 shows in cell editing of target SNCA exon 1 TIS by guide RNAs of the current disclosure (SEQ ID NO: 338—top; SEQ ID NO: 329—bottom) via ADAR1 (left) or ADAR1+ADAR2 (right).

FIG. 11 shows in cell editing of target SNCA exon 1 TIS by guide RNAs of the current disclosure (SEQ ID NO: 336—top; SEQ ID NO: 380—bottom) via ADAR1 (left) or ADAR1+ADAR2 (right).

FIG. 12 shows in cell editing of target SNCA exon 1 TIS by guide RNAs of the current disclosure (SEQ ID NO: 309—top; SEQ ID NO: 359—middle; SEQ ID NO: 357—bottom) via ADAR1 (left) or ADAR1+ADAR2 (right).

FIG. 13 shows in cell editing of target SNCA exon 1 TIS by guide RNAs of the current disclosure (SEQ ID NO: 320—top; SEQ ID NO: 373—bottom) via ADAR1 (left) or ADAR1+ADAR2 (right).

FIG. 14 shows in cell editing of target SNCA exon 1 TIS by guide RNAs of the current disclosure (SEQ ID NO: 315—top; SEQ ID NO: 321—bottom) via ADAR1 (left) or ADAR1+ADAR2 (right).

FIG. 15 shows in cell editing of target SNCA exon 1 TIS by guide RNAs of the current disclosure (SEQ ID NO: 378—top; SEQ ID NO: 320—middle; SEQ ID NO: 351—bottom) via ADAR1 (left) or ADAR1+ADAR2 (right).

FIG. 16 shows in cell editing of target SNCA exon 1 TIS by guide RNAs of the current disclosure (SEQ ID NO: 312—top; SEQ ID NO: 393—bottom) via ADAR1 (left) or ADAR1+ADAR2 (right).

FIG. 17 shows in cell editing of target SNCA exon 1 TIS by guide RNAs of the current disclosure (SEQ ID NO: 323—top; SEQ ID NO: 332—bottom) via ADAR1 (left) or ADAR1+ADAR2 (right).

FIG. 18 shows in cell editing of target SNCA exon 1 TIS by guide RNAs of the current disclosure (SEQ ID NO: 374—top; SEQ ID NO: 363—middle; SEQ ID NO: 366—bottom) via ADAR1 (left) or ADAR1+ADAR2 (right).

FIG. 19 shows in cell editing of target SNCA exon 1 TIS by guide RNAs of the current disclosure (SEQ ID NO: 369—top; SEQ ID NO: 355—middle; SEQ ID NO: 349—bottom) via ADAR1 (left) or ADAR1+ADAR2 (right).

FIG. 20 shows in cell editing of target SNCA exon 1 TIS by guide RNAs of the current disclosure (SEQ ID NO: 295—top; SEQ ID NO: 371—middle; SEQ ID NO: 319—bottom) via ADAR1 (left) or ADAR1+ADAR2 (right).

FIG. 21 shows in cell editing of target SNCA exon 1 TIS by guide RNAs of the current disclosure (SEQ ID NO: 325—top; SEQ ID NO: 219—middle; SEQ ID NO: 330—bottom) via ADAR1 (left) or ADAR1+ADAR2 (right).

FIG. 22 shows in cell editing of target SNCA exon 1 TIS by guide RNAs of the current disclosure (SEQ ID NO: 340—top; SEQ ID NO: 384—middle; SEQ ID NO: 343—bottom) via ADAR1 (left) or ADAR1+ADAR2 (right).

FIG. 23 shows in cell editing of target SNCA exon 1 TIS by guide RNAs of the current disclosure (SEQ ID NO: 376—top; SEQ ID NO: 242—bottom) via ADAR1 (left) or ADAR1+ADAR2 (right).

FIG. 24 shows in cell editing of target SNCA exon 1 TIS by guide RNAs of the current disclosure (SEQ ID NO: 345—top; SEQ ID NO: 306—middle; SEQ ID NO: 334—bottom) via ADAR1 (left) or ADAR1+ADAR2 (right).

FIG. 25 shows in cell editing of target SNCA exon 1 TIS by guide RNAs of the current disclosure (SEQ ID NO: 347—top; SEQ ID NO: 327—bottom) via ADAR1 (left) or ADAR1+ADAR2 (right).

FIG. 26 shows in cell editing of target SNCA exon 1 TIS by guide RNAs of the current disclosure (SEQ ID NO: 341) via ADAR1 (left) or ADAR1+ADAR2 (right).

FIG. 27 shows two biological replicates for in cell editing of target SNCA exon 1 TIS by a guide RNA of the current disclosure (SEQ ID NO: 365) via ADAR1 (left) or ADAR1+ADAR2 (right).

FIG. 28 shows two biological replicates for in cell editing of target SNCA exon 1 TIS by a guide RNA of the current disclosure (SEQ ID NO: 303) via ADAR1 (left) or ADAR1+ADAR2 (right).

FIG. 29 shows two biological replicates for in cell editing of target SNCA exon 1 TIS by a guide RNA of the current disclosure (SEQ ID NO: 318) via ADAR1 (left) or ADAR1+ADAR2 (right).

FIG. 30 shows two biological replicates for in cell editing of target SNCA exon 1 TIS by a guide RNA of the current disclosure (SEQ ID NO: 350) via ADAR1 (left) or ADAR1+ADAR2 (right).

FIG. 31 shows two biological replicates for in cell editing of target SNCA exon 1 TIS by a guide RNA of the current disclosure (SEQ ID NO: 361) via ADAR1 (left) or ADAR1+ADAR2 (right).

FIG. 32 shows two biological replicates for in cell editing of target SNCA exon 1 TIS by a guide RNA of the current disclosure (SEQ ID NO: 367) via ADAR1 (left) or ADAR1+ADAR2 (right).

FIG. 33 shows two biological replicates for in cell editing of target SNCA exon 1 TIS by a guide RNA of the current disclosure (SEQ ID NO: 356) via ADAR1 (left) or ADAR1+ADAR2 (right).

FIG. 34 shows two biological replicates for in cell editing of target SNCA exon 1 TIS by a guide RNA of the current disclosure (SEQ ID NO: 353) via ADAR1 (left) or ADAR1+ADAR2 (right).

### DETAILED DESCRIPTION

### RNA Editing

RNA editing can refer to a process by which RNA is enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from: cytidine to give rise to uridine, or C-to-U editing; or from adenosine to inosine, or A-to-I editing). RNA editing can be used to correct mutations (e.g., correction of a missense mutation) in order to restore protein expression and to introduce mutations or edit coding or non-coding regions of RNA to inhibit RNA translation and effect protein knockdown.

Described herein are engineered guide RNAs that facilitate RNA editing by an RNA editing entity (e.g., an adenosine Deaminase Acting on RNA (ADAR)) or biologically active fragments thereof. For example, engineered guide RNAs of the present disclosure can facilitate editing of a transcription initiation site (e.g. the Codon 1 transcription initiation site) of a target SNCA mRNA (for example, an engineered guide RNA of any one of SEQ ID NO: 12-384). In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1with three dsRBDs; ADAR2 and ADAR3 each with two dsRBDs) in their N-terminal region followed by a C-terminal deaminase domain.

The engineered guide RNAs (e.g. an engineered guide RNA of any one of SEQ ID NO: 12-384 as recited in Table 2) of the present disclosure facilitate RNA editing (for example, of an SNCA Codon 1 transcription initiation site) by endogenous ADAR enzymes. In some embodiments, exogenous ADAR can be delivered alongside the engineered guide RNAs disclosed herein to facilitate RNA editing. In some embodiments, the ADAR is human ADAR1. In some embodiments, the ADAR is human ADAR2. In some embodiments, the ADAR is human ADAR3. In some embodiments, the ADAR is human ADAR1, human ADAR2, human ADAR2, or any combination thereof.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence or a non-coding sequence of an RNA. For example, a target region in a coding sequence of an RNA can be a translation initiation site (TIS). In some embodiments, the target region in a non-coding sequence of an RNA can be a polyadenylation (polyA) signal sequence.

TIS. In some embodiments, the engineered guide RNAs of the present disclosure target the adenosine at a translation initiation site (TIS). In some embodiments, an engineered guide RNA of the present disclosure (e.g. an engineered guide RNA of any one of SEQ ID NO: 12-384 as recited in Table 2) can target the Codon 1 TIS of Exon 2 corresponding to the canonical TIS at nucleotide position 226 of SNCA transcript variant 1 (NCBI Reference Sequence: NM_000345.4). The engineered guide RNAs facilitate ADAR-mediated RNA editing of the TIS (AUG) to GUG. This results in inhibition of RNA translation and, thereby, protein knockdown. Protein knockdown can also be referred to as reduced expression of wild-type protein. In some embodiments, engineered guide RNAs of the present disclosure targeting the canonical TIS at Codon 1 of Exon 2 (nucleotide position 226 of NCBI Reference Sequence: NM_000345.4) of SNCA can be multiplexed with one or more additional engineered guide RNAs targeting a different TIS of SNCA, such as the Codon 5 translation initiate site of Exon 2. Alternatively, or in addition, one or more engineered guide RNAs of the present disclosure targeting the canonical TIS at Codon 1 of Exon 2 (nucleotide position 226 of NCBI Reference Sequence: NM_000345.4) of SNCA may be multiplexed with or more engineered guide RNAs targeting a different sequence of SNCA, such as the 5′UTR region of SNCA (e.g., a Kozak sequence, an internal ribosomal entry site (IRES), or an iron response element (IRE) of the 5′ UTR). In each of these cases, the multiplexed engineered guide RNAs can be delivered together in the same viral vector or the each of the distinct engineered guide RNAs can be delivered together but in separate vectors.

3′UTR. In some embodiments, the engineered guide RNAs of the present disclosure target one or more adenosines in the 3′ untranslated region (3′UTR). In some embodiments, an engineered guide RNA facilitates ADAR-mediated RNA editing of the one or more adenosines in the 3′UTR, thereby reducing mRNA export from the nucleus and inhibiting translation, thereby resulting in protein knockdown.

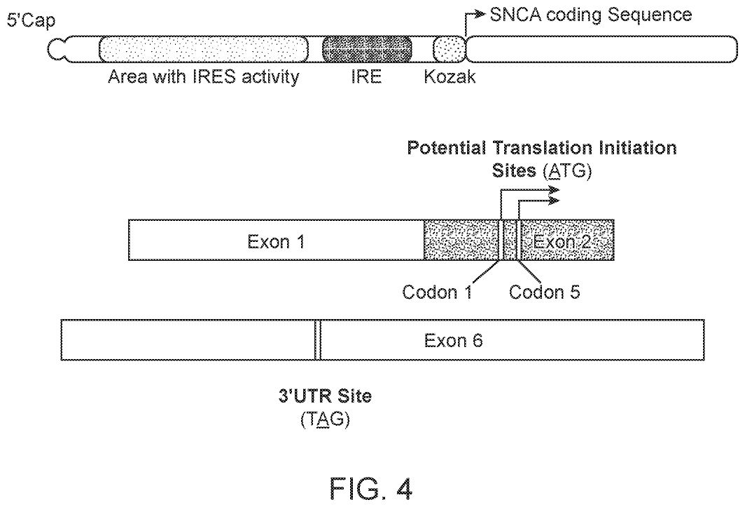

5′UTR. In some embodiments, the engineered guide RNAs of the present disclosure target one or more adenosines in the 5′ untranslated region (5′ UTR). FIG. 4 provides a schematic of the 5′ UTR, along with structures within the 5′ UTR that can be targeted by a guide RNA of the present disclosure. In some embodiments, an engineered guide RNA of the present disclosure can target a Kozak sequence of the 5′ UTR. In some embodiments, an engineered guide RNA of the present disclosure can target an internal ribosomal entry site (IRES) of the 5′ UTR. In some embodiments, an engineered guide RNA of the present disclosure can target an iron response element (IRE) of the 5′ UTR. In some embodiments, an engineered guide RNA facilitates ADAR-mediated RNA editing of one or more adenosines the 5′UTR (including one or more adenosines present in one or more structures of the 5′ UTR). In some instances, extensive or hyper editing of a plurality of adenosines can be facilitated via an engineered guide RNA of the present disclosure, which can result in ribosomal stalling of the mRNA transcript, thereby resulting in protein knockdown.

PolyA Signal Sequence. In some embodiments, the engineered guide RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered guide RNAs, varying in their respective sequences, of the present disclosure can be multiplexed to target adenosines in the one or more polyA signal sequences. In both cases, the engineered guide RNAs of the present disclosure facilitated ADAR-mediated RNA editing of adenosines to inosines (read as guanosines by cellular machinery) in the polyA signal sequence, resulting in protein knockdown.

### Engineered Guide RNAs

Disclosed herein are engineered guide RNAs (e.g. an engineered guide RNA of any one of SEQ ID NO: 12-384 as recited in Table 2) and engineered polynucleotides encoding the same for site-specific, selective editing of a target RNA (for example, an SNCA Codon 1 TIS of Exon 2 corresponding to the canonical TIS at nucleotide position 226 of SNCA transcript variant 1 (NCBI Reference Sequence: NM_000345.4)) via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule (e.g. an SNCA Codon 1 TIS of Exon 2 corresponding to the canonical TIS at nucleotide position 226 of SNCA transcript variant 1 (NCBI Reference Sequence: NM_000345.4)). A targeting sequence can also be referred to as a “targeting domain” or a “targeting region”.

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

In some cases, an engineered guide RNA targeting SNCA can comprise multiple targeting sequences. In some instances, one or more target sequence domains in the engineered guide RNA can bind to one or more regions of a target SNCA RNA. For example, a first targeting sequence can be configured to be at least partially complementary to a first region of a target RNA (e.g., a first exon of a pre-mRNA), while a second targeting sequence can be configured to be at least partially complementary to a second region of a target RNA (e.g. a second exon of a pre-mRNA). In some instances, multiple target sequences can be operatively linked to provide continuous hybridization of multiple regions of a target RNA. In some instances, multiple target sequences can provide non-continuous hybridization of multiple regions of a target RNA. A “non-continuous” overlap or hybridization refers to hybridization of a first region of a target SNCA RNA by a first targeting sequence, along with hybridization of a second region of a target SNCA RNA by a second targeting sequence, where the first region and the second region of the target SNCA RNA are discontinuous (e.g., where there is intervening sequence between the first and the second region of the target RNA). For example, a targeting sequence can be configured to bind to a portion of a first exon and can comprise an internal asymmetric loop (e.g., an oligo tether) that is configured to bind to a portion of a second exon, while the intervening sequence between the portion of exon 1 and the portion of exon 2 is not hybridized by either the targeting sequence or the oligo tether. Use of an engineered guide RNA as described herein configured for non-continuous hybridization can provide a number of benefits. For instance, such a guide can potentially target pre-mRNA during transcription (or shortly thereafter), which can then facilitate chemical modification using a deaminase (e.g., ADAR) co-transcriptionally and thus increase the overall efficiency of the chemical modification. Further, the use of oligo tethers to provide non-continuous hybridization while skipping intervening sequence can result in shorter, more specific guide RNA with fewer off-target editing.

In some instances, an engineered guide RNA configured for non-continuous hybridization to a target SNCA RNA (e.g., an engineered guide RNA comprising a targeting sequence with an oligo tether) can be configured to bind distinct regions or a target SNCA RNA separated by intervening sequence. In some instances, the intervening sequence can be at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 nucleotides. In some instances, the targeting sequence and oligo tether can target distinct non-continuous regions of the same intron or exon. In some instances, the targeting sequence and oligo tether can target distinct non-continuous regions of adjacent exons or introns. In some instances, the targeting sequence and oligo tether can target distinct non-continuous regions of distal exons or introns.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can facilitate editing of a base of a nucleotide of in a target sequence of a target RNA that results in modulating the expression of a polypeptide encoded by the target RNA. Said modulation can be increased expression of the polypeptide or decreased expression of the polypeptide. In some cases, an engineered guide can be configured to facilitate an editing of a base of a nucleotide or polynucleotide of a region of an RNA by an RNA editing entity (e.g., ADAR). In order to facilitate editing, an engineered guide RNA of the disclosure can recruit an RNA editing entity (e.g., ADAR). Various RNA editing entity recruiting domains can be utilized. In some examples, a recruiting domain comprises: Glutamate ionotropic receptor AMPA type subunit 2 (GluR2), an Alu sequence, or, in the case of recruiting APOBEC, an APOBEC recruiting domain.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence hybridizes to a target sequence of a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some examples, a recruiting domain can comprise at least about 90%, 95%, 96%, 97%, 98%, or 99% sequence homology and/or length to SEQ ID NO: 1.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of engineered guide RNAs. In some cases, a recruiting domain can be on an N-terminus, middle, or C-terminus of an engineered guide RNA. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribo- and deoxyribonucleotides can in some cases not be excluded.

C. Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA (for example, an SNCA Codon 1 TIS). The resulting dsRNA substrate is also referred to herein as a “guide-target RNA scaffold.”

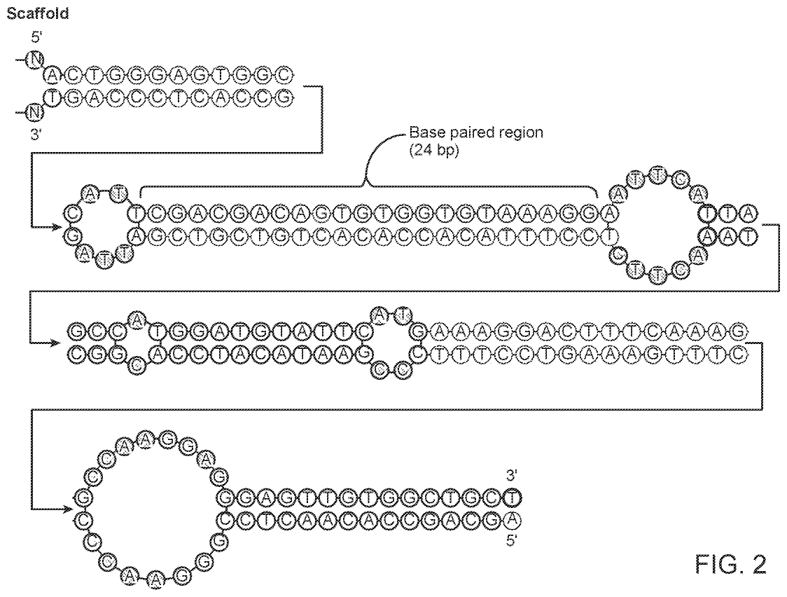

FIG. 2 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to-37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence can be absent, an engineered guide RNA can be still capable of associating with a subject RNA editing entity (e.g., ADAR) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some embodiments, an asymmetric bulge can be a 1/O asymmetric bulge. In some embodiments, a 1/O asymmetric bulge can be a U deletion. A “U deletion” refers to a 1/O asymmetric bulge in which a U nucleotide of an engineered guide RNA that would be situated opposite a non-target A of a target RNA in the guide-target RNA scaffold is deleted from the engineered guide RNA. In some instances, a 1/O asymmetric bulge comprising a U deletion can reduce editing of the non-target A, relative to a comparable guide RNA lacking the U deletion.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides there between. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

The present disclosure provides engineered guide RNAs (for example, an engineered guide RNA of any one of SEQ ID NO: 12-384 as recited in Table 2) that target a sequence of an SNCA target RNA (for example, the Codon 1 TIS of Exon 2 corresponding to the canonical TIS at nucleotide position 226 of SNCA transcript variant 1 (NCBI Reference Sequence: NM_000345.4)).

In some cases, an engineered guide RNA comprises one or more structural features that manifest as latent structures which result in editing of a target adenosine (defined as position 0) in a target sequence of an SNCA RNA (e.g. Codon 1 TIS). In some embodiments, the one or more structural features comprises a first 6/6 symmetric internal loop and a second symmetric 6/6 internal loop. In some embodiments, the one or more structural features comprises: a first 6/6 symmetric internal loop at a position selected from the group consisting of: 32, 30, 28, 26, and 24, relative to the target adenosine at position 0.

In some cases, the one or more structural features comprises the first 6/6 symmetric internal loop is at position 32, relative to the target adenosine at position 0. In some cases, the one or more structural features further comprises at least one structural feature selected from the group consisting of: a 6/6 symmetric internal loop at position −6 relative to position 0, an A/C mismatch at position 0; a G/G mismatch at position 6 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 32 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 336 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 32 relative to position 0.

In some cases, an engineered guide RNA of the present disclosure to a target SNCA RNA has a sequence of SEQ ID NO: 336 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 32 relative to position 0.

In some cases, the one or more structural features comprises the first 6/6 symmetric internal loop is at position 32, relative to the target adenosine at position 0. In some cases, the one or more structural features further comprises at least one structural feature selected from the group consisting of: a 6/6 symmetric internal loop at position −6 relative to position 0, an A/C mismatch at position 0, a U/C mismatch at position 13 relative to position 0, an A/C mismatch at position 15 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 1 nucleotide mismatch at position 15 relative to position 0, and a 6 nucleotide symmetric internal loop at position 32 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 350 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 15 relative to position 0, and a 6 nucleotide symmetric internal loop at position 32 relative to position 0.

In some cases, an engineered guide RNA of the present disclosure to a target SNCA RNA has a sequence of SEQ ID NO: 350 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 15 relative to position 0, and a 6 nucleotide symmetric internal loop at position 32 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −8 relative to position 0, an A/C mismatch at position 0, a G/G mismatch at position 6 relative to position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a 1 nucleotide mismatch at position 0, a 1 nucleotide mismatch at position 6 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 293 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a 1 nucleotide mismatch at position 6 relative to position 0, and a 6 nucleotide symmetric internal loop at position 30 relative to position 0.

In some cases, an engineered guide RNA of the present disclosure to a target SNCA RNA has a sequence of SEQ ID NO: 293 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a 1 nucleotide mismatch at position 6 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof.

In some cases, the structural feature formed upon hybridization of an engineered guide RNA of the present disclosure to a target SNCA RNA comprises a 6 nucleotide symmetric internal loop at position −18 relative to position 0, a 3 nucleotide symmetric bulge at position −6 relative to position 0, a 1 nucleotide mismatch at position 0, a 1 nucleotide mismatch at position 10 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 303 and, the guide-target RNA scaffold formed upon hybridization of said engineered guide RNA to the target SNCA RNA comprises a 6 nucleotide symmetric internal loop at position −18 relative to position 0, a 3 nucleotide symmetric bulge at position −6 relative to position 0, a 1 nucleotide mismatch at position 0, a 1 nucleotide mismatch at position 10 relative to position 0, and a 6 nucleotide symmetric internal loop at position 30 relative to position 0.

In some cases, an engineered guide RNA of the present disclosure to a target SNCA RNA has a sequence of SEQ ID NO: 303 and, the guide-target RNA scaffold formed upon hybridization of said engineered guide RNA to the target SNCA RNA comprises a 6 nucleotide symmetric internal loop at position −18 relative to position 0, a 3 nucleotide symmetric bulge at position −6 relative to position 0, a 1 nucleotide mismatch at position 0, a 1 nucleotide mismatch at position 10 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −10 relative to position 0, an A/C mismatch at position 0, a 2/2 symmetric bulge at position 4 relative to position 0, a C/C mismatch at position 11 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 1 nucleotide mismatch at position 0, a 2 nucleotide symmetric bulge at position 4 relative to position 0, a 1 nucleotide mismatch at position 11 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 306 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 2 nucleotide symmetric bulge at position 4 relative to position 0, a 1 nucleotide mismatch at position 11 relative to position 0, and a 6 nucleotide symmetric internal loop at position 30 relative to position 0.

In some cases, an engineered guide RNA of the present disclosure to a target SNCA RNA has a sequence of SEQ ID NO: 306 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 2 nucleotide symmetric bulge at position 4 relative to position 0, a 1 nucleotide mismatch at position 11 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −14 relative to position 0, a 4/4 symmetric bulge at position −5 relative to position 0, an A/C mismatch at position 0, a A/A mismatch at position 4 relative to position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 4 nucleotide symmetric bulge at position −5 relative to position 0, a 1 nucleotide mismatch at position 0, a 1 nucleotide mismatch at position 4 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 309 and, the guide-target RNA scaffold formed upon hybridization of said engineered guide RNA to the target SNCA RNA comprises a 6 nucleotide symmetric internal loop at position −14 relative to position 0, a 4 nucleotide symmetric bulge at position −5 relative to position 0, a 1 nucleotide mismatch at position 0, a 1 nucleotide mismatch at position 4 relative to position 0, and a 6 nucleotide symmetric internal loop at position 30 relative to position 0.

In some cases, an engineered guide RNA of the present disclosure to a target SNCA RNA has a sequence of SEQ ID NO: 309 and, the guide-target RNA scaffold formed upon hybridization of said engineered guide RNA to the target SNCA RNA comprises a 6 nucleotide symmetric internal loop at position −14 relative to position 0, a 4 nucleotide symmetric bulge at position −5 relative to position 0, a 1 nucleotide mismatch at position 0, a 1 nucleotide mismatch at position 4 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 2 nucleotide symmetric bulge at position 5 relative to position 0 and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 315 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 2 nucleotide symmetric bulge at position 5 relative to position 0 and a 6 nucleotide symmetric internal loop at position 30 relative to position 0.

In some cases, an engineered guide RNA of the present disclosure to a target SNCA RNA has a sequence of SEQ ID NO: 315 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 2 nucleotide symmetric bulge at position 5 relative to position 0 and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −6 relative to position 0, an A/C mismatch at position 0, a C/U mismatch at position 11 relative to position 0, a G/A mismatch at position 19 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 1 nucleotide mismatch at position 11 relative to position 0, a 1 nucleotide mismatch at position 19 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 320 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 11 relative to position 0, a 1 nucleotide mismatch at position 19 relative to position 0, and a 6 nucleotide symmetric internal loop at position 30 relative to position 0.

In some cases, an engineered guide RNA of the present disclosure to a target SNCA RNA has a sequence of SEQ ID NO: 320 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 11 relative to position 0, a 1 nucleotide mismatch at position 19 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −16 relative to position 0, a 1/O asymmetric bulge at position −4 relative to position 0, an A/C mismatch at position 0, a 2/2 symmetric bulge at position 5 relative to position 0, a U/G Wobble at position 7 relative to position 0, and any combination thereof.

In some cases, the structural feature formed upon hybridization of an engineered guide RNA of the present disclosure to a target SNCA RNA comprises a 6 nucleotide symmetric internal loop at position −16 relative to position 0, a I/O nucleotide asymmetric bulge at position −4 relative to position 0, a 1 nucleotide mismatch at position 0, a 2 nucleotide symmetric bulge at position 5 relative to position 0, a wobble base pair at position 7 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 321 and, the guide-target RNA scaffold formed upon hybridization of said engineered guide RNA to the target SNCA RNA comprises a 6 nucleotide symmetric internal loop at position −16 relative to position 0, a I/O nucleotide asymmetric bulge at position −4 relative to position 0, a 1 nucleotide mismatch at position 0, a 2 nucleotide symmetric bulge at position 5 relative to position 0, a wobble base pair at position 7 relative to position 0, and a 6 nucleotide symmetric internal loop at position 30 relative to position 0.

In some cases, an engineered guide RNA of the present disclosure to a target SNCA RNA has a sequence of SEQ ID NO: 321 and, the guide-target RNA scaffold formed upon hybridization of said engineered guide RNA to the target SNCA RNA comprises a 6 nucleotide symmetric internal loop at position −16 relative to position 0, a I/O nucleotide asymmetric bulge at position −4 relative to position 0, a 1 nucleotide mismatch at position 0, a 2 nucleotide symmetric bulge at position 5 relative to position 0, a wobble base pair at position 7 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 2/0 asymmetric bulge at position −6 relative to position 0, an A/C mismatch at position 0, a U/C mismatch at position 5 relative to position 0, a A/G mismatch at position 12 relative to position 0, and any combination thereof.

In some cases, the structural feature formed upon hybridization of an engineered guide RNA of the present disclosure to a target SNCA RNA comprises a 2 nucleotide asymmetric bulge at position −6 relative to position 0, a 1 nucleotide mismatch at position 0, a 1 nucleotide mismatch at position 5 relative to position 0, a 1 nucleotide mismatch at position 12 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 325 and, the guide-target RNA scaffold formed upon hybridization of said engineered guide RNA to the target SNCA RNA comprises a 2 nucleotide asymmetric bulge at position −6 relative to position 0, a 1 nucleotide mismatch at position 0, a 1 nucleotide mismatch at position 5 relative to position 0, a 1 nucleotide mismatch at position 12 relative to position 0, and a 6 nucleotide symmetric internal loop at position 30 relative to position 0.

In some cases, an engineered guide RNA of the present disclosure to a target SNCA RNA has a sequence of SEQ ID NO: 325 and, the guide-target RNA scaffold formed upon hybridization of said engineered guide RNA to the target SNCA RNA comprises a 2 nucleotide asymmetric bulge at position −6 relative to position 0, a 1 nucleotide mismatch at position 0, a 1 nucleotide mismatch at position 5 relative to position 0, a 1 nucleotide mismatch at position 12 relative to position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −14 relative to position 0, a 2/0 asymmetric bulge at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 2/0 nucleotide asymmetric bulge at position −5 relative to position 0, a 1 nucleotide mismatch at position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 338 and, the guide-target RNA scaffold formed upon hybridization of said engineered guide RNA to the target SNCA RNA comprises a 6 nucleotide symmetric internal loop at position −14 relative to position 0, a 2/0 nucleotide asymmetric bulge at position −5 relative to position 0, a 1 nucleotide mismatch at position 0, and a 6 nucleotide symmetric internal loop at position 30 relative to position 0.

In some cases, an engineered guide RNA of the present disclosure to a target SNCA RNA has a sequence of SEQ ID NO: 338 and, the guide-target RNA scaffold formed upon hybridization of said engineered guide RNA to the target SNCA RNA comprises a 6 nucleotide symmetric internal loop at position −14 relative to position 0, a 2/0 nucleotide asymmetric bulge at position −5 relative to position 0, a 1 nucleotide mismatch at position 0, and a 6 nucleotide symmetric internal loop at position 30 relative to position 0.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −14 relative to position 0, a U/G Wobble at position −6 relative to position 0, a 2/0 asymmetric bulge at position −3 relative to position 0, an A/C mismatch at position 0, a G/A mismatch at position 19 relative to position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a wobble base pair at position −6 relative to position 0, a 2/0 nucleotide asymmetric bulge at position −3 relative to position 0, a 1 nucleotide mismatch at position 0, a 1 nucleotide mismatch at position 19 relative to position 0, and a 6 nucleotide symmetric internal loop at position 30 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 349 and, the guide-target RNA scaffold formed upon hybridization of said engineered guide RNA to the target SNCA RNA comprises a 6 nucleotide symmetric internal loop at position −14 relative to position 0, a wobble base pair at position −6 relative to position 0, a 2/0 nucleotide asymmetric bulge at position −3 relative to position 0, a 1 nucleotide mismatch at position 0, a 1 nucleotide mismatch at position 19 relative to position 0, and a 6 nucleotide symmetric internal loop at position 30 relative to position 0.

In some cases, an engineered guide RNA of the present disclosure to a target SNCA RNA has a sequence of SEQ ID NO: 349 and, the guide-target RNA scaffold formed upon hybridization of said engineered guide RNA to the target SNCA RNA comprises a 6 nucleotide symmetric internal loop at position −14 relative to position 0, a wobble base pair at position −6 relative to position 0, a 2/0 nucleotide asymmetric bulge at position −3 relative to position 0, a 1 nucleotide mismatch at position 0, a 1 nucleotide mismatch at position 19 relative to position 0, and a 6 nucleotide symmetric internal loop at position 30 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −8 relative to position 0, an A/C mismatch at position 0, a G/U Wobble at position 2 relative to position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a 1 nucleotide mismatch at position 0, a wobble base pair at position 2 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 318 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a wobble base pair at position 2 relative to position 0, and a 6 nucleotide symmetric internal loop at position 28 relative to position 0.

In some cases, an engineered guide RNA of the present disclosure to a target SNCA RNA has a sequence of SEQ ID NO: 318 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a wobble base pair at position 2 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −16 relative to position 0, a 4/1 asymmetric bulge at position −5 relative to position 0, an A/C mismatch at position 0, a G/U Wobble at position 6 relative to position 0, and any combination thereof.

In some cases, the structural feature formed upon hybridization of an engineered guide RNA of the present disclosure to a target SNCA RNA comprises a 6 nucleotide symmetric internal loop at position −16 relative to position 0, a 4/1 asymmetric bulge at position −5 relative to position 0, a 1 nucleotide mismatch at position 0, a wobble base pair at position 6 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 319 and, the guide-target RNA scaffold formed upon hybridization of said engineered guide RNA to the target SNCA RNA comprises a 6 nucleotide symmetric internal loop at position −16 relative to position 0, a 4/1 asymmetric bulge at position −5 relative to position 0, a 1 nucleotide mismatch at position 0, a wobble base pair at position 6 relative to position 0, and a 6 nucleotide symmetric internal loop at position 28 relative to position 0.

In some cases, an engineered guide RNA of the present disclosure to a target SNCA RNA has a sequence of SEQ ID NO: 319 and, the guide-target RNA scaffold formed upon hybridization of said engineered guide RNA to the target SNCA RNA comprises a 6 nucleotide symmetric internal loop at position −16 relative to position 0, a 4/1 asymmetric bulge at position −5 relative to position 0, a 1 nucleotide mismatch at position 0, a wobble base pair at position 6 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, an A/C mismatch at position 0, a 2/2 symmetric bulge at position 5 relative to position 0, a C/U mismatch at position 11 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 1 nucleotide mismatch at position 0, a 2 nucleotide symmetric bulge at position 5 relative to position 0, a 1 nucleotide mismatch at position 11 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 329 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 2 nucleotide symmetric bulge at position 5 relative to position 0, a 1 nucleotide mismatch at position 11 relative to position 0, and a 6 nucleotide symmetric internal loop at position 28 relative to position 0.

In some cases, an engineered guide RNA of the present disclosure to a target SNCA RNA has a sequence of SEQ ID NO: 329 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 2 nucleotide symmetric bulge at position 5 relative to position 0, a 1 nucleotide mismatch at position 11 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −16 relative to position 0, a 2/0 asymmetric bulge at position −4 relative to position 0, an A/C mismatch at position 0, a U/C mismatch at position 7 relative to position 0, and any combination thereof.

In some cases, the structural feature formed upon hybridization of an engineered guide RNA of the present disclosure to a target SNCA RNA comprises a 6 nucleotide symmetric internal loop at position −16 relative to position 0, a 2/0 nucleotide asymmetric bulge at position −4 relative to position 0, a 1 nucleotide mismatch at position 0, a 1 nucleotide mismatch at position 7 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 334 and, the guide-target RNA scaffold formed upon hybridization of said engineered guide RNA to the target SNCA RNA comprises a 6 nucleotide symmetric internal loop at position −16 relative to position 0, a 2/0 nucleotide asymmetric bulge at position −4 relative to position 0, a 1 nucleotide mismatch at position 0, a 1 nucleotide mismatch at position 7 relative to position 0, and a 6 nucleotide symmetric internal loop at position 28 relative to position 0.

In some cases, an engineered guide RNA of the present disclosure to a target SNCA RNA has a sequence of SEQ ID NO: 334 and, the guide-target RNA scaffold formed upon hybridization of said engineered guide RNA to the target SNCA RNA comprises a 6 nucleotide symmetric internal loop at position −16 relative to position 0, a 2/0 nucleotide asymmetric bulge at position −4 relative to position 0, a 1 nucleotide mismatch at position 0, a 1 nucleotide mismatch at position 7 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, an A/C mismatch at position 0, a U/C mismatch at position 10 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 1 nucleotide mismatch at position 0, a 1 nucleotide mismatch at position 10 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 347 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 1 nucleotide mismatch at position 10 relative to position 0, and a 6 nucleotide symmetric internal loop at position 28 relative to position 0.

In some cases, an engineered guide RNA of the present disclosure to a target SNCA RNA has a sequence of SEQ ID NO: 347 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position 0, a 1 nucleotide mismatch at position 10 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: an A/C mismatch at position 0, a G/G mismatch at position 6 relative to position 0, and any combination thereof.

In some cases, the structural feature formed upon hybridization of an engineered guide RNA of the present disclosure to a target SNCA RNA comprises a 1 nucleotide mismatch at position 0, a 1 nucleotide mismatch at position 6 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 351 and, the guide-target RNA scaffold formed upon hybridization of said engineered guide RNA to the target SNCA RNA comprises a 1 nucleotide mismatch at position 0, a 1 nucleotide mismatch at position 6 relative to position 0, and a 6 nucleotide symmetric internal loop at position 28 relative to position 0.

In some cases, an engineered guide RNA of the present disclosure to a target SNCA RNA has a sequence of SEQ ID NO: 351 and, the guide-target RNA scaffold formed upon hybridization of said engineered guide RNA to the target SNCA RNA comprises a 1 nucleotide mismatch at position 0, a 1 nucleotide mismatch at position 6 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, a 2/2 symmetric bulge at position −6 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 2 nucleotide symmetric bulge at position 6 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 353 and, the guide-target RNA scaffold formed upon hybridization of said engineered guide RNA to the target SNCA RNA comprises a 6 nucleotide symmetric internal loop at position −10 relative to position 0, a 2 nucleotide symmetric bulge at position 6 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 353 and, the guide-target RNA scaffold formed upon hybridization of said engineered guide RNA to the target SNCA RNA comprises a 6 nucleotide symmetric internal loop at position −10 relative to position 0, a 2 nucleotide symmetric bulge at position 6 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −18 relative to position 0, a 2/0 asymmetric bulge at position −3 relative to position 0, an A/C mismatch at position 0, a 0/2 asymmetric bulge at position 18 relative to position 0, and any combination thereof.

In some cases, the structural feature formed upon hybridization of an engineered guide RNA of the present disclosure to a target SNCA RNA comprises a 6 nucleotide symmetric internal loop at position −18 relative to position 0, a 2/0 asymmetric bulge at position −3 relative to position 0, a 1 nucleotide mismatch at position 0, a 0/2 nucleotide asymmetric bulge at position 18 relative to position 0, and a 6 nucleotide symmetric internal bulge at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 355 and, the guide-target RNA scaffold formed upon hybridization of said engineered guide RNA to the target SNCA RNA comprises a 6 nucleotide symmetric internal loop at position −18 relative to position 0, a 2/0 asymmetric bulge at position −3 relative to position 0, a 1 nucleotide mismatch at position 0, a 0/2 nucleotide asymmetric bulge at position 18 relative to position 0, and a 6 nucleotide symmetric internal bulge at position 28 relative to position 0.

In some cases, an engineered guide RNA of the present disclosure to a target SNCA RNA has a sequence of SEQ ID NO: 355 and, the guide-target RNA scaffold formed upon hybridization of said engineered guide RNA to the target SNCA RNA comprises a 6 nucleotide symmetric internal loop at position −18 relative to position 0, a 2/0 asymmetric bulge at position −3 relative to position 0, a 1 nucleotide mismatch at position 0, a 0/2 nucleotide asymmetric bulge at position 18 relative to position 0, and a 6 nucleotide symmetric internal bulge at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −8 relative to position 0, a 2/1 asymmetric bulge at position −2 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a 2/1 nucleotide asymmetric bulge at position −2 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 357 and, the guide-target RNA scaffold formed upon hybridization of said engineered guide RNA to the target SNCA RNA comprises a 6 nucleotide symmetric internal loop at position −8 relative to position 0, a 2/1 nucleotide asymmetric bulge at position −2 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 357 and, the guide-target RNA scaffold formed upon hybridization of said engineered guide RNA to the target SNCA RNA comprises a 6 nucleotide symmetric internal loop at position −8 relative to position 0, a 2/1 nucleotide asymmetric bulge at position −2 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, a 2/1 asymmetric bulge at position −6 relative to position 0, an A/C mismatch at position 0, a U/C mismatch at position 13 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 2/1 nucleotide asymmetric bulge at position −6 relative to position 0, a 1 nucleotide mismatch at position 0, a 1 nucleotide mismatch at position 13 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 359 and, the guide-target RNA scaffold formed upon hybridization of said engineered guide RNA to the target SNCA RNA comprises a 6 nucleotide symmetric internal loop at position −10 relative to position 0, a 2/1 nucleotide asymmetric bulge at position −6 relative to position 0, a 1 nucleotide mismatch at position 0, a 1 nucleotide mismatch at position 13 relative to position 0, and a 6 nucleotide symmetric internal loop at position 28 relative to position 0.

In some cases, an engineered guide RNA of the present disclosure to a target SNCA RNA has a sequence of SEQ ID NO: 359 and, the guide-target RNA scaffold formed upon hybridization of said engineered guide RNA to the target SNCA RNA comprises a 6 nucleotide symmetric internal loop at position −10 relative to position 0, a 2/1 nucleotide asymmetric bulge at position −6 relative to position 0, a 1 nucleotide mismatch at position 0, a 1 nucleotide mismatch at position 13 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, a 0/1 asymmetric bulge at position −6 relative to position 0, an A/C mismatch at position 0, a A/A mismatch at position 4 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 0/1 nucleotide asymmetric bulge at position −6 relative to position 0, a 1 nucleotide mismatch at position 0, a 1 nucleotide mismatch at position 4 relative to position 0, and a 28 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 361 and, the guide-target RNA scaffold formed upon hybridization of said engineered guide RNA to the target SNCA RNA comprises a 6 nucleotide symmetric internal loop at position −10 relative to position 0, a 0/1 nucleotide asymmetric bulge at position −6 relative to position 0, a 1 nucleotide mismatch at position 0, a 1 nucleotide mismatch at position 4 relative to position 0, and a 28 nucleotide symmetric internal loop at position 28 relative to position 0.

In some cases, an engineered guide RNA of the present disclosure to a target SNCA RNA has a sequence of SEQ ID NO: 361 and, the guide-target RNA scaffold formed upon hybridization of said engineered guide RNA to the target SNCA RNA comprises a 6 nucleotide symmetric internal loop at position −10 relative to position 0, a 0/1 nucleotide asymmetric bulge at position −6 relative to position 0, a 1 nucleotide mismatch at position 0, a 1 nucleotide mismatch at position 4 relative to position 0, and a 28 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a G/G mismatch at position −3 relative to position 0, an A/C mismatch at position 0, and any combination thereof.

In some cases, the structural feature formed upon hybridization of an engineered guide RNA of the present disclosure to a target SNCA RNA comprises a 1 nucleotide mismatch at position −3 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 363 and, the guide-target RNA scaffold formed upon hybridization of said engineered guide RNA to the target SNCA RNA comprises a 1 nucleotide mismatch at position −3 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 363 and, the guide-target RNA scaffold formed upon hybridization of said engineered guide RNA to the target SNCA RNA comprises a 1 nucleotide mismatch at position −3 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, a 2/0 asymmetric bulge at position −4 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 2/0 nucleotide asymmetric bulge at position −4 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 365 and, the guide-target RNA scaffold formed upon hybridization of said engineered guide RNA to the target SNCA RNA comprises a 6 nucleotide symmetric internal loop at position −10 relative to position 0, a 2/0 nucleotide asymmetric bulge at position −4 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 365 and, the guide-target RNA scaffold formed upon hybridization of said engineered guide RNA to the target SNCA RNA comprises a 6 nucleotide symmetric internal loop at position −10 relative to position 0, a 2/0 nucleotide asymmetric bulge at position −4 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −16 relative to position 0, a 4/3 asymmetric bulge at position −3 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −16 relative to position 0, a 4/3 nucleotide asymmetric bulge at position −3 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 366 and, the guide-target RNA scaffold formed upon hybridization of said engineered guide RNA to the target SNCA RNA comprises a 6 nucleotide symmetric internal loop at position −16 relative to position 0, a 4/3 nucleotide asymmetric bulge at position −3 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 366 and, the guide-target RNA scaffold formed upon hybridization of said engineered guide RNA to the target SNCA RNA comprises a 6 nucleotide symmetric internal loop at position −16 relative to position 0, a 4/3 nucleotide asymmetric bulge at position −3 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −4 relative to position 0, an A/C mismatch at position 0, a 2/1 asymmetric bulge at position 4 relative to position 0, and any combination thereof.

In some cases, the structural feature formed upon hybridization of an engineered guide RNA of the present disclosure to a target SNCA RNA comprises a 6 nucleotide symmetric internal loop at position −4 relative to position 0, a 1 nucleotide mismatch at position 0, a 2/1 nucleotide asymmetric bulge at position 4 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 369 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 2/1 nucleotide asymmetric bulge at position 4 relative to position 0, and a 6 nucleotide symmetric internal loop at position 28 relative to position 0.

In some cases, an engineered guide RNA of the present disclosure to a target SNCA RNA has a sequence of SEQ ID NO: 369 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 2/1 nucleotide asymmetric bulge at position 4 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −4 relative to position 0, an A/C mismatch at position 0, a A/A mismatch at position 12 relative to position 0, and any combination thereof.

In some cases, the structural feature formed upon hybridization of an engineered guide RNA of the present disclosure to a target SNCA RNA comprises a 6 nucleotide symmetric internal loop at position −4 relative to position 0, a 1 nucleotide mismatch at position 0, a 1 nucleotide mismatch at position 12 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 374 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 1 nucleotide mismatch at position 12 relative to position 0, and a 6 nucleotide symmetric internal loop at position 28 relative to position 0.

In some cases, an engineered guide RNA of the present disclosure to a target SNCA RNA has a sequence of SEQ ID NO: 374 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 1 nucleotide mismatch at position 12 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −4 relative to position 0, an A/C mismatch at position 0, a C/C mismatch at position 11 relative to position 0, and any combination thereof.

In some cases, the structural feature formed upon hybridization of an engineered guide RNA of the present disclosure to a target SNCA RNA comprises a 6 nucleotide symmetric internal loop at position −4 relative to position 0, a 1 nucleotide mismatch at position 0, a 1 nucleotide mismatch at position 11 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 376 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 1 nucleotide mismatch at position 11 relative to position 0, and a 6 nucleotide symmetric internal loop at position 28 relative to position 0.

In some cases, an engineered guide RNA of the present disclosure to a target SNCA RNA has a sequence of SEQ ID NO: 376 and, the guide-target RNA scaffold formed upon hybridization of said engineered guide RNA to the target SNCA RNA comprises a 6 nucleotide symmetric internal loop at position −4 relative to position 0, a 1 nucleotide mismatch at position 0, a 1 nucleotide mismatch at position 11 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −6 relative to position 0, an A/C mismatch at position 0, a G/U Wobble at position 3 relative to position 0, a U/C mismatch at position 13 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a wobble base pair at position 3 relative to position 0, a 1 nucleotide mismatch at position 13 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 378 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a wobble base pair at position 3 relative to position 0, a 1 nucleotide mismatch at position 13 relative to position 0, and a 6 nucleotide symmetric internal loop at position 28 relative to position 0.

In some cases, an engineered guide RNA of the present disclosure to a target SNCA RNA has a sequence of SEQ ID NO: 378 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a wobble base pair at position 3 relative to position 0, a 1 nucleotide mismatch at position 13 relative to position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −8 relative to position 0, an A/C mismatch at position 0, a C/U mismatch at position 11 relative to position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a 1 nucleotide mismatch at position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 380 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, and a 6 nucleotide symmetric internal loop at position 28 relative to position 0.

In some cases, an engineered guide RNA of the present disclosure to a target SNCA RNA has a sequence of SEQ ID NO: 380 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, and a 6 nucleotide symmetric internal loop at position 28 relative to position 0.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −14 relative to position 0, a 3/3 symmetric bulge at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 3 nucleotide symmetric bulge at position −5 relative to position 0, a 1 nucleotide mismatch at position 0, a 6 nucleotide symmetric internal loop at position 28 nucleotides downstream of the target A.

In some cases, an engineered guide RNA of the present disclosure to a target SNCA RNA has at least 80%, 85%, 90%, 92%, 95%, 97%, or 99% sequence identity to a guide RNA comprising SEQ ID NO: 382 and, the guide-target RNA scaffold formed upon hybridization of said engineered guide RNA to the target SNCA RNA comprises a 6 nucleotide symmetric internal loop at position −14 relative to position 0, a 3 nucleotide symmetric bulge at position −5 relative to position 0, a 1 nucleotide mismatch at position 0, a 6 nucleotide symmetric internal loop at position 28 nucleotides downstream of the target A.

In some cases, an engineered guide RNA of the present disclosure to a target SNCA RNA has a sequence of SEQ ID NO: 382 and, the guide-target RNA scaffold formed upon hybridization of said engineered guide RNA to the target SNCA RNA comprises a 6 nucleotide symmetric internal loop at position −14 relative to position 0, a 3 nucleotide symmetric bulge at position −5 relative to position 0, a 1 nucleotide mismatch at position 0, a 6 nucleotide symmetric internal loop at position 28 nucleotides downstream of the target A.

In some cases, the one or more structural features comprises the first 6/6 symmetric internal loop is at position 28, relative to the target adenosine at position 0. In some cases, the one or more structural features further comprises at least one structural feature selected from the group consisting of: a 6/6 symmetric internal loop at position −10 relative to position 0, a 3/3 symmetric bulge at position −5 relative to position 0, an A/C mismatch at position 0, a U/G Wobble at position 10 relative to position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 3 nucleotide symmetric bulge at position −5 relative to position 0, a 1 nucleotide mismatch at position 0, a wobble base pair at position 10 relative to position 0, and a 6 nucleotide symmetric internal loop at position 28 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 384 and, the guide-target RNA scaffold formed upon hybridization of said engineered guide RNA to the target SNCA RNA comprises a 6 nucleotide symmetric internal loop at position −10 relative to position 0, a 3 nucleotide symmetric bulge at position −5 relative to position 0, a 1 nucleotide mismatch at position 0, a wobble base pair at position 10 relative to position 0, and a 6 nucleotide symmetric internal loop at position 28 relative to position 0.

In some cases, an engineered guide RNA of the present disclosure to a target SNCA RNA has a sequence of SEQ ID NO: 384 and, the guide-target RNA scaffold formed upon hybridization of said engineered guide RNA to the target SNCA RNA comprises a 6 nucleotide symmetric internal loop at position −10 relative to position 0, a 3 nucleotide symmetric bulge at position −5 relative to position 0, a 1 nucleotide mismatch at position 0, a wobble base pair at position 10 relative to position 0, and a 6 nucleotide symmetric internal loop at position 28 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −12 relative to position 0, a 3/2 asymmetric bulge at position −4 relative to position 0, an A/C mismatch at position 0, a U/G Wobble at position 13 relative to position 0, and any combination thereof.

In some cases, the structural feature formed upon hybridization of an engineered guide RNA of the present disclosure to a target SNCA RNA comprises a 6 nucleotide symmetric internal loop at position −12 relative to position 0, a 3/2 nucleotide asymmetric bulge at position −4 relative to position 0, a 1 nucleotide mismatch at position 0, a wobble base pair at position 13 relative to position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 299 and, the guide-target RNA scaffold formed upon hybridization of said engineered guide RNA to the target SNCA RNA comprises a 6 nucleotide symmetric internal loop at position −12 relative to position 0, a 3/2 nucleotide asymmetric bulge at position −4 relative to position 0, a 1 nucleotide mismatch at position 0, a wobble base pair at position 13 relative to position 0, and a 6 nucleotide symmetric internal loop at position 26 relative to position 0.

In some cases, an engineered guide RNA of the present disclosure to a target SNCA RNA has a sequence of SEQ ID NO: 299 and, the guide-target RNA scaffold formed upon hybridization of said engineered guide RNA to the target SNCA RNA comprises a 6 nucleotide symmetric internal loop at position −12 relative to position 0, a 3/2 nucleotide asymmetric bulge at position −4 relative to position 0, a 1 nucleotide mismatch at position 0, a wobble base pair at position 13 relative to position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −14 relative to position 0, an A/A mismatch at position −7 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 1 nucleotide mismatch at position −7 relative to position 0, a 1 nucleotide mismatch at position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 312 and, the guide-target RNA scaffold formed upon hybridization of said engineered guide RNA to the target SNCA RNA comprises a 6 nucleotide symmetric internal loop at position −14 relative to position 0, a 1 nucleotide mismatch at position −7 relative to position 0, a 1 nucleotide mismatch at position 0, and a 6 nucleotide symmetric internal loop at position 26 relative to position 0.

In some cases, an engineered guide RNA of the present disclosure to a target SNCA RNA has a sequence of SEQ ID NO: 312 and, the guide-target RNA scaffold formed upon hybridization of said engineered guide RNA to the target SNCA RNA comprises a 6 nucleotide symmetric internal loop at position −14 relative to position 0, a 1 nucleotide mismatch at position −7 relative to position 0, a 1 nucleotide mismatch at position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −12 relative to position 0, a 2/1 asymmetric bulge at position −2 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −12 relative to position 0, a 2/1 nucleotide asymmetric bulge at position −2 relative to position 0, a 1 nucleotide mismatch at position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 323 and, the guide-target RNA scaffold formed upon hybridization of said engineered guide RNA to the target SNCA RNA comprises a 6 nucleotide symmetric internal loop at position −12 relative to position 0, a 2/1 nucleotide asymmetric bulge at position −2 relative to position 0, a 1 nucleotide mismatch at position 0, and a 6 nucleotide symmetric internal loop at position 26 relative to position 0.

In some cases, an engineered guide RNA of the present disclosure to a target SNCA RNA has a sequence of SEQ ID NO: 323 and, the guide-target RNA scaffold formed upon hybridization of said engineered guide RNA to the target SNCA RNA comprises a 6 nucleotide symmetric internal loop at position −12 relative to position 0, a 2/1 nucleotide asymmetric bulge at position −2 relative to position 0, a 1 nucleotide mismatch at position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −8 relative to position 0, a U/G Wobble at position −6 relative to position 0, an A/C mismatch at position 0, a U/U mismatch at position 9 relative to position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a wobble base pair at position −6 relative to position 0, a 1 nucleotide mismatch at position 0, a 1 nucleotide mismatch at position 9 relative to position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 327 and, the guide-target RNA scaffold formed upon hybridization of said engineered guide RNA to the target SNCA RNA comprises a 6 nucleotide symmetric internal loop at position −8 relative to position 0, a wobble base pair at position −6 relative to position 0, a 1 nucleotide mismatch at position 0, a 1 nucleotide mismatch at position 9 relative to position 0, and a 6 nucleotide symmetric internal loop at position 26 relative to position 0.

In some cases, an engineered guide RNA of the present disclosure to a target SNCA RNA has a sequence of SEQ ID NO: 327 and, the guide-target RNA scaffold formed upon hybridization of said engineered guide RNA to the target SNCA RNA comprises a 6 nucleotide symmetric internal loop at position −8 relative to position 0, a wobble base pair at position −6 relative to position 0, a 1 nucleotide mismatch at position 0, a 1 nucleotide mismatch at position 9 relative to position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −16 relative to position 0, a 0/1 asymmetric bulge at position −7 relative to position 0, an A/C mismatch at position 0, a C/U mismatch at position 11 relative to position 0, and any combination thereof.

In some cases, the structural feature formed upon hybridization of an engineered guide RNA of the present disclosure to a target SNCA RNA comprises a 6 nucleotide symmetric internal loop at position −16 relative to position 0, a 0/1 nucleotide asymmetric bulge at position −7 relative to position 0, a 1 nucleotide mismatch at position 0, a 1 nucleotide mismatch at position 11 relative to position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 341 and, the guide-target RNA scaffold formed upon hybridization of said engineered guide RNA to the target SNCA RNA comprises a 6 nucleotide symmetric internal loop at position −16 relative to position 0, a 0/1 nucleotide asymmetric bulge at position −7 relative to position 0, a 1 nucleotide mismatch at position 0, a 1 nucleotide mismatch at position 11 relative to position 0, and a 6 nucleotide symmetric internal loop at position 26 relative to position 0.

In some cases, an engineered guide RNA of the present disclosure to a target SNCA RNA has a sequence of SEQ ID NO: 341 and, the guide-target RNA scaffold formed upon hybridization of said engineered guide RNA to the target SNCA RNA comprises a 6 nucleotide symmetric internal loop at position −16 relative to position 0, a 0/1 nucleotide asymmetric bulge at position −7 relative to position 0, a 1 nucleotide mismatch at position 0, a 1 nucleotide mismatch at position 11 relative to position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −10 relative to position 0, a U/C mismatch at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 1 nucleotide mismatch at position −5 relative to position 0, a 1 nucleotide mismatch at position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 343 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position −5 relative to position 0, a 1 nucleotide mismatch at position 0, and a 6 nucleotide symmetric internal loop at position 26 relative to position 0.

In some cases, an engineered guide RNA of the present disclosure to a target SNCA RNA has a sequence of SEQ ID NO: 343 and, the guide-target RNA scaffold formed upon hybridization of said engineered guide RNA to the target SNCA RNA comprises a 6 nucleotide symmetric internal loop at position −10 relative to position 0, a 1 nucleotide mismatch at position −5 relative to position 0, a 1 nucleotide mismatch at position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 2 nucleotide symmetric bulge at position 5 relative to position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 356 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 2 nucleotide symmetric bulge at position 5 relative to position 0, and a 6 nucleotide symmetric internal loop at position 26 relative to position 0.

In some cases, an engineered guide RNA of the present disclosure to a target SNCA RNA has a sequence of SEQ ID NO: 356 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 2 nucleotide symmetric bulge at position 5 relative to position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −14 relative to position 0, a 3/3 symmetric bulge at position −4 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 3 nucleotide symmetric bulge at position 4 relative to position 0, a 1 nucleotide mismatch at position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 367 and, the guide-target RNA scaffold formed upon hybridization of said engineered guide RNA to the target SNCA RNA comprises a 6 nucleotide symmetric internal loop at position −14 relative to position 0, a 3 nucleotide symmetric bulge at position 4 relative to position 0, a 1 nucleotide mismatch at position 0, and a 6 nucleotide symmetric internal loop at position 26 relative to position 0.

In some cases, an engineered guide RNA of the present disclosure to a target SNCA RNA has a sequence of SEQ ID NO: 367 and, the guide-target RNA scaffold formed upon hybridization of said engineered guide RNA to the target SNCA RNA comprises a 6 nucleotide symmetric internal loop at position −14 relative to position 0, a 3 nucleotide symmetric bulge at position 4 relative to position 0, a 1 nucleotide mismatch at position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −10 relative to position 0, a 2/2 symmetric bulge at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −10 relative to position 0, a 2 nucleotide symmetric bulge at position −5 relative to position 0, a 1 nucleotide mismatch at position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 371 and, the guide-target RNA scaffold formed upon hybridization of said engineered guide RNA to the target SNCA RNA comprises a 6 nucleotide symmetric internal loop at position −10 relative to position 0, a 2 nucleotide symmetric bulge at position −5 relative to position 0, a 1 nucleotide mismatch at position 0, and a 6 nucleotide symmetric internal loop at position 26 relative to position 0.

In some cases, an engineered guide RNA of the present disclosure to a target SNCA RNA has a sequence of SEQ ID NO: 371 and, the guide-target RNA scaffold formed upon hybridization of said engineered guide RNA to the target SNCA RNA comprises a 6 nucleotide symmetric internal loop at position −10 relative to position 0, a 2 nucleotide symmetric bulge at position −5 relative to position 0, a 1 nucleotide mismatch at position 0, and a 6 nucleotide symmetric internal loop at position 26 relative to position 0.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a 4/4 symmetric bulge at position −5 relative to position 0, an A/C mismatch at position 0, a 0/1 asymmetric bulge at position 5 relative to position 0, an A/C mismatch at position 17 relative to position 0, and any combination thereof.

In some cases, the structural feature formed upon hybridization of an engineered guide RNA of the present disclosure to a target SNCA RNA comprises a 6 nucleotide symmetric internal loop at position −20 relative to position 0, a 4 nucleotide symmetric bulge at position −5 relative to position 0, a 1 nucleotide mismatch at position 0, a 0/1 nucleotide asymmetric bulge at position 5 relative to position 0, a 1 nucleotide mismatch at position 17 relative to position 0, and a 6 nucleotide symmetric internal loop at position 26 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 373 and, the guide-target RNA scaffold formed upon hybridization of said engineered guide RNA to the target SNCA RNA comprises a 6 nucleotide symmetric internal loop at position −20 relative to position 0, a 4 nucleotide symmetric bulge at position −5 relative to position 0, a 1 nucleotide mismatch at position 0, a 0/1 nucleotide asymmetric bulge at position 5 relative to position 0, a 1 nucleotide mismatch at position 17 relative to position 0, and a 6 nucleotide symmetric internal loop at position 26 relative to position 0.

In some cases, an engineered guide RNA of the present disclosure to a target SNCA RNA has a sequence of SEQ ID NO: 373 and, the guide-target RNA scaffold formed upon hybridization of said engineered guide RNA to the target SNCA RNA comprises a 6 nucleotide symmetric internal loop at position −20 relative to position 0, a 4 nucleotide symmetric bulge at position −5 relative to position 0, a 1 nucleotide mismatch at position 0, a 0/1 nucleotide asymmetric bulge at position 5 relative to position 0, a 1 nucleotide mismatch at position 17 relative to position 0, and a 6 nucleotide symmetric internal loop at position 26 relative to position 0.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −6 relative to position 0, an A/C mismatch at position 0, a G/G mismatch at position 6 relative to position 0, and any combination thereof.

In some cases, the structural feature formed upon hybridization of an engineered guide RNA of the present disclosure to a target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 295 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 24 relative to position 0.

In some cases, an engineered guide RNA of the present disclosure to a target SNCA RNA has a sequence of SEQ ID NO: 295 and, the guide-target RNA scaffold formed upon hybridization of said engineered guide RNA to the target SNCA RNA comprises a 6 nucleotide symmetric internal loop at position −6 relative to position 0, a 1 nucleotide mismatch at position 0, a 1 nucleotide mismatch at position 6 relative to position 0, and a 6 nucleotide symmetric internal loop at position 24 relative to position 0.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −18 relative to position 0, a U/C mismatch at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −18 relative to position 0, a 1 nucleotide mismatch at position −5 relative to position 0, a 1 nucleotide mismatch at position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 330 and, the guide-target RNA scaffold formed upon hybridization of said engineered guide RNA to the target SNCA RNA comprises a 6 nucleotide symmetric internal loop at position −18 relative to position 0, a 1 nucleotide mismatch at position −5 relative to position 0, a 1 nucleotide mismatch at position 0, and a 6 nucleotide symmetric internal loop at position 24 relative to position 0.

In some cases, an engineered guide RNA of the present disclosure to a target SNCA RNA has a sequence of SEQ ID NO: 330 and, the guide-target RNA scaffold formed upon hybridization of said engineered guide RNA to the target SNCA RNA comprises a 6 nucleotide symmetric internal loop at position −18 relative to position 0, a 1 nucleotide mismatch at position −5 relative to position 0, a 1 nucleotide mismatch at position 0, and a 6 nucleotide symmetric internal loop at position 24 relative to position 0.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −14 relative to position 0, a U/C mismatch at position −5 relative to position 0, an A/C mismatch at position 0, and any combination thereof.

In some cases, the structural feature formed upon hybridization of an engineered guide RNA of the present disclosure to a target SNCA RNA comprises a 6 nucleotide symmetric internal loop at position −14 relative to position 0, a 1 nucleotide mismatch at position −5 relative to position 0, a 1 nucleotide mismatch at position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 332 and, the guide-target RNA scaffold formed upon hybridization of said engineered guide RNA to the target SNCA RNA comprises a 6 nucleotide symmetric internal loop at position −14 relative to position 0, a 1 nucleotide mismatch at position −5 relative to position 0, a 1 nucleotide mismatch at position 0, and a 6 nucleotide symmetric internal loop at position 24 relative to position 0.

In some cases, an engineered guide RNA of the present disclosure to a target SNCA RNA has a sequence of SEQ ID NO: 332 and, the guide-target RNA scaffold formed upon hybridization of said engineered guide RNA to the target SNCA RNA comprises a 6 nucleotide symmetric internal loop at position −14 relative to position 0, a 1 nucleotide mismatch at position −5 relative to position 0, a 1 nucleotide mismatch at position 0, and a 6 nucleotide symmetric internal loop at position 24 relative to position 0.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −8 relative to position 0, an A/C mismatch at position 0, a A/C mismatch at position 4 relative to position 0, and any combination thereof.

In some cases, the structural feature formed upon hybridization of an engineered guide RNA of the present disclosure to a target SNCA RNA comprises a 6 nucleotide symmetric internal loop at position −8 relative to position 0, a 1 nucleotide mismatch at position 0, a 1 nucleotide mismatch at position 4 relative to position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 340 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a 1 nucleotide mismatch at position 4 relative to position 0, and a 6 nucleotide symmetric internal loop at position 24 relative to position 0.

In some cases, an engineered guide RNA of the present disclosure to a target SNCA RNA has a sequence of SEQ ID NO: 340 and, the guide-target RNA scaffold formed upon hybridization of said engineered guide RNA to the target SNCA RNA comprises a 6 nucleotide symmetric internal loop at position −8 relative to position 0, a 1 nucleotide mismatch at position 0, a 1 nucleotide mismatch at position 4 relative to position 0, and a 6 nucleotide symmetric internal loop at position 24 relative to position 0.

In some cases, the one or more structural features comprises the first 6/6 symmetric internal loop is at position 24, relative to the target adenosine at position 0. In some cases, the one or more structural features further comprises at least one structural feature selected from the group consisting of: a 8/8 symmetric internal loop at position −6 relative to position 0, an A/C mismatch at position 0, a G/A mismatch at position 6 relative to position 0, a U/G Wobble at position 7 relative to position 1, and any combination thereof.

In some cases, the structural feature formed upon hybridization of an engineered guide RNA of the present disclosure to a target SNCA RNA comprises a 8 nucleotide symmetric internal loop at position −6 relative to position 0, a 1 nucleotide mismatch at position 0, a 1 nucleotide mismatch at position 6 relative to position 0, a wobble base pair at position 7 relative to position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 345 and, the guide-target RNA scaffold formed upon hybridization of said engineered guide RNA to the target SNCA RNA comprises a 8 nucleotide symmetric internal loop at position −6 relative to position 0, a 1 nucleotide mismatch at position 0, a 1 nucleotide mismatch at position 6 relative to position 0, a wobble base pair at position 7 relative to position 0, and a 6 nucleotide symmetric internal loop at position 24 relative to position 0.

In some cases, an engineered guide RNA of the present disclosure to a target SNCA RNA has a sequence of SEQ ID NO: 345 and, the guide-target RNA scaffold formed upon hybridization of said engineered guide RNA to the target SNCA RNA comprises a 8 nucleotide symmetric internal loop at position −6 relative to position 0, a 1 nucleotide mismatch at position 0, a 1 nucleotide mismatch at position 6 relative to position 0, a wobble base pair at position 7 relative to position 0, and a 6 nucleotide symmetric internal loop at position 24 relative to position 0.

In some cases, an engineered guide RNA of the present disclosure to a target SNCA RNA has at least 80%, 85%, 90%, 92%, 95%, 97%, or 99% sequence identity to a guide RNA comprising SEQ ID NO: 365; or the engineered guide RNA comprises the sequence of SEQ ID NO: 365. In some cases, an engineered guide RNA of the present disclosure to a target SNCA RNA has at least 80%, 85%, 90%, 92%, 95%, 97%, or 99% sequence identity to a guide RNA comprising SEQ ID NO: 303; or the engineered guide RNA comprises the sequence of SEQ ID NO: 303. In some cases, an engineered guide RNA of the present disclosure to a target SNCA RNA has at least 80%, 85%, 90%, 92%, 95%, 97%, or 99% sequence identity to a guide RNA comprising SEQ ID NO: 318; or the engineered guide RNA comprises the sequence of SEQ ID NO: 318. In some cases, an engineered guide RNA of the present disclosure to a target SNCA RNA has at least 80%, 85%, 90%, 92%, 95%, 97%, or 99% sequence identity to a guide RNA comprising SEQ ID NO: 350; or the engineered guide RNA comprises the sequence of SEQ ID NO: 350. In some cases, an engineered guide RNA of the present disclosure to a target SNCA RNA has at least 80%, 85%, 90%, 92%, 95%, 97%, or 99% sequence identity to a guide RNA comprising SEQ ID NO: 361; or the engineered guide RNA comprises the sequence of SEQ ID NO: 361. In some cases, an engineered guide RNA of the present disclosure to a target SNCA RNA has at least 80%, 85%, 90%, 92%, 95%, 97%, or 99% sequence identity to a guide RNA comprising SEQ ID NO: 367; or the engineered guide RNA comprises the sequence of SEQ ID NO: 367. In some cases, an engineered guide RNA of the present disclosure to a target SNCA RNA has at least 80%, 85%, 90%, 92%, 95%, 97%, or 99% sequence identity to a guide RNA comprising SEQ ID NO: 353; or the engineered guide RNA comprises the sequence of SEQ ID NO: 353.

### Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both sequences.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl. In some embodiments, a circular engineered guide RNA can comprise a guide RNA from any one of SEQ ID NOs: 12-384 as recited in Table 2 that target SNCA Codon 1 TIS of Exon 2.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. Circular guide RNAs are described in PCT/US2021/034301, which is incorporated by reference in its entirety.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g. increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features. Promoters and snRNA components are described in PCT/US2021/028618, which is incorporated by reference in its entirety.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 385 or RNA: UAGGCUUUCUGGCUUUUUACCGGAAAGCCCCU (SEQ ID NO: 386). In some cases, a mouse U7 hairpin sequence comprises CAGGTTTTCTGACTTCGGTCGGAAAACCCCT (SEQ ID NO: 387 or RNA: CAGGUUUUCUGACUUCGGUCGGAAAACCCCU SEQ ID NO: 1593). In some embodiments, the SmOPT sequence has a sequence of AATTTTTGGAG (SEQ ID NO: 388 or RNA: AAUUUUUGGAG SEQ ID NO: 389). In some embodiments, a guide RNA from any one of SEQ ID NOs: 12-384 as recited in Table 2 that target SNCA Codon 1 TIS of Exon 2 can comprise a guide RNA comprising a U7 hairpin sequence (e.g., a human or a mouse U7 hairpin sequence), an SmOPT sequence, or a combination thereof. In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

Also disclosed herein are promoters for driving the expression of a guide RNA disclosed herein. In some cases, the promoters for driving expression can be 5′ to the guide RNA sequence disclosed herein. In some cases, a promoter can comprise a U1 promoter, a U7 promoter, a U6 promoter or any combination thereof. In some cases, a promoter can comprise a CMV promoter. In some cases, a U7 promoter, or a U6 promoter can be a mouse U7 promoter, or a mouse U6 promoter. In some cases, a U1 promoter, a U7 promoter, or a U6 promoter can be a human U1 promoter, a human U7 promoter, or a human U6 promoter. In some cases, a human U6 promoter can comprise a sequence with at least about: 70%, 75%, 80%, 85%, 90%, 95%, or 99% sequence identity to: GAGGGCCTATTTCCCATGATTCCTTCATATTTGCATATACGATACAAGGCTGTTA GAGAGATAATTAGAATTAATTTGACTGTAAACACAAAGATATTAGTACAAAATA CGTGACGTAGAAAGTAATAATTTCTTGGGTAGTTTGCAGTTTTAAAATTATGTTTT AAAATGGACTATCATATGCTTACCGTAACTTGAAAGTATTTCGATTTCTTGGCTTT ATATATCTTGTGGAAAGGACGAAACACC (SEQ ID NO: 390). In some cases, a mouse U6 promoter can comprise a sequence with at least about: 70%, 75%, 80%, 85%, 90%, 95%, or 99% sequence identity to: GTACTGAGTCGCCCAGTCTCAGATAGATCCGACGCCGCCATCTCTAGGCCCGCGC CGGCCCCCTCGCACAGACTTGTGGGAGAAGCTCGGCTACTCCCCTGCCCCGGTTA ATTTGCATATAATATTTCCTAGTAACTATAGAGGCTTAATGTGCGATAAAAGACA GATAATCTGTTCTTTTTAATACTAGCTACATTTTACATGATAGGCTTGGATTTCTA TAAGAGATACAAATACTAAATTATTATTTTAAAAAACAGCACAAAAGGAAACTC ACCCTAACTGTAAAGTAATTGTGTGTTTTGAGACTATAAATATCCCTTGGAGAAA AGCCTTGTTTG (SEQ ID NO: 391). In some cases, a human U7 promoter can comprise a sequence with at least about: 70%, 75%, 80%, 85%, 90%, 95%, or 99% sequence identity to: TTAACAACAACGAAGGGGCTGTGACTGGCTGCTTTCTCAACCAATCAGCACCGA ACTCATTTGCATGGGCTGAGAACAAATGTTCGCGAACTCTAGAAATGAATGACTT AAGTAAGTTCCTTAGAATATTATTTTTCCTACTGAAAGTTACCACATGCGTCGTTG TTTATACAGTAATAGGAACAAGAAAAAAGTCACCTAAGCTCACCCTCATCAATT GTGGAGTTCCTTTATATCCCATCTTCTCTCCAAACACATACGCA (SEQ ID NO: 392). In some cases, a mouse U7 promoter can comprise a sequence with at least about: 70%, 75%, 80%, 85%, 90%, 95%, or 99% sequence identity to: TTAACAACATAGGAGCTGTGATTGGCTGTTTTCAGCCAATCAGCACTGACTCATT TGCATAGCCTTTACAAGCGGTCACAAACTCAAGAAACGAGCGGTTTTAATAGTCT TTTAGAATATTGTTTATCGAACCGAATAAGGAACTGTGCTTTGTGATTCACATAT CAGTGGAGGGGTGTGGAAATGGCACCTTGATCTCACCCTCATCGAAAGTGGAGT TGATGTCCTTCCCTGGCTCGCTACAGACGCACTTCCGC (SEQ ID NO: 393). In some cases, a human U1 promoter can comprise a sequence with at least about: 70%, 75%, 80%, 85%, 90%, 95%, or 99% sequence identity to: TAAGGACCAGCTTCTTTGGGAGAGAACAGACGCAGGGGCGGGAGGGAAAAAGG GAGAGGCAGACGTCACTTCCTCTTGGCGACTCTGGCAGCAGATTGGTCGGTTGAG TGGCAGAAAGGCAGACGGGGACTGGGCAAGGCACTGTCGGTGACATCACGGAC AGGGCGACTTCTATGTAGATGAGGCAGCGCAGAGGCTGCTGCTTCGCCACTTGCT GCTTCGCCACGAAGGGAGTTCCCGTGCCCTGGGAGCGGGTTCAGGACCGCTGAT CGGAAGTGAGAATCCCAGCTGTGTGTCAGGGCTGGAAAGGGCTCGGGAGTGCGC GGGGCAAGTGACCGTGTGTGTAAAGAGTGAGGCGTATGAGGCTGTGTCGGGGCA GAGCCCGAAGATCTC (SEQ ID NO: 394). In some cases, a CMV promoter can comprise a sequence with at least about: 70%, 75%, 80%, 85%, 90%, 95%, or 99% sequence identity

(SEQ ID NO: 395)
ATACGCGTTGACATTGATTATTGACTAGTTATTAATAGTAATCAATTACG
GGGTCATTAGTTCATAGCCCATATATGGAGTTCCGCGTTACATAACTTAC
GGTAAATGGCCCGCCTGGCTGACCGCCCAACGACCCCCGCCCATTGACGT
CAATAATGACGTATGTTCCCATAGTAACGCCAATAGGGACTTTCCATTGA
CGTCAATGGGTGGAGTATTTACGGTAAACTGCCCACTTGGCAGTACATCA
AGTGTATCATATGCCAAGTACGCCCCCTATTGACGTCAATGACGGTAAAT
GGCCCGCCTGGCATTATGCCCAGTACATGACCTTATGGGACTTTCCTACT
TGGCAGTACATCTACGTATTAGTCATCGCTATTACCATGGTGATGCGGTT
TTGGCAGTACATCAATGGGCGTGGATAGCGGTTTGACTCACGGGGATTTC
CAAGTCTCCACCCCATTGACGTCAATGGGAGTTTGTTTTGGCACCAAAAT
CAACGGGACTTTCCAAAATGTCGTAACAACTCCGCCCCATTGACGCAAAT
GGGCGGTAGGCGTGTACGGTGGGAGGTCTATATAAGCAGAGCTCGTTTAG
TGAACCGTCAGATCGCCTGGAGACGCCATCCACGCTGTTTTGACCTCCAT
AGAAGACACCGGGACCGATCCAGCCTCCGGACTCTAGAGGATCGAACC.

### Targets and Methods of Treatment

The present disclosure provides for compositions of engineered guide RNAs or engineered polynucleotides encoding guide RNAs and methods of use thereof, such as methods of treatment. In some embodiments, the engineered polynucleotides of the present disclosure encode for guide RNAs targeting a coding sequence of an RNA (e.g., a TIS). In some embodiments, the engineered polynucleotides of the present disclosure encode guide RNAs targeting a non-coding sequence of an RNA (e.g., a polyA sequence). In some embodiments, the present disclosure provides compositions of one or more than one engineered polynucleotide encoding more than one engineered guide RNAs targeting the TIS, the polyA sequence, or any other part of a coding sequence or non-coding sequence. The engineered guide RNAs disclosed herein facilitate ADAR-mediated RNA editing of adenosines in the TIS, the polyA sequence, any part of a coding sequence of an RNA, any part of a non-coding sequence of an RNA, or any combination thereof.

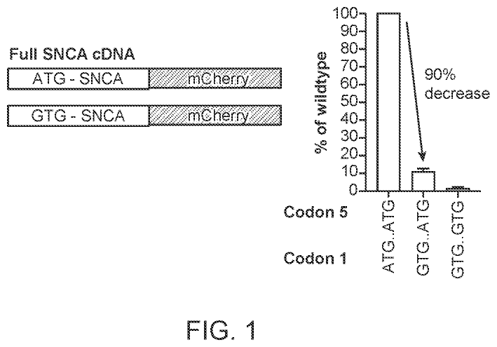

The present disclosure provides for engineered guide RNAs that facilitate SNCA RNA editing when contacted with SNCA RNA to knockdown expression of alpha-synuclein protein. Knockdown via an engineered guide RNA of the present disclosure result in a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of alpha-synuclein protein, relative to an amount prior to contacting the engineered guide RNA with the SNCA RNA. Alpha-synucleinopathies are characterized by alpha-synuclein dysfunction, overexpression and/or aggregation and are linked to neurodegenerative diseases by both genetic and neuropathological evidence. The gene encoding alpha-synuclein protein is referred to as SNCA. In Parkinson's disease (PD), SNCA genetic duplications and variants that promote alpha-synuclein aggregation (e.g., A53T) lead to early-onset and severe forms of disease. Thus, the engineered guide RNAs of the present disclosure can target SNCA for RNA editing, thereby, driving a reduction in alpha-synuclein synthesis and promoting clearance of aggregation. In some embodiments, the present disclosure provides compositions of engineered guide RNAs that target SNCA and facilitated ADAR-mediated RNA editing of SNCA to reduce pathogenic levels of alpha-synuclein by targeting key adenosines for deamination that are present in the translational initiation sites (TISs) or 3′UTR. In some embodiments, the engineered guide RNAs of the present disclosure target a coding sequence in SNCA. For example, the coding sequence can be a translation initiation site (TIS) (AUG) of SNCA and the engineered guide RNA can facilitate ADAR-mediated RNA editing of AUG to GUG. For example, as shown in FIG. 1, hardwired A to G mutations of the TIS in Codon 1 result in a ˜90% reduction in alpha-synuclein protein levels and translation is nearly completely abrogated in hardwired A to G mutations of the TISs (ATG to GTG) in Codon 1 and Codon 5. Thus, engineered guide RNAs of the present disclosure targeting these sites in SNCA are capable of facilitating edits that result in inhibition of translation and a reduction in expression of the alpha-synuclein protein. In some embodiments, the TIS targeted by the engineered guide RNAs of the present disclosure is in Codon 1 of SNCA. In some embodiments, the TIS targeted by the engineered guide RNAs of the present disclosure is in Codon 5 of SNCA. In some embodiments, one or more than one engineered guide RNAs can target the TIS in Codon 1 and Codon 5. In some embodiments, the engineered guide RNAs of the present disclosure target any key adenosine in the SNCA native TIS. For example, in some embodiments, the engineered guide RNAs target the AUG at position 265 in Exon 2 of SNCA to facilitate ADAR-mediated editing to GUG, thereby hampering translation and reducing alpha-synuclein expression. In some embodiments, the engineered guide RNAs target key adenosines in the 3′UTR of SNCA to facilitate ADAR-mediated editing of an A to a G, thereby hampering translation and reducing alpha-synuclein expression. Engineered guide RNAs targeting the 3′UTR of SNCA for A to G editing can result in inhibition of mRNA export from the nucleus, thereby preventing accessibility for protein translation and resulting in reduced alpha-synuclein expression. In some embodiments, engineered guide RNAs target the 3′UTR and facilitate ADAR-mediated RNA editing of the 3′UTR, resulting in mRNA knockdown and reduced alpha-synuclein expression. Assays to determine successful RNA editing can include NGS, Sanger sequencing, qPCR, ddPCR, fluorometric Western blots, and an alpha-synuclein specific sandwich ELISA. In some embodiments, any of the engineered guide RNAs disclosed herein are packaged in an AAV vector and are virally delivered.

As disclosed herein, editing of a target sequence of an SNCA RNA by an engineered guide RNA via ADAR can be used to reduce expression of alpha-synuclein protein. A reduction in alpha-synuclein can be utilized to treat a disease of condition associated with alpha-synuclein. In some embodiments, the disease or condition is a synucleinopathy. Editing of a target SNCA RNA as described herein, with concomitant reduction in alpha-synuclein levels, can be utilized to reduce or prevent aggregation of alpha-synuclein protein. Thus, one or more symptoms associated with aggregation of alpha-synuclein (e.g. synucleinopathies) can be treated by administration of an engineered guide RNA described herein.

As disclosed herein, administration of an engineered guide RNA described herein that targets SNCA RNA to a subject can be used to treat a disease or condition associated with alpha-synuclein, including treatment one of one or more symptoms associated with the disease or condition. In some embodiments, the disease or condition can be Parkinson's disease. In some embodiments, one or more symptoms of Parkinson's disease can be treated by administration of an engineered guide RNA targeting SNCA RNA as described herein. For example, administration of an engineered guide RNA can be sufficient to reduce resting tremors, muscle stiffness, difficulty standing, difficulty walking, difficulty with bodily movements, involuntary movements, muscle rigidity, problems with coordination, rhythmic muscle contractions, slow bodily movement, bradykinesia, slow shuffling gait, or any combination thereof. In some embodiments, treatment of Parkinson's disease comprises improvement in cognitive function. For example, a subject administered an engineered guide RNA targeting SNCA of the present disclosure can display an increase in a cognitive aptitude or motor skill test, relative to performance prior to the administering. In some embodiments, a subject can show improvement in a Unified Parkinson's Disease Rating Scale (UPDRS) test, such as an MDS-UPDRS test. In some embodiments, a subject can be assessed via an imaging technique such as an MRI or CAT scan in order to monitor the progression of the disease or condition. For example, MRI imaging can be used to visualize neurons of a subject over a treatment duration to monitor the progression of treatment. In some embodiments, neuronal cells in the substantia nigra can be monitored for degradation throughout the treatment duration.

As disclosed herein, administration of an engineered guide RNA of the present disclosure can be used to reduce alpha-synuclein protein levels through knockdownin order to treat a disease or condition associated with alpha-synuclein. While a reduction is obtained through administration, residual alpha-synuclein can still be present after the administering. In some cases, the presence of reduced alpha-synuclein protein levels trets the disease or condition, without reducing the level of alpha-synuclein protein levels to zero. Such levels can be determined in an in vitro assay using a sample obtained from a subject. In some instances, the levels can be determined in vivo using, for example, an imaging technique such as MRI as described above. Treatment can result in improvement in certain biomarkers in subject. For example, treatment can result in reduction of SNCA in CSF, reduction of SNCA in blood, reduced levels of Neurofilament A in CSF, or any combination thereof.

In some embodiments, the engineered guide RNAs target a non-coding sequence in SNCA. The non-coding sequence can be a polyA signal sequence and the engineered guide RNA can facilitate ADAR-mediated RNA editing of one or more adenosines in the polyA signal sequence of SNCA. In some embodiments, engineered guide RNAs of the present disclosure can be multiplexed to target more than one polyA signal sequences in SNCA. In some embodiments, engineered guide RNAs of the present disclosure can be multiplexed to target the TIS and one or more polyA signal sequences in SNCA. In some embodiments, engineered guide RNAs of the present disclosure targeting the canonical TIS at Codon 1 of Exon 2 (nucleotide position 226 of NCBI Reference Sequence: NM_000345.4) of SNCA can be multiplexed with one or more additional engineered guide RNAs targeting a different TIS of SNCA, such as the Codon 5 translation initiate site of Exon 2. Alternatively, or in addition, one or more engineered guide RNAs of the present disclosure targeting the canonical TIS at Codon 1 of Exon 2 (nucleotide position 226 of NCBI Reference Sequence: NM_000345.4) of SNCA may be multiplexed with or more engineered guide RNAs targeting a different sequence of SNCA, such as the 5′UTR region of SNCA (e.g., a Kozak sequence, an internal ribosomal entry site (IRES), or an iron response element (IRE) of the 5′ UTR). In some embodiments, engineered guide RNAs can be multiplexed to target a non-coding sequence and a coding sequence in SNCA. The engineered guide RNAs of the present disclosure facilitated ADAR-mediated RNA editing of SNCA, thereby, effecting protein knockdown. In each of these cases, the multiplexed engineered guide RNAs can be delivered together in the same viral vector or the each of the distinct engineered guide RNAs can be delivered together but in separate vectors.

The present disclosure, in some embodiments, provides engineered guide RNAs that facilitate edits at multiple adenosines. Hydrolytic deamination of multiple adenosines in an RNA can be referred to as hyper-editing. In some cases, hyper-editing can occur in cis (e.g. in an Alu element) or in trans (e.g. in a target RNA by an engineered guide RNA). In some cases, hyper-editing can comprise editing in the polyA signal sequence of the SNCA target RNA. In some cases, hyper-editing can introduce edits in at least 2 or more nucleotides of a subject target RNA. In some cases, hyper-editing can introduce at least or at most about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or at least or at most about 100 edits in a region of a target RNA. In an embodiment, hyper-editing can occur in an untranslated region, translated region, 3′UTR, 5′UTR, or any combinations thereof.

In some embodiments, the engineered guide RNAs of the present disclosure facilitate ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5% editing of a target adenosine. In some embodiments, the engineered guide RNAs of the present disclosure can facilitate at least 10% editing of a target adenosine. 15% editing of a target adenosine. In some embodiments, the engineered guide RNAs of the present disclosure can facilitate at least 20% editing of a target adenosine. In some embodiments, the engineered guide RNAs of the present disclosure can facilitate at least 25% editing of a target adenosine. In some embodiments, the engineered guide RNAs of the present disclosure can facilitate at least 30% editing of a target adenosine. In some embodiments, the engineered guide RNAs of the present disclosure can facilitate at least 35% editing of a target adenosine. In some embodiments, the engineered guide RNAs of the present disclosure can facilitate at least 40% editing of a target adenosine. In some embodiments, the engineered guide RNAs of the present disclosure can facilitate at least 45% editing of a target adenosine. In some embodiments, the engineered guide RNAs of the present disclosure can facilitate at least 50% editing of a target adenosine. In some embodiments, the engineered guide RNAs of the present disclosure can facilitate at least 55% editing of a target adenosine. In some embodiments, the engineered guide RNAs of the present disclosure can facilitate at least 60% editing of a target adenosine. In some embodiments, the engineered guide RNAs of the present disclosure can facilitate at least 65% editing of a target adenosine. In some embodiments, the engineered guide RNAs of the present disclosure can facilitate at least 70% editing of a target adenosine. In some embodiments, the engineered guide RNAs of the present disclosure can facilitate at least 75% editing of a target adenosine. In some embodiments, the engineered guide RNAs of the present disclosure can facilitate at least 80% editing of a target adenosine. In some embodiments, the engineered guide RNAs of the present disclosure can facilitate at least 85% editing of a target adenosine. In some embodiments, the engineered guide RNAs of the present disclosure can facilitate at least 90% editing of a target adenosine. In some embodiments, the engineered guide RNAs of the present disclosure can facilitate at least 95% editing of a target adenosine. In some embodiments, the engineered guide RNAs of the present disclosure can facilitate 100% editing of a target adenosine. In some embodiments, the engineered guide RNAs of the present disclosure can facilitate from 5 to 20% editing of a target adenosine. In some embodiments, the engineered guide RNAs of the present disclosure can facilitate from 20 to 40% editing of a target adenosine. In some embodiments, the engineered guide RNAs of the present disclosure can facilitate from 40 to 60% editing of a target adenosine. In some embodiments, the engineered guide RNAs of the present disclosure can facilitate from 60 to 80% editing of a target adenosine. In some embodiments, the engineered guide RNAs of the present disclosure can facilitate from 80 to 100% editing of a target adenosine. In some embodiments, the engineered guide RNAs of the present disclosure can facilitate from 60 to 80% editing of a target adenosine. In some embodiments, the engineered guide RNAs of the present disclosure can facilitate from 70 to 90% editing of a target adenosine. In some embodiments, the engineered guide RNAs of the present disclosure can facilitate at least 70% or more editing of a target adenosine. In some embodiments, the engineered guide RNAs of the present disclosure can facilitate at least 80% or more editing of a target adenosine. In some embodiments, the engineered guide RNAs of the present disclosure can facilitate up to 90% or more editing of a target adenosine. Optionally, additionally, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 30% editing of an off-target adenosine. In some embodiments, the engineered guide RNAs of the present disclosure can facilitate these levels of on-target RNA editing while maintaining less than 25% editing of an off-target adenosine. In some embodiments, the engineered guide RNAs of the present disclosure can facilitate these levels of on-target RNA editing while maintaining less than 20% editing of an off-target adenosine. In some embodiments, the engineered guide RNAs of the present disclosure can facilitate these levels of on-target RNA editing while maintaining less than 15% editing of an off-target adenosine. In some embodiments, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 9% editing of an off-target adenosine. In some embodiments, the engineered guide RNAs of the present disclosure can facilitate these levels of on-target RNA editing while maintaining less than 8% editing of an off-target adenosine. In some embodiments, the engineered guide RNAs of the present disclosure can facilitate these levels of on-target RNA editing while maintaining less than 7% editing of an off-target adenosine. In some embodiments, the engineered guide RNAs of the present disclosure can facilitate these levels of on-target RNA editing while maintaining less than 6% editing of an off-target adenosine. In some embodiments, the engineered guide RNAs of the present disclosure can facilitate these levels of on-target RNA editing while maintaining less than 5% editing of an off-target adenosine. In some embodiments, the engineered guide RNAs of the present disclosure can facilitate these levels of on-target RNA editing while maintaining less than 4% editing of an off-target adenosine. In some embodiments, the engineered guide RNAs of the present disclosure can facilitate these levels of on-target RNA editing while maintaining less than 3% editing of an off-target adenosine. In some embodiments, the engineered guide RNAs of the present disclosure can facilitate these levels of on-target RNA editing while maintaining less than 2% editing of an off-target adenosine. In some embodiments, the engineered guide RNAs of the present disclosure can facilitate these levels of on-target RNA editing while maintaining less than 1% editing of an off-target adenosine. In some embodiments, the engineered guide RNAs of the present disclosure can facilitate these levels of on-target RNA editing while maintaining 0% editing of an off-target adenosine. In some embodiments, the engineered guide RNAs of the present disclosure can facilitate at least 70% editing of a target adenosine while maintaining less than 30% editing of an off-target adenosine. In some embodiments, the engineered guide RNAs of the present disclosure can facilitate at least 70% editing of a target adenosine while maintaining less than 29% editing of an off-target adenosine. In some embodiments, the engineered guide RNAs of the present disclosure can facilitate at least 70% editing of a target adenosine while maintaining less than 28% editing of an off-target adenosine. In some embodiments, the engineered guide RNAs of the present disclosure can facilitate at least 70% editing of a target adenosine while maintaining less than 27% editing of an off-target adenosine. In some embodiments, the engineered guide RNAs of the present disclosure can facilitate at least 70% editing of a target adenosine while maintaining less than 26% editing of an off-target adenosine. In some embodiments, the engineered guide RNAs of the present disclosure can facilitate at least 70% editing of a target adenosine while maintaining less than 25% editing of an off-target adenosine. In some embodiments, the engineered guide RNAs of the present disclosure can facilitate at least 70% editing of a target adenosine while maintaining less than 24% editing of an off-target adenosine. In some embodiments, the engineered guide RNAs of the present disclosure can facilitate at least 70% editing of a target adenosine while maintaining less than 23% editing of an off-target adenosine. In some embodiments, the engineered guide RNAs of the present disclosure can facilitate at least 70% editing of a target adenosine while maintaining less than 22% editing of an off-target adenosine. In some embodiments, the engineered guide RNAs of the present disclosure can facilitate at least 70% editing of a target adenosine while maintaining less than 21% editing of an off-target adenosine. In some embodiments, the engineered guide RNAs of the present disclosure can facilitate at least 70% editing of a target adenosine while maintaining less than 20% editing of an off-target adenosine. In some embodiments, the engineered guide RNAs of the present disclosure can facilitate at least 70% editing of a target adenosine while maintaining less than 19% editing of an off-target adenosine. In some embodiments, the engineered guide RNAs of the present disclosure can facilitate at least 70% editing of a target adenosine while maintaining less than 18% editing of an off-target adenosine. In some embodiments, the engineered guide RNAs of the present disclosure can facilitate at least 70% editing of a target adenosine while maintaining less than 17% editing of an off-target adenosine. In some embodiments, the engineered guide RNAs of the present disclosure can facilitate at least 70% editing of a target adenosine while maintaining less than 16% editing of an off-target adenosine. In some embodiments, the engineered guide RNAs of the present disclosure can facilitate at least 70% editing of a target adenosine while maintaining less than 15% editing of an off-target adenosine. In some embodiments, the engineered guide RNAs of the present disclosure can facilitate at least 70% editing of a target adenosine while maintaining less than 14% editing of an off-target adenosine. In some embodiments, the engineered guide RNAs of the present disclosure can facilitate at least 70% editing of a target adenosine while maintaining less than 13% editing of an off-target adenosine. In some embodiments, the engineered guide RNAs of the present disclosure can facilitate at least 70% editing of a target adenosine while maintaining less than 12% editing of an off-target adenosine. In some embodiments, the engineered guide RNAs of the present disclosure can facilitate at least 70% editing of a target adenosine while maintaining less than 11% editing of an off-target adenosine. In some embodiments, the engineered guide RNAs of the present disclosure can facilitate at least 70% editing of a target adenosine while maintaining less than 10% editing of an off-target adenosine. In some embodiments, the engineered guide RNAs of the present disclosure can facilitate at least 70% editing of a target adenosine while maintaining less than 9% editing of an off-target adenosine. In some embodiments, the engineered guide RNAs of the present disclosure can facilitate at least 70% editing of a target adenosine while maintaining less than 8% editing of an off-target adenosine. In some embodiments, the engineered guide RNAs of the present disclosure can facilitate at least 70% editing of a target adenosine while maintaining less than 7% editing of an off-target adenosine. In some embodiments, the engineered guide RNAs of the present disclosure can facilitate at least 70% editing of a target adenosine while maintaining less than 6% editing of an off-target adenosine. In some embodiments, the engineered guide RNAs of the present disclosure can facilitate at least 70% editing of a target adenosine while maintaining less than 5% editing of an off-target adenosine. In some embodiments, the engineered guide RNAs of the present disclosure can facilitate at least 70% editing of a target adenosine while maintaining less than 4% editing of an off-target adenosine. In some embodiments, the engineered guide RNAs of the present disclosure can facilitate at least 70% editing of a target adenosine while maintaining less than 3% editing of an off-target adenosine. In some embodiments, the engineered guide RNAs of the present disclosure can facilitate at least 70% editing of a target adenosine while maintaining less than 2% editing of an off-target adenosine. In some embodiments, the engineered guide RNAs of the present disclosure can facilitate at least 70% editing of a target adenosine while maintaining less than 1% editing of an off-target adenosine. In some embodiments, the engineered guide RNAs of the present disclosure can facilitate at least 70% editing of a target adenosine while maintaining 0% editing of an off-target adenosine.

In some embodiments, the engineered guide RNAs of the present disclosure facilitate ADAR-mediated RNA editing of SNCA, which results in knockdown of protein levels. The knockdown in protein levels is quantitated as a reduction in expression of the alpha-synuclein protein. The engineered guide RNAs of the present disclosure can facilitate from 1% to 100% alpha-synuclein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% alpha-synuclein knockdown. In some embodiments, the engineered guide RNAs of the present disclosure facilitate from 30% to 60% alpha-synuclein knockdown. Alpha-synuclein knockdown can be measured by an assay comparing a sample or subject treated with the engineered guide RNA to a control sample or subject not treated with the engineered guide RNA.

An engineered guide RNA of the present disclosure can be used in a method of treating a disorder in a subject in need thereof. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., a synucleinopathy such as Parkinson's disease). In some embodiments, an engineered guide RNA of the present disclosure can be used to treat a condition associated with one or more mutations.

### Pharmaceutical Compositions

The compositions described herein (e.g., compositions comprising an engineered guide RNA or an engineered polynucleotide) can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a trigylceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

### Delivery

An engineered guide RNA of the present disclosure (such as an engineered guide RNA with a polynucleotide sequence of any one of SEQ ID NO: 12-384 as recited in Table 2 that target the SNCA Codon 1 TIS) or an engineered polynucleotide of the present disclosure (e.g., an engineered polynucleotide encoding an engineered guide RNA) can be delivered via a delivery vehicle. In some embodiments, the delivery vehicle is a vector. A vector can facilitate delivery of the engineered guide RNA into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the viral vector can be of a specific serotype. In some embodiments, the viral vector can be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, and AAVhu68 serotype, a derivative of any of these serotypes, or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding an engineered guide RNA) in an AAV vector. In some examples, methods of producing the delivery vectors described herein comprise, (a) introducing into a cell: (i) a polynucleotide comprising a promoter and an engineered guide RNA disclosed herein; and (ii) a viral genome comprising a Replication (Rep) gene and Capsid (Cap) gene that encodes a wild-type AAV capsid protein or modified version thereof, (b) expressing in the cell the wild-type AAV capsid protein or modified version thereof; (c) assembling an AAV particle; and (d) packaging the engineered guide RNA disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/6, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system comprises can be a liposome or polymeric nanoparticle. In some embodiments, the engineered polynucleotide or a non-viral vector comprising the engineered polynucleotide is delivered to a cell by hydrodynamic injection or ultrasound.

### Administration

Administration can refer to methods that can be used to enable the delivery of a composition described herein (e.g., comprising an engineered guide RNA or an engineered polynucleotide encoding the same) to the desired site of biological action. For example, an engineered guide RNA (such as an engineered guide RNA with a polynucleotide sequence of any one of SEQ ID NO: 12-384 as recited in Table 2 that targets the SNCA Codon 1 TIS) can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms can be achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat (including prevent) a disease in the subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein.

A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g., physiological pH, physiological temperature, physiological salt concentration, etc.).

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A composition comprising an engineered guide RNA, wherein: a) the engineered guide RNA, upon hybridization to a sequence of a target SNCA RNA, forms a guide-target RNA scaffold with the sequence of the target SNCA RNA; b) formation of the guide-target RNA scaffold substantially forms one or more structural features selected from the group consisting of: a bulge, an internal loop, and a hairpin; and c) the structural feature is not present within the engineered guide RNA prior to the hybridization of the engineered guide RNA to the SNCA target RNA; and d) upon hybridization of the engineered guide RNA to the sequence of the target SNCA RNA, the engineered guide RNA facilitates RNA editing of one or more target adenosines in the sequence of the target SNCA RNA by an RNA editing entity. Embodiment 2. The composition of embodiment 1, wherein the sequence of the target SNCA RNA is within the 3′ untranslated region (UTR). Embodiment 3. The composition of embodiment 1, wherein the sequence of the target SNCA RNA is within the 5′ untranslated region (UTR). Embodiment 4. The composition of embodiment 3, wherein the sequence of the target SNCA RNA in the 5′ UTR is a Kozak sequence. Embodiment 5. The composition of embodiment 3, wherein the sequence of the target SNCA RNA in the 5′ UTR is an internal ribosomal entry site (IRES). Embodiment 6. The composition of embodiment 3, wherein the sequence of the target SNCA RNA in the 5′ UTR is an iron response element (TRE). Embodiment 7. The composition of embodiment 1, wherein the sequence of the target SNCA RNA comprises a translation initiation site. Embodiment 8. The composition of embodiment 7, wherein the translation initiate site is at position 265 in SNCA Exon 2. Embodiment 9. The composition of embodiment 7, wherein the translation initiation site is the SNCA Codon 1 translation initiation site of Exon 2, the Codon 5 translation initiate site of Exon 2, or both. Embodiment 10. The composition of embodiment 7, wherein the translation initiation site is the SNCA Codon 1 translation initiation site of Exon 2. Embodiment 11. The composition of embodiment 7, wherein the translation initiation site is the SNCA Codon 1 translation initiation site of Exon 2 corresponding to position 226 of the SNCA transcript variant 1 of accession number NM_000345.4. Embodiment 12. The composition of embodiment 7, wherein the translation initiation site is the SNCA Codon 5 translation initiation site of Exon 2. Embodiment 13. The composition of any one of embodiments 9-11, wherein the one or more structural features comprises: a first 6/6 symmetric internal loop at a position selected from the group consisting of: 32, 30, 28, 26, and 24, relative to the target adenosine at position 0. Embodiment 14. The composition of embodiment 13, wherein the first 6/6 symmetric internal loop is at position 32, relative to the target adenosine at position 0. Embodiment 15. The composition of embodiment 14, wherein the one or more structural features further comprises at least one structural feature selected from the group consisting of: a 6/6 symmetric internal loop at position −6 relative to position 0, an A/C mismatch at position 0; a G/G mismatch at position 6 relative to position 0, and any combination thereof. Embodiment 16. The composition of embodiment 15, wherein the one or more structural features further comprise a 6/6 symmetric internal loop at position −6 relative to position 0, an A/C mismatch at position 0, and a G/G mismatch at position 6 relative to position 0. Embodiment 17. The composition of embodiment 15 or embodiment 16, wherein the engineered guide RNA comprises at least about: 80%, 85%, 90%, 92%, 95%, 97%, or 99% sequence identity to SEQ ID NO: 336. Embodiment 18. The composition of embodiment 17, wherein the engineered guide RNA comprises SEQ ID NO: 336. Embodiment 19. The composition of embodiment 14, wherein the one or more structural features further comprises at least one structural feature selected from the group consisting of: a 6/6 symmetric internal loop at position −6 relative to position 0, an A/C mismatch at position 0, a U/C mismatch at position 13 relative to position 0, an A/C mismatch at position 15 relative to position 0, and any combination thereof. Embodiment 20. The composition of embodiment 19, wherein the one or more structural features comprise further a 6/6 symmetric internal loop at position −6 relative to position 0, an A/C mismatch at position 0, a U/C mismatch at position 13 relative to position 0, and an A/C mismatch at position 15 relative to position 0. Embodiment 21. The composition of embodiment 19 or embodiment 20, wherein the engineered guide RNA comprises at least about: 80%, 85%, 90%, 92%, 95%, 97%, or 99% sequence identity to SEQ ID NO: 350. Embodiment 22. The composition of embodiment 21, wherein the engineered guide RNA comprises SEQ ID NO: 350. Embodiment 23. The composition of embodiment 13, wherein the first 6/6 symmetric internal loop is at position 30, relative to the target adenosine at position 0. Embodiment 24. The composition of embodiment 23, wherein the one or more structural features further comprises at least one structural feature selected from the group consisting of: a 6/6 symmetric internal loop at position −8 relative to position 0, an A/C mismatch at position 0, a G/G mismatch at position 6 relative to position 0, and any combination thereof. Embodiment 25. The composition of embodiment 24, wherein the one or more structural features further comprise a 6/6 symmetric internal loop at position −8 relative to position 0, an A/C mismatch at position 0, and a G/G mismatch at position 6 relative to position 0. Embodiment 26. The composition of embodiment 24 or embodiment 25, wherein the engineered guide RNA comprises at least about: 80%, 85%, 90%, 92%, 95%, 97%, or 99% sequence identity to SEQ ID NO: 293. Embodiment 27. The composition of embodiment 26, wherein the engineered guide RNA comprises SEQ ID NO: 293. Embodiment 28. The composition of embodiment 23, wherein the one or more structural features further comprises at least one structural feature selected from the group consisting of: a 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof. Embodiment 29. The composition of embodiment 28, wherein the one or more structural features further comprise a 6/6 symmetric internal loop at position −18 relative to position 0, a 3/3 symmetric bulge at position −6 relative to position 0, an A/C mismatch at position 0, a G/G mismatch at position 6 relative to position 0, and a U/C mismatch at position 10 relative to position 0. Embodiment 30. The composition of embodiment 28 or embodiment 29, wherein the engineered guide RNA comprises at least about: 80%, 85%, 90%, 92%, 95%, 97%, or 99% sequence identity to SEQ ID NO: 303. Embodiment 31. The composition of embodiment 30, wherein the engineered guide RNA comprises SEQ ID NO: 303. Embodiment 32. The composition of embodiment 23, wherein the one or more structural features further comprises at least one structural feature selected from the group consisting of: a 6/6 symmetric internal loop at position −10 relative to position 0, an A/C mismatch at position 0, a 2/2 symmetric bulge at position 4 relative to position 0, a C/C mismatch at position 11 relative to position 0, and any combination thereof. Embodiment 33. The composition of embodiment 32, wherein the one or more structural features further comprise a 6/6 symmetric internal loop at position −10 relative to position 0, an A/C mismatch at position 0, a 2/2 symmetric bulge at position 4 relative to position 0, and a C/C mismatch at position 11 relative to position 0. Embodiment 34. The composition of embodiment 32 or embodiment 33, wherein the engineered guide RNA comprises at least about: 80%, 85%, 90%, 92%, 95%, 97%, or 99% sequence identity to SEQ ID NO: 306. Embodiment 35. The composition of embodiment 34, wherein the engineered guide RNA comprises SEQ ID NO: 306. Embodiment 36. The composition of embodiment 23, wherein the one or more structural features further comprises at least one structural feature selected from the group consisting of: a 6/6 symmetric internal loop at position −14 relative to position 0, a 4/4 symmetric bulge at position −5 relative to position 0, an A/C mismatch at position 0, a A/A mismatch at position 4 relative to position 0, and any combination thereof. Embodiment 37. The composition of embodiment 36, wherein the one or more structural features further comprise a 6/6 symmetric internal loop at position −14 relative to position 0, a 4/4 symmetric bulge at position −5 relative to position 0, an A/C mismatch at position 0, and a A/A mismatch at position 4 relative to position 0. Embodiment 38. The composition of embodiment 36 or embodiment 37, wherein the engineered guide RNA comprises at least about: 80%, 85%, 90%, 92%, 95%, 97%, or 99% sequence identity to SEQ ID NO: 309. Embodiment 39. The composition of embodiment 38, wherein the engineered guide RNA comprises SEQ ID NO: 309. Embodiment 40. The composition of embodiment 23, wherein the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof. Embodiment 41. The composition of embodiment 40, wherein the one or more structural features further comprise a 6/6 symmetric internal loop at position −6 relative to position 0, an A/C mismatch at position 0, and a 2/2 symmetric bulge at position 5 relative to position 0. Embodiment 42. The composition of embodiment 40 or embodiment 41, wherein the engineered guide RNA comprises at least about: 80%, 85%, 90%, 92%, 95%, 97%, or 99% sequence identity to SEQ ID NO: 315. Embodiment 43. The composition of embodiment 42, wherein the engineered guide RNA comprises SEQ ID NO: 315. Embodiment 44. The composition of embodiment 23, wherein the one or more structural features further comprises at least one structural feature selected from the group consisting of: a 6/6 symmetric internal loop at position −6 relative to position 0, an A/C mismatch at position 0, a C/U mismatch at position 11 relative to position 0, a G/A mismatch at position 19 relative to position 0, and any combination thereof. Embodiment 45. The composition of embodiment 44, wherein the one or more structural features further comprise a 6/6 symmetric internal loop at position −6 relative to position 0, an A/C mismatch at position 0, a C/U mismatch at position 11 relative to position 0, and a G/A mismatch at position 19 relative to position 0. Embodiment 46. The composition of embodiment 44 or embodiment 45, wherein the engineered guide RNA comprises at least about: 80%, 85%, 90%, 92%, 95%, 97%, or 99% sequence identity to SEQ ID NO: 320. Embodiment 47. The composition of embodiment 46, wherein the engineered guide RNA comprises SEQ ID NO: 320. Embodiment 48. The composition of embodiment 23, wherein the one or more structural features further comprises at least one structural feature selected from the group consisting of: a 6/6 symmetric internal loop at position −16 relative to position 0, a 1/O asymmetric bulge at position −4 relative to position 0, an A/C mismatch at position 0, a 2/2 symmetric bulge at position 5 relative to position 0, a U/G Wobble at position 7 relative to position 0, and any combination thereof. Embodiment 49. The composition of embodiment 48, wherein the one or more structural features further comprise a 6/6 symmetric internal loop at position −16 relative to position 0, a 1/O asymmetric bulge at position −4 relative to position 0, an A/C mismatch at position 0, a 2/2 symmetric bulge at position 5 relative to position 0, and a U/G Wobble at position 7 relative to position 0. Embodiment 50. The composition of embodiment 48 or embodiment 49, wherein the engineered guide RNA comprises at least about: 80%, 85%, 90%, 92%, 95%, 97%, or 99% sequence identity to SEQ ID NO: 321. Embodiment 51. The composition of embodiment 50, wherein the engineered guide RNA comprises SEQ ID NO: 321. Embodiment 52. The composition of embodiment 23, wherein the one or more structural features further comprises at least one structural feature selected from the group consisting of: a 2/0 asymmetric bulge at position −6 relative to position 0, an A/C mismatch at position 0, a U/C mismatch at position 5 relative to position 0, a A/G mismatch at position 12 relative to position 0, and any combination thereof. Embodiment 53. The composition of embodiment 52, wherein the one or more structural features further comprise a 2/0 asymmetric bulge at position −6 relative to position 0, an A/C mismatch at position 0, a U/C mismatch at position 5 relative to position 0, and a A/G mismatch at position 12 relative to position 0. Embodiment 54. The composition of embodiment 52 or embodiment 53, wherein the engineered guide RNA comprises at least about: 80%, 85%, 90%, 92%, 95%, 97%, or 99% sequence identity to SEQ ID NO: 325. Embodiment 55. The composition of embodiment 54, wherein the engineered guide RNA comprises SEQ ID NO: 325. Embodiment 56. The composition of embodiment 23, wherein the one or more structural features further comprises at least one structural feature selected from the group consisting of: a 6/6 symmetric internal loop at position −14 relative to position 0, a 2/0 asymmetric bulge at position −5 relative to position 0, an A/C mismatch at position 0, and any combination thereof. Embodiment 57. The composition of embodiment 56, wherein the one or more structural features further comprise at least one structural feature selected from the group consisting of: a 6/6 symmetric internal loop at position −14 relative to position 0, a 2/0 asymmetric bulge at position −5 relative to position 0, and an A/C mismatch at position 0. Embodiment 58. The composition of embodiment 56 or embodiment 57, wherein the engineered guide RNA comprises at least about: 80%, 85%, 90%, 92%, 95%, 97%, or 99% sequence identity to SEQ ID NO: 338. Embodiment 59. The composition of embodiment 58, wherein the engineered guide RNA comprises SEQ ID NO: 338. Embodiment 60. The composition of embodiment 23, wherein the one or more structural features further comprises at least one structural feature selected from the group consisting of: a 6/6 symmetric internal loop at position −14 relative to position 0, a U/G Wobble at position −6 relative to position 0, a 2/0 asymmetric bulge at position −3 relative to position 0, an A/C mismatch at position 0, a G/A mismatch at position 19 relative to position 0, and any combination thereof. Embodiment 61. The composition of embodiment 60, wherein the one or more structural features further comprise a 6/6 symmetric internal loop at position −14 relative to position 0, a U/G Wobble at position −6 relative to position 0, a 2/0 asymmetric bulge at position −3 relative to position 0, an A/C mismatch at position 0, and a G/A mismatch at position 19 relative to position 0. Embodiment 62. The composition of embodiment 60 or embodiment 61, wherein the engineered guide RNA comprises at least about: 80%, 85%, 90%, 92%, 95%, 97%, or 99% sequence identity to SEQ ID NO: 349. Embodiment 63. The composition of embodiment 62, wherein the engineered guide RNA comprises SEQ ID NO: 349. Embodiment 64. The composition of embodiment 13, wherein the first 6/6 symmetric internal loop is at position 28, relative to the target adenosine at position 0. Embodiment 65. The composition of embodiment 64, wherein the one or more structural features further comprises at least one structural feature selected from the group consisting of: a 6/6 symmetric internal loop at position −8 relative to position 0, an A/C mismatch at position 0, a G/U Wobble at position 2 relative to position 0, and any combination thereof. Embodiment 66. The composition of embodiment 65, wherein the one or more structural features further comprise a 6/6 symmetric internal loop at position −8 relative to position 0, an A/C mismatch at position 0, and a G/U Wobble at position 2 relative to position 0. Embodiment 67. The composition of embodiment 65 or embodiment 66, wherein the engineered guide RNA comprises at least about: 80%, 85%, 90%, 92%, 95%, 97%, or 99% sequence identity to SEQ ID NO: 318. Embodiment 68. The composition of embodiment 67, wherein the engineered guide RNA comprises SEQ ID NO: 318. Embodiment 69. The composition of embodiment 64, wherein the one or more structural features further comprises at least one structural feature selected from the group consisting of: a 6/6 symmetric internal loop at position −16 relative to position 0, a 4/1 asymmetric bulge at position −5 relative to position 0, an A/C mismatch at position 0, a G/U Wobble at position 6 relative to position 0, and any combination thereof. Embodiment 70. The composition of embodiment 69, wherein the one or more structural features further comprise a 6/6 symmetric internal loop at position −16 relative to position 0, a 4/1 asymmetric bulge at position −5 relative to position 0, an A/C mismatch at position 0, and a G/U Wobble at position 6 relative to position 0. Embodiment 71. The composition of embodiment 69 or embodiment 70, wherein the engineered guide RNA comprises at least about: 80%, 85%, 90%, 92%, 95%, 97%, or 99% sequence identity to SEQ ID NO: 319. Embodiment 72. The composition of embodiment 71, wherein the engineered guide RNA comprises SEQ ID NO: 319. Embodiment 73. The composition of embodiment 64, wherein the one or more structural features further comprises at least one structural feature selected from the group consisting of: a 6/6 symmetric internal loop at position −10 relative to position 0, an A/C mismatch at position 0, a 2/2 symmetric bulge at position 5 relative to position 0, a C/U mismatch at position 11 relative to position 0, and any combination thereof. Embodiment 74. The composition of embodiment 73, wherein the one or more structural features further comprise a 6/6 symmetric internal loop at position −10 relative to position 0, an A/C mismatch at position 0, a 2/2 symmetric bulge at position 5 relative to position 0, and a C/U mismatch at position 11 relative to position 0. Embodiment 75. The composition of embodiment 73 or embodiment 74, wherein the engineered guide RNA comprises at least about: 80%, 85%, 90%, 92%, 95%, 97%, or 99% sequence identity to SEQ ID NO: 329. Embodiment 76. The composition of embodiment 75, wherein the engineered guide RNA comprises SEQ ID NO: 329. Embodiment 77. The composition of embodiment 64, wherein the one or more structural features further comprises at least one structural feature selected from the group consisting of: a 6/6 symmetric internal loop at position −16 relative to position 0, a 2/0 asymmetric bulge at position −4 relative to position 0, an A/C mismatch at position 0, a U/C mismatch at position 7 relative to position 0, and any combination thereof. Embodiment 78. The composition of embodiment 77, wherein the one or more structural features further comprise a 6/6 symmetric internal loop at position −16 relative to position 0, a 2/0 asymmetric bulge at position −4 relative to position 0, an A/C mismatch at position 0, and a U/C mismatch at position 7 relative to position 0. Embodiment 79. The composition of embodiment 77 or embodiment 78, wherein the engineered guide RNA comprises at least about: 80%, 85%, 90%, 92%, 95%, 97%, or 99% sequence identity to SEQ ID NO: 334. Embodiment 80. The composition of embodiment 79, wherein the engineered guide RNA comprises SEQ ID NO: 334. Embodiment 81. The composition of embodiment 64, wherein the one or more structural features further comprises at least one structural feature selected from the group consisting of: a 6/6 symmetric internal loop at position −10 relative to position 0, an A/C mismatch at position 0, a U/C mismatch at position 10 relative to position 0, and any combination thereof. Embodiment 82. The composition of embodiment 81, wherein the one or more structural features further comprise a 6/6 symmetric internal loop at position −10 relative to position 0, an A/C mismatch at position 0, and a U/C mismatch at position 10 relative to position 0. Embodiment 83. The composition of embodiment 81 or embodiment 82, wherein the engineered guide RNA comprises at least about: 80%, 85%, 90%, 92%, 95%, 97%, or 99% sequence identity to SEQ ID NO: 347. Embodiment 84. The composition of embodiment 83, wherein the engineered guide RNA comprises SEQ ID NO: 347. Embodiment 85. The composition of embodiment 64, wherein the one or more structural features further comprises at least one structural feature selected from the group consisting of: an A/C mismatch at position 0, a G/G mismatch at position 6 relative to position 0, and any combination thereof. Embodiment 86. The composition of embodiment 85, wherein the one or more structural features further comprise an A/C mismatch at position 0, and a G/G mismatch at position 6 relative to position 0. Embodiment 87. The composition of embodiment 85 or embodiment 86, wherein the engineered guide RNA comprises at least about: 80%, 85%, 90%, 92%, 95%, 97%, or 99% sequence identity to SEQ ID NO: 351. Embodiment 88. The composition of embodiment 87, wherein the engineered guide RNA comprises SEQ ID NO: 351. Embodiment 89. The composition of embodiment 64, wherein the one or more structural features further comprises at least one structural feature selected from the group consisting of: a 6/6 symmetric internal loop at position −10 relative to position 0, a 2/2 symmetric bulge at position −6 relative to position 0, an A/C mismatch at position 0, and any combination thereof. Embodiment 90. The composition of embodiment 89, wherein the one or more structural features further comprise a 6/6 symmetric internal loop at position −10 relative to position 0, a 2/2 symmetric bulge at position −6 relative to position 0, and an A/C mismatch at position 0. Embodiment 91. The composition of embodiment 89 or embodiment 90, wherein the engineered guide RNA comprises at least about: 80%, 85%, 90%, 92%, 95%, 97%, or 99% sequence identity to SEQ ID NO: 353. Embodiment 92. The composition of embodiment 91, wherein the engineered guide RNA comprises SEQ ID NO: 353. Embodiment 93. The composition of embodiment 64, wherein the one or more structural features further comprises at least one structural feature selected from the group consisting of: a 6/6 symmetric internal loop at position −18 relative to position 0, a 2/0 asymmetric bulge at position −3 relative to position 0, an A/C mismatch at position 0, a 0/2 asymmetric bulge at position 18 relative to position 0, and any combination thereof. Embodiment 94. The composition of embodiment 93, wherein the one or more structural features further comprise a 6/6 symmetric internal loop at position −18 relative to position 0, a 2/0 asymmetric bulge at position −3 relative to position 0, and an A/C mismatch at position 0, a 0/2 asymmetric bulge at position 18 relative to position 0. Embodiment 95. The composition of embodiment 93 or embodiment 94, wherein the engineered guide RNA comprises at least about: 80%, 85%, 90%, 92%, 95%, 97%, or 99% sequence identity to SEQ ID NO: 355. Embodiment 96. The composition of embodiment 95, wherein the engineered guide RNA comprises SEQ ID NO: 355. Embodiment 97. The composition of embodiment 64, wherein the one or more structural features further comprises at least one structural feature selected from the group consisting of: a 6/6 symmetric internal loop at position −8 relative to position 0, a 2/1 asymmetric bulge at position −2 relative to position 0, an A/C mismatch at position 0, and any combination thereof. Embodiment 98. The composition of embodiment 97, wherein the one or more structural features further comprise a 6/6 symmetric internal loop at position −8 relative to position 0, a 2/1 asymmetric bulge at position −2 relative to position 0, and an A/C mismatch at position 0. Embodiment 99. The composition of embodiment 97 or embodiment 98, wherein the engineered guide RNA comprises at least about: 80%, 85%, 90%, 92%, 95%, 97%, or 99% sequence identity to SEQ ID NO: 357. Embodiment 100. The composition of embodiment 99, wherein the engineered guide RNA comprises SEQ ID NO: 357. Embodiment 101. The composition of embodiment 64, wherein the one or more structural features further comprises at least one structural feature selected from the group consisting of: a 6/6 symmetric internal loop at position −10 relative to position 0, a 2/1 asymmetric bulge at position −6 relative to position 0, an A/C mismatch at position 0, a U/C mismatch at position 13 relative to position 0, and any combination thereof. Embodiment 102. The composition of embodiment 101, wherein the one or more structural features further comprise a 6/6 symmetric internal loop at position −10 relative to position 0, a 2/1 asymmetric bulge at position −6 relative to position 0, an A/C mismatch at position 0, and a U/C mismatch at position 13 relative to position 0. Embodiment 103. The composition of embodiment 101 or embodiment 102, wherein the engineered guide RNA comprises at least about: 80%, 85%, 90%, 92%, 95%, 97%, or 99% sequence identity to SEQ ID NO: 359. Embodiment 104. The composition of embodiment 103, wherein the engineered guide RNA comprises SEQ ID NO: 359. Embodiment 105. The composition of embodiment 64, wherein the one or more structural features further comprises at least one structural feature selected from the group consisting of: a 6/6 symmetric internal loop at position −10 relative to position 0, a 0/1 asymmetric bulge at position −6 relative to position 0, an A/C mismatch at position 0, a A/A mismatch at position 4 relative to position 0, and any combination thereof. Embodiment 106. The composition of embodiment 105, wherein the one or more structural features further comprise a 6/6 symmetric internal loop at position −10 relative to position 0, a 0/1 asymmetric bulge at position −6 relative to position 0, an A/C mismatch at position 0, and a A/A mismatch at position 4 relative to position 0. Embodiment 107. The composition of embodiment 105 or embodiment 106, wherein the engineered guide RNA comprises at least about: 80%, 85%, 90%, 92%, 95%, 97%, or 99% sequence identity to SEQ ID NO: 361. Embodiment 108. The composition of embodiment 107, wherein the engineered guide RNA comprises SEQ ID NO: 361. Embodiment 109. The composition of embodiment 64, wherein the one or more structural features further comprises at least one structural feature selected from the group consisting of: a G/G mismatch at position −3 relative to position 0, an A/C mismatch at position 0, and any combination thereof. Embodiment 110. The composition of embodiment 109, wherein the one or more structural features further comprise a G/G mismatch at position −3 relative to position 0, and an A/C mismatch at position 0. Embodiment 111. The composition of embodiment 109 or embodiment 110, wherein the engineered guide RNA comprises at least about: 80%, 85%, 90%, 92%, 95%, 97%, or 99% sequence identity to SEQ ID NO: 363. Embodiment 112. The composition of embodiment 111, wherein the engineered guide RNA comprises SEQ ID NO: 363. Embodiment 113. The composition of embodiment 64, wherein the one or more structural features further comprises at least one structural feature selected from the group consisting of: a 6/6 symmetric internal loop at position −10 relative to position 0, a 2/0 asymmetric bulge at position −4 relative to position 0, an A/C mismatch at position 0, and any combination thereof. Embodiment 114. The composition of embodiment 113, wherein the one or more structural features further comprise a 6/6 symmetric internal loop at position −10 relative to position 0, a 2/0 asymmetric bulge at position −4 relative to position 0, and an A/C mismatch at position 0. Embodiment 115. The composition of embodiment 113 or embodiment 114, wherein the engineered guide RNA comprises at least about: 80%, 85%, 90%, 92%, 95%, 97%, or 99% sequence identity to SEQ ID NO: 365. Embodiment 116. The composition of embodiment 115, wherein the engineered guide RNA comprises SEQ ID NO: 365. Embodiment 117. The composition of embodiment 64, wherein the one or more structural features further comprises at least one structural feature selected from the group consisting of: a 6/6 symmetric internal loop at position −16 relative to position 0, a 4/3 asymmetric bulge at position −3 relative to position 0, an A/C mismatch at position 0, and any combination thereof. Embodiment 118. The composition of embodiment 117, wherein the one or more structural features further comprise a 6/6 symmetric internal loop at position −16 relative to position 0, a 4/3 asymmetric bulge at position −3 relative to position 0, and an A/C mismatch at position 0. Embodiment 119. The composition of embodiment 117 or embodiment 118, wherein the engineered guide RNA comprises at least about: 80%, 85%, 90%, 92%, 95%, 97%, or 99% sequence identity to SEQ ID NO: 366. Embodiment 120. The composition of embodiment 119, wherein the engineered guide RNA comprises SEQ ID NO: 366. Embodiment 121. The composition of embodiment 64, wherein the one or more structural features further comprises at least one structural feature selected from the group consisting of: a 6/6 symmetric internal loop at position −4 relative to position 0, an A/C mismatch at position 0, a 2/1 asymmetric bulge at position 4 relative to position 0, and any combination thereof. Embodiment 122. The composition of embodiment 121, wherein the one or more structural further comprise a 6/6 symmetric internal loop at position −4 relative to position 0, an A/C mismatch at position 0, and a 2/1 asymmetric bulge at position 4 relative to position 0. Embodiment 123. The composition of embodiment 121 or embodiment 122, wherein the engineered guide RNA comprises at least about: 80%, 85%, 90%, 92%, 95%, 97%, or 99% sequence identity to SEQ ID NO: 369. Embodiment 124. The composition of embodiment 123, wherein the engineered guide RNA comprises SEQ ID NO: 369. Embodiment 125. The composition of embodiment 64, wherein the one or more structural features further comprises at least one structural feature selected from the group consisting of: a 6/6 symmetric internal loop at position −4 relative to position 0, an A/C mismatch at position 0, a A/A mismatch at position 12 relative to position 0, and any combination thereof. Embodiment 126. The composition of embodiment 125, wherein the one or more structural features further comprise a 6/6 symmetric internal loop at position −4 relative to position 0, an A/C mismatch at position 0, and a A/A mismatch at position 12 relative to position 0. Embodiment 127. The composition of embodiment 125 or embodiment 126, wherein the engineered guide RNA comprises at least about: 80%, 85%, 90%, 92%, 95%, 97%, or 99% sequence identity to SEQ ID NO: 374. Embodiment 128. The composition of embodiment 127, wherein the engineered guide RNA comprises SEQ ID NO: 374. Embodiment 129. The composition of embodiment 64, wherein the one or more structural features further comprises at least one structural feature selected from the group consisting of: a 6/6 symmetric internal loop at position −4 relative to position 0, an A/C mismatch at position 0, a C/C mismatch at position 11 relative to position 0, and any combination thereof. Embodiment 130. The composition of embodiment 129, wherein the one or more structural features further comprise a 6/6 symmetric internal loop at position −4 relative to position 0, an A/C mismatch at position 0, and a C/C mismatch at position 11 relative to position 0. Embodiment 131. The composition of embodiment 129 or embodiment 130, wherein the engineered guide RNA comprises at least about: 80%, 85%, 90%, 92%, 95%, 97%, or 99% sequence identity to SEQ ID NO: 376. Embodiment 132. The composition of embodiment 131, wherein the engineered guide RNA comprises SEQ ID NO: 376. Embodiment 133. The composition of embodiment 64, wherein the one or more structural features further comprises at least one structural feature selected from the group consisting of: a 6/6 symmetric internal loop at position −6 relative to position 0, an A/C mismatch at position 0, a G/U Wobble at position 3 relative to position 0, a U/C mismatch at position 13 relative to position 0, and any combination thereof. Embodiment 134. The composition of embodiment 133, wherein the one or more structural features further comprise a 6/6 symmetric internal loop at position −6 relative to position 0, an A/C mismatch at position 0, a G/U Wobble at position 3 relative to position 0, and a U/C mismatch at position 13 relative to position 0. Embodiment 135. The composition of embodiment 133 or embodiment 134, wherein the engineered guide RNA comprises at least about: 80%, 85%, 90%, 92%, 95%, 97%, or 99% sequence identity to SEQ ID NO: 378. Embodiment 136. The composition of embodiment 135, wherein the engineered guide RNA comprises SEQ ID NO: 378. Embodiment 137. The composition of embodiment 64, wherein the one or more structural features further comprises at least one structural feature selected from the group consisting of: a 6/6 symmetric internal loop at position −8 relative to position 0, an A/C mismatch at position 0, a C/U mismatch at position 11 relative to position 0, and any combination thereof. Embodiment 138. The composition of embodiment 137, wherein the one or more structural features further comprise a 6/6 symmetric internal loop at position −8 relative to position 0, an A/C mismatch at position 0, and a C/U mismatch at position 11 relative to position 0. Embodiment 139. The composition of embodiment 137 or embodiment 138, wherein the engineered guide RNA comprises at least about: 80%, 85%, 90%, 92%, 95%, 97%, or 99% sequence identity to SEQ ID NO: 380. Embodiment 140. The composition of embodiment 139, wherein the engineered guide RNA comprises SEQ ID NO: 380. Embodiment 141. The composition of embodiment 64, wherein the one or more structural features further comprises at least one structural feature selected from the group consisting of: a 6/6 symmetric internal loop at position −14 relative to position 0, a 3/3 symmetric bulge at position −5 relative to position 0, an A/C mismatch at position 0, and any combination thereof. Embodiment 142. The composition of embodiment 141, wherein the one or more structural features further comprise a 6/6 symmetric internal loop at position −14 relative to position 0, a 3/3 symmetric bulge at position −5 relative to position 0, and an A/C mismatch at position 0. Embodiment 143. The composition of embodiment 141 or embodiment 142, wherein the engineered guide RNA comprises at least about: 80%, 85%, 90%, 92%, 95%, 97%, or 99% sequence identity to SEQ ID NO: 382. Embodiment 144. The composition of embodiment 143, wherein the engineered guide RNA comprises SEQ ID NO: 382. Embodiment 145. The composition of embodiment 64, wherein the one or more structural features further comprises at least one structural feature selected from the group consisting of: a 6/6 symmetric internal loop at position −10 relative to position 0, a 3/3 symmetric bulge at position −5 relative to position 0, an A/C mismatch at position 0, a U/G Wobble at position 10 relative to position 0, and any combination thereof. Embodiment 146. The composition of embodiment 145, wherein the one or more structural features further comprise a 6/6 symmetric internal loop at position −10 relative to position 0, a 3/3 symmetric bulge at position −5 relative to position 0, an A/C mismatch at position 0, and a U/G Wobble at position 10 relative to position 0. Embodiment 147. The composition of embodiment 145 or embodiment 146, wherein the engineered guide RNA comprises at least about: 80%, 85%, 90%, 92%, 95%, 97%, or 99% sequence identity to SEQ ID NO: 384. Embodiment 148. The composition of embodiment 147, wherein the engineered guide RNA comprises SEQ ID NO: 384. Embodiment 149. The composition of embodiment 13, wherein the first 6/6 symmetric internal loop is at position 26, relative to the target adenosine at position 0. Embodiment 150. The composition of embodiment 149, wherein the one or more structural features further comprises at least one structural feature selected from the group consisting of: a 6/6 symmetric internal loop at position −12 relative to position 0, a 3/2 asymmetric bulge at position −4 relative to position 0, an A/C mismatch at position 0, a U/G Wobble at position 13 relative to position 0, and any combination thereof. Embodiment 151. The composition of embodiment 150, wherein the one or more structural features further comprise a 6/6 symmetric internal loop at position −12 relative to position 0, a 3/2 asymmetric bulge at position −4 relative to position 0, an A/C mismatch at position 0, and a U/G Wobble at position 13 relative to position 0. Embodiment 152. The composition of embodiment 150 or embodiment 151, wherein the engineered guide RNA comprises at least about: 80%, 85%, 90%, 92%, 95%, 97%, or 99% sequence identity to SEQ ID NO: 299. Embodiment 153. The composition of embodiment 152, wherein the engineered guide RNA comprises SEQ ID NO: 299. Embodiment 154. The composition of embodiment 149, wherein the one or more structural features further comprises at least one structural feature selected from the group consisting of: a 6/6 symmetric internal loop at position −14 relative to position 0, an A/A mismatch at position −7 relative to position 0, an A/C mismatch at position 0, and any combination thereof. Embodiment 155. The composition of embodiment 154, wherein the one or more structural features further comprise a 6/6 symmetric internal loop at position −14 relative to position 0, an A/A mismatch at position −7 relative to position 0, and an A/C mismatch at position 0. Embodiment 156. The composition of embodiment 154 or embodiment 155, wherein the engineered guide RNA comprises at least about: 80%, 85%, 90%, 92%, 95%, 97%, or 99% sequence identity to SEQ ID NO: 312. Embodiment 157. The composition of embodiment 156, wherein the engineered guide RNA comprises SEQ ID NO: 312. Embodiment 158. The composition of embodiment 149, wherein the one or more structural features further comprises at least one structural feature selected from the group consisting of: a 6/6 symmetric internal loop at position −12 relative to position 0, a 2/1 asymmetric bulge at position −2 relative to position 0, an A/C mismatch at position 0, and any combination thereof. Embodiment 159. The composition of embodiment 158, wherein the one or more structural features further comprise a 6/6 symmetric internal loop at position −12 relative to position 0, a 2/1 asymmetric bulge at position −2 relative to position 0, and an A/C mismatch at position 0. Embodiment 160. The composition of embodiment 158 or embodiment 159, wherein the engineered guide RNA comprises at least about: 80%, 85%, 90%, 92%, 95%, 97%, or 99% sequence identity to SEQ ID NO: 323. Embodiment 161. The composition of embodiment 160, wherein the engineered guide RNA comprises SEQ ID NO: 323. Embodiment 162. The composition of embodiment 149, wherein the one or more structural features further comprises at least one structural feature selected from the group consisting of: a 6/6 symmetric internal loop at position −8 relative to position 0, a U/G Wobble at position −6 relative to position 0, an A/C mismatch at position 0, a U/U mismatch at position 9 relative to position 0, and any combination thereof. Embodiment 163. The composition of embodiment 162, wherein the one or more structural features further comprise a 6/6 symmetric internal loop at position −8 relative to position 0, a U/G Wobble at position −6 relative to position 0, an A/C mismatch at position 0, and a U/U mismatch at position 9 relative to position 0. Embodiment 164. The composition of embodiment 162 or embodiment 163, wherein the engineered guide RNA comprises at least about: 80%, 85%, 90%, 92%, 95%, 97%, or 99% sequence identity to SEQ ID NO: 327. Embodiment 165. The composition of embodiment 164, wherein the engineered guide RNA comprises SEQ ID NO: 327. Embodiment 166. The composition of embodiment 149, wherein the one or more structural features further comprises at least one structural feature selected from the group consisting of: a 6/6 symmetric internal loop at position −16 relative to position 0, a 0/1 asymmetric bulge at position −7 relative to position 0, an A/C mismatch at position 0, a C/U mismatch at position 11 relative to position 0, and any combination thereof. Embodiment 167. The composition of embodiment 166, wherein the one or more structural features further comprise a 6/6 symmetric internal loop at position −16 relative to position 0, a 0/1 asymmetric bulge at position −7 relative to position 0, an A/C mismatch at position 0, and a C/U mismatch at position 11 relative to position 0. Embodiment 168. The composition of embodiment 166 or embodiment 167, wherein the engineered guide RNA comprises at least about: 80%, 85%, 90%, 92%, 95%, 97%, or 99% sequence identity to SEQ ID NO: 341. Embodiment 169. The composition of embodiment 168, wherein the engineered guide RNA comprises SEQ ID NO: 341. Embodiment 170. The composition of embodiment 149, wherein the one or more structural features further comprises at least one structural feature selected from the group consisting of: a 6/6 symmetric internal loop at position −10 relative to position 0, a U/C mismatch at position −5 relative to position 0, an A/C mismatch at position 0, and any combination thereof. Embodiment 171. The composition of embodiment 170, wherein the one or more structural features further comprise a 6/6 symmetric internal loop at position −10 relative to position 0, a U/C mismatch at position −5 relative to position 0, and an A/C mismatch at position 0. Embodiment 172. The composition of embodiment 170 or embodiment 171, wherein the engineered guide RNA comprises at least about: 80%, 85%, 90%, 92%, 95%, 97%, or 99% sequence identity to SEQ ID NO: 343. Embodiment 173. The composition of embodiment 172, wherein the engineered guide RNA comprises SEQ ID NO: 343. Embodiment 174. The composition of embodiment 149, wherein the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof. Embodiment 175. The composition of embodiment 174, wherein the one or more structural features further comprise a 6/6 symmetric internal loop at position −6 relative to position 0, an A/C mismatch at position 0, and a 2/2 symmetric bulge at position 5 relative to position 0. Embodiment 176. The composition of embodiment 174 or embodiment 175, wherein the engineered guide RNA comprises at least about: 80%, 85%, 90%, 92%, 95%, 97%, or 99% sequence identity to SEQ ID NO: 356. Embodiment 177. The composition of embodiment 176, wherein the engineered guide RNA comprises SEQ ID NO: 356. Embodiment 178. The composition of embodiment 149, wherein the one or more structural features further comprises at least one structural feature selected from the group consisting of: a 6/6 symmetric internal loop at position −14 relative to position 0, a 3/3 symmetric bulge at position −4 relative to position 0, an A/C mismatch at position 0, and any combination thereof. Embodiment 179. The composition of embodiment 178, wherein the one or more structural features further comprise a 6/6 symmetric internal loop at position −14 relative to position 0, a 3/3 symmetric bulge at position −4 relative to position 0, and an A/C mismatch at position 0. Embodiment 180. The composition of embodiment 178 or embodiment 179, wherein the engineered guide RNA comprises at least about: 80%, 85%, 90%, 92%, 95%, 97%, or 99% sequence identity to SEQ ID NO: 367. Embodiment 181. The composition of embodiment 180, wherein the engineered guide RNA comprises SEQ ID NO: 367. Embodiment 182. The composition of embodiment 149, wherein the one or more structural features further comprises at least one structural feature selected from the group consisting of: a 6/6 symmetric internal loop at position −10 relative to position 0, a 2/2 symmetric bulge at position −5 relative to position 0, an A/C mismatch at position 0, and any combination thereof. Embodiment 183. The composition of embodiment 182, wherein the one or more structural features further comprise a 6/6 symmetric internal loop at position −10 relative to position 0, a 2/2 symmetric bulge at position −5 relative to position 0, and an A/C mismatch at position 0. Embodiment 184. The composition of embodiment 182 or embodiment 183, wherein the engineered guide RNA comprises at least about: 80%, 85%, 90%, 92%, 95%, 97%, or 99% sequence identity to SEQ ID NO: 371. Embodiment 185. The composition of embodiment 184, wherein the engineered guide RNA comprises SEQ ID NO: 371. Embodiment 186. The composition of embodiment 149, wherein the one or more structural features further comprises at least one structural feature selected from the group consisting of: a 6/6 symmetric internal loop at position −20 relative to position 0, a 4/4 symmetric bulge at position −5 relative to position 0, an A/C mismatch at position 0, a 0/1 asymmetric bulge at position 5 relative to position 0, an A/C mismatch at position 17 relative to position 0, and any combination thereof. Embodiment 187. The composition of embodiment 186, wherein the one or more structural features further comprise a 6/6 symmetric internal loop at position −20 relative to position 0, a 4/4 symmetric bulge at position −5 relative to position 0, an A/C mismatch at position 0, a 0/1 asymmetric bulge at position 5 relative to position 0, and an A/C mismatch at position 17 relative to position 0. Embodiment 188. The composition of embodiment 186 or embodiment 187, wherein the engineered guide RNA comprises at least about: 80%, 85%, 90%, 92%, 95%, 97%, or 99% sequence identity to SEQ ID NO: 373. Embodiment 189. The composition of embodiment 188, wherein the engineered guide RNA comprises SEQ ID NO: 373. Embodiment 190. The composition of embodiment 13, wherein the first 6/6 symmetric internal loop is at position 24, relative to the target adenosine at position 0. Embodiment 191. The composition of embodiment 190, wherein the one or more structural features further comprises at least one structural feature selected from the group consisting of: a 6/6 symmetric internal loop at position −6 relative to position 0, an A/C mismatch at position 0, a G/G mismatch at position 6 relative to position 0, and any combination thereof. Embodiment 192. The composition of embodiment 191, wherein the one or more structural features further comprise a 6/6 symmetric internal loop at position −6 relative to position 0, an A/C mismatch at position 0, and a G/G mismatch at position 6 relative to position 0. Embodiment 193. The composition of embodiment 191 or embodiment 192, wherein the engineered guide RNA comprises at least about: 80%, 85%, 90%, 92%, 95%, 97%, or 99% sequence identity to SEQ ID NO: 295. Embodiment 194. The composition of embodiment 193, wherein the engineered guide RNA comprises SEQ ID NO: 295. Embodiment 195. The composition of embodiment 190, wherein the one or more structural features further comprises at least one structural feature selected from the group consisting of: a 6/6 symmetric internal loop at position −18 relative to position 0, a U/C mismatch at position −5 relative to position 0, an A/C mismatch at position 0, and any combination thereof. Embodiment 196. The composition of embodiment 195, wherein the one or more structural features further comprise a 6/6 symmetric internal loop at position −18 relative to position 0, a U/C mismatch at position −5 relative to position 0, and an A/C mismatch at position 0. Embodiment 197. The composition of embodiment 195 or embodiment 196, wherein the engineered guide RNA comprises at least about: 80%, 85%, 90%, 92%, 95%, 97%, or 99% sequence identity to SEQ ID NO: 330. Embodiment 198. The composition of embodiment 197, wherein the engineered guide RNA comprises SEQ ID NO: 330. Embodiment 199. The composition of embodiment 190, wherein the one or more structural features further comprises at least one structural feature selected from the group consisting of: a 6/6 symmetric internal loop at position −14 relative to position 0, a U/C mismatch at position −5 relative to position 0, an A/C mismatch at position 0, and any combination thereof. Embodiment 200. The composition of embodiment 199, wherein the one or more structural features further comprise a 6/6 symmetric internal loop at position −14 relative to position 0, a U/C mismatch at position −5 relative to position 0, and an A/C mismatch at position 0. Embodiment 201. The composition of embodiment 199 or embodiment 200, wherein the engineered guide RNA comprises at least about: 80%, 85%, 90%, 92%, 95%, 97%, or 99% sequence identity to SEQ ID NO: 332. Embodiment 202. The composition of embodiment 201, wherein the engineered guide RNA comprises SEQ ID NO: 332. Embodiment 203. The composition of embodiment 190, wherein the one or more structural features further comprises at least one structural feature selected from the group consisting of: a 6/6 symmetric internal loop at position −8 relative to position 0, an A/C mismatch at position 0, a A/C mismatch at position 4 relative to position 0, and any combination thereof. Embodiment 204. The composition of embodiment 203, wherein the one or more structural features further comprise a 6/6 symmetric internal loop at position −8 relative to position 0, an A/C mismatch at position 0, and a A/C mismatch at position 4 relative to position 0. Embodiment 205. The composition of embodiment 203 or embodiment 204, wherein the engineered guide RNA comprises at least about: 80%, 85%, 90%, 92%, 95%, 97%, or 99% sequence identity to SEQ ID NO: 340. Embodiment 206. The composition of embodiment 205, wherein the engineered guide RNA comprises SEQ ID NO: 340. Embodiment 207. The composition of embodiment 190, wherein the one or more structural features further comprises at least one structural feature selected from the group consisting of: a 8/8 symmetric internal loop at position −6 relative to position 0, an A/C mismatch at position 0, a G/A mismatch at position 6 relative to position 0, a U/G Wobble at position 7 relative to position 1, and any combination thereof. Embodiment 208. The composition of embodiment 207, wherein the one or more structural features further comprise a 8/8 symmetric internal loop at position −6 relative to position 0, an A/C mismatch at position 0, a G/A mismatch at position 6 relative to position 0, and a U/G Wobble at position 7 relative to position 1. Embodiment 209. The composition of embodiment 207 or embodiment 208, wherein the engineered guide RNA comprises at least about: 80%, 85%, 90%, 92%, 95%, 97%, or 99% sequence identity to SEQ ID NO: 345. Embodiment 210. The composition of embodiment 209, wherein the engineered guide RNA comprises SEQ ID NO: 345. Embodiment 211. The composition of any one of embodiments 1-210, wherein the one or more structural features comprises at least a first 6/6 symmetric internal loop and at least a second 6/6 symmetric loop. Embodiment 212. The composition of any one of embodiments 1-210, wherein the one or more structural features comprises the bulge, and wherein the bulge is a symmetric bulge. Embodiment 213. The composition of any one of embodiments 1-210, wherein the one or more structural features comprises the bulge, and wherein the bulge is an asymmetric bulge. Embodiment 214. The composition of any one of embodiments 1-213, wherein the one or more structural features comprises the internal loop, and wherein the internal loop is a symmetric internal loop. Embodiment 215. The composition of any one of embodiments 1-213, wherein the one or more structural features comprises the internal loop, and wherein the internal loop is an asymmetric internal loop. Embodiment 216. The composition of any one of embodiments 1-215, wherein the guide-target RNA scaffold comprises a Wobble base pair. Embodiment 217. The composition of any one of embodiments 1-216, wherein the one or more structural features comprises the hairpin, and wherein the hairpin is a recruitment hairpin or a non-recruitment hairpin. Embodiment 218. The composition of any one of embodiments 1-217, wherein upon hybridization of the engineered guide RNA to the sequence of the target SNCA RNA, the engineered guide RNA facilitates RNA editing of one or more adenosines in the sequence of the target SNCA RNA by an RNA editing entity. Embodiment 219. The composition of embodiment 218, wherein the RNA editing entity comprises ADAR1, ADAR2, ADAR3, or any combination thereof. Embodiment 220. The composition of any one of embodiments 1-219, wherein the engineered guide RNA comprises at least 80%, at least 85%, at least 90%, at least 95%, at least 99%, or 100% sequence identity to any one of SEQ ID NO: 2-SEQ ID NO: 11. Embodiment 221. The composition of any one of embodiments 1-220, wherein the engineered guide RNA is encoded by an engineered polynucleotide. Embodiment 222. The composition of embodiment 221, wherein the engineered polynucleotide is comprised in or on a vector. Embodiment 223. The composition of embodiment 222, wherein the vector is a viral vector, and wherein the engineered polynucleotide is encapsidated in the viral vector. Embodiment 224. The composition of embodiment 223, wherein the viral vector is an adeno-associated viral (AAV) vector, a derivative thereof. Embodiment 225. The composition of embodiment 224, wherein the viral vector is an adeno-associated viral (AAV) and wherein the AAV vector is AAV1, AAV2, AAV3, AAV4, AAV5, AAV6, AAV7, AAV8, AAV9, AAV10, AAV11, or a derivative, a chimera, or a variant of any of these. Embodiment 226. The composition of any one of embodiments 224-225, wherein the AAV vector is a recombinant AAV (rAAV) vector, a hybrid AAV vector, a chimeric AAV vector, a self-complementary AAV (scAAV) vector, or any combination thereof. Embodiment 227. The composition of any one of embodiments 1-226, wherein the engineered guide RNA has at least 80%, 85%, 90%, 92%, 95%, 97%, or 99% sequence identity to any one of SEQ ID NO: 12-SEQ ID NO: 384. Embodiment 228. The composition of any one of embodiments 1-226, wherein the engineered guide RNA has a sequence of any one of SEQ ID NO: 12-SEQ ID NO 384. Embodiment 229. A pharmaceutical composition comprising: a) the composition of any one of embodiments 1-228; and b) a pharmaceutically acceptable: excipient, carrier, or diluent. Embodiment 230. A method of treating a disease or a condition in a subject in need thereof, the method comprising administering to the subject a therapeutically effective amount of the composition of any one of embodiments 1-228 or the pharmaceutical composition of embodiment 229. Embodiment 231. The method of embodiment 230, wherein the disease or condition comprises a synucleinopathy. Embodiment 232. The method of embodiment 231, wherein the synucleinopathy comprises Parkinson's disease. Embodiment 233. The method of any one of embodiments 230-232, wherein the subject is a human or a non-human animal. Embodiment 234. The method of any one of embodiments 230-233, wherein the pharmaceutical composition or the composition is in unit dose form. Embodiment 235. The method of any one of embodiments 230-234, wherein the administering is sufficient to treat one or more symptoms of the disease of condition. Embodiment 236. The method of embodiment 235, wherein the disease or condition is a synucleinopathy. Embodiment 237. The method of embodiment 236, wherein the one of more symptoms treated comprises muscle tone rigidity, bradykinesia, resting tremor, or any combination thereof. Embodiment 238. The method of embodiment 236-237, wherein the administering is sufficient to reduce aggregation of alpha-synuclein protein, relative to: (a)

a level of aggregation prior to the administering; (b) a level of accumulated aggregation in the subject in the absence of the administering; or (c) both. Embodiment 239. A method of treating Parkinson's disease in a subject in need thereof, the method comprising administering to the subject the composition of any one of embodiments 1-228 in an amount sufficient to treat the Parkinson's disease in the subject. Embodiment 240. The method of embodiment 239, wherein the administering is sufficient to treat one or more symptoms of the Parkinson's disease in the subject, relative to prior to the administering. Embodiment 241. The method of embodiment 240, wherein the one of more symptoms treated comprises muscle tone rigidity, bradykinesia, resting tremor, or any combination thereof. Embodiment 242. The method of any one of embodiments 239-241, wherein the subject after the administering displays an increased Unified Parkinson's Disease Rating Scale (UPDRS) score, relative to a UPDRS score prior to the administering. Embodiment 243. A method of editing an SNCA RNA, the method comprising contacting the SNCA RNA with the composition of any one of embodiments 1-228 and an RNA editing entity, thereby editing the SNCA RNA. Embodiment 244. The method of embodiment 243, wherein the editing comprises editing one or more adenosines within the 3′ untranslated region (UTR) of the SNCA RNA. Embodiment 245. The method of embodiment 243, wherein the editing comprises editing one or more adenosines within the 5′ untranslated region (UTR) of the SNCA RNA. Embodiment 246. The method of embodiment 243, wherein the editing comprises editing one or more adenosines of a transcription initiation site (TIS) of the SNCA RNA. Embodiment 247. The method of embodiment 246, wherein the translation initiation site is the SNCA Codon 1 translation initiation site of Exon 2, the Codon 5 translation initiate site of Exon 2, or both. Embodiment 248. The method of any one of embodiments 248-247, wherein the SNCA RNA comprises a pre-mRNA transcript of SNCA. Embodiment 249. The method of embodiment 248, wherein at least 40%, at least 50%, at least 60%, at least 70%, at least 80%, at least 85%, at least 90%, at least 95%, or at least 99% of the pre-mRNA transcripts of SNCA have at least one edit. Embodiment 250. The method of embodiment 243, wherein the editing of SNCA RNA facilitates a protein knockdown. Embodiment 251. The method of embodiment 250, wherein the protein knockdown comprises a reduction of at least 10%, relative to an amount of protein present prior to the contacting. Embodiment 252. The method of embodiment 250, wherein the protein knockdown comprises a reduction of from about 10% to about 25%, relative to an amount of protein present prior to the contacting. Embodiment 253. The method of embodiment 250, wherein the protein knockdown comprises a reduction of at least 50%, relative to an amount of protein present prior to the contacting. Embodiment 254. The method of embodiment 250, wherein the protein knockdown comprises a knockdown of alpha-synuclein. Embodiment 255. The method of any one of embodiments 250-254, wherein the knockdown is measured in an in vitro assay. Embodiment 256. The method of any one of embodiments 250-254, wherein the knockdown is measured in an in vivo assay. Embodiment 257. The method of any one of embodiments 250-254, wherein the knockdown is measured in a human subject

### EXAMPLES

The following illustrative examples are representative of embodiments of the stimulation, systems, and methods described herein and are not meant to be limiting in any way.

### Example 1

### Engineered Guide RNAs for Editing SNCA TIS

This example describes engineered guide RNAs for editing SNCA RNA to knockdown expression of the alpha-synuclein protein. Engineered guide RNAs of the present disclosure are designed to target a translation initiation site (TIS) of SNCA (e.g., in Codon 1, Codon 5, or both) RNA and facilitate ADAR-mediated RNA editing of AUG (the TIS) to GUG, thus, inhibiting SNCA translation. Editing results in knockdown of the alpha-synuclein protein. Engineered guide RNAs are packaged and delivered in AAV virus and are administered to a subject in need thereof. Upon administration to of the engineered guide RNAs, in vitro or in vivo, the engineered guide RNAs edit the SNCA TIS, thereby reducing alpha-synuclein protein expression. Upon administration to a subject having a synucleinopathy (e.g., Parkinson's disease), the engineered guide RNAs are therapeutically effective and reduce symptoms and/or cure the synucleinopathy.

### Example 2

### Engineered Guide RNAs for Editing SNCA 3′UTR

This example describes engineered guide RNAs for editing SNCA RNA to knockdown expression of the alpha-synuclein protein. Engineered guide RNAs of the present disclosure are designed to target the 3′UTR of SNCA RNA and facilitate ADAR-mediated A to G RNA editing, thus, leading to inhibited mRNA export from the nucleus and reduced SNCA translation. Upon administration to of the engineered guide RNAs, in vitro or in vivo, the engineered guide RNAs edit the SNCA 3′UTR region. Editing results in knockdown of the alpha-synuclein protein. Engineered guide RNAs are packaged and delivered in AAV virus and are administered to a subject in need thereof. Upon administration to a subject having a synucleinopathy (e.g., Parkinson's disease), the engineered guide RNAs are therapeutically effective and reduce symptoms and/or cure the synucleinopathy.

### Example 3

Engineered Guide RNAs Targeting SNCA mRNA

This example describes engineered guide RNAs that target SNCA mRNA. Self-annealing RNA structures comprising the engineered guide RNA sequences of TABLE 1 and the sequences of the regions targeted by the engineered guide RNAs were contacted with an RNA editing entity (e.g., a recombinant ADAR1 and/or ADAR2) under conditions that allow for the editing of the regions targeted by the guide RNAs. The regions targeted by the engineered guide RNAs were subsequently assessed for editing using next generation sequencing (NGS). The engineered guide RNAs of TABLE 1 showed specific editing of the A nucleotide at translation initiation start site (TIS; the A in the ATG start coding with genomic coordinates: hg38 chr4: 89835667 strand −1) of SNCA mRNA. Percent on-target editing is calculated by the following formula: the number of reads containing “G” at the target/the total number of reads. Specificity is calculated by the following formula: (percent on target editing+100)/(sum of off target editing percentage at selected off-targets sites+100).

TABLE 1
Exemplary guide RNAs that target SNCA mRNA

| SEQ | | | |
|---|---|---|---|
| ID | | | |
| NO | Guide RNA Sequence | Structural Features (target/guide) | Metrics |
| SEQ | GAAAGTACTTTGAT | 1/1 A/C mismatch at 0 position | ADAR2 on target: |
| ID | GAATACATCCACGG | 1/1 G/G mismatch at +14 position | 95.68% |
| NO: | CTAATGAATTCCTTT | 1/1 G/A mismatch at +19 position | ADAR2 |
| 2 | AC | | specificity: 1.95 |
| SEQ | GAAAGTCCTTTCAA | 4/4 symmetric bulge at −6 position | ADAR2 on target: |
| ID | GAATACATCCACGG | (UCAU-UACU) | 94.39% |
| NO: | CTATACTATTCCTTT | 1/1 A/C mismatch at 0 position | ADAR2 |
| 3 | AC | 1/1 A/A mismatch at +12 position | specificity: 1.94 |
| SEQ | GAAAGTCCTTTGATG | 1/1 A/C mismatch at 0 position | ADAR2 on target: |
| ID | CATACATCCACGGCT | 1/1 U/C mismatch at +10 position | 95.92% |
| NO: | AATGAATTCCTTTAC | 1/1 G/G mismatch at +14 position | ADAR2 |
| 4 | | | specificity: 1.93 |
| SEQ | GAAAGTCCTTTGATG | 4/4 symmetric bulge at −6 position | ADAR2 on target: |
| ID | CATACATCCACGGCT | (UCAU-UACU) | 94.10% |
| NO: | ATACTATTCCTTTAC | 1/1 A/C mismatch at 0 position | ADAR2 |
| 5 | | 1/1 U/C mismatch at +10 position | specificity: 1.93 |
| | | 1/1 G/G mismatch at +14 position | |
| SEQ | GAAAGTCCTTTGAG | 4/4 symmetric bulge at −6 position | ADAR2 on target: |
| ID | GCATACATCCACGG | (UCAU-UACU) | 88.96% |
| NO: | CTATACTATTCCTTT | 1/1 A/C mismatch at 0 position | ADAR2 |
| 6 | AC | 1/1 U/C mismatch at +10 position | specificity: 1.88 |
| | | 1/1 A/G mismatch at +12 position | |
| | | 1/1 G/G mismatch at +14 position | |
| SEQ | GAAAGTCCTTTGAG | 4/4 symmetric bulge at −6 position | ADAR2 on target: |
| ID | GAATACATCCACGG | (UCAU-UACU) | 93.74% |
| NO: | CTATACTATTCCTTT | 1/1 A/C mismatch at 0 position | ADAR2 |
| 7 | AC | 1/1 A/G mismatch at +12 position | specificity: 1.93 |
| | | 1/1 G/G mismatch at +14 position | |
| SEQ | GAAAGTCCTTTGAA | 4/4 symmetric bulge at −6 position | ADAR2 on target: |
| ID | GCATACATCCACGG | (UCAU-UACU) | 84.65% |
| NO: | CTATACTATTCCTTT | 1/1 A/C mismatch at 0 position | ADAR2 |
| 8 | AC | 1/1 U/C mismatch at +10 position | specificity: 1.84 |
| | | 1/1 A/A mismatch at +12 position | |
| | | 1/1 G/G mismatch at +14 position | |
| SEQ | GAAAGTACTTTCAC | 4/4 symmetric bulge at −6 position | ADAR2 on target: |
| ID | GAATACATCCACGG | (UCAU-UACU) | 89.43% |
| NO: | CTATACTATTCCTTT | 1/1 A/C mismatch at 0 position | ADAR2 |
| 9 | AC | 1/1 A/C mismatch at +12 position | specificity: 1.88 |
| | | 1/1 G/A mismatch at +19 position | |
| SEQ | GAAAGTCCTTTCACG | 4/4 symmetric bulge at −6 position | ADAR2 on target: |
| ID | CATACATCCACGGCT | (UCAU-UACU) | 93.20% |
| NO: | ATACTATTCCTTTAC | 1/1 A/C mismatch at 0 position | ADAR2 |
| 10 | | 1/1 U/C mismatch at +10 position | specificity: 1.93 |
| | | 1/1 A/C mismatch at +12 position | |
| SEQ | GAAAGTCCTTTCAA | 4/4 symmetric bulge at −6 position | ADAR2 on target: |
| ID | GCATACATCCACGG | (UCAU-UACU) | 90.62% |
| NO: | CTATACTATTCCTTT | 1/1 A/C mismatch at 0 position | ADAR2 |
| 11 | AC | 1/1 U/C mismatch at +10 position | specificity: 1.91 |
| | | 1/1 A/A mismatch at +12 position | |

### Example 4

### Engineered Guide RNA Compositions Targeting the SNCA Codon 1 TIS

This example describes sequences of engineered guide RNAs that target the Codon 1 TIS of Exon 2 corresponding to the canonical TIS at nucleotide position 226 of SNCA transcript variant 1 (NCBI Reference Sequence: NM_000345.4). Self-annealing RNA structures, which comprised (i) the engineered guide RNAs shown in TABLE 2 and (ii) the RNA sequences of the SNCA region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing by next generation sequencing (NGS). Engineered guide RNAs that displayed greater than 50% on-target editing of the SNCA TIS for ADAR1 and/or ADAR2, as quantified at a read depth of >200, are shown in TABLE 2. All polynucleotide sequences encoding for the engineered guide RNA of TABLE 2, are also encompassed herein, which are represented by each of the sequences shown in TABLE 2, with a T substituted for each U. For each sequence, the structural features formed in the double stranded RNA substrate upon hybridization of the guide RNA to the target SNCA RNA, are shown in the second column of TABLE 2. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, in SEQ ID NO: 2, “−33_4-4_bulge-symmetric_UUCG-ACAU” is read as a structural feature formed in a guide-target RNA scaffold (target SNCA RNA sequence hybridized to an engineered guide RNA of SEQ ID NO: 2), where
a. the structural feature starts 33 nucleotides upstream (5′) (the −33 position) from the target A (0 position) of the target RNA sequence
b. four contiguous bases from the target RNA sequence and four contiguous bases from the engineered guide RNA form the structural feature
c. the structural feature is a symmetric bulge
d. a sequence of UUCG from the target RNA side and a sequence of ACAU from the engineered guide RNA side participate in forming the symmetric bulge.

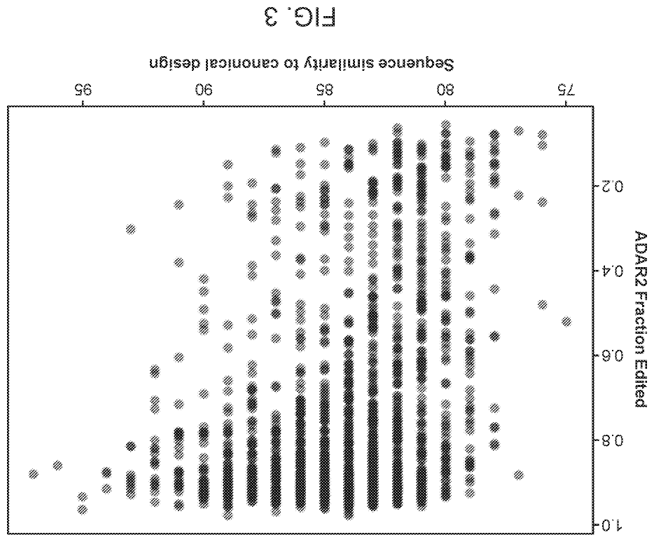

For reference, FIG. 2 can be used as an aid to visualize the structural features and the nomenclature disclosed herein. FIG. 3 is a plot showing, on the x-axis, the sequence similarity of the SNCA TIS-targeting engineered guide RNAs of the present disclosure to a canonical guide RNA design and, on the y-axis, the edited fraction by an ADAR2 enzyme. Table 2 further includes the amount of on target editing achieved via ADAR1 or ADAR2 separately, as well as ADAR1 and ADAR2. The specificity of each guide was also calculated for each engineered guide via ADAR1, ADAR2, and ADAR1+ADAR2. Specificity as provided in Table 2 was calculated using the formula: Specificity=(fraction on-target editing+1)/(sum(non-synonymous off-target editing)). These data highlight the diverse sequence space represented by the SNCA TIS-targeting engineered guide RNAs of the present disclosure, which have a range of different structural features that drive sequence diversity and which exhibit high on-target editing efficiency.

TABLE 2
Engineered Guide RNAs Targeting the SNCA Codon 1 TIS

| SEQ | | | ADAR1 | ADAR2 | | | | |
|---|---|---|---|---|---|---|---|---|
| ID | | Engineered Guide RNA | on- | on- | ADAR1/2 | ADAR1 | ADAR2 | ADAR1/2 |
| NO | Structural Features (target/guide) | Sequence | target | target | on-target | specificity | specificity | specificity |
| 12 | −33_4-4_bulge-symmetric_UUCG- | GCCACAUGAGGGUCCUUGG | 0.501 | 0.91 | 0.908 | 0.739 | 0.641 | 0.7 |
| | ACAU | CCUUUGAAAGUCCUUUCAU | | | | | | |
| | −20_4-4_bulge-symmetric_UGGU- | GAAUACAGCCACGGCUAAU | | | | | | |
| | UAGU | UGACAUGGCUUACUAGUCA | | | | | | |
| | −10_6-6_internal_loop- | CUGUCGUACAUUGGCCACU | | | | | | |
| | symmetric_AGGAAU-CAUGGC | CCCAGU | | | | | | |
| | −6_0-1_bulge-asymmetric_-U | | | | | | | |
| | 0_1-1_mismatch_A-C | | | | | | | |
| | 4_1-1_mismatch_A-G | | | | | | | |
| | 38_6-6_internal_loop- | | | | | | | |
| | symmetric_GGGAGU-UGAGGG | | | | | | | |
| 13 | −33_4-4_bulge-symmetric_UUCG- | GCCACAUGGAGGUCCUUGG | 0.506 | 0.541 | 0.725 | 0.862 | 0.579 | 0.673 |
| | ACAU | CCUUUGAAAGUCCUUUCAU | | | | | | |
| | −20_4-4_bulge-symmetric_UGGU- | GAAUACAACCACGGCGUCG | | | | | | |
| | UAGU | CUAUUCCUUUACUAGUCAC | | | | | | |
| | −4_6-6_internal_loop- | UGUCGUACAUUGGCCACUC | | | | | | |
| | symmetric_UCAUUA-GUCGCU | CCAGU | | | | | | |
| | 0_1-1_mismatch_A-C | | | | | | | |
| | 4_1-1_mismatch_A-A | | | | | | | |
| | 38_6-6_internal_loop- | | | | | | | |
| | symmetric_GGGAGU-UGGAGG | | | | | | | |
| 14 | −33_4-4_bulge-symmetric_UUCG- | GCCACACAGACCUCCUCUAC | 0.115 | 0.747 | 0.735 | 1.02 | 1.554 | 1.482 |
| | ACAU | GGUUGAAAGUCCUUUCAUG | | | | | | |
| | −20_4-4_bulge-symmetric_UGGU- | AAUACUCUCCACGGCAAUG | | | | | | |
| | UAGU | AAUGAAUUGACUAGUCACU | | | | | | |
| | −12 6-6_internal_loop- | GUCGUACAUUGGCCACUCCC | | | | | | |
| | symmetric_AAAGGA-GAAUUG | AGU | | | | | | |
| | −4_1-0_bulge-asymmetric_A- | | | | | | | |
| | 0_1-1_mismatch_A-C | | | | | | | |
| | 5_1-2_bulge-asymmetric_U-UC | | | | | | | |
| | 28_6-6_internal_loop- | | | | | | | |
| | symmetric_AGGCCA-CUACGG | | | | | | | |
| | 40_4-4_bulge-symmetric_GAGU- | | | | | | | |
| | CAGA | | | | | | | |
| 15 | −33_4-4_bulge-symmetric_UUCG- | GCCACACAGACCUCCUUGGC | 0.087 | 0.62 | 0.661 | 0.982 | 1.414 | 1.433 |
| | ACAU | AAAGCUAAGUCCUUUCAUG | | | | | | |
| | −20_4-4_bulge-symmetric_UGGU- | AAUAAUUCCACGGCUGAUC | | | | | | |
| | UAGU | CGGGGCUUUACUAGUCACU | | | | | | |
| | −8_6-6_internal_loop- | GUCGUACAUUGGCCACUCCC | | | | | | |
| | symmetric_GAAUUC-CCGGGG | AGU | | | | | | |
| | −5_1-1_wobble_U-G | | | | | | | |
| | 0_1-1_mismatch_A-C | | | | | | | |
| | 5_2-2_bulge-symmetric_UG-AU | | | | | | | |
| | 24_6-6_internal_loop- | | | | | | | |
| | symmetric_UCAAAG-AAAGCU | | | | | | | |
| | 40_4-4_bulge-symmetric_GAGU- | | | | | | | |
| | CAGA | | | | | | | |
| 16 | −33_4-4_bulge-symmetric_UUCG- | GCCACACAGACCUCGAUCUA | 0.177 | 0.758 | 0.803 | 1.047 | 1.287 | 1.412 |
| | ACAU | CUUUGAAAGUCCUUUCAUG | | | | | | |
| | −20_4-4_bulge-symmetric_UGGU- | GCUACAUCCACGGCUCAAG | | | | | | |
| | UAGU | AAUGUACAGACUAGUCACU | | | | | | |
| | −12_6-6_internal_loop- | GUCGUACAUUGGCCACUCCC | | | | | | |
| | symmetric_AAAGGA-GUACAG | AGU | | | | | | |
| | −5_3-3_bulge-symmetric_AUU-CAA | | | | | | | |
| | 0_1-1_mismatch_A-C | | | | | | | |
| | 9_1-1_mismatch_U-C | | | | | | | |
| | 10_1-1_wobble_U-G | | | | | | | |
| | 30_6-6_internal_loop- | | | | | | | |
| | symmetric_GCCAAG-GAUCUA | | | | | | | |
| | 40_4-4_bulge-symmetric_GAGU- | | | | | | | |
| | CAGA | | | | | | | |
| 17 | −33_4-4_bulge-symmetric_UUCG- | GCCACACAGACCUCCUAAAG | 0.193 | 0.739 | 0.732 | 1.052 | 1.464 | 1.42 |
| | ACAU | AGUUGAAAGUCCUUUCAUG | | | | | | |
| | −20_4-4_bulge-symmetric_UGGU- | UAUACAUCCACGACUCCUG | | | | | | |
| | UAGU | ACCAGUGUUACUAGUCACU | | | | | | |
| | −10_6-6_internal_loop- | GUCGUACAUUGGCCACUCCC | | | | | | |
| | symmetric_AGGAAU-CCAGUG | AGU | | | | | | |
| | −5_2-2_bulge-symmetric_UU-CC | | | | | | | |
| | −2_1-1_mismatch_C-A | | | | | | | |
| | 0_1-1_mismatch_A-C | | | | | | | |
| | 10_1-1_mismatch_U-U | | | | | | | |
| | 28_6-6_internal_loop- | | | | | | | |
| | symmetric_AGGCCA-AAAGAG | | | | | | | |
| | 40_4-4_bulge-symmetric_GAGU- | | | | | | | |
| | CAGA | | | | | | | |
| 18 | −33_4-4_bulge-symmetric_UUCG- | GCCACACAGACCUCCUUGUA | 0.014 | 0.809 | 0.842 | 0.943 | 1.663 | 1.643 |
| | ACAU | AGCAGAAAGUCUUUCAUGA | | | | | | |
| | −23_1-0_bulge-asymmetric_U- | UUAGCCCCACGGCUAAUGA | | | | | | |
| | −22_1-1_wobble_G-U | AUUCAUCAGAUAGUCACUG | | | | | | |
| | −21_1-1_mismatch_G-G | UCGUACAUUGGCCACUCCCA | | | | | | |
| | −19_1-1_wobble_G-U | GU | | | | | | |
| | −14_4-5_internal_loop- | | | | | | | |
| | asymmetric_AAAG-AUCAG | | | | | | | |
| | 0_1-1_mismatch_A-C | | | | | | | |
| | 4_3-3_bulge-symmetric_AUG-GCC | | | | | | | |
| | 9_1-1_mismatch_U-U | | | | | | | |
| | 19_1-0_bulge-asymmetric_G- | | | | | | | |
| | 26_6-6_internal_loop- | | | | | | | |
| | symmetric_AAAGGC-UAAGCA | | | | | | | |
| | 40_4-4_bulge-symmetric_GAGU- | | | | | | | |
| | CAGA | | | | | | | |
| 19 | −33_4-4_bulge-symmetric_UUCG- | GCCACACGAGGAUCCUUGG | 0.122 | 0.923 | 0.903 | 0.364 | 0.484 | 0.494 |
| | ACAU | CCUUUGAAAGUCCUCUCAU | | | | | | |
| | −23_0-4_bulge-asymmetric_-AAGU | GAAUACACGCACGGCUAUU | | | | | | |
| | −20_1-0_bulge-asymmetric_U- | GAAUUCCUCCCAAAGUCAC | | | | | | |
| | −16_3-0_bulge-asymmetric_UAA- | UGUCGUACAUUGGCCACUC | | | | | | |
| | −6_1-1_mismatch_U-U | CCAGU | | | | | | |
| | 0_1-1_mismatch_A-C | | | | | | | |
| | 3_2-2_bulge-symmetric_GA-CG | | | | | | | |
| | 16_1-1_mismatch_A-C | | | | | | | |
| | 38_6-6_internal_loop- | | | | | | | |
| | symmetric_GGGAGU-CGAGGA | | | | | | | |
| 20 | −33_4-4_bulge-symmetric_UUCG- | GCCACACAGACCUCCUUGAA | 0.15 | 0.911 | 0.894 | 1.02 | 1.568 | 1.523 |
| | ACAU | UGCAGAAAGUCCUUUCAUG | | | | | | |
| | −14_10−10_internal_loop- | CUUACAUCCACGGCUAAUU | | | | | | |
| | symmetric_UGGUGUAAAG- | AAUUCGACGAAUAGUCACU | | | | | | |
| | GACGAAUAGU | GUCGUACAUUGGCCACUCCC | | | | | | |
| | −8_1-1_mismatch_C-U | AGU | | | | | | |
| | 0_1-1_mismatch_A-C | | | | | | | |
| | 9_2-2_bulge-symmetric_UU-CU | | | | | | | |
| | 26_6-6_internal_loop- | | | | | | | |
| | symmetric_AAAGGC-AAUGCA | | | | | | | |
| | 40_4-4_bulge-symmetric_GAGU- | | | | | | | |
| | CAGA | | | | | | | |
| 21 | −33_4-4_bulge-symmetric_UUCG- | GCCACACAGACCUCCUUGUU | 0.027 | 0.613 | 0.652 | 0.958 | 1.446 | 1.453 |
| | ACAU | AAGGGAAAGUCCUUUCCCG | | | | | | |
| | −20_4-4_bulge-symmetric_UGGU- | AAUACAUCCACGGCUAAUU | | | | | | |
| | UAGU | CUCGACUUUACUAGUCACU | | | | | | |
| | −8_6-6_internal_loop- | GUCGUACAUUGGCCACUCCC | | | | | | |
| | symmetric_GAAUUC-UCUCGA | AGU | | | | | | |
| | 0_1-1_mismatch_A-C | | | | | | | |
| | 12_2-2_bulge-symmetric_AU-CC | | | | | | | |
| | 26_6-6_internal_loop- | | | | | | | |
| | symmetric_AAAGGC-UUAAGG | | | | | | | |
| | 40_4-4_bulge-symmetric_GAGU- | | | | | | | |
| | CAGA | | | | | | | |
| 22 | −33_4-4_bulge-symmetric_UUCG- | GCCACACAGACCUCCUGAAU | 0.287 | 0.886 | 0.882 | 1.137 | 1.628 | 1.582 |
| | ACAU | GAUUGAAAGUCCUUUCAUG | | | | | | |
| | −23_0-2_bulge-asymmetric_-GU | AAUACAUCCACGGCAUGAU | | | | | | |
| | −22_1-1_wobble_G-U | CUGAGUUACUAGUCACUGU | | | | | | |
| | −19_1-1_wobble_G-U | CGUACAUUGGCCACUCCCAG | | | | | | |
| | −15_4-4_bulge-symmetric_UAAA- | U | | | | | | |
| | GAGU | | | | | | | |
| | −14_1-1_wobble_G-U | | | | | | | |
| | −10_2-0_bulge-asymmetric_AU- | | | | | | | |
| | −4_2-0_bulge-asymmetric_UA- | | | | | | | |
| | 0_1-1_mismatch_A-C | | | | | | | |
| | 28_6-6_internal_loop- | | | | | | | |
| | symmetric_AGGCCA-GAAUGA | | | | | | | |
| | 40_4-4_bulge-symmetric_GAGU- | | | | | | | |
| | CAGA | | | | | | | |
| 23 | −33_4-4_bulge-symmetric_UUCG- | GCCACACAGACCUCCUUGUA | 0.039 | 0.932 | 0.907 | 0.92 | 1.552 | 1.456 |
| | ACAU | AUCAGAAAGUCCUUUAAUG | | | | | | |
| | −23_0-1_bulge-asymmetric_-U | AAUACCUCCACGGCUUCUCA | | | | | | |
| | −23_1-1_wobble_U-G | AUUCCUCAUUCGGUCACUG | | | | | | |
| | −22_1-1_mismatch_G-G | UCGUACAUUGGCCACUCCCA | | | | | | |
| | −20_1-1_mismatch_U-U | GU | | | | | | |
| | −19_1-1_wobble_G-U | | | | | | | |
| | −16_2-1_bulge-asymmetric_AA-C | | | | | | | |
| | −5_4-4_bulge-symmetric_CAUU- | | | | | | | |
| | UCUC | | | | | | | |
| | 0_1-1_mismatch_A-C | | | | | | | |
| | 5_1-1_mismatch_U-C | | | | | | | |
| | 14_1-1_mismatch_G-A | | | | | | | |
| | 26_6-6_internal_loop- | | | | | | | |
| | symmetric_AAAGGC-UAAUCA | | | | | | | |
| | 40_4-4_bulge-symmetric_GAGU- | | | | | | | |
| | CAGA | | | | | | | |
| 24 | −33_4-4_bulge-symmetric_UUCG- | GCCACACAGACCUCCUUGAU | 0.029 | 0.521 | 0.522 | 0.922 | 1.341 | 1.266 |
| | ACAU | ACACGAAAGUCCUUUAAUG | | | | | | |
| | −23_1-0_bulge-asymmetric_U- | AACGACAUCCACGGCUAAA | | | | | | |
| | −22_1-1_wobble_G-U | GAAUUCAUUGUAUAGUCAC | | | | | | |
| | −21_1-1_mismatch_G-G | UGUCGUACAUUGGCCACUC | | | | | | |
| | −19_1-1_wobble_G-U | CCAGU | | | | | | |
| | −14_3-4_bulge-asymmetric_AAG- | | | | | | | |
| | AUUG | | | | | | | |
| | −7_1-1_mismatch_A-A | | | | | | | |
| | 0_1-1_mismatch_A-C | | | | | | | |
| | 8_1-2_bulge-asymmetric_A-CG | | | | | | | |
| | 14_1-1_mismatch_G-A | | | | | | | |
| | 26_6-6_internal_loop- | | | | | | | |
| | symmetric_AAAGGC-AUACAC | | | | | | | |
| | 40_4-4_bulge-symmetric_GAGU- | | | | | | | |
| | CAGA | | | | | | | |
| 25 | −33_4-4_bulge-symmetric_UUCG- | GCCACACAGACCAGGAUCGC | 0.114 | 0.928 | 0.923 | 0.951 | 1.498 | 1.475 |
| | ACAU | CUUUGAAAGUCCUUCCCUG | | | | | | |
| | −20_4-4_bulge-symmetric_UGGU- | AAUACAUCCACGGCUACGA | | | | | | |
| | UAGU | CAGUCCUUUACUAGUCACU | | | | | | |
| | −6_6-6_internal_loop- | GUCGUACAUUGGCCACUCCC | | | | | | |
| | symmetric_AUUCAU-CGACAG | AGU | | | | | | |
| | 0_1-1_mismatch_A-C | | | | | | | |
| | 13_1-1_mismatch_U-C | | | | | | | |
| | 15_1-1_mismatch_A-C | | | | | | | |
| | 32_6-6_internal_loop- | | | | | | | |
| | symmetric_CAAGGA-AGGAUC | | | | | | | |
| | 40_4-4_bulge-symmetric_GAGU- | | | | | | | |
| | CAGA | | | | | | | |
| 26 | −33_4-4_bulge-symmetric_UUCG- | GCCACACAGACCUCAUUCGA | 0.064 | 0.704 | 0.79 | 0.951 | 1.449 | 1.527 |
| | ACAU | CUUUGAAAGUCCUUUCAGG | | | | | | |
| | −20_4-4_bulge-symmetric_UGGU- | AAUAACUCCACGGCACGGU | | | | | | |
| | UAGU | CAUUCCUUUACUAGUCACU | | | | | | |
| | −4_6-6_internal_loop- | GUCGUACAUUGGCCACUCCC | | | | | | |
| | symmetric_UCAUUA-ACGGUC | AGU | | | | | | |
| | 0_1-1_mismatch_A-C | | | | | | | |
| | 5_2-2_bulge-symmetric_UG-AC | | | | | | | |
| | 12_1-1_mismatch_A-G | | | | | | | |
| | 30_6-6_internal_loop- | | | | | | | |
| | symmetric_GCCAAG-AUUCGA | | | | | | | |
| | 40_4-4_bulge-symmetric_GAGU- | | | | | | | |
| | CAGA | | | | | | | |
| 27 | −33_4-4_bulge-symmetric_UUCG- | GCCACACAGACCUCCUUGAU | 0.026 | 0.94 | 0.932 | 0.936 | 1.755 | 1.707 |
| | ACAU | AGCAGAAAGUCAAUUUAUG | | | | | | |
| | −20_4-4_bulge-symmetric_UGGU- | AAUACAUCCACGGCUAUUU | | | | | | |
| | UAGU | AUGUCCUUUACUAGUCACU | | | | | | |
| | −6_6-6_internal_loop- | GUCGUACAUUGGCCACUCCC | | | | | | |
| | symmetric_AUUCAU-UUUAUG | AGU | | | | | | |
| | 0_1-1_mismatch_A-C | | | | | | | |
| | 14_1-1_wobble_G-U | | | | | | | |
| | 17_2-2_bulge-symmetric_AG-AA | | | | | | | |
| | 26_6-6_internal_loop- | | | | | | | |
| | symmetric_AAAGGC-AUAGCA | | | | | | | |
| | 40_4-4_bulge-symmetric_GAGU- | | | | | | | |
| | CAGA | | | | | | | |
| 28 | −33_4-4_bulge-symmetric_UUCG- | GCCACACAGACCUCCUCUUU | 0.025 | 0.853 | 0.867 | 0.931 | 1.63 | 1.605 |
| | ACAU | UGUUGAAAGUAGGUUCAUG | | | | | | |
| | −20_4-4_bulge-symmetric_UGGU- | ACACAUCCACGGCUACGUU | | | | | | |
| | UAGU | UCUCCUUUACUAGUCACUG | | | | | | |
| | −6_6-6_internal_loop- | UCGUACAUUGGCCACUCCCA | | | | | | |
| | symmetric_AUUCAU-CGUUUC | GU | | | | | | |
| | 0_1-1_mismatch_A-C | | | | | | | |
| | 8_2-1_bulge-asymmetric_AU-C | | | | | | | |
| | 17_3-3_bulge-symmetric_AGG-AGG | | | | | | | |
| | 28_6-6_internal_loop- | | | | | | | |
| | symmetric_AGGCCA-CUUUUG | | | | | | | |
| | 40_4-4_bulge-symmetric_GAGU- | | | | | | | |
| | CAGA | | | | | | | |
| 29 | −33_4-4_bulge-symmetric_UUCG- | GCCACACAGACCUCCUAUUC | 0.017 | 0.65 | 0.662 | 0.943 | 1.441 | 1.384 |
| | ACAU | GGUUGAAAGUGUUUCAUCA | | | | | | |
| | −22_2-3_bulge-asymmetric_UG-AGU | UACAUCCACGGCUAACGAA | | | | | | |
| | −21_1-1_wobble_G-U | UUCAGAUCAUAGUCACUGU | | | | | | |
| | −14_5-4_internal_loop- | CGUACAUUGGCCACUCCCAG | | | | | | |
| | asymmetric_UAAAG-AGAU | U | | | | | | |
| | −7_1-1_mismatch_A-C | | | | | | | |
| | 0_1-1_mismatch_A-C | | | | | | | |
| | 10_2-1_bulge-asymmetric_UC-C | | | | | | | |
| | 18_2-1_bulge-asymmetric_GG-G | | | | | | | |
| | 28_6-6_internal_loop- | | | | | | | |
| | symmetric_AGGCCA-AUUCGG | | | | | | | |
| | 40_4-4_bulge-symmetric_GAGU- | | | | | | | |
| | CAGA | | | | | | | |
| 30 | −33_4-4_bulge-symmetric_UUCG- | GCCACACAGACCUCCUAUAU | 0.036 | 0.832 | 0.843 | 0.938 | 1.523 | 1.544 |
| | ACAU | UCUUGAAAGUCCUUGCAUG | | | | | | |
| | −20_4-4_bulge-symmetric_UGGU- | AAUGCAUUCACGGCUAUCA | | | | | | |
| | UAGU | GGCUCCUUUACUAGUCACU | | | | | | |
| | −6_6-6_internal_loop- | GUCGUACAUUGGCCACUCCC | | | | | | |
| | symmetric_AUUCAU-UCAGGC | AGU | | | | | | |
| | 0_1-1_mismatch_A-C | | | | | | | |
| | 3 1-1_wobble_G-U | | | | | | | |
| | 7_1-1_wobble_U-G | | | | | | | |
| | 15_1-1_mismatch_A-G | | | | | | | |
| | 28_6-6_internal_loop- | | | | | | | |
| | symmetric_AGGCCA-AUAUUC | | | | | | | |
| | 40_4-4_bulge-symmetric_GAGU- | | | | | | | |
| | CAGA | | | | | | | |
| 31 | −33_4-4_bulge-symmetric_UUCG- | GCCACACAGACCUCAUCCUG | 0.122 | 0.8 | 0.767 | 0.967 | 1.513 | 1.519 |
| | ACAU | CUUUGAAAGUCCUUUCAUG | | | | | | |
| | −23_0-2_bulge-asymmetric_-GU | AAUCUCUCCACGGCUAAGA | | | | | | |
| | −22_1-1_wobble_G-U | AAUAUUAAGACUAGUCACU | | | | | | |
| | −19_1-1_mismatch_G-G | GUCGUACAUUGGCCACUCCC | | | | | | |
| | −17_1-1_mismatch_A-A | AGU | | | | | | |
| | −11_4-3_bulge-asymmetric_GGAA- | | | | | | | |
| | AUA | | | | | | | |
| | −7_1-0_bulge-asymmetric_A- | | | | | | | |
| | 0_1-1_mismatch_A-C | | | | | | | |
| | 5_3-3_bulge-symmetric_UGU-CUC | | | | | | | |
| | 30_6-6_internal_loop- | | | | | | | |
| | symmetric_GCCAAG-AUCCUG | | | | | | | |
| | 40_4-4_bulge-symmetric_GAGU- | | | | | | | |
| | CAGA | | | | | | | |
| 32 | −33_4-4_bulge-symmetric_UUCG- | GCCACACAGGAAAACUUGG | 0.059 | 0.723 | 0.781 | 0.872 | 1.131 | 1.143 |
| | ACAU | CCUUUGAAAGUCCUUGUAU | | | | | | |
| | −20_4-4_bulge-symmetric_UGGU- | GAACCAACCACGGCUACUA | | | | | | |
| | UAGU | AGAUCCUUUACUAGUCACU | | | | | | |
| | −6_6-6_internal_loop- | GUCGUACAUUGGCCACUCCC | | | | | | |
| | symmetric_AUUCAU-CUAAGA | AGU | | | | | | |
| | 0_1-1_mismatch_A-C | | | | | | | |
| | 4_1-1_mismatch_A-A | | | | | | | |
| | 7_2-1_bulge-asymmetric_UA-C | | | | | | | |
| | 14_1-1_wobble_G-U | | | | | | | |
| | 15_1-1_mismatch_A-G | | | | | | | |
| | 36_8-8_internal_loop- | | | | | | | |
| | symmetric_GAGGGAGU- | | | | | | | |
| | CAGGAAAA | | | | | | | |
| 33 | −33_4-4_bulge-symmetric_UUCG- | GCCACACAGACCUCCUUGUA | 0.153 | 0.689 | 0.692 | 1.027 | 1.486 | 1.42 |
| | ACAU | UGACGAAAGUCCUUUCAUG | | | | | | |
| | −20_4-4_bulge-symmetric_UGGU- | AAUACAAUCCACGGCUAAU | | | | | | |
| | UAGU | GACGCGUAUUACUAGUCAC | | | | | | |
| | −10_6-6_internal_loop- | UGUCGUACAUUGGCCACUC | | | | | | |
| | symmetric_AGGAAU-CGCGUA | CCAGU | | | | | | |
| | 0_1-1_mismatch_A-C | | | | | | | |
| | 5_0-1_bulge-asymmetric_-A | | | | | | | |
| | 26_6-6_internal_loop- | | | | | | | |
| | symmetric_AAAGGC-UAUGAC | | | | | | | |
| | 40_4-4_bulge-symmetric_GAGU- | | | | | | | |
| | CAGA | | | | | | | |
| 34 | −33_4-4_bulge-symmetric_UUCG- | GCCACACAGUAUGGCUUGG | 0.155 | 0.954 | 0.941 | 0.546 | 0.736 | 0.795 |
| | ACAU | CCUUUGAAAGUCCUUUGAU | | | | | | |
| | −20_6-6_internal_loop- | GAAUACAUCCACGGCUCUU | | | | | | |
| | symmetric_UGUGGU-UGUGGU | GAAUUCCUUUACUGUGGUC | | | | | | |
| | −5_2-2_bulge-symmetric_UU-CU | UGUCGUACAUUGGCCACUC | | | | | | |
| | 0_1-1_mismatch_A-C | CCAGU | | | | | | |
| | 14_1-1_mismatch_G-G | | | | | | | |
| | 36_8-8_internal_loop- | | | | | | | |
| | symmetric_GAGGGAGU- | | | | | | | |
| | CAGUAUGG | | | | | | | |
| 35 | −33_4-4_bulge-symmetric_UUCG- | GCCACACAGACCUCCUAAUA | 0.07 | 0.932 | 0.9 | 0.955 | 1.66 | 1.583 |
| | ACAU | AGUUGAAAGUCUUUUCAUG | | | | | | |
| | −20_4-4_bulge-symmetric_UGGU- | AAUACAUUCACGGCUAAUG | | | | | | |
| | UAGU | ACGCGAAUUACUAGUCACU | | | | | | |
| | −10_6-6_internal_loop- | GUCGUACAUUGGCCACUCCC | | | | | | |
| | symmetric_AGGAAU-CGCGAA | AGU | | | | | | |
| | 0_1-1_mismatch_A-C | | | | | | | |
| | 3 1-1_wobble_G-U | | | | | | | |
| | 18_1-1_wobble_G-U | | | | | | | |
| | 28_6-6_internal_loop- | | | | | | | |
| | symmetric_AGGCCA-AAUAAG | | | | | | | |
| | 40_4-4_bulge-symmetric_GAGU- | | | | | | | |
| | CAGA | | | | | | | |
| 36 | −33_4-4_bulge-symmetric_UUCG- | GCCACAUGAGGAUCCUUGG | 0.213 | 0.733 | 0.755 | 0.69 | 0.9 | 0.859 |
| | ACAU | CCUUUGAAAGUCCUUUCAU | | | | | | |
| | −23_0-2_bulge-asymmetric_-GU | GCAUAGUUCCACGGCUAAA | | | | | | |
| | −22_1-1_wobble_G-U | GAAUGUGCGAACUAGUCAC | | | | | | |
| | −12_8-6_internal_loop- | UGUCGUACAUUGGCCACUC | | | | | | |
| | asymmetric_GUAAAGGA-GUGCGA | CCAGU | | | | | | |
| | −7_1-1_mismatch_A-A | | | | | | | |
| | 0_1-1_mismatch_A-C | | | | | | | |
| | 5_2-2_bulge-symmetric_UG-GU | | | | | | | |
| | 10_1-1_mismatch_U-C | | | | | | | |
| | 38_6-6_internal_loop- | | | | | | | |
| | symmetric_GGGAGU-UGAGGA | | | | | | | |
| 37 | −33_4-4_bulge-symmetric_UUCG- | GCCACACAGACCGGUGACGC | 0.096 | 0.935 | 0.931 | 0.808 | 1.235 | 1.212 |
| | ACAU | CUUUGAAAGUACUUUCAUU | | | | | | |
| | −21_3-4_bulge-asymmetric_UGG- | AAUACAUCCACGGCUAAUG | | | | | | |
| | AAGU | AAUUCCUGUCAAAGUCACU | | | | | | |
| | −16_3-2_bulge-asymmetric_UAA-GU | GUCGUACAUUGGCCACUCCC | | | | | | |
| | 0_1-1_mismatch_A-C | AGU | | | | | | |
| | 11_1-1_mismatch_C-U | | | | | | | |
| | 19_1-1_mismatch_G-A | | | | | | | |
| | 32_6-6_internal_loop- | | | | | | | |
| | symmetric_CAAGGA-GGUGAC | | | | | | | |
| | 40_4-4_bulge-symmetric_GAGU- | | | | | | | |
| | CAGA | | | | | | | |
| 38 | −33_4-4_bulge-symmetric_UUCG- | GCCACACAGACCUCCUAGUA | 0.26 | 0.829 | 0.859 | 1.17 | 1.624 | 1.655 |
| | ACAU | GGUUGAAAGUCCUUUCAUU | | | | | | |
| | −23_0-2_bulge-asymmetric_-GU | AAUAACUCCACGGCUAAUG | | | | | | |
| | −22_1-1_wobble_G-U | AUCUGAGUUACUAGUCACU | | | | | | |
| | −19_1-1_wobble_G-U | GUCGUACAUUGGCCACUCCC | | | | | | |
| | −15_4-4_bulge-symmetric_UAAA- | AGU | | | | | | |
| | GAGU | | | | | | | |
| | −14_1-1_wobble_G-U | | | | | | | |
| | −10_2-0_bulge-asymmetric_AU- | | | | | | | |
| | 0_1-1_mismatch_A-C | | | | | | | |
| | 5_2-2_bulge-symmetric_UG-AC | | | | | | | |
| | 11_1-1_mismatch_C-U | | | | | | | |
| | 28_6-6_internal_loop- | | | | | | | |
| | symmetric_AGGCCA-AGUAGG | | | | | | | |
| | 40_4-4_bulge-symmetric_GAGU- | | | | | | | |
| | CAGA | | | | | | | |
| 39 | −33_4-4_bulge-symmetric_UUCG- | GCCACACAGACCAAAGAUG | 0.086 | 0.919 | 0.921 | 0.812 | 1.583 | 1.529 |
| | ACAU | CCUUUGAAAGUCCUUUCAG | | | | | | |
| | −20_6-6_internal_loop- | GAAUACAUCCACGGCUAAU | | | | | | |
| | symmetric_UGUGGU-UGAGGU | GAAUUCCUUUACUGAGGUC | | | | | | |
| | 0_1-1_mismatch_A-C | UGUCGUACAUUGGCCACUC | | | | | | |
| | 12_1-1_mismatch_A-G | CCAGU | | | | | | |
| | 32_6-6_internal_loop- | | | | | | | |
| | symmetric_CAAGGA-AAAGAU | | | | | | | |
| | 40_4-4_bulge-symmetric_GAGU- | | | | | | | |
| | CAGA | | | | | | | |
| 40 | −33_4-4_bulge-symmetric_UUCG- | GCCACACAGGAAAACUUGG | 0.324 | 0.841 | 0.837 | 0.798 | 0.824 | 0.877 |
| | ACAU | CCUUUGAAAGUCCUUUCAU | | | | | | |
| | −20_4-4_bulge-symmetric_UGGU- | UAAUACAUCCACGGCUAAU | | | | | | |
| | UAGU | CCGCGGCUUUACUAGUCAC | | | | | | |
| | −8_6-6_internal_loop- | UGUCGUACAUUGGCCACUC | | | | | | |
| | symmetric_GAAUUC-CCGCGG | CCAGU | | | | | | |
| | 0_1-1_mismatch_A-C | | | | | | | |
| | 11_1-1_mismatch_C-U | | | | | | | |
| | 36_8-8_internal_loop- | | | | | | | |
| | symmetric_GAGGGAGU- | | | | | | | |
| | CAGGAAAA | | | | | | | |
| 41 | −33_4-4_bulge-symmetric_UUCG- | GCCACACAGAGGGAACUGG | 0.241 | 0.792 | 0.781 | 0.906 | 0.9 | 1.013 |
| | ACAU | CCUUUGAAAGUUCUUUCAU | | | | | | |
| | −20_4-4_bulge-symmetric_UGGU- | GAAUACAUCGCACGGCUAC | | | | | | |
| | UAGU | AAAGGUCCUUUACUAGUCA | | | | | | |
| | −6_6-6_internal_loop- | CUGUCGUACAUUGGCCACU | | | | | | |
| | symmetric_AUUCAU-CAAAGG | CCCAGU | | | | | | |
| | 0_1-1_mismatch_A-C | | | | | | | |
| | 2_0-1_bulge-asymmetric_-G | | | | | | | |
| | 19_1-1_wobble_G-U | | | | | | | |
| | 34_10−10_internal_loop- | | | | | | | |
| | symmetric_AGGAGGGAGU- | | | | | | | |
| | CAGAGGGAAC | | | | | | | |
| 42 | −33_4-4_bulge-symmetric_UUCG- | GCCACACAAGGGCGCUUGG | 0.306 | 0.817 | 0.85 | 0.571 | 0.651 | 0.689 |
| | ACAU | CCUUUGAAAGUCCUUUCAU | | | | | | |
| | −20_4-4_bulge-symmetric_UGGU- | AAAUACAUCCACGGCUAAU | | | | | | |
| | UAGU | CUUAGGCUUUACUAGUCAC | | | | | | |
| | −8_6-6_internal_loop- | UGUCGUACAUUGGCCACUC | | | | | | |
| | symmetric_GAAUUC-CUUAGG | CCAGU | | | | | | |
| | 0_1-1_mismatch_A-C | | | | | | | |
| | 11_1-1_mismatch_C-A | | | | | | | |
| | 36_8-8_internal_loop- | | | | | | | |
| | symmetric_GAGGGAGU- | | | | | | | |
| | CAAGGGCG | | | | | | | |
| 43 | −33_4-4_bulge-symmetric_UUCG- | GCCACACAGACCUCCUUGAA | 0.038 | 0.808 | 0.838 | 0.903 | 1.558 | 1.558 |
| | ACAU | UGGGGAAAGUAAAUUCAUG | | | | | | |
| | −16_8-8_internal_loop- | AAUACAUCCACGGCCGGAA | | | | | | |
| | symmetric_UGGUGUAA- | UUCCUCAGGUAGUCACUGU | | | | | | |
| | CAGGUAGU | CGUACAUUGGCCACUCCCAG | | | | | | |
| | −4_4-2_bulge-asymmetric_AUUA-CG | U | | | | | | |
| | 0_1-1_mismatch_A-C | | | | | | | |
| | 17_3-3_bulge-symmetric_AGG-AAA | | | | | | | |
| | 26_6-6_internal_loop- | | | | | | | |
| | symmetric_AAAGGC-AAUGGG | | | | | | | |
| | 40_4-4_bulge-symmetric_GAGU- | | | | | | | |
| | CAGA | | | | | | | |
| 44 | −33_4-4_bulge-symmetric_UUCG- | GCCACAUAGGGAUCCUUGG | 0.105 | 0.694 | 0.724 | 0.622 | 0.977 | 0.898 |
| | ACAU | CCUUUGAAAGUCCUUUCAG | | | | | | |
| | −20_4-4_bulge-symmetric_UGGU- | GAAUACAUCCACGGCUAAU | | | | | | |
| | UAGU | CUGCGGCUUUACUAGUCAC | | | | | | |
| | −8_6-6_internal_loop- | UGUCGUACAUUGGCCACUC | | | | | | |
| | symmetric_GAAUUC-CUGCGG | CCAGU | | | | | | |
| | 0_1-1_mismatch_A-C | | | | | | | |
| | 12_1-1_mismatch_A-G | | | | | | | |
| | 38_6-6_internal_loop- | | | | | | | |
| | symmetric_GGGAGU-UAGGGA | | | | | | | |
| 45 | −33_4-4_bulge-symmetric_UUCG- | GCCACACAGAAGAGGAUGG | 0.367 | 0.916 | 0.896 | 0.902 | 1.121 | 1.191 |
| | ACAU | CCUUUGAAAGUCCUUUCAU | | | | | | |
| | −20_4-4_bulge-symmetric_UGGU- | UAAUACAUCCACGGCUAAU | | | | | | |
| | UAGU | CUUCGACUUUACUAGUCAC | | | | | | |
| | −8_6-6_internal_loop- | UGUCGUACAUUGGCCACUC | | | | | | |
| | symmetric_GAAUUC-CUUCGA | CCAGU | | | | | | |
| | 0_1-1_mismatch_A-C | | | | | | | |
| | 11_1-1_mismatch_C-U | | | | | | | |
| | 34_10−10_internal_loop- | | | | | | | |
| | symmetric_AGGAGGGAGU- | | | | | | | |
| | CAGAAGAGGA | | | | | | | |
| 46 | −33_4-4_bulge-symmetric_UUCG- | GCCACACAGAAGCAUAUGG | 0.364 | 0.929 | 0.924 | 0.887 | 1.098 | 1.211 |
| | ACAU | CCUUUGAAAGUCCUUUCAU | | | | | | |
| | −18_6-6_internal_loop- | GAAUACAUCCACGGCUAAC | | | | | | |
| | symmetric_UGGUGU-UGGAAU | AAUUCCUUUUGGAAUCACU | | | | | | |
| | −7_2-1_bulge-asymmetric_CA-C | GUCGUACAUUGGCCACUCCC | | | | | | |
| | 0_1-1_mismatch_A-C | AGU | | | | | | |
| | 34_10−10_internal_loop- | | | | | | | |
| | symmetric_AGGAGGGAGU- | | | | | | | |
| | CAGAAGCAUA | | | | | | | |
| 47 | −33_4-4_bulge-symmetric_UUCG- | GCCACACAGACCUCCUUGUA | 0.085 | 0.897 | 0.891 | 0.926 | 1.385 | 1.27 |
| | ACAU | GGGGGAAAGUCCUUUCAUA | | | | | | |
| | −14_10−10_internal_loop- | AAGACAUCCACGGCUAAUG | | | | | | |
| | symmetric_UGGUGUAAAG- | AAUUCGCGGUGUAGUCACU | | | | | | |
| | GCGGUGUAGU | GUCGUACAUUGGCCACUCCC | | | | | | |
| | 0_1-1_mismatch_A-C | AGU | | | | | | |
| | 8_1-1_mismatch_A-G | | | | | | | |
| | 11_1-1_mismatch_C-A | | | | | | | |
| | 26_6-6_internal_loop- | | | | | | | |
| | symmetric_AAAGGC-UAGGGG | | | | | | | |
| | 40_4-4_bulge-symmetric_GAGU- | | | | | | | |
| | CAGA | | | | | | | |
| 48 | −33_4-4_bulge-symmetric_UUCG- | GCCACACAGACCUCGCGCAA | 0.277 | 0.917 | 0.896 | 1.123 | 1.616 | 1.576 |
| | ACAU | CUUUGAAAGUCCUUUCAUG | | | | | | |
| | −18_6-6_internal_loop- | CAUAGAUCCACGGCUACAU | | | | | | |
| | symmetric_UGGUGU-UGGAGU | AAUUCCUUUUGGAGUCACU | | | | | | |
| | −6_3-3_bulge-symmetric_CAU-CAU | GUCGUACAUUGGCCACUCCC | | | | | | |
| | 0_1-1_mismatch_A-C | AGU | | | | | | |
| | 6_1-1_mismatch_G-G | | | | | | | |
| | 10_1-1_mismatch_U-C | | | | | | | |
| | 30_6-6_internal_loop- | | | | | | | |
| | symmetric_GCCAAG-GCGCAA | | | | | | | |
| | 40_4-4_bulge-symmetric_GAGU- | | | | | | | |
| | CAGA | | | | | | | |
| 49 | −33_4-4_bulge-symmetric_UUCG- | GCCACACAGACCUCCUUGGC | 0.084 | 0.932 | 0.937 | 0.971 | 1.738 | 1.697 |
| | ACAU | AGCCUUAAGUCGUUUCAUG | | | | | | |
| | −23_0-1_bulge-asymmetric_-U | AAUAGAUCCACGGCUAAAA | | | | | | |
| | −23_1-1_wobble_U-G | AUUCCUGAUUCGGUCACUG | | | | | | |
| | −22_1-1_mismatch_G-G | UCGUACAUUGGCCACUCCCA | | | | | | |
| | −20_1-1_mismatch_U-U | GU | | | | | | |
| | −19_1-1_wobble_G-U | | | | | | | |
| | −16_2-1_bulge-asymmetric_AA-G | | | | | | | |
| | −7_2-1_bulge-asymmetric_CA-A | | | | | | | |
| | 0_1-1_mismatch_A-C | | | | | | | |
| | 6_1-1_mismatch_G-G | | | | | | | |
| | 18_1-1_mismatch_G-G | | | | | | | |
| | 24_6-6_internal_loop- | | | | | | | |
| | symmetric_UCAAAG-AGCCUU | | | | | | | |
| | 40_4-4_bulge-symmetric_GAGU- | | | | | | | |
| | CAGA | | | | | | | |
| 50 | −33_4-4_bulge-symmetric_UUCG- | GCCACACAGACCUCCUUGAA | 0.096 | 0.552 | 0.618 | 0.961 | 1.349 | 1.329 |
| | ACAU | GAUGGAAAGUCCUUUCAUG | | | | | | |
| | −20_4-4_bulge-symmetric_UGGU- | AAGACAUCCACGGCUAUCA | | | | | | |
| | UAGU | AAGCUUUACUAGUCACUGU | | | | | | |
| | −6_8-6_internal_loop- | CGUACAUUGGCCACUCCCAG | | | | | | |
| | asymmetric_GAAUUCAU-UCAAAG | U | | | | | | |
| | 0_1-1_mismatch_A-C | | | | | | | |
| | 8_1-1_mismatch_A-G | | | | | | | |
| | 26_6-6_internal_loop- | | | | | | | |
| | symmetric_AAAGGC-AAGAUG | | | | | | | |
| | 40_4-4_bulge-symmetric_GAGU- | | | | | | | |
| | CAGA | | | | | | | |
| 51 | −33_4-4_bulge-symmetric_UUCG- | GCCACACAGACCUCCUCUCA | 0.049 | 0.904 | 0.882 | 0.957 | 1.589 | 1.573 |
| | ACAU | UGUUGAAAGUCGGUUCAUG | | | | | | |
| | −23_1-0_bulge-asymmetric_U- | ACUACAUCCACGGCUAAUG | | | | | | |
| | −22_1-1_wobble_G-U | AAUUCAACCGAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_4-5_internal_loop- | | | | | | | |
| | asymmetric_AAAG-AACCG | | | | | | | |
| | 0_1-1_mismatch_A-C | | | | | | | |
| | 9_1-1_mismatch_U-C | | | | | | | |
| | 17_2-2_bulge-symmetric_AG-GG | | | | | | | |
| | 28_6-6_internal_loop- | | | | | | | |
| | symmetric_AGGCCA-CUCAUG | | | | | | | |
| | 40_4-4_bulge-symmetric_GAGU- | | | | | | | |
| | CAGA | | | | | | | |
| 52 | −33_4-4_bulge-symmetric_UUCG- | GCCACACAGACCUCGGACAA | 0.353 | 0.693 | 0.814 | 1.184 | 1.307 | 1.422 |
| | ACAU | CUUUGAAAGUCCUUUCAUG | | | | | | |
| | −23_0-4_bulge-asymmetric_-UAGU | AAUACAACCACGGCUCAAC | | | | | | |
| | −23_1-1_wobble_U-G | AAUUCGAACCGUAGUCACU | | | | | | |
| | −14_6-2_internal_loop- | GUCGUACAUUGGCCACUCCC | | | | | | |
| | asymmetric_GUAAAG-GA | AGU | | | | | | |
| | −5_4-4_bulge-symmetric_CAUU- | | | | | | | |
| | CAAC | | | | | | | |
| | 0_1-1_mismatch_A-C | | | | | | | |
| | 4_1-1_mismatch_A-A | | | | | | | |
| | 30_6-6_internal_loop- | | | | | | | |
| | symmetric_GCCAAG-GGACAA | | | | | | | |
| | 40_4-4_bulge-symmetric_GAGU- | | | | | | | |
| | CAGA | | | | | | | |
| 53 | −33_4-4_bulge-symmetric_UUCG- | GCCACACAGACCGGGUCAGC | 0.119 | 0.92 | 0.901 | 0.946 | 1.27 | 1.36 |
| | ACAU | CUUUGAAAGUCCGUUCAUG | | | | | | |
| | −23_0-4_bulge-asymmetric_-UAGU | AAUACAUCCACGGCUAUAG | | | | | | |
| | −23_1-1_wobble_U-G | AAUUCGCCCCGUAGUCACU | | | | | | |
| | −20_1-1_mismatch_U-C | GUCGUACAUUGGCCACUCCC | | | | | | |
| | −18_1-1_wobble_U-G | AGU | | | | | | |
| | −14_4-0_bulge-asymmetric_AAAG- | | | | | | | |
| | −6_2-2_bulge-symmetric_AU-UA | | | | | | | |
| | 0_1-1_mismatch_A-C | | | | | | | |
| | 17_1-1_mismatch_A-G | | | | | | | |
| | 32_6-6_internal_loop- | | | | | | | |
| | symmetric_CAAGGA-GGGUCA | | | | | | | |
| | 40_4-4_bulge-symmetric_GAGU- | | | | | | | |
| | CAGA | | | | | | | |
| 54 | −33_4-4_bulge-symmetric_UUCG- | GCCACACAGACCUCCUUGAU | 0.033 | 0.746 | 0.786 | 0.926 | 1.522 | 1.523 |
| | ACAU | GGAGGAAAGUAGCAUCAUG | | | | | | |
| | −20_4-4_bulge-symmetric_UGGU- | UUUACAUCCACGGCUAUGC | | | | | | |
| | UAGU | CUGUCCUUUACUAGUCACU | | | | | | |
| | −6_6-6_internal_loop- | GUCGUACAUUGGCCACUCCC | | | | | | |
| | symmetric_AUUCAU-UGCCUG | AGU | | | | | | |
| | 0_1-1_mismatch_A-C | | | | | | | |
| | 9_2-2_bulge-symmetric_UU-UU | | | | | | | |
| | 16_4-4_bulge-symmetric_AAGG- | | | | | | | |
| | AGCA | | | | | | | |
| | 26_6-6_internal_loop- | | | | | | | |
| | symmetric_AAAGGC-AUGGAG | | | | | | | |
| | 40_4-4_bulge-symmetric_GAGU- | | | | | | | |
| | CAGA | | | | | | | |
| 55 | −33_4-4_bulge-symmetric_UUCG- | GCCACACAGACCUCCUUGGC | 0.031 | 0.493 | 0.537 | 0.936 | 1.295 | 1.321 |
| | ACAU | AUACUUAAGUCCUUUCAUC | | | | | | |
| | −20_4-4_bulge-symmetric_UGGU- | AUACAUCCACGGCUAAUUU | | | | | | |
| | UAGU | UGGGCUUUACUAGUCACUG | | | | | | |
| | −8_6-6_internal_loop- | UCGUACAUUGGCCACUCCCA | | | | | | |
| | symmetric_GAAUUC-UUUGGG | GU | | | | | | |
| | 0_1-1_mismatch_A-C | | | | | | | |
| | 10_2-1_bulge-asymmetric_UC-C | | | | | | | |
| | 24_6-6_internal_loop- | | | | | | | |
| | symmetric_UCAAAG-AUACUU | | | | | | | |
| | 40_4-4_bulge-symmetric_GAGU- | | | | | | | |
| | CAGA | | | | | | | |
| 56 | −33_4-4_bulge-symmetric_UUCG- | GCCACACAGACCUCCUAUUA | 0.107 | 0.948 | 0.96 | 0.969 | 1.684 | 1.728 |
| | ACAU | GGUUGAAAGUACUUUCAUG | | | | | | |
| | −20_4-4_bulge-symmetric_UGGU- | AAUACAUCCACGGCUAAUU | | | | | | |
| | UAGU | UAAAACUUUACUAGUCACU | | | | | | |
| | −8_6-6_internal_loop- | GUCGUACAUUGGCCACUCCC | | | | | | |
| | symmetric_GAAUUC-UUAAAA | AGU | | | | | | |
| | 0_1-1_mismatch_A-C | | | | | | | |
| | 19_1-1_mismatch_G-A | | | | | | | |
| | 28_6-6_internal_loop- | | | | | | | |
| | symmetric_AGGCCA-AUUAGG | | | | | | | |
| | 40_4-4_bulge-symmetric_GAGU- | | | | | | | |
| | CAGA | | | | | | | |
| 57 | −33_4-4_bulge-symmetric_UUCG- | GCCACACAGAAAGAACUGG | 0.24 | 0.952 | 0.891 | 0.692 | 0.807 | 0.976 |
| | ACAU | CCUUUGAAAGUCCUUUCAU | | | | | | |
| | −16_8-8_internal_loop- | GAAUACACCCACGACUAAU | | | | | | |
| | symmetric_UGGUGUAA- | GAAUUCCUACCUGAGUCAC | | | | | | |
| | ACCUGAGU | UGUCGUACAUUGGCCACUC | | | | | | |
| | −2_1-1_mismatch_C-A | CCAGU | | | | | | |
| | 0_1-1_mismatch_A-C | | | | | | | |
| | 4_1-1_mismatch_A-C | | | | | | | |
| | 34_10−10_internal_loop- | | | | | | | |
| | symmetric_AGGAGGGAGU- | | | | | | | |
| | CAGAAAGAAC | | | | | | | |
| 58 | −33_4-4_bulge-symmetric_UUCG- | GCCACAUGGGAGUCCUUGG | 0.056 | 0.815 | 0.8 | 0.61 | 0.754 | 0.773 |
| | ACAU | CCUUUGAAAGUCCUUUAAU | | | | | | |
| | −20_4-4_bulge-symmetric_UGGU- | GAAUACAUCCACGGAAAUG | | | | | | |
| | UAGU | AUAUAGCUUACUAGUCACU | | | | | | |
| | −10_6-6_internal_loop- | GUCGUACAUUGGCCACUCCC | | | | | | |
| | symmetric_AGGAAU-UAUAGC | AGU | | | | | | |
| | −3_2-1_bulge-asymmetric_AG-A | | | | | | | |
| | 0_1-1_mismatch_A-C | | | | | | | |
| | 14_1-1_mismatch_G-A | | | | | | | |
| | 38_6-6_internal_loop- | | | | | | | |
| | symmetric_GGGAGU-UGGGAG | | | | | | | |
| 59 | −33_4-4_bulge-symmetric_UUCG- | GCCACACAGACCUCAGACAG | 0.132 | 0.913 | 0.908 | 0.993 | 1.431 | 1.472 |
| | ACAU | CUUUGAAAGUCCUAUCAUG | | | | | | |
| | −20_4-4_bulge-symmetric_UGGU- | AAUAGAUCCACGGCUAAUG | | | | | | |
| | UAGU | ACGGUAGUUACUAGUCACU | | | | | | |
| | −10_6-6_internal_loop- | GUCGUACAUUGGCCACUCCC | | | | | | |
| | symmetric_AGGAAU-CGGUAG | AGU | | | | | | |
| | 0_1-1_mismatch_A-C | | | | | | | |
| | 6_1-1_mismatch_G-G | | | | | | | |
| | 16_1-1_mismatch_A-A | | | | | | | |
| | 30_6-6_internal_loop- | | | | | | | |
| | symmetric_GCCAAG-AGACAG | | | | | | | |
| | 40_4-4_bulge-symmetric_GAGU- | | | | | | | |
| | CAGA | | | | | | | |
| 60 | −33_4-4_bulge-symmetric_UUCG- | GCCACAUAGCGGUCCUUGG | 0.434 | 0.916 | 0.93 | 0.679 | 0.628 | 0.641 |
| | ACAU | CCUUUGAAAGUCCUUUCAU | | | | | | |
| | −20_4-4_bulge-symmetric_UGGU- | GAAUACAACCACGGCUAAU | | | | | | |
| | UAGU | UUAGCGCUUUACUAGUCAC | | | | | | |
| | −8_6-6_internal_loop- | UGUCGUACAUUGGCCACUC | | | | | | |
| | symmetric_GAAUUC-UUAGCG | CCAGU | | | | | | |
| | 0_1-1_mismatch_A-C | | | | | | | |
| | 4_1-1_mismatch_A-A | | | | | | | |
| | 38_6-6_internal_loop- | | | | | | | |
| | symmetric_GGGAGU-UAGCGG | | | | | | | |
| 61 | −33_4-4_bulge-symmetric_UUCG- | GCCACACAGACCUCCUUGAA | 0.115 | 0.922 | 0.891 | 0.944 | 1.636 | 1.538 |
| | ACAU | GGGCGAAAGUCGUUUCAUG | | | | | | |
| | −21_3-3_bulge-symmetric_UGG-AGU | AAUACAUCCACGGCUAAAG | | | | | | |
| | −19_1-1_wobble_G-U | AAUUCCUCGUUAAGUCACU | | | | | | |
| | −16_3-3_bulge-symmetric_UAA-CGU | GUCGUACAUUGGCCACUCCC | | | | | | |
| | −7_1-1_mismatch_A-A | AGU | | | | | | |
| | 0_1-1_mismatch_A-C | | | | | | | |
| | 18_1-1_mismatch_G-G | | | | | | | |
| | 26_6-6_internal_loop- | | | | | | | |
| | symmetric_AAAGGC-AAGGGC | | | | | | | |
| | 40_4-4_bulge-symmetric_GAGU- | | | | | | | |
| | CAGA | | | | | | | |
| 62 | −33_4-4_bulge-symmetric_UUCG- | GCCACACAGACCUCGACUUA | 0.03 | 0.841 | 0.848 | 0.933 | 1.623 | 1.633 |
| | ACAU | CUUUGAAAGUCCCUCAUGA | | | | | | |
| | −16_8-8_internal_loop- | AUACAUCCACGGAAUGAAU | | | | | | |
| | symmetric_UGGUGUAA- | UCCUAGUAGAGUCACUGUC | | | | | | |
| | AGUAGAGU | GUACAUUGGCCACUCCCAG | | | | | | |
| | −3_2-0_bulge-asymmetric_AG- | U | | | | | | |
| | 0_1-1_mismatch_A-C | | | | | | | |
| | 16_2-1_bulge-asymmetric_AA-C | | | | | | | |
| | 30_6-6_internal_loop- | | | | | | | |
| | symmetric_GCCAAG-GACUUA | | | | | | | |
| | 40_4-4_bulge-symmetric_GAGU- | | | | | | | |
| | CAGA | | | | | | | |
| 63 | −33_4-4_bulge-symmetric_UUCG- | GCCACACAGACCUCCUUGGC | 0.026 | 0.921 | 0.929 | 0.923 | 1.663 | 1.636 |
| | ACAU | AGAAAUAAGUCCUUGGAUG | | | | | | |
| | −16_8-8_internal_loop- | AAUACAUCCACGGCUAAUG | | | | | | |
| | symmetric_UGGUGUAA- | AAUUCCUCGUUGAGUCACU | | | | | | |
| | CGUUGAGU | GUCGUACAUUGGCCACUCCC | | | | | | |
| | 0_1-1_mismatch_A-C | AGU | | | | | | |
| | 14_2-2_bulge-symmetric_GA-GG | | | | | | | |
| | 24_6-6_internal_loop- | | | | | | | |
| | symmetric_UCAAAG-AGAAAU | | | | | | | |
| | 40_4-4_bulge-symmetric_GAGU- | | | | | | | |
| | CAGA | | | | | | | |
| 64 | −33_4-4_bulge-symmetric_UUCG- | GCCACACAGACCUCCUUGUU | 0.024 | 0.526 | 0.581 | 0.931 | 1.335 | 1.341 |
| | ACAU | GAUGGAAAGUCCGUUCAUA | | | | | | |
| | −23_0-1_bulge-asymmetric_-U | AAUACAUCCACGACCCAUG | | | | | | |
| | −23_1-1_wobble_U-G | AAUUCCUGGGUCGGUCACU | | | | | | |
| | −22_1-1_mismatch_G-G | GUCGUACAUUGGCCACUCCC | | | | | | |
| | −16_5-4_internal_loop- | AGU | | | | | | |
| | asymmetric_UGUAA-GGGU | | | | | | | |
| | −4_2-2_bulge-symmetric_UA-CC | | | | | | | |
| | −2_1-1_mismatch_C-A | | | | | | | |
| | 0_1-1_mismatch_A-C | | | | | | | |
| | 11_1-1_mismatch_C-A | | | | | | | |
| | 17_1-1_mismatch_A-G | | | | | | | |
| | 26_6-6_internal_loop- | | | | | | | |
| | symmetric_AAAGGC-UUGAUG | | | | | | | |
| | 40_4-4_bulge-symmetric_GAGU- | | | | | | | |
| | CAGA | | | | | | | |
| 65 | −33_4-4_bulge-symmetric_UUCG- | GCCACACAGACCUCCUUGUA | 0.019 | 0.596 | 0.662 | 0.924 | 1.368 | 1.408 |
| | ACAU | GGUAGAAAGUCCGAGCAGG | | | | | | |
| | −23_0-1_bulge-asymmetric_-U | AAUACAUCCACGGCAAUGA | | | | | | |
| | −23_1-1_wobble_U-G | AUUCCUAGUUCGGUCACUG | | | | | | |
| | −22_1-1_mismatch_G-G | UCGUACAUUGGCCACUCCCA | | | | | | |
| | −16_5-4_internal_loop- | GU | | | | | | |
| | asymmetric_UGUAA-AGUU | | | | | | | |
| | −4_1-0_bulge-asymmetric_A- | | | | | | | |
| | 0_1-1_mismatch_A-C | | | | | | | |
| | 12_1-1_mismatch_A-G | | | | | | | |
| | 15_3-3_bulge-symmetric_AAA-GAG | | | | | | | |
| | 26_6-6_internal_loop- | | | | | | | |
| | symmetric_AAAGGC-UAGGUA | | | | | | | |
| | 40_4-4_bulge-symmetric_GAGU- | | | | | | | |
| | CAGA | | | | | | | |
| 66 | −33_4-4_bulge-symmetric_UUCG- | GCCACACAGAAAAACUUGG | 0.046 | 0.857 | 0.896 | 0.87 | 1.082 | 1.243 |
| | ACAU | CCUUUGAAAGUCCUUUUAU | | | | | | |
| | −20_4-4_bulge-symmetric_UGGU- | GCCUACUUCCACGGCUACGU | | | | | | |
| | UAGU | CAGUCCUUUACUAGUCACU | | | | | | |
| | −6_6-6_internal_loop- | GUCGUACAUUGGCCACUCCC | | | | | | |
| | symmetric_AUUCAU-CGUCAG | AGU | | | | | | |
| | 0_1-1_mismatch_A-C | | | | | | | |
| | 5_1-1_mismatch_U-U | | | | | | | |
| | 9_2-2_bulge-symmetric_UU-CC | | | | | | | |
| | 14_1-1_wobble_G-U | | | | | | | |
| | 36_8-8_internal_loop- | | | | | | | |
| | symmetric_GAGGGAGU- | | | | | | | |
| | CAGAAAAA | | | | | | | |
| 67 | −33_4-4_bulge-symmetric_UUCG- | GCCACAUGGGAGUCCUUGG | 0.173 | 0.898 | 0.894 | 0.6 | 0.669 | 0.692 |
| | ACAU | CCUUUGAAAGUCCUUUCAU | | | | | | |
| | −20_4-4_bulge-symmetric_UGGU- | GAUACAUCCACGGCUAAUC | | | | | | |
| | UAGU | CUUGGCUUUACUAGUCACU | | | | | | |
| | −8_6-6_internal_loop- | GUCGUACAUUGGCCACUCCC | | | | | | |
| | symmetric_GAAUUC-CCUUGG | AGU | | | | | | |
| | 0_1-1_mismatch_A-C | | | | | | | |
| | 10_1-0_bulge-asymmetric_U- | | | | | | | |
| | 38_6-6_internal_loop- | | | | | | | |
| | symmetric_GGGAGU-UGGGAG | | | | | | | |
| 68 | −33_4-4_bulge-symmetric_UUCG- | GCCACACAGACCUCCUACAC | 0.018 | 0.828 | 0.846 | 0.941 | 1.632 | 1.613 |
| | ACAU | GGUUGAAAGUCCAUUCAUG | | | | | | |
| | −20_4-4_bulge-symmetric_UGGU- | AACAUCCACGGCUAAUUUC | | | | | | |
| | UAGU | AAGCUUUACUAGUCACUGU | | | | | | |
| | −8_6-6_internal_loop- | CGUACAUUGGCCACUCCCAG | | | | | | |
| | symmetric_GAAUUC-UUCAAG | U | | | | | | |
| | 0_1-1_mismatch_A-C | | | | | | | |
| | 8_2-0_bulge-asymmetric_AU- | | | | | | | |
| | 17_1-1_mismatch_A-A | | | | | | | |
| | 28_6-6_internal_loop- | | | | | | | |
| | symmetric_AGGCCA-ACACGG | | | | | | | |
| | 40_4-4_bulge-symmetric_GAGU- | | | | | | | |
| | CAGA | | | | | | | |
| 69 | −33_4-4_bulge-symmetric_UUCG- | GCCACACAGACCUCCUGAAU | 0.08 | 0.919 | 0.924 | 0.958 | 1.664 | 1.664 |
| | ACAU | ACUUGAAAGUCCUGUCAUG | | | | | | |
| | −20_4-4_bulge-symmetric_UGGU- | AAUACAUCCACGACUACCG | | | | | | |
| | UAGU | UCAUCCUUUACUAGUCACU | | | | | | |
| | −6_6-6_internal_loop- | GUCGUACAUUGGCCACUCCC | | | | | | |
| | symmetric_AUUCAU-CCGUCA | AGU | | | | | | |
| | −2_1-1_mismatch_C-A | | | | | | | |
| | 0_1-1_mismatch_A-C | | | | | | | |
| | 16_1-1_mismatch_A-G | | | | | | | |
| | 28_6-6_internal_loop- | | | | | | | |
| | symmetric_AGGCCA-GAAUAC | | | | | | | |
| | 40_4-4_bulge-symmetric_GAGU- | | | | | | | |
| | CAGA | | | | | | | |
| 70 | −33_4-4_bulge-symmetric_UUCG- | GCCACACAGACCUCCUUGAU | 0.333 | 0.935 | 0.918 | 1.157 | 1.679 | 1.653 |
| | ACAU | GGCGGAAAGUCCUUUCAUG | | | | | | |
| | −22_2-3_bulge-asymmetric_UG-AGU | AAUACAUCCACGGCUAAAG | | | | | | |
| | −21_1-1_wobble_G-U | AAUUCGAUCCAUAGUCACU | | | | | | |
| | −14_5-4_internal_loop- | GUCGUACAUUGGCCACUCCC | | | | | | |
| | asymmetric_UAAAG-GAUC | AGU | | | | | | |
| | −7_1-1_mismatch_A-A | | | | | | | |
| | 0_1-1_mismatch_A-C | | | | | | | |
| | 26_6-6_internal_loop- | | | | | | | |
| | symmetric_AAAGGC-AUGGCG | | | | | | | |
| | 40_4-4_bulge-symmetric_GAGU- | | | | | | | |
| | CAGA | | | | | | | |
| 71 | −33_4-4_bulge-symmetric_UUCG- | GCCACACAGACCUCCUUGGC | 0.045 | 0.928 | 0.907 | 0.908 | 1.714 | 1.695 |
| | ACAU | GCGAUUAAGUCCCCUUCAU | | | | | | |
| | −18_6-6_internal_loop- | GAAUACAUCCACGGGUACU | | | | | | |
| | symmetric_UGGUGU-UGUUGU | GAAUUCCUUUUGUUGUCAC | | | | | | |
| | −6_1-1_mismatch_U-C | UGUCGUACAUUGGCCACUC | | | | | | |
| | −3_1-1_mismatch_G-G | CCAGU | | | | | | |
| | 0_1-1_mismatch_A-C | | | | | | | |
| | 17_1-2_bulge-asymmetric_A-CC | | | | | | | |
| | 24_6-6_internal_loop- | | | | | | | |
| | symmetric_UCAAAG-GCGAUU | | | | | | | |
| | 40_4-4_bulge-symmetric_GAGU- | | | | | | | |
| | CAGA | | | | | | | |
| 72 | −33_4-4_bulge-symmetric_UUCG- | GCCACACAGACCUCCUUGGC | 0.03 | 0.619 | 0.684 | 0.932 | 1.427 | 1.439 |
| | ACAU | AGCGAUAAGUCCGUUCAUG | | | | | | |
| | −20_4-4_bulge-symmetric_UGGU- | AAAAGAUCCACGGCUAAUC | | | | | | |
| | UAGU | CACGACUUUACUAGUCACU | | | | | | |
| | −8_6-6_internal_loop- | GUCGUACAUUGGCCACUCCC | | | | | | |
| | symmetric_GAAUUC-CCACGA | AGU | | | | | | |
| | 0_1-1_mismatch_A-C | | | | | | | |
| | 6_1-1_mismatch_G-G | | | | | | | |
| | 8_1-1_mismatch_A-A | | | | | | | |
| | 17_1-1_mismatch_A-G | | | | | | | |
| | 24_6-6_internal_loop- | | | | | | | |
| | symmetric_UCAAAG-AGCGAU | | | | | | | |
| | 40_4-4_bulge-symmetric_GAGU- | | | | | | | |
| | CAGA | | | | | | | |
| 73 | −33_4-4_bulge-symmetric_UUCG- | GCCACACAGACCUCCUCUUA | 0.034 | 0.606 | 0.65 | 0.95 | 1.433 | 1.449 |
| | ACAU | GAUUGAAAGUGUUCAUGAA | | | | | | |
| | −23_0-4_bulge-asymmetric_-UAGU | UAGAUCACACGGCUAAUGA | | | | | | |
| | −23_1-1_wobble_U-G | AUUCACACUGUAGUCACUG | | | | | | |
| | −22_1-1_wobble_G-U | UCGUACAUUGGCCACUCCCA | | | | | | |
| | −14_4-0_bulge-asymmetric_AAAG- | GU | | | | | | |
| | 0_1-1_mismatch_A-C | | | | | | | |
| | 2_0-1_bulge-asymmetric_-A | | | | | | | |
| | 6_1-1_mismatch_G-G | | | | | | | |
| | 17_3-1_bulge-asymmetric_AGG-G | | | | | | | |
| | 28_6-6_internal_loop- | | | | | | | |
| | symmetric_AGGCCA-CUUAGA | | | | | | | |
| | 40_4-4_bulge-symmetric_GAGU- | | | | | | | |
| | CAGA | | | | | | | |
| 74 | −33_4-4_bulge-symmetric_UUCG- | GCCACACAAGAUAGCUUGG | 0.034 | 0.825 | 0.815 | 0.822 | 1.013 | 0.987 |
| | ACAU | CCUUUGAAAGUCCUUGGAU | | | | | | |
| | −20_4-4_bulge-symmetric_UGGU- | UAAUGCAUCCACGGCUAAU | | | | | | |
| | UAGU | UCUUAGCUUUACUAGUCAC | | | | | | |
| | −8_6-6_internal_loop- | UGUCGUACAUUGGCCACUC | | | | | | |
| | symmetric_GAAUUC-UCUUAG | CCAGU | | | | | | |
| | 0_1-1_mismatch_A-C | | | | | | | |
| | 7_1-1_wobble_U-G | | | | | | | |
| | 11_1-1_mismatch_C-U | | | | | | | |
| | 14_2-2_bulge-symmetric_GA-GG | | | | | | | |
| | 36_8-8_internal_loop- | | | | | | | |
| | symmetric_GAGGGAGU- | | | | | | | |
| | CAAGAUAG | | | | | | | |
| 75 | −33_4-4_bulge-symmetric_UUCG- | GCCACACAGAGGAACUUGG | 0.057 | 0.777 | 0.8 | 0.887 | 1.097 | 1.084 |
| | ACAU | CCUUUGAAAGUCCCUUCAA | | | | | | |
| | −20_4-4_bulge-symmetric_UGGU- | UACAUCCACGACUAUAGUU | | | | | | |
| | UAGU | GUCCUUUACUAGUCACUGU | | | | | | |
| | −6_6-6_internal_loop- | CGUACAUUGGCCACUCCCAG | | | | | | |
| | symmetric_AUUCAU-UAGUUG | U | | | | | | |
| | −2_1-1_mismatch_C-A | | | | | | | |
| | 0_1-1_mismatch_A-C | | | | | | | |
| | 10_3-0_bulge-asymmetric_UCA- | | | | | | | |
| | 17_1-1_mismatch_A-C | | | | | | | |
| | 36_8-8_internal_loop- | | | | | | | |
| | symmetric_GAGGGAGU- | | | | | | | |
| | CAGAGGAA | | | | | | | |
| 76 | −33_4-4_bulge-symmetric_UUCG- | GCCACACAAUAGAACUUGG | 0.304 | 0.725 | 0.741 | 0.784 | 1.023 | 0.997 |
| | ACAU | CCUUUGAAAGUCCUUUCAU | | | | | | |
| | −20_4-4_bulge-symmetric_UGGU- | GAAUAAAUCCACGGCUAAU | | | | | | |
| | UAGU | AACAGGCUUUACUAGUCAC | | | | | | |
| | −8_6-6_internal_loop- | UGUCGUACAUUGGCCACUC | | | | | | |
| | symmetric_GAAUUC-AACAGG | CCAGU | | | | | | |
| | 0_1-1_mismatch_A-C | | | | | | | |
| | 6_1-1_mismatch_G-A | | | | | | | |
| | 36_8-8_internal_loop- | | | | | | | |
| | symmetric_GAGGGAGU- | | | | | | | |
| | CAAUAGAA | | | | | | | |
| 77 | −33_4-4_bulge-symmetric_UUCG- | GCCACACAGACCUCGAACGA | 0.401 | 0.881 | 0.88 | 1.27 | 1.516 | 1.585 |
| | ACAU | CUUUGAAAGUCCUUUCAUG | | | | | | |
| | −20_4-4_bulge-symmetric_UGGU- | AAUAGAUCCACGGCUAAUU | | | | | | |
| | UAGU | AUAAGCUUUACUAGUCACU | | | | | | |
| | −8_6-6_internal_loop- | GUCGUACAUUGGCCACUCCC | | | | | | |
| | symmetric_GAAUUC-UAUAAG | AGU | | | | | | |
| | 0_1-1_mismatch_A-C | | | | | | | |
| | 6_1-1_mismatch_G-G | | | | | | | |
| | 30_6-6_internal_loop- | | | | | | | |
| | symmetric_GCCAAG-GAACGA | | | | | | | |
| | 40_4-4_bulge-symmetric_GAGU- | | | | | | | |
| | CAGA | | | | | | | |
| 78 | −8_6-6_internal_loop- | GCCACACAGACCUCGAACGA | 0.661 | 0.902 | 0.841 | 1.38 | 1.465 | 1.491 |
| | symmetric_GAAUUC-UAUAAG | CUUUGAAAGUCCUUUCAUG | | | | | | |
| | 0_1-1_mismatch_A-C | AAUAGAUCCACGGCUAAUU | | | | | | |
| | 6_1-1_mismatch_G-G | AUAAGCUUUACACCACACU | | | | | | |
| | 30_6-6_internal_loop- | GUCGUCGAAUGGCCACUCCC | | | | | | |
| | symmetric_GCCAAG-GAACGA | AGU | | | | | | |
| | 40_4-4_bulge-symmetric_GAGU- | | | | | | | |
| | CAGA | | | | | | | |
| 79 | −33_4-4_bulge-symmetric_UUCG- | GCCACACAGACCUCCUUGGC | 0.12 | 0.805 | 0.835 | 1.019 | 1.621 | 1.5' |
| | ACAU | GAUCACAAGUCCUUUCAUG | | | | | | |
| | −20_4-4_bulge-symmetric_UGGU- | AAUAAAUCCACGGUUAAUG | | | | | | |
| | UAGU | ACCAGUGUUACUAGUCACU | | | | | | |
| | −10_6-6_internal_loop- | GUCGUACAUUGGCCACUCCC | | | | | | |
| | symmetric_AGGAAU-CCAGUG | AGU | | | | | | |
| | −3_1-1_wobble_G-U | | | | | | | |
| | 0_1-1_mismatch_A-C | | | | | | | |
| | 6_1-1_mismatch_G-A | | | | | | | |
| | 24_6-6_internal_loop- | | | | | | | |
| | symmetric_UCAAAG-GAUCAC | | | | | | | |
| | 40_4-4_bulge-symmetric_GAGU- | | | | | | | |
| | CAGA | | | | | | | |
| 80 | −33_4-4_bulge-symmetric_UUCG- | GCCACACAGACCUCCUGAUU | 0.044 | 0.853 | 0.851 | 0.928 | 1.439 | 1.432 |
| | ACAU | AAUUGAAAGUCCUUUCAAG | | | | | | |
| | −23_1-0_bulge-asymmetric_U- | AAUACACACACGGGUAAUG | | | | | | |
| | −22_1-1_wobble_G-U | AAUUCGCAUUGUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −18_1-1_wobble_U-G | | | | | | | |
| | −15_1-1_mismatch_A-A | | | | | | | |
| | −13_0-1_bulge-asymmetric_-G | | | | | | | |
| | −3 1-1_mismatch_G-G | | | | | | | |
| | 0_1-1_mismatch_A-C | | | | | | | |
| | 3_2-2_bulge-symmetric_GA-CA | | | | | | | |
| | 12_1-1_mismatch_A-A | | | | | | | |
| | 28_6-6_internal_loop- | | | | | | | |
| | symmetric_AGGCCA-GAUUAA | | | | | | | |
| | 40_4-4_bulge-symmetric_GAGU- | | | | | | | |
| | CAGA | | | | | | | |
| 81 | −33_4-4_bulge-symmetric_UUCG- | GCCACACAGACCAGGUAUG | 0.042 | 0.596 | 0.654 | 0.956 | 1.407 | 1.418 |
| | ACAU | CCUUUGAAAGUAGUUCAUG | | | | | | |
| | −18_6-6_internal_loop- | AAUACAACCACGGCUACAC | | | | | | |
| | symmetric_UGGUGU-CUUGGU | AAUUCCUUUCUUGGUCACU | | | | | | |
| | −6_3-3_bulge-symmetric_CAU-CAC | GUCGUACAUUGGCCACUCCC | | | | | | |
| | 0_1-1_mismatch_A-C | AGU | | | | | | |
| | 4_1-1_mismatch_A-A | | | | | | | |
| | 17_3-2_bulge-asymmetric_AGG-AG | | | | | | | |
| | 32_6-6_internal_loop- | | | | | | | |
| | symmetric_CAAGGA-AGGUAU | | | | | | | |
| | 40_4-4_bulge-symmetric_GAGU- | | | | | | | |
| | CAGA | | | | | | | |
| 82 | −33_4-4_bulge-symmetric_UUCG- | GCCACACAGAAUAAGCUGG | 0.311 | 0.95 | 0.904 | 0.703 | 0.854 | 0.977 |
| | ACAU | CCUUUGAAAGUCCUUUCAU | | | | | | |
| | −23_0-1_bulge-asymmetric_-U | GAAUACACCCACGACUAAU | | | | | | |
| | −23_1-1_wobble_U-G | GAAUUCCUGCAAUGGUCAC | | | | | | |
| | −22_1-1_mismatch_G-G | UGUCGUACAUUGGCCACUC | | | | | | |
| | −21_1-1_wobble_G-U | CCAGU | | | | | | |
| | −16_4-3_bulge-asymmetric_GUAA- | | | | | | | |
| | GCA | | | | | | | |
| | −2_1-1_mismatch_C-A | | | | | | | |
| | 0_1-1_mismatch_A-C | | | | | | | |
| | 4_1-1_mismatch_A-C | | | | | | | |
| | 34_10−10_internal_loop- | | | | | | | |
| | symmetric_AGGAGGGAGU- | | | | | | | |
| | CAGAAUAAGC | | | | | | | |
| 83 | −33_4-4_bulge-symmetric_UUCG- | GCCACACAGACCUCCUUGGC | 0.041 | 0.765 | 0.78 | 0.908 | 1.505 | 1.504 |
| | ACAU | GGCGCCAAGUCCUUUCAUG | | | | | | |
| | −23_0-2_bulge-asymmetric_-GU | AAUACAUCAACGGCUAAUG | | | | | | |
| | −22_1-1_wobble_G-U | AUCGAAAUUACUAGUCACU | | | | | | |
| | −19_1-1_wobble_G-U | GUCGUACAUUGGCCACUCCC | | | | | | |
| | −14_5-5_internal_loop- | AGU | | | | | | |
| | symmetric_UAAAG-GAAAU | | | | | | | |
| | −10_2-0_bulge-asymmetric_AU- | | | | | | | |
| | 0→2_3-3_bulge-symmetric_AUG- | | | | | | | |
| | AAC | | | | | | | |
| | 24_6-6_internal_loop- | | | | | | | |
| | symmetric_UCAAAG-GGCGCC | | | | | | | |
| | 40_4-4_bulge-symmetric_GAGU- | | | | | | | |
| | CAGA | | | | | | | |
| 84 | −33_4-4_bulge-symmetric_UUCG- | GCCACACAGACCUCCUUGGC | 0.034 | 0.741 | 0.759 | 0.936 | 1.521 | 1.493 |
| | ACAU | CUCCCUCCGUCCUCUCAUAA | | | | | | |
| | −23_0-2_bulge-asymmetric_-GU | AUACAUCCACGGCCAUGAA | | | | | | |
| | −22_1-1_wobble_G-U | UGGAGACACUAGUCACUGU | | | | | | |
| | −12 6-4_internal_loop- | CGUACAUUGGCCACUCCCAG | | | | | | |
| | asymmetric_AAAGGA-GGAG | U | | | | | | |
| | −4_2-1_bulge-asymmetric_UA-C | | | | | | | |
| | 0_1-1_mismatch_A-C | | | | | | | |
| | 11_1-1_mismatch_C-A | | | | | | | |
| | 16_1-1_mismatch_A-C | | | | | | | |
| | 22_6-6_internal_loop- | | | | | | | |
| | symmetric_UUUCAA-CCCUCC | | | | | | | |
| | 40_4-4_bulge-symmetric_GAGU- | | | | | | | |
| | CAGA | | | | | | | |
| 85 | −33_4-4_bulge-symmetric_UUCG- | GCCACACAGACCUCCUUGUU | 0.048 | 0.667 | 0.674 | 0.975 | 1.506 | 1.521 |
| | ACAU | GAAGGAAAGUCCUUCGUGA | | | | | | |
| | −20_4-4_bulge-symmetric_UGGU- | AUACAUCCACGGCUAAUCU | | | | | | |
| | UAGU | GGGGCUUUACUAGUCACUG | | | | | | |
| | −8_6-6_internal_loop- | UCGUACAUUGGCCACUCCCA | | | | | | |
| | symmetric_GAAUUC-CUGGGG | GU | | | | | | |
| | 0_1-1_mismatch_A-C | | | | | | | |
| | 13 1-1_wobble_U-G | | | | | | | |
| | 17_1-0_bulge-asymmetric_A- | | | | | | | |
| | 26_6-6_internal_loop- | | | | | | | |
| | symmetric_AAAGGC-UUGAAG | | | | | | | |
| | 40_4-4_bulge-symmetric_GAGU- | | | | | | | |
| | CAGA | | | | | | | |
| 86 | −33_4-4_bulge-symmetric_UUCG- | GCCACACAGACCUCCUUGUA | 0.171 | 0.833 | 0.816 | 0.886 | 1.332 | 1.275 |
| | ACAU | GGAGGAAAGUCCUUUCAUG | | | | | | |
| | −20_4-4_bulge-symmetric_UGGU- | AAUACAUGCACGGCUAAUG | | | | | | |
| | UAGU | AUCGGUGUUACUAGUCACU | | | | | | |
| | −10_6-6_internal_loop- | GUCGUACAUUGGCCACUCCC | | | | | | |
| | symmetric_AGGAAU-UCGGUG | AGU | | | | | | |
| | 0_1-1_mismatch_A-C | | | | | | | |
| | 3_1-1_mismatch_G-G | | | | | | | |
| | 26_6-6_internal_loop- | | | | | | | |
| | symmetric_AAAGGC-UAGGAG | | | | | | | |
| | 40_4-4_bulge-symmetric_GAGU- | | | | | | | |
| | CAGA | | | | | | | |
| 87 | −33_4-4_bulge-symmetric_UUCG- | GCCACACAGACCUCCUUGGC | 0.029 | 0.887 | 0.853 | 0.913 | 1.646 | 1.601 |
| | ACAU | GGAAGCAAGUCCUGCCAUG | | | | | | |
| | −23_1-0_bulge-asymmetric_U- | CAUACAUUCACGGCUAAUG | | | | | | |
| | −22_1-1_wobble_G-U | AAUUCGAUGGAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_4-5_internal_loop- | | | | | | | |
| | asymmetric_AAAG-GAUGG | | | | | | | |
| | 0_1-1_mismatch_A-C | | | | | | | |
| | 3_1-1_wobble_G-U | | | | | | | |
| | 10_1-1_mismatch_U-C | | | | | | | |
| | 15_2-2_bulge-symmetric_AA-GC | | | | | | | |
| | 24_6-6_internal_loop- | | | | | | | |
| | symmetric_UCAAAG-GGAAGC | | | | | | | |
| | 40_4-4_bulge-symmetric_GAGU- | | | | | | | |
| | CAGA | | | | | | | |
| 88 | −33_4-4_bulge-symmetric_UUCG- | GCCACACAGACCUCCUAUAA | 0.019 | 0.693 | 0.658 | 0.942 | 1.52 | 1.504 |
| | ACAU | ACUUGAAAGUCCAUUCAUG | | | | | | |
| | −14_10−10_internal_loop- | AAUAGUCCCACGGCUAUUG | | | | | | |
| | symmetric_UGGUGUAAAG- | AAUUCACGUCGUAGUCACU | | | | | | |
| | ACGUCGUAGU | GUCGUACAUUGGCCACUCCC | | | | | | |
| | −6_1-1_mismatch_U-U | AGU | | | | | | |
| | 0_1-1_mismatch_A-C | | | | | | | |
| | 4_3-3_bulge-symmetric_AUG-GUC | | | | | | | |
| | 17_1-1_mismatch_A-A | | | | | | | |
| | 28_6-6_internal_loop- | | | | | | | |
| | symmetric_AGGCCA-AUAAAC | | | | | | | |
| | 40_4-4_bulge-symmetric_GAGU- | | | | | | | |
| | CAGA | | | | | | | |
| 89 | −33_4-4_bulge-symmetric_UUCG- | GCCACACAGAAAGGGCUGG | 0.326 | 0.772 | 0.821 | 0.846 | 0.963 | 1.076 |
| | ACAU | CCUUUGAAAGUCCUUUCAU | | | | | | |
| | −22_2-5_internal_loop- | GAAUACAUCCACGGCUAAA | | | | | | |
| | asymmetric_UG-GUAGU | UGAAUUCGCCACGUAGUCA | | | | | | |
| | −14_5-2_internal_loop- | CUGUCGUACAUUGGCCACU | | | | | | |
| | asymmetric_UAAAG-GC | CCCAGU | | | | | | |
| | −4_0-1_bulge-asymmetric_-A | | | | | | | |
| | 0_1-1_mismatch_A-C | | | | | | | |
| | 34_10−10_internal_loop- | | | | | | | |
| | symmetric_AGGAGGGAGU- | | | | | | | |
| | CAGAAAGGGC | | | | | | | |
| 90 | −33_4-4_bulge-symmetric_UUCG- | GCCACACAGACCUCCUUGGC | 0.044 | 0.666 | 0.678 | 0.934 | 1.478 | 1.461 |
| | ACAU | GGUCUCAAGUCAGCUUUCA | | | | | | |
| | −23_1-0_bulge-asymmetric_U- | UGAUUACACCCACGGCUAC | | | | | | |
| | −22_1-1_wobble_G-U | GGAAUUCGGCUGAUAGUCA | | | | | | |
| | −21_1-1_mismatch_G-G | CUGUCGUACAUUGGCCACU | | | | | | |
| | −19_1-1_wobble_G-U | CCCAGU | | | | | | |
| | −14_4-5_internal_loop- | | | | | | | |
| | asymmetric_AAAG-GGCUG | | | | | | | |
| | −6_2-2_bulge-symmetric_AU-CG | | | | | | | |
| | 0_1-1_mismatch_A-C | | | | | | | |
| | 4_1-1_mismatch_A-C | | | | | | | |
| | 9_1-1_mismatch_U-U | | | | | | | |
| | 18_0-2_bulge-asymmetric_-AG | | | | | | | |
| | 24_6-6_internal_loop- | | | | | | | |
| | symmetric_UCAAAG-GGUCUC | | | | | | | |
| | 40_4-4_bulge-symmetric_GAGU- | | | | | | | |
| | CAGA | | | | | | | |
| 91 | −33_4-4_bulge-symmetric_UUCG- | GCCACACAGACCUCCUCCAA | 0.05 | 0.749 | 0.729 | 0.951 | 1.509 | 1.492 |
| | ACAU | UGUUGAAAGUACUUUCAUG | | | | | | |
| | −23_0-2_bulge-asymmetric_-GU | AAUACAUCCACGGCGACUG | | | | | | |
| | −20_1-0_bulge-asymmetric_U- | AAUUCCUGUCCCAGUCACU | | | | | | |
| | −16_3-2_bulge-asymmetric_UAA-GU | GUCGUACAUUGGCCACUCCC | | | | | | |
| | −4_3-3_bulge-symmetric_UUA-GAC | AGU | | | | | | |
| | 0_1-1_mismatch_A-C | | | | | | | |
| | 19_1-1_mismatch_G-A | | | | | | | |
| | 28_6-6_internal_loop- | | | | | | | |
| | symmetric_AGGCCA-CCAAUG | | | | | | | |
| | 40_4-4_bulge-symmetric_GAGU- | | | | | | | |
| | CAGA | | | | | | | |
| 92 | −33_4-4_bulge-symmetric_UUCG- | GCCACACAGACCUCCUUGUA | 0.023 | 0.879 | 0.853 | 0.893 | 1.513 | 1.487 |
| | ACAU | UUACGAAAGUGCUUUGAUG | | | | | | |
| | −23_0-4_bulge-asymmetric_-UAGU | AAUACAUCCACUGCUAAUG | | | | | | |
| | −23_1-1_wobble_U-G | AAUUCAGCCCGUAGUCACU | | | | | | |
| | −20_1-0_bulge-asymmetric_U- | GUCGUACAUUGGCCACUCCC | | | | | | |
| | −18_1-1_wobble_U-G | AGU | | | | | | |
| | −14_4-1_bulge-asymmetric_AAAG-A | | | | | | | |
| | −1->0_2-2_bulge-symmetric_CA-CU | | | | | | | |
| | 14_1-1_mismatch_G-G | | | | | | | |
| | 19_1-1_mismatch_G-G | | | | | | | |
| | 26_6-6_internal_loop- | | | | | | | |
| | symmetric_AAAGGC-UAUUAC | | | | | | | |
| | 40_4-4_bulge-symmetric_GAGU- | | | | | | | |
| | CAGA | | | | | | | |
| 93 | −33_4-4_bulge-symmetric_UUCG- | GCCACACAGACCAUAGACGC | 0.193 | 0.934 | 0.939 | 0.896 | 1.261 | 1.351 |
| | ACAU | CUUUGAAAGUGCUUUCAUG | | | | | | |
| | −18_6-6_internal_loop- | AAUACAUCCACGGCUACUG | | | | | | |
| | symmetric_UGGUGU-CUUGGU | AAUUCCUUUCUUGGUCACU | | | | | | |
| | −6_1-1_mismatch_U-C | GUCGUACAUUGGCCACUCCC | | | | | | |
| | 0_1-1_mismatch_A-C | AGU | | | | | | |
| | 19_1-1_mismatch_G-G | | | | | | | |
| | 32_6-6_internal_loop- | | | | | | | |
| | symmetric_CAAGGA-AUAGAC | | | | | | | |
| | 40_4-4_bulge-symmetric_GAGU- | | | | | | | |
| | CAGA | | | | | | | |
| 94 | −33_4-4_bulge-symmetric_UUCG- | GCCACACAGACCUCCUUGGC | 0.065 | 0.919 | 0.91 | 0.957 | 1.698 | 1.683 |
| | ACAU | GGCAUCAAGUAAUUUCAUG | | | | | | |
| | −20_4-4_bulge-symmetric_UGGU- | AAUACAUCCACGGCUACGA | | | | | | |
| | UAGU | CGGUCCUUUACUAGUCACU | | | | | | |
| | −6_6-6_internal_loop- | GUCGUACAUUGGCCACUCCC | | | | | | |
| | symmetric_AUUCAU-CGACGG | AGU | | | | | | |
| | 0_1-1_mismatch_A-C | | | | | | | |
| | 18_2-2_bulge-symmetric_GG-AA | | | | | | | |
| | 24_6-6_internal_loop- | | | | | | | |
| | symmetric_UCAAAG-GGCAUC | | | | | | | |
| | 40_4-4_bulge-symmetric_GAGU- | | | | | | | |
| | CAGA | | | | | | | |
| 95 | −33_4-4_bulge-symmetric_UUCG- | GCCACACAGACCGGAAAUG | 0.036 | 0.529 | 0.578 | 0.914 | 1.32 | 1.311 |
| | ACAU | CCUUUGAAAGUCCCGCCAU | | | | | | |
| | −20_4-4_bulge-symmetric_UGGU- | GAAUAAUUCCACGGCUAUG | | | | | | |
| | UAGU | GACGAUGGUUACUAGUCAC | | | | | | |
| | −10_6-6_internal_loop- | UGUCGUACAUUGGCCACUC | | | | | | |
| | symmetric_AGGAAU-CGAUGG | CCAGU | | | | | | |
| | −6_2-2_bulge-symmetric_AU-UG | | | | | | | |
| | 0_1-1_mismatch_A-C | | | | | | | |
| | 5_2-2_bulge-symmetric_UG-AU | | | | | | | |
| | 15_3-3_bulge-symmetric_AAA-CGC | | | | | | | |
| | 32_6-6_internal_loop- | | | | | | | |
| | symmetric_CAAGGA-GGAAAU | | | | | | | |
| | 40_4-4_bulge-symmetric_GAGU- | | | | | | | |
| | CAGA | | | | | | | |
| 96 | −33_4-4_bulge-symmetric_UUCG- | GCCACACAGACCUCCUUGGC | 0.023 | 0.759 | 0.755 | 0.924 | 1.507 | 1.44 |
| | ACAU | GAUGCUAAGUAGCUUCAUG | | | | | | |
| | −23_0-4_bulge-asymmetric_-UAGU | AAUACCUCCACGUAUAAUG | | | | | | |
| | −19_1-1_mismatch_G-G | AAUUCAGACCAUAGUCACU | | | | | | |
| | −14_4-0_bulge-asymmetric_AAAG- | GUCGUACAUUGGCCACUCCC | | | | | | |
| | −2_2-2_bulge-symmetric_GC-UA | AGU | | | | | | |
| | 0_1-1_mismatch_A-C | | | | | | | |
| | 5_1-1_mismatch_U-C | | | | | | | |
| | 17_3-3_bulge-symmetric_AGG-AGC | | | | | | | |
| | 24_6-6_internal_loop- | | | | | | | |
| | symmetric_UCAAAG-GAUGCU | | | | | | | |
| | 40_4-4_bulge-symmetric_GAGU- | | | | | | | |
| | CAGA | | | | | | | |
| 97 | −33_4-4_bulge-symmetric_UUCG- | GCCACACAGGAUAGCUUGG | 0.292 | 0.899 | 0.871 | 0.705 | 0.826 | 0.829 |
| | ACAU | CCUUUGAAAGUCCUUUCAU | | | | | | |
| | −20_4-4_bulge-symmetric_UGGU- | GAAUACUUCCACGGCUAAU | | | | | | |
| | UAGU | GACCAAUGUUACUAGUCAC | | | | | | |
| | −10_6-6_internal_loop- | UGUCGUACAUUGGCCACUC | | | | | | |
| | symmetric_AGGAAU-CCAAUG | CCAGU | | | | | | |
| | 0_1-1_mismatch_A-C | | | | | | | |
| | 5_1-1_mismatch_U-U | | | | | | | |
| | 36_8-8_internal_loop- | | | | | | | |
| | symmetric_GAGGGAGU- | | | | | | | |
| | CAGGAUAG | | | | | | | |
| 98 | −33_4-4_bulge-symmetric_UUCG- | GCCACACAGACCUCCUUGGC | 0.033 | 0.888 | 0.883 | 0.921 | 1.638 | 1.596 |
| | ACAU | GGCGCCAAGUGAUUUGAUG | | | | | | |
| | −20_4-4_bulge-symmetric_UGGU- | AAUACAUCCACGGCUAGUU | | | | | | |
| | UAGU | UCCAACUUUACUAGUCACU | | | | | | |
| | −8_6-6_internal_loop- | GUCGUACAUUGGCCACUCCC | | | | | | |
| | symmetric_GAAUUC-UUCCAA | AGU | | | | | | |
| | −6_1-1_wobble_U-G | | | | | | | |
| | 0_1-1_mismatch_A-C | | | | | | | |
| | 14_1-1_mismatch_G-G | | | | | | | |
| | 18_2-2_bulge-symmetric_GG-GA | | | | | | | |
| | 24_6-6_internal_loop- | | | | | | | |
| | symmetric_UCAAAG-GGCGCC | | | | | | | |
| | 40_4-4_bulge-symmetric_GAGU- | | | | | | | |
| | CAGA | | | | | | | |
| 99 | −33_4-4_bulge-symmetric_UUCG- | GCCACACAGACCUCCUUGGC | 0.025 | 0.835 | 0.813 | 0.916 | 1.552 | 1.472 |
| | ACAU | ACGGUUAAGUCCUUCGAUG | | | | | | |
| | −20_4-4_bulge-symmetric_UGGU- | AAUACGUCCACGGCUUUUG | | | | | | |
| | UAGU | AAUGCAUCGACUAGUCACU | | | | | | |
| | −12_6-6_internal_loop- | GUCGUACAUUGGCCACUCCC | | | | | | |
| | symmetric_AAAGGA-GCAUCG | AGU | | | | | | |
| | −5_2-2_bulge-symmetric_UU-UU | | | | | | | |
| | 0_1-1_mismatch_A-C | | | | | | | |
| | 5_1-1_wobble_U-G | | | | | | | |
| | 14_2-2_bulge-symmetric_GA-CG | | | | | | | |
| | 24_6-6_internal_loop- | | | | | | | |
| | symmetric_UCAAAG-ACGGUU | | | | | | | |
| | 40_4-4_bulge-symmetric_GAGU- | | | | | | | |
| | CAGA | | | | | | | |
| 100 | −33_4-4_bulge-symmetric_UUCG- | GCCACACAGACCUCCUUGUA | 0.054 | 0.938 | 0.909 | 0.958 | 1.734 | 1.653 |
| | ACAU | AACAGAAAGUCCUUUGGUG | | | | | | |
| | −16_8-8_internal_loop- | AAUACGUCCACGGCUAAUG | | | | | | |
| | symmetric_UGGUGUAA- | AAUUCCUCUUUUGGUCACU | | | | | | |
| | CUUUUGGU | GUCGUACAUUGGCCACUCCC | | | | | | |
| | 0_1-1_mismatch_A-C | AGU | | | | | | |
| | 5_1-1_wobble_U-G | | | | | | | |
| | 13_1-1_wobble_U-G | | | | | | | |
| | 14_1-1_mismatch_G-G | | | | | | | |
| | 26_6-6_internal_loop- | | | | | | | |
| | symmetric_AAAGGC-UAAACA | | | | | | | |
| | 40_4-4_bulge-symmetric_GAGU- | | | | | | | |
| | CAGA | | | | | | | |
| 101 | −33_4-4_bulge-symmetric_UUCG- | GCCACACAGACCUCCUUGAU | 0.177 | 0.901 | 0.889 | 0.964 | 1.635 | 1.579 |
| | ACAU | AGUGGAAAGUCCUUUCAUG | | | | | | |
| | −20_4-4_bulge-symmetric_UGGU- | AAUACAUCCACGGGUAAUC | | | | | | |
| | UAGU | GCCGACUUUACUAGUCACU | | | | | | |
| | −8_6-6_internal_loop- | GUCGUACAUUGGCCACUCCC | | | | | | |
| | symmetric_GAAUUC-CGCCGA | AGU | | | | | | |
| | −3_1-1_mismatch_G-G | | | | | | | |
| | 0_1-1_mismatch_A-C | | | | | | | |
| | 26_6-6_internal_loop- | | | | | | | |
| | symmetric_AAAGGC-AUAGUG | | | | | | | |
| | 40_4-4_bulge-symmetric_GAGU- | | | | | | | |
| | CAGA | | | | | | | |
| 102 | −33_4-4_bulge-symmetric_UUCG- | GCCACACAGACCUCAAGAU | 0.248 | 0.945 | 0.928 | 1.104 | 1.55 | 1.561 |
| | ACAU | ACUUUGAAAGUCCUUUCAU | | | | | | |
| | −20_6-6_internal_loop- | GCAUACAUUCACGGCUAAU | | | | | | |
| | symmetric_UGUGGU-UGAUUU | GAAUUCCUUUACUGAUUUC | | | | | | |
| | 0_1-1_mismatch_A-C | UGUCGUACAUUGGCCACUC | | | | | | |
| | 3_1-1_wobble_G-U | CCAGU | | | | | | |
| | 10_1-1_mismatch_U-C | | | | | | | |
| | 30_6-6_internal_loop- | | | | | | | |
| | symmetric_GCCAAG-AAGAUA | | | | | | | |
| | 40_4-4_bulge-symmetric_GAGU- | | | | | | | |
| | CAGA | | | | | | | |
| 103 | −33_4-4_bulge-symmetric_UUCG- | GCCACACAGACCUCCUCAUG | 0.088 | 0.929 | 0.909 | 0.941 | 1.63 | 1.638 |
| | ACAU | AGUUGAAAGUCCUUUCAUG | | | | | | |
| | −23_0-1_bulge-asymmetric_-U | AAUACAUCCACGGACUUGA | | | | | | |
| | −23_1-1_wobble_U-G | AUUCCUGGAAUGGUCACUG | | | | | | |
| | −22_1-1_mismatch_G-G | UCGUACAUUGGCCACUCCCA | | | | | | |
| | −21_1-1_wobble_G-U | GU | | | | | | |
| | −16_4-3_bulge-asymmetric_GUAA- | | | | | | | |
| | GGA | | | | | | | |
| | −3_4-3_bulge-asymmetric_UUAG- | | | | | | | |
| | ACU | | | | | | | |
| | 0_1-1_mismatch_A-C | | | | | | | |
| | 28_6-6_internal_loop- | | | | | | | |
| | symmetric_AGGCCA-CAUGAG | | | | | | | |
| | 40_4-4_bulge-symmetric_GAGU- | | | | | | | |
| | CAGA | | | | | | | |
| 104 | −33_4-4_bulge-symmetric_UUCG- | GCCACAUGGGCAUCCUUGG | 0.4 | 0.926 | 0.912 | 0.86 | 0.74 | 0.794 |
| | ACAU | CCUUUGAAAGUCCUUUCAU | | | | | | |
| | −20_4-4_bulge-symmetric_UGGU- | GUAUACAUCCACGGCAGUU | | | | | | |
| | UAGU | ACAUUCCUUUACUAGUCAC | | | | | | |
| | −4_6-6_internal_loop- | UGUCGUACAUUGGCCACUC | | | | | | |
| | symmetric_UCAUUA-AGUUAC | CCAGU | | | | | | |
| | 0_1-1_mismatch_A-C | | | | | | | |
| | 10_1-1_mismatch_U-U | | | | | | | |
| | 38_6-6_internal_loop- | | | | | | | |
| | symmetric_GGGAGU-UGGGCA | | | | | | | |
| 105 | −33_4-4_bulge-symmetric_UUCG- | GCCACACAGUAGGGCUUGG | 0.159 | 0.925 | 0.876 | 0.543 | 0.774 | 0.783 |
| | ACAU | CCUUUGAAAGUCCUUUCAU | | | | | | |
| | −23_0-2_bulge-asymmetric_-GU | GAAUACAUUCACGGCUAAU | | | | | | |
| | −22_1-1_wobble_G-U | GAAUAAAGGCACUAGUCAC | | | | | | |
| | −18_1-1_wobble_U-G | UGUCGUACAUUGGCCACUC | | | | | | |
| | −12_6-4_internal_loop- | CCAGU | | | | | | |
| | asymmetric_AAAGGA-AAAG | | | | | | | |
| | 0_1-1_mismatch_A-C | | | | | | | |
| | 3 1-1_wobble_G-U | | | | | | | |
| | 36_8-8_internal_loop- | | | | | | | |
| | symmetric_GAGGGAGU- | | | | | | | |
| | CAGUAGGG | | | | | | | |
| 106 | −33_4-4_bulge-symmetric_UUCG- | GCCACACAGACCUCCUUGGC | 0.047 | 0.808 | 0.799 | 0.96 | 1.627 | 1.578 |
| | ACAU | GACAACAAGUCCUCUCAUG | | | | | | |
| | −20_4-4_bulge-symmetric_UGGU- | AAUAAUUCCACGGCUACCG | | | | | | |
| | UAGU | UUCUCCUUUACUAGUCACU | | | | | | |
| | −6_6-6_internal_loop- | GUCGUACAUUGGCCACUCCC | | | | | | |
| | symmetric_AUUCAU-CCGUUC | AGU | | | | | | |
| | 0_1-1_mismatch_A-C | | | | | | | |
| | 5_2-2_bulge-symmetric_UG-AU | | | | | | | |
| | 16_1-1_mismatch_A-C | | | | | | | |
| | 24_6-6_internal_loop- | | | | | | | |
| | symmetric_UCAAAG-GACAAC | | | | | | | |
| | 40_4-4_bulge-symmetric_GAGU- | | | | | | | |
| | CAGA | | | | | | | |
| 107 | −33_4-4_bulge-symmetric_UUCG- | GCCACAUGGGGGUCCUUGG | 0.254 | 0.843 | 0.835 | 0.487 | 0.701 | 0.671 |
| | ACAU | CCUUUGAAAGUCCUUUCAU | | | | | | |
| | −20_4-4_bulge-symmetric_UGGU- | GAAUAUAUCCACGGCUAAU | | | | | | |
| | UAGU | GACGAUUAUUACUAGUCAC | | | | | | |
| | −10_6-6_internal_loop- | UGUCGUACAUUGGCCACUC | | | | | | |
| | symmetric_AGGAAU-CGAUUA | CCAGU | | | | | | |
| | 0_1-1_mismatch_A-C | | | | | | | |
| | 6_1-1_wobble_G-U | | | | | | | |
| | 38_6-6_internal_loop- | | | | | | | |
| | symmetric_GGGAGU-UGGGGG | | | | | | | |
| 108 | −33_4-4_bulge-symmetric_UUCG- | GCCACACAGAGGCGAAUGG | 0.139 | 0.889 | 0.87 | 0.822 | 1.104 | 1.215 |
| | ACAU | CCUUUGAAAGUCCUUUCAU | | | | | | |
| | −23_0-2_bulge-asymmetric_-GU | GAAUACAUCCACGGAAAUG | | | | | | |
| | −22_1-1_wobble_G-U | AAUGGCAUAACUAGUCACU | | | | | | |
| | −12_8-6_internal_loop- | GUCGUACAUUGGCCACUCCC | | | | | | |
| | asymmetric_GUAAAGGA-GGCAUA | AGU | | | | | | |
| | −3_2-1_bulge-asymmetric_AG-A | | | | | | | |
| | 0_1-1_mismatch_A-C | | | | | | | |
| | 34_10−10_internal_loop- | | | | | | | |
| | symmetric_AGGAGGGAGU- | | | | | | | |
| | CAGAGGCGAA | | | | | | | |
| 109 | −33_4-4_bulge-symmetric_UUCG- | GCCACACAGACCUCCUUGUA | 0.202 | 0.838 | 0.871 | 1.07 | 1.589 | 1.634 |
| | ACAU | GUGCGAAAGUCCUUUCAUG | | | | | | |
| | −20_4-4_bulge-symmetric_UGGU- | AAUAAGAUCCACGGCUACG | | | | | | |
| | UAGU | UAACUCCUUUACUAGUCAC | | | | | | |
| | −6_6-6_internal_loop- | UGUCGUACAUUGGCCACUC | | | | | | |
| | symmetric_AUUCAU-CGUAAC | CCAGU | | | | | | |
| | 0_1-1_mismatch_A-C | | | | | | | |
| | 6_1-2_bulge-asymmetric_G-AG | | | | | | | |
| | 26_6-6_internal_loop- | | | | | | | |
| | symmetric_AAAGGC-UAGUGC | | | | | | | |
| | 40_4-4_bulge-symmetric_GAGU- | | | | | | | |
| | CAGA | | | | | | | |
| 110 | −33_4-4_bulge-symmetric_UUCG- | GCCACACAGACCUCCUAAUA | 0.076 | 0.905 | 0.913 | 0.913 | 1.561 | 1.65 |
| | ACAU | AAUUGAAAGUCCUUUCAAG | | | | | | |
| | −18_6-6_internal_loop- | AAUACAUCCACGGCUAAUG | | | | | | |
| | symmetric_UGGUGU-UUGUUU | AAUUCCUUUUUGUUUCACU | | | | | | |
| | 0_1-1_mismatch_A-C | GUCGUACAUUGGCCACUCCC | | | | | | |
| | 12_1-1_mismatch_A-A | AGU | | | | | | |
| | 28_6-6_internal_loop- | | | | | | | |
| | symmetric_AGGCCA-AAUAAA | | | | | | | |
| | 40_4-4_bulge-symmetric_GAGU- | | | | | | | |
| | CAGA | | | | | | | |
| 111 | −33_4-4_bulge-symmetric_UUCG- | GCCACACAGAAGUAGCUGG | 0.067 | 0.741 | 0.723 | 0.878 | 1.449 | 1.378 |
| | ACAU | CCUUUGAAAGUCCUGCAUG | | | | | | |
| | −23_0-2_bulge-asymmetric_-GU | AAUACAUCCACGGCUAAAG | | | | | | |
| | −22_1-1_wobble_G-U | AAUGGCAGAACUAGUCACU | | | | | | |
| | −12_8-6_internal_loop- | GUCGUACAUUGGCCACUCCC | | | | | | |
| | asymmetric_GUAAAGGA-GGCAGA | AGU | | | | | | |
| | −7_1-1_mismatch_A-A | | | | | | | |
| | 0_1-1_mismatch_A-C | | | | | | | |
| | 15_2-1_bulge-asymmetric_AA-G | | | | | | | |
| | 34_10−10_internal_loop- | | | | | | | |
| | symmetric_AGGAGGGAGU- | | | | | | | |
| | CAGAAGUAGC | | | | | | | |
| 112 | −33_4-4_bulge-symmetric_UUCG- | GCCACACAGACCUCCUUGAA | 0.021 | 0.624 | 0.64 | 0.931 | 1.4 | 1.365 |
| | ACAU | UAGGGAAAGUCCAUAUUCA | | | | | | |
| | −20_4-4_bulge-symmetric_UGGU- | UGAAUACCCCCACGGCUAA | | | | | | |
| | UAGU | UGAAUGGACCGACUAGUCA | | | | | | |
| | −12 6-6_internal_loop- | CUGUCGUACAUUGGCCACU | | | | | | |
| | symmetric_AAAGGA-GGACCG | CCCAGU | | | | | | |
| | 0_1-1_mismatch_A-C | | | | | | | |
| | 4_2-2_bulge-symmetric_AU-CC | | | | | | | |
| | 17_1-3_bulge-asymmetric_A-AUA | | | | | | | |
| | 26_6-6_internal_loop- | | | | | | | |
| | symmetric_AAAGGC-AAUAGG | | | | | | | |
| | 40_4-4_bulge-symmetric_GAGU- | | | | | | | |
| | CAGA | | | | | | | |
| 113 | −33_4-4_bulge-symmetric_UUCG- | GCCACACAGACCAAUGCUGC | 0.06 | 0.728 | 0.792 | 0.921 | 1.176 | 1.22 |
| | ACAU | CUUUGAAAGUCCUUUGCAU | | | | | | |
| | −20_4-4_bulge-symmetric_UGGU- | CAUUACAUCCACGGCUAAU | | | | | | |
| | UAGU | AGGCGGCUUUACUAGUCAC | | | | | | |
| | −8_6-6_internal_loop- | UGUCGUACAUUGGCCACUC | | | | | | |
| | symmetric_GAAUUC-AGGCGG | CCAGU | | | | | | |
| | 0_1-1_mismatch_A-C | | | | | | | |
| | 9_3-3_bulge-symmetric_UUC-CAU | | | | | | | |
| | 14_0-1_bulge-asymmetric_-G | | | | | | | |
| | 32_6-6_internal_loop- | | | | | | | |
| | symmetric_CAAGGA-AAUGCU | | | | | | | |
| | 40_4-4_bulge-symmetric_GAGU- | | | | | | | |
| | CAGA | | | | | | | |
| 114 | −33_4-4_bulge-symmetric_UUCG- | GCCACACAGACCUCCUCUCA | 0.022 | 0.928 | 0.919 | 0.923 | 1.705 | 1.664 |
| | ACAU | UCUUGAAAGUCGACUUUCA | | | | | | |
| | −18_6-6_internal_loop- | UGAAUACAUCCACGGAAUG | | | | | | |
| | symmetric_UGGUGU-UGUAAU | AAUUCCUUUUGUAAUCACU | | | | | | |
| | −3_2-0_bulge-asymmetric_AG- | GUCGUACAUUGGCCACUCCC | | | | | | |
| | 0_1-1_mismatch_A-C | AGU | | | | | | |
| | 18_0-2_bulge-asymmetric_-GA | | | | | | | |
| | 28_6-6_internal_loop- | | | | | | | |
| | symmetric_AGGCCA-CUCAUC | | | | | | | |
| | 40_4-4_bulge-symmetric_GAGU- | | | | | | | |
| | CAGA | | | | | | | |
| 115 | −33_4-4_bulge-symmetric_UUCG- | GCCACACAGACCUCCUCUCG | 0.016 | 0.716 | 0.791 | 0.939 | 1.544 | 1.603 |
| | ACAU | ACUUGAAAGUCCCUUUCAU | | | | | | |
| | −20_4-4_bulge-symmetric_UGGU- | GAAUUUCUCCACGGCUACG | | | | | | |
| | UAGU | ACUGUCCUUUACUAGUCAC | | | | | | |
| | −6_6-6_internal_loop- | UGUCGUACAUUGGCCACUC | | | | | | |
| | symmetric_AUUCAU-CGACUG | CCAGU | | | | | | |
| | 0_1-1_mismatch_A-C | | | | | | | |
| | 5_3-3_bulge-symmetric_UGU-UUC | | | | | | | |
| | 19_0-1_bulge-asymmetric_-C | | | | | | | |
| | 28_6-6_internal_loop- | | | | | | | |
| | symmetric_AGGCCA-CUCGAC | | | | | | | |
| | 40_4-4_bulge-symmetric_GAGU- | | | | | | | |
| | CAGA | | | | | | | |
| 116 | −33_4-4_bulge-symmetric_UUCG- | GCCACACAGAGGCAGAUGG | 0.103 | 0.898 | 0.908 | 0.899 | 1.058 | 1.083 |
| | ACAU | CCUUUGAAAGUCCUGUCAU | | | | | | |
| | −20_4-4_bulge-symmetric_UGGU- | UAAUACAGCCACGGCAUGC | | | | | | |
| | UAGU | UUAUUCCUUUACUAGUCAC | | | | | | |
| | −4_6-6_internal_loop- | UGUCGUACAUUGGCCACUC | | | | | | |
| | symmetric_UCAUUA-AUGCUU | CCAGU | | | | | | |
| | 0_1-1_mismatch_A-C | | | | | | | |
| | 4_1-1_mismatch_A-G | | | | | | | |
| | 11_1-1_mismatch_C-U | | | | | | | |
| | 16_1-1_mismatch_A-G | | | | | | | |
| | 34_10−10_internal_loop- | | | | | | | |
| | symmetric_AGGAGGGAGU- | | | | | | | |
| | CAGAGGCAGA | | | | | | | |
| 117 | −33_4-4_bulge-symmetric_UUCG- | GCCACACAGACCUCCUUGUA | 0.033 | 0.775 | 0.765 | 0.913 | 1.564 | 1.514 |
| | ACAU | UAUCGAAAGUCCUUUAAUG | | | | | | |
| | −20_4-4_bulge-symmetric_UGGU- | AAUAGAAUCCACGGCUAAU | | | | | | |
| | UAGU | CCUUGGCUUUACUAGUCAC | | | | | | |
| | −8_6-6_internal_loop- | UGUCGUACAUUGGCCACUC | | | | | | |
| | symmetric_GAAUUC-CCUUGG | CCAGU | | | | | | |
| | 0_1-1_mismatch_A-C | | | | | | | |
| | 6_1-2_bulge-asymmetric_G-GA | | | | | | | |
| | 14_1-1_mismatch_G-A | | | | | | | |
| | 26_6-6_internal_loop- | | | | | | | |
| | symmetric_AAAGGC-UAUAUC | | | | | | | |
| | 40_4-4_bulge-symmetric_GAGU- | | | | | | | |
| | CAGA | | | | | | | |
| 118 | −33_4-4_bulge-symmetric_UUCG- | GCCACACAAGAGGGCUUGG | 0.051 | 0.931 | 0.905 | 0.818 | 1.043 | 1.119 |
| | ACAU | CCUUUGAAAGUCCUUUGUU | | | | | | |
| | −18_6-6_internal_loop- | UAAUACAUCCACGGCUUAU | | | | | | |
| | symmetric_UGGUGU-CUUUUU | GAAUUCCUUUCUUUUUCAC | | | | | | |
| | −5_1-1_mismatch_U-U | UGUCGUACAUUGGCCACUC | | | | | | |
| | 0_1-1_mismatch_A-C | CCAGU | | | | | | |
| | 11_4-4_bulge-symmetric_CAUG- | | | | | | | |
| | GUUU | | | | | | | |
| | 36_8-8_internal_loop- | | | | | | | |
| | symmetric_GAGGGAGU- | | | | | | | |
| | CAAGAGGG | | | | | | | |
| 119 | −33_4-4_bulge-symmetric_UUCG- | GCCACACAGACCUCGCAUCA | 0.091 | 0.898 | 0.888 | 0.985 | 1.545 | 1.603 |
| | ACAU | CUUUGAAAGUACUUUCAUU | | | | | | |
| | −20_4-4_bulge-symmetric_UGGU- | AAUACAUCCACGGCUACGU | | | | | | |
| | UAGU | CGCUCCUUUACUAGUCACU | | | | | | |
| | −6_6-6_internal_loop- | GUCGUACAUUGGCCACUCCC | | | | | | |
| | symmetric_AUUCAU-CGUCGC | AGU | | | | | | |
| | 0_1-1_mismatch_A-C | | | | | | | |
| | 11_1-1_mismatch_C-U | | | | | | | |
| | 19_1-1_mismatch_G-A | | | | | | | |
| | 30_6-6_internal_loop- | | | | | | | |
| | symmetric_GCCAAG-GCAUCA | | | | | | | |
| | 40_4-4_bulge-symmetric_GAGU- | | | | | | | |
| | CAGA | | | | | | | |
| 120 | −33_4-4_bulge-symmetric_UUCG- | GCCACACAGACCUCCUUGGC | 0.197 | 0.867 | 0.855 | 1.056 | 1.612 | 1.515 |
| | ACAU | AAGUCCAAGUCCUUUCAUC | | | | | | |
| | −20_4-4_bulge-symmetric_UGGU- | AAUACAUCCACGGCCAUGA | | | | | | |
| | UAGU | CGCGGGUUACUAGUCACUG | | | | | | |
| | −10_6-6_internal_loop- | UCGUACAUUGGCCACUCCCA | | | | | | |
| | symmetric_AGGAAU-CGCGGG | GU | | | | | | |
| | −4_2-1_bulge-asymmetric_UA-C | | | | | | | |
| | 0_1-1_mismatch_A-C | | | | | | | |
| | 11_1-1_mismatch_C-C | | | | | | | |
| | 24_6-6_internal_loop- | | | | | | | |
| | symmetric_UCAAAG-AAGUCC | | | | | | | |
| | 40_4-4_bulge-symmetric_GAGU- | | | | | | | |
| | CAGA | | | | | | | |
| 121 | −33_4-4_bulge-symmetric_UUCG- | GCCACACAGACCUCCUCACU | 0.022 | 0.667 | 0.692 | 0.938 | 1.456 | 1.461 |
| | ACAU | AGUUGAAAGUGAUUCCUUG | | | | | | |
| | −20_4-4_bulge-symmetric_UGGU- | AAUACCUCCACGGCUAAUG | | | | | | |
| | UAGU | AAUUCCUUUACUAGUCACU | | | | | | |
| | 0_1-1_mismatch_A-C | GUCGUACAUUGGCCACUCCC | | | | | | |
| | 5_1-1_mismatch_U-C | AGU | | | | | | |
| | 13_1-2_bulge-asymmetric_U-CU | | | | | | | |
| | 17_3-2_bulge-asymmetric_AGG-GA | | | | | | | |
| | 28_6-6_internal_loop- | | | | | | | |
| | symmetric_AGGCCA-CACUAG | | | | | | | |
| | 40_4-4_bulge-symmetric_GAGU- | | | | | | | |
| | CAGA | | | | | | | |
| 122 | −33_4-4_bulge-symmetric_UUCG- | GCCACACAGACCAGAAGUG | 0.452 | 0.943 | 0.923 | 1.176 | 1.121 | 1.198 |
| | ACAU | CCUUUGAAAGUCCUUUCAU | | | | | | |
| | −20_4-4_bulge-symmetric_UGGU- | GAAUACAACCACGGCUAAU | | | | | | |
| | UAGU | UUGGUACUUUACUAGUCAC | | | | | | |
| | −8_6-6_internal_loop- | UGUCGUACAUUGGCCACUC | | | | | | |
| | symmetric_GAAUUC-UUGGUA | CCAGU | | | | | | |
| | 0_1-1_mismatch_A-C | | | | | | | |
| | 4_1-1_mismatch_A-A | | | | | | | |
| | 32_6-6_internal_loop- | | | | | | | |
| | symmetric_CAAGGA-AGAAGU | | | | | | | |
| | 40_4-4_bulge-symmetric_GAGU- | | | | | | | |
| | CAGA | | | | | | | |
| 123 | −33_4-4_bulge-symmetric_UUCG- | GCCACACAGGGGAGCUUGG | 0.089 | 0.869 | 0.865 | 0.729 | 0.984 | 0.952 |
| | ACAU | CCUUUGAAAGUCCGUUCAU | | | | | | |
| | −20_4-4_bulge-symmetric_UGGU- | GAAUUCAUCCACGGCUAUC | | | | | | |
| | UAGU | AACGUCCUUUACUAGUCAC | | | | | | |
| | −6_6-6_internal_loop- | UGUCGUACAUUGGCCACUC | | | | | | |
| | symmetric_AUUCAU-UCAACG | CCAGU | | | | | | |
| | 0_1-1_mismatch_A-C | | | | | | | |
| | 7_1-1_mismatch_U-U | | | | | | | |
| | 17_1-1_mismatch_A-G | | | | | | | |
| | 36_8-8_internal_loop- | | | | | | | |
| | symmetric_GAGGGAGU- | | | | | | | |
| | CAGGGGAG | | | | | | | |
| 124 | −33_4-4_bulge-symmetric_UUCG- | GCCACACAGACCUCCUAAUA | 0.137 | 0.808 | 0.822 | 1.026 | 1.582 | 1.603 |
| | ACAU | GGUUGAAAGUCCUUUCGUA | | | | | | |
| | −20_4-4_bulge-symmetric_UGGU- | AAUACAUCCACGGCUAAUA | | | | | | |
| | UAGU | GAUGAGGCUUACUAGUCAC | | | | | | |
| | −10_6-6_internal_loop- | UGUCGUACAUUGGCCACUC | | | | | | |
| | symmetric_AGGAAU-UGAGGC | CCAGU | | | | | | |
| | −7_0-1_bulge-asymmetric_-A | | | | | | | |
| | 0_1-1_mismatch_A-C | | | | | | | |
| | 11_1-1_mismatch_C-A | | | | | | | |
| | 13_1-1_wobble_U-G | | | | | | | |
| | 28_6-6_internal_loop- | | | | | | | |
| | symmetric_AGGCCA-AAUAGG | | | | | | | |
| | 40_4-4_bulge-symmetric_GAGU- | | | | | | | |
| | CAGA | | | | | | | |
| 125 | −33_4-4_bulge-symmetric_UUCG- | GCCACACAGACCUCGACACA | 0.024 | 0.549 | 0.604 | 0.93 | 1.321 | 1.339 |
| | ACAU | CUUUGAAAGUCGUAACAUG | | | | | | |
| | −23_0-4_bulge-asymmetric_-UAGU | AAUACCCCCACGGCUAUGG | | | | | | |
| | −20_1-1_wobble_U-G | AAUUCAUGCCAUAGUCACU | | | | | | |
| | −19_1-1_wobble_G-U | GUCGUACAUUGGCCACUCCC | | | | | | |
| | −14_4-0_bulge-asymmetric_AAAG- | AGU | | | | | | |
| | −6_2-2_bulge-symmetric_AU-UG | | | | | | | |
| | 0_1-1_mismatch_A-C | | | | | | | |
| | 4_2-2_bulge-symmetric_AU-CC | | | | | | | |
| | 15_2-2_bulge-symmetric_AA-AA | | | | | | | |
| | 18_1-1_mismatch_G-G | | | | | | | |
| | 30_6-6_internal_loop- | | | | | | | |
| | symmetric_GCCAAG-GACACA | | | | | | | |
| | 40_4-4_bulge-symmetric_GAGU- | | | | | | | |
| | CAGA | | | | | | | |
| 126 | −33_4-4_bulge-symmetric_UUCG- | GCCACACAGACCUCCUUGGC | 0.233 | 0.832 | 0.827 | 1.09 | 1.541 | 1.57 |
| | ACAU | GCGCAUAAGUCCUUUCAUG | | | | | | |
| | −20_4-4_bulge-symmetric_UGGU- | AAUACCUCCACGGCUAAUC | | | | | | |
| | UAGU | GCCCGCUUUACUAGUCACU | | | | | | |
| | −8_6-6_internal_loop- | GUCGUACAUUGGCCACUCCC | | | | | | |
| | symmetric_GAAUUC-CGCCCG | AGU | | | | | | |
| | 0_1-1_mismatch_A-C | | | | | | | |
| | 5_1-1_mismatch_U-C | | | | | | | |
| | 24_6-6_internal_loop- | | | | | | | |
| | symmetric_UCAAAG-GCGCAU | | | | | | | |
| | 40_4-4_bulge-symmetric_GAGU- | | | | | | | |
| | CAGA | | | | | | | |
| 127 | −33_4-4_bulge-symmetric_UUCG- | GCCACACAGACCUCCUUGAU | 0.118 | 0.919 | 0.94 | 1.031 | 1.738 | 1.768 |
| | ACAU | GGCGGAAAGUCCCUUCAUG | | | | | | |
| | −20_6-6_internal_loop- | AAUACUAUCCACGGCUCUA | | | | | | |
| | symmetric_UGUGGU-UGGAGU | CAAUUCCUUUACUGGAGUC | | | | | | |
| | −5_4-4_bulge-symmetric_CAUU- | UGUCGUACAUUGGCCACUC | | | | | | |
| | CUAC | CCAGU | | | | | | |
| | 0_1-1_mismatch_A-C | | | | | | | |
| | 5_0-1_bulge-asymmetric_-U | | | | | | | |
| | 17_1-1_mismatch_A-C | | | | | | | |
| | 26_6-6_internal_loop- | | | | | | | |
| | symmetric_AAAGGC-AUGGCG | | | | | | | |
| | 40_4-4_bulge-symmetric_GAGU- | | | | | | | |
| | CAGA | | | | | | | |
| 128 | −33_4-4_bulge-symmetric_UUCG- | GCCACACAGACCUCCUAUAG | 0.377 | 0.895 | 0.887 | 1.199 | 1.571 | 1.605 |
| | ACAU | AGUUGAAAGUCCUUUCAUG | | | | | | |
| | −20_4-4_bulge-symmetric_UGGU- | AAUACAUCUACGGCUAAUC | | | | | | |
| | UAGU | GCCGACUUUACUAGUCACU | | | | | | |
| | −8_6-6_internal_loop- | GUCGUACAUUGGCCACUCCC | | | | | | |
| | symmetric_GAAUUC-CGCCGA | AGU | | | | | | |
| | 0_1-1_mismatch_A-C | | | | | | | |
| | 2_1-1_wobble_G-U | | | | | | | |
| | 28_6-6_internal_loop- | | | | | | | |
| | symmetric_AGGCCA-AUAGAG | | | | | | | |
| | 40_4-4_bulge-symmetric_GAGU- | | | | | | | |
| | CAGA | | | | | | | |
| 129 | −33_4-4_bulge-symmetric_UUCG- | GCCACACAGACCUCCUCAUG | 0.242 | 0.93 | 0.915 | 1.071 | 1.577 | 1.634 |
| | ACAU | AAUUGAAAGUCCUUUCAUG | | | | | | |
| | −18_6-6_internal_loop- | AAUACAGACACGGCUAAUG | | | | | | |
| | symmetric_UGGUGU-UGUUUU | AAUUCCUUUUGUUUUCACU | | | | | | |
| | 0_1-1_mismatch_A-C | GUCGUACAUUGGCCACUCCC | | | | | | |
| | 3_2-2_bulge-symmetric_GA-GA | AGU | | | | | | |
| | 28_6-6_internal_loop- | | | | | | | |
| | symmetric_AGGCCA-CAUGAA | | | | | | | |
| | 40_4-4_bulge-symmetric_GAGU- | | | | | | | |
| | CAGA | | | | | | | |
| 130 | −33_4-4_bulge-symmetric_UUCG- | GCCACACGGGCAUCCUUGGC | 0.35 | 0.868 | 0.883 | 0.614 | 0.704 | 0.683 |
| | ACAU | CUUUGAAAGUCCUUUCAUG | | | | | | |
| | −23_0-2_bulge-asymmetric_-GU | AAUACAUCCACGGCUAACG | | | | | | |
| | −22_1-1_wobble_G-U | ACCGGGCUUACUAGUCACU | | | | | | |
| | −19_1-1_wobble_G-U | GUCGUACAUUGGCCACUCCC | | | | | | |
| | −15_4-5_internal_loop- | AGU | | | | | | |
| | asymmetric_UAAA-GGGCU | | | | | | | |
| | −10_3-0_bulge-asymmetric_AAU- | | | | | | | |
| | −7_1-1_mismatch_A-C | | | | | | | |
| | 0_1-1_mismatch_A-C | | | | | | | |
| | 38_6-6_internal_loop- | | | | | | | |
| | symmetric_GGGAGU-CGGGCA | | | | | | | |
| 131 | −33_4-4_bulge-symmetric_UUCG- | GCCACACAGACCUCCUUGAU | 0.115 | 0.931 | 0.907 | 0.952 | 1.661 | 1.652 |
| | ACAU | UACAGAAAGUCCUUUCAUG | | | | | | |
| | −23_0-1_bulge-asymmetric_-U | AAUACAGACACGGCUAAUG | | | | | | |
| | −23_1-1_wobble_U-G | AAUUCCUCAAAUGGUCACU | | | | | | |
| | −22_1-1_mismatch_G-G | GUCGUACAUUGGCCACUCCC | | | | | | |
| | −21_1-1_wobble_G-U | AGU | | | | | | |
| | −16_4-3_bulge-asymmetric_GUAA- | | | | | | | |
| | CAA | | | | | | | |
| | 0_1-1_mismatch_A-C | | | | | | | |
| | 3_2-2_bulge-symmetric_GA-GA | | | | | | | |
| | 26_6-6_internal_loop- | | | | | | | |
| | symmetric_AAAGGC-AUUACA | | | | | | | |
| | 40_4-4_bulge-symmetric_GAGU- | | | | | | | |
| | CAGA | | | | | | | |
| 132 | −33_4-4_bulge-symmetric_UUCG- | GCCACACAGACCUCCUAUAG | 0.062 | 0.914 | 0.897 | 0.929 | 1.403 | 1.427 |
| | ACAU | ACUUGAAAGUCGUUUGAUG | | | | | | |
| | −23_0-2_bulge-asymmetric_-GU | AAUACAUCCACGGCUAAUG | | | | | | |
| | −22_1-1_wobble_G-U | ACCGGUAUUACUAGUCACU | | | | | | |
| | −19_1-1_wobble_G-U | GUCGUACAUUGGCCACUCCC | | | | | | |
| | −18_0-1_bulge-asymmetric_-U | AGU | | | | | | |
| | −15_2-2_bulge-symmetric_AA-GG | | | | | | | |
| | −10_3-0_bulge-asymmetric_AAU- | | | | | | | |
| | 0_1-1_mismatch_A-C | | | | | | | |
| | 14_1-1_mismatch_G-G | | | | | | | |
| | 18_1-1_mismatch_G-G | | | | | | | |
| | 28_6-6_internal_loop- | | | | | | | |
| | symmetric_AGGCCA-AUAGAC | | | | | | | |
| | 40_4-4_bulge-symmetric_GAGU- | | | | | | | |
| | CAGA | | | | | | | |
| 133 | −33_4-4_bulge-symmetric_UUCG- | GCCACACAGACCUCCUCAAA | 0.055 | 0.913 | 0.907 | 0.891 | 1.359 | 1.358 |
| | ACAU | ACUUGAAAGUCCUUAGAUG | | | | | | |
| | −20_4-4_bulge-symmetric_UGGU- | AAUACAUCCACGGGUAAUG | | | | | | |
| | UAGU | AAUUCCUUUACUAGUCACU | | | | | | |
| | −3_1-1_mismatch_G-G | GUCGUACAUUGGCCACUCCC | | | | | | |
| | 0_1-1_mismatch_A-C | AGU | | | | | | |
| | 14_2-2_bulge-symmetric_GA-AG | | | | | | | |
| | 28_6-6_internal_loop- | | | | | | | |
| | symmetric_AGGCCA-CAAAAC | | | | | | | |
| | 40_4-4_bulge-symmetric_GAGU- | | | | | | | |
| | CAGA | | | | | | | |
| 134 | −33_4-4_bulge-symmetric_UUCG- | GCCACACAGAGGCGCUUGG | 0.106 | 0.824 | 0.824 | 0.824 | 1.172 | 1.18 |
| | ACAU | CCUUUGAAAGUCCUUUCUG | | | | | | |
| | −20_4-4_bulge-symmetric_UGGU- | AAUCCAUCCACGGCUAAUC | | | | | | |
| | UAGU | UACGGCUUUACUAGUCACU | | | | | | |
| | −8_6-6_internal_loop- | GUCGUACAUUGGCCACUCCC | | | | | | |
| | symmetric_GAAUUC-CUACGG | AGU | | | | | | |
| | 0_1-1_mismatch_A-C | | | | | | | |
| | 7_1-1_mismatch_U-C | | | | | | | |
| | 13_1-0_bulge-asymmetric_U- | | | | | | | |
| | 36_8-8_internal_loop- | | | | | | | |
| | symmetric_GAGGGAGU- | | | | | | | |
| | CAGAGGCG | | | | | | | |
| 135 | −33_4-4_bulge-symmetric_UUCG- | GCCACACAGACCAAGCUCGC | 0.039 | 0.892 | 0.883 | 0.935 | 1.618 | 1.59 |
| | ACAU | CUUUGAAAGUCCAUCAUGA | | | | | | |
| | −23_1-0_bulge-asymmetric_U- | AUAGAUCCACGGCUAACUA | | | | | | |
| | −22_1-1_wobble_G-U | AUUCCUCUUGUAGUCACUG | | | | | | |
| | −21_1-1_mismatch_G-G | UCGUACAUUGGCCACUCCCA | | | | | | |
| | −19_1-1_wobble_G-U | GU | | | | | | |
| | −18_1-1_wobble_U-G | | | | | | | |
| | −15_0-1_bulge-asymmetric_-C | | | | | | | |
| | −7_2-2_bulge-symmetric_CA-CU | | | | | | | |
| | 0_1-1_mismatch_A-C | | | | | | | |
| | 6_1-1_mismatch_G-G | | | | | | | |
| | 16_2-1_bulge-asymmetric_AA-A | | | | | | | |
| | 32_2-1_bulge-asymmetric_CA-C | | | | | | | |
| | 36_3-3_bulge-symmetric_GAG-AAG | | | | | | | |
| | 41_2-2_bulge-symmetric_AG-GA | | | | | | | |
| | 43_0-1_bulge-asymmetric_-C | | | | | | | |
| 136 | −33_4-4_bulge-symmetric_UUCG- | GCCACACAGAGGCGAAUGG | 0.046 | 0.794 | 0.8 | 0.836 | 1.311 | 1.195 |
| | ACAU | CCUUUGAAAGUAGUUUAAU | | | | | | |
| | −23_1-0_bulge-asymmetric_U- | GAAUACAUCCACGGCUACG | | | | | | |
| | −22_1-1_wobble_G-U | AAUUCGGGGUAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_3-4_bulge-asymmetric_AAG- | | | | | | | |
| | GGGG | | | | | | | |
| | −6_2-1_bulge-asymmetric_AU-C | | | | | | | |
| | 0_1-1_mismatch_A-C | | | | | | | |
| | 14_1-1_mismatch_G-A | | | | | | | |
| | 18_2-2_bulge-symmetric_GG-AG | | | | | | | |
| | 34_10−10_internal_loop- | | | | | | | |
| | symmetric_AGGAGGGAGU- | | | | | | | |
| | CAGAGGCGAA | | | | | | | |
| 137 | −33_4-4_bulge-symmetric_UUCG- | GCCACACAGAAGAAUCUGG | 0.052 | 0.868 | 0.89 | 0.883 | 1.424 | 1.468 |
| | ACAU | CCUUUGAAAGUCCUUUCUG | | | | | | |
| | −23_0-1_bulge-asymmetric_-U | UUAUACAUCCACGAUAAUG | | | | | | |
| | −23_1-1_wobble_U-G | AAUUCCUCGUUCGGUCACU | | | | | | |
| | −22_1-1_mismatch_G-G | GUCGUACAUUGGCCACUCCC | | | | | | |
| | −16_5-4_internal_loop- | AGU | | | | | | |
| | asymmetric_UGUAA-CGUU | | | | | | | |
| | −2_2-1_bulge-asymmetric_GC-A | | | | | | | |
| | 0_1-1_mismatch_A-C | | | | | | | |
| | 10_1-2_bulge-asymmetric_U-UU | | | | | | | |
| | 13_1-0_bulge-asymmetric_U- | | | | | | | |
| | 34_10−10_internal_loop- | | | | | | | |
| | symmetric_AGGAGGGAGU- | | | | | | | |
| | CAGAAGAAUC | | | | | | | |
| 138 | −33_4-4_bulge-symmetric_UUCG- | GCCACACAGACCUCCUACAA | 0.014 | 0.803 | 0.827 | 0.953 | 1.604 | 1.612 |
| | ACAU | GGUUGAAAGUCCUUACAUG | | | | | | |
| | −18_6-6_internal_loop- | AAUUCAUCCACGUGUAUUG | | | | | | |
| | symmetric_UGGUGU-UGAGGU | AAUUCCUUUUGAGGUCACU | | | | | | |
| | −6_1-1_mismatch_U-U | GUCGUACAUUGGCCACUCCC | | | | | | |
| | −2_2-2_bulge-symmetric_GC-UG | AGU | | | | | | |
| | 0_1-1_mismatch_A-C | | | | | | | |
| | 7_1-1_mismatch_U-U | | | | | | | |
| | 15_1-1_mismatch_A-A | | | | | | | |
| | 28_6-6_internal_loop- | | | | | | | |
| | symmetric_AGGCCA-ACAAGG | | | | | | | |
| | 40_4-4_bulge-symmetric_GAGU- | | | | | | | |
| | CAGA | | | | | | | |
| 139 | −33_4-4_bulge-symmetric_UUCG- | GCCACACAGACCUCCUUGGC | 0.093 | 0.854 | 0.873 | 0.93 | 1.673 | 1.631 |
| | ACAU | GACGCCAAGUCCUUUCAUG | | | | | | |
| | −20_4-4_bulge-symmetric_UGGU- | AAUACAUCCACGGAUAAUU | | | | | | |
| | UAGU | CUUAGCUUUACUAGUCACU | | | | | | |
| | −8_6-6_internal_loop- | GUCGUACAUUGGCCACUCCC | | | | | | |
| | symmetric_GAAUUC-UCUUAG | AGU | | | | | | |
| | −3_1-1_mismatch_G-A | | | | | | | |
| | 0_1-1_mismatch_A-C | | | | | | | |
| | 24_6-6_internal_loop- | | | | | | | |
| | symmetric_UCAAAG-GACGCC | | | | | | | |
| | 40_4-4_bulge-symmetric_GAGU- | | | | | | | |
| | CAGA | | | | | | | |
| 140 | −33_4-4_bulge-symmetric_UUCG- | GCCACACAGACCUCCUCAUU | 0.064 | 0.932 | 0.928 | 0.965 | 1.722 | 1.669 |
| | ACAU | GAUUGAAAGUAAUUUCAUG | | | | | | |
| | −20_4-4_bulge-symmetric_UGGU- | AAUACAUCCACGGCUACUA | | | | | | |
| | UAGU | CUGUCCUUUACUAGUCACU | | | | | | |
| | −6_6-6_internal_loop- | GUCGUACAUUGGCCACUCCC | | | | | | |
| | symmetric_AUUCAU-CUACUG | AGU | | | | | | |
| | 0_1-1_mismatch_A-C | | | | | | | |
| | 18_2-2_bulge-symmetric_GG-AA | | | | | | | |
| | 28_6-6_internal_loop- | | | | | | | |
| | symmetric_AGGCCA-CAUUGA | | | | | | | |
| | 40_4-4_bulge-symmetric_GAGU- | | | | | | | |
| | CAGA | | | | | | | |
| 141 | −33_4-4_bulge-symmetric_UUCG- | GCCACACAGACCUCCUCUCA | 0.022 | 0.725 | 0.786 | 0.937 | 1.524 | 1.558 |
| | ACAU | GAUUGAAAGUACUUUCAUG | | | | | | |
| | −23_1-0_bulge-asymmetric_U- | AACCUUCCACGGCUACGAA | | | | | | |
| | −22_1-1_wobble_G-U | AUUCGGUUUGUAGUCACUG | | | | | | |
| | −21_1-1_mismatch_G-G | UCGUACAUUGGCCACUCCCA | | | | | | |
| | −19_1-1_wobble_G-U | GU | | | | | | |
| | −18_1-1_wobble_U-G | | | | | | | |
| | −14_1-2_bulge-asymmetric_G-GG | | | | | | | |
| | −6_3-3_bulge-symmetric_CAU-CGA | | | | | | | |
| | 0_1-1_mismatch_A-C | | | | | | | |
| | 5_4-3_bulge-asymmetric_UGUA- | | | | | | | |
| | CCU | | | | | | | |
| | 19_1-1_mismatch_G-A | | | | | | | |
| | 28_6-6_internal_loop- | | | | | | | |
| | symmetric_AGGCCA-CUCAGA | | | | | | | |
| | 40_4-4_bulge-symmetric_GAGU- | | | | | | | |
| | CAGA | | | | | | | |
| 142 | −33_4-4_bulge-symmetric_UUCG- | GCCACACAGACCUCCUUGAU | 0.051 | 0.916 | 0.918 | 0.916 | 1.687 | 1.669 |
| | ACAU | ACGCGAAAGUAAUUUCAUG | | | | | | |
| | −16_8-8_internal_loop- | AAUACAUCCACGGCUACGA | | | | | | |
| | symmetric_UGGUGUAA- | AUUCCUGGUUGAGUCACUG | | | | | | |
| | GGUUGAGU | UCGUACAUUGGCCACUCCCA | | | | | | |
| | −6_2-1_bulge-asymmetric_AU-C | GU | | | | | | |
| | 0_1-1_mismatch_A-C | | | | | | | |
| | 18_2-2_bulge-symmetric_GG-AA | | | | | | | |
| | 26_6-6_internal_loop- | | | | | | | |
| | symmetric_AAAGGC-AUACGC | | | | | | | |
| | 40_4-4_bulge-symmetric_GAGU- | | | | | | | |
| | CAGA | | | | | | | |
| 143 | −33_4-4_bulge-symmetric_UUCG- | GCCACACAGACCUCCUUGUU | 0.354 | 0.773 | 0.794 | 1.225 | 1.538 | 1.543 |
| | ACAU | AUCGGAAAGUCCUUUCAUG | | | | | | |
| | −20_4-4_bulge-symmetric_UGGU- | AAUACAACCACGGCUAAUA | | | | | | |
| | UAGU | CCCGGCUUUACUAGUCACU | | | | | | |
| | −8_6-6_internal_loop- | GUCGUACAUUGGCCACUCCC | | | | | | |
| | symmetric_GAAUUC-ACCCGG | AGU | | | | | | |
| | 0_1-1_mismatch_A-C | | | | | | | |
| | 4_1-1_mismatch_A-A | | | | | | | |
| | 26_6-6_internal_loop- | | | | | | | |
| | symmetric_AAAGGC-UUAUCG | | | | | | | |
| | 40_4-4_bulge-symmetric_GAGU- | | | | | | | |
| | CAGA | | | | | | | |
| 144 | −33_4-4_bulge-symmetric_UUCG- | GCCACACAGACCUCGACUAA | 0.279 | 0.929 | 0.917 | 1.146 | 1.697 | 1.692 |
| | ACAU | CUUUGAAAGUCCUUUCAUG | | | | | | |
| | −20_4-4_bulge-symmetric_UGGU- | AAUAGCUCCACGGCUACUA | | | | | | |
| | UAGU | GUGUCCUUUACUAGUCACU | | | | | | |
| | −6_6-6_internal_loop- | GUCGUACAUUGGCCACUCCC | | | | | | |
| | symmetric_AUUCAU-CUAGUG | AGU | | | | | | |
| | 0_1-1_mismatch_A-C | | | | | | | |
| | 5_2-2_bulge-symmetric_UG-GC | | | | | | | |
| | 30_6-6_internal_loop- | | | | | | | |
| | symmetric_GCCAAG-GACUAA | | | | | | | |
| | 40_4-4_bulge-symmetric_GAGU- | | | | | | | |
| | CAGA | | | | | | | |
| 145 | −33_4-4_bulge-symmetric_UUCG- | GCCACACAGACCUCCUGUUA | 0.079 | 0.883 | 0.84 | 0.96 | 1.57 | 1.517 |
| | ACAU | UGUUGAAAGUAGUUUCAUG | | | | | | |
| | −20_4-4_bulge-symmetric_UGGU- | AAUACAGCCACGACUAAUG | | | | | | |
| | UAGU | AAUGCGACGACUAGUCACU | | | | | | |
| | −12_6-6_internal_loop- | GUCGUACAUUGGCCACUCCC | | | | | | |
| | symmetric_AAAGGA-GCGACG | AGU | | | | | | |
| | −2_1-1_mismatch_C-A | | | | | | | |
| | 0_1-1_mismatch_A-C | | | | | | | |
| | 4_1-1_mismatch_A-G | | | | | | | |
| | 18_2-2_bulge-symmetric_GG-AG | | | | | | | |
| | 28_6-6_internal_loop- | | | | | | | |
| | symmetric_AGGCCA-GUUAUG | | | | | | | |
| | 40_4-4_bulge-symmetric_GAGU- | | | | | | | |
| | CAGA | | | | | | | |
| 146 | −33_4-4_bulge-symmetric_UUCG- | GCCACACAGACCUCCUGUUA | 0.09 | 0.935 | 0.907 | 0.991 | 1.498 | 1.528 |
| | ACAU | UCUUGAAAGUCGUUUCAUG | | | | | | |
| | −16_8-8_internal_loop- | AAUACAACCACGGCUAAUG | | | | | | |
| | symmetric_UGGUGUAA- | AAUUCCUAGUAGAGUCACU | | | | | | |
| | AGUAGAGU | GUCGUACAUUGGCCACUCCC | | | | | | |
| | 0_1-1_mismatch_A-C | AGU | | | | | | |
| | 4_1-1_mismatch_A-A | | | | | | | |
| | 18_1-1_mismatch_G-G | | | | | | | |
| | 28_6-6_internal_loop- | | | | | | | |
| | symmetric_AGGCCA-GUUAUC | | | | | | | |
| | 40_4-4_bulge-symmetric_GAGU- | | | | | | | |
| | CAGA | | | | | | | |
| 147 | −33_4-4_bulge-symmetric_UUCG- | GCCACACAAUAGGACUUGG | 0.131 | 0.908 | 0.887 | 0.822 | 1.121 | 1.149 |
| | ACAU | CCUUUGAAAGUCCUUUCUU | | | | | | |
| | −20_4-4_bulge-symmetric_UGGU- | GAAUACAUCCACGGCAAUG | | | | | | |
| | UAGU | ACGAUACUUACUAGUCACU | | | | | | |
| | −10_6-6_internal_loop- | GUCGUACAUUGGCCACUCCC | | | | | | |
| | symmetric_AGGAAU-CGAUAC | AGU | | | | | | |
| | −4_1-0_bulge-asymmetric_A- | | | | | | | |
| | 0_1-1_mismatch_A-C | | | | | | | |
| | 13_1-1_mismatch_U-U | | | | | | | |
| | 36_8-8_internal_loop- | | | | | | | |
| | symmetric_GAGGGAGU- | | | | | | | |
| | CAAUAGGA | | | | | | | |
| 148 | −33_4-4_bulge-symmetric_UUCG- | GCCACACAGACCAUAGCCGC | 0.023 | 0.709 | 0.783 | 0.92 | 1.414 | 1.429 |
| | ACAU | CUUUGAAAGUACUUUCAUG | | | | | | |
| | −23_0-1_bulge-asymmetric_-U | ACACAUCCACGGAAAUGAA | | | | | | |
| | −23_1-1_wobble_U-G | UUCCUCAUUCGGUCACUGU | | | | | | |
| | −22_1-1_mismatch_G-G | CGUACAUUGGCCACUCCCAG | | | | | | |
| | −20_1-1_mismatch_U-U | U | | | | | | |
| | −19_1-1_wobble_G-U | | | | | | | |
| | −16_2-1_bulge-asymmetric_AA-C | | | | | | | |
| | −3_2-1_bulge-asymmetric_AG-A | | | | | | | |
| | 0_1-1_mismatch_A-C | | | | | | | |
| | 8_2-1_bulge-asymmetric_AU-C | | | | | | | |
| | 19_1-1_mismatch_G-A | | | | | | | |
| | 32_6-6_internal_loop- | | | | | | | |
| | symmetric_CAAGGA-AUAGCC | | | | | | | |
| | 40_4-4_bulge-symmetric_GAGU- | | | | | | | |
| | CAGA | | | | | | | |
| 149 | −33_4-4_bulge-symmetric_UUCG- | GCCACACAGAAAGGACUGG | 0.193 | 0.919 | 0.895 | 0.911 | 1.055 | 1.176 |
| | ACAU | CCUUUGAAAGUCCUUUCAU | | | | | | |
| | −20_4-4_bulge-symmetric_UGGU- | UAAUACAAACACGGCUACG | | | | | | |
| | UAGU | ACAGUCCUUUACUAGUCAC | | | | | | |
| | −6_6-6_internal_loop- | UGUCGUACAUUGGCCACUC | | | | | | |
| | symmetric_AUUCAU-CGACAG | CCAGU | | | | | | |
| | 0_1-1_mismatch_A-C | | | | | | | |
| | 3_2-2_bulge-symmetric_GA-AA | | | | | | | |
| | 11_1-1_mismatch_C-U | | | | | | | |
| | 34_10−10_internal_loop- | | | | | | | |
| | symmetric_AGGAGGGAGU- | | | | | | | |
| | CAGAAAGGAC | | | | | | | |
| 150 | −33_4-4_bulge-symmetric_UUCG- | GCCACACAGACCGGACAAGC | 0.027 | 0.684 | 0.789 | 0.927 | 1.489 | 1.514 |
| | ACAU | CUUUGAAAGUAACUUCAUA | | | | | | |
| | −20_4-4_bulge-symmetric_UGGU- | UCAUCCACGGCUACGUUUC | | | | | | |
| | UAGU | UCCUUUACUAGUCACUGUC | | | | | | |
| | −6_6-6_internal_loop- | GUACAUUGGCCACUCCCAG | | | | | | |
| | symmetric_AUUCAU-CGUUUC | U | | | | | | |
| | 0_1-1_mismatch_A-C | | | | | | | |
| | 7_5-2_internal_loop- | | | | | | | |
| | asymmetric_UAUUC-AU | | | | | | | |
| | 17_3-3_bulge-symmetric_AGG-AAC | | | | | | | |
| | 32_6-6_internal_loop- | | | | | | | |
| | symmetric_CAAGGA-GGACAA | | | | | | | |
| | 40_4-4_bulge-symmetric_GAGU- | | | | | | | |
| | CAGA | | | | | | | |
| 151 | −33_4-4_bulge-symmetric_UUCG- | GCCACACAGACCUCCUCCAG | 0.039 | 0.885 | 0.888 | 0.913 | 1.657 | 1.645 |
| | ACAU | AGUUGAAAGUCCUUUUAUG | | | | | | |
| | −20_4-4_bulge-symmetric_UGGU- | AAUACAUCCACGGCUAAUU | | | | | | |
| | UAGU | CCUAGCUUUACUAGUCACU | | | | | | |
| | −8_6-6_internal_loop- | GUCGUACAUUGGCCACUCCC | | | | | | |
| | symmetric_GAAUUC-UCCUAG | AGU | | | | | | |
| | 0_1-1_mismatch_A-C | | | | | | | |
| | 14_1-1_wobble_G-U | | | | | | | |
| | 28_6-6_internal_loop- | | | | | | | |
| | symmetric_AGGCCA-CCAGAG | | | | | | | |
| | 40_4-4_bulge-symmetric_GAGU- | | | | | | | |
| | CAGA | | | | | | | |
| 152 | −33_4-4_bulge-symmetric_UUCG- | GCCACACAGACCUCGACCGA | 0.17 | 0.474 | 0.611 | 1.046 | 0.965 | 1.1 |
| | ACAU | CUUUGAAAGUCCUUUCAUG | | | | | | |
| | −20_4-4_bulge-symmetric_UGGU- | CCUACAUCCACGGCUAAUCC | | | | | | |
| | UAGU | CGAACUUUACUAGUCACUG | | | | | | |
| | −8_6-6_internal_loop- | UCGUACAUUGGCCACUCCCA | | | | | | |
| | symmetric_GAAUUC-CCCGAA | GU | | | | | | |
| | 0_1-1_mismatch_A-C | | | | | | | |
| | 9_2-2_bulge-symmetric_UU-CC | | | | | | | |
| | 30_6-6_internal_loop- | | | | | | | |
| | symmetric_GCCAAG-GACCGA | | | | | | | |
| | 40_4-4_bulge-symmetric_GAGU- | | | | | | | |
| | CAGA | | | | | | | |
| 153 | −33_4-4_bulge-symmetric_UUCG- | GCCACACAGACCUCCUUGGC | 0.024 | 0.63 | 0.661 | 0.922 | 1.447 | 1.433 |
| | ACAU | AGGCACAAGUCGAUUCAUG | | | | | | |
| | −20_4-4_bulge-symmetric_UGGU- | UUACAUCCACGGCUACGUC | | | | | | |
| | UAGU | UACGCUUUACUAGUCACUG | | | | | | |
| | −6_8-8_internal_loop- | UCGUACAUUGGCCACUCCCA | | | | | | |
| | symmetric_GAAUUCAU- | GU | | | | | | |
| | CGUCUACG | | | | | | | |
| | 0_1-1_mismatch_A-C | | | | | | | |
| | 9_2-1_bulge-asymmetric_UU-U | | | | | | | |
| | 17_2-2_bulge-symmetric_AG-GA | | | | | | | |
| | 24_6-6_internal_loop- | | | | | | | |
| | symmetric_UCAAAG-AGGCAC | | | | | | | |
| | 40_4-4_bulge-symmetric_GAGU- | | | | | | | |
| | CAGA | | | | | | | |
| 154 | −33_4-4_bulge-symmetric_UUCG- | GCCACACAGACCUCCUAUCU | 0.018 | 0.542 | 0.555 | 0.927 | 1.384 | 1.373 |
| | ACAU | AGUUGAAAGUCGAGCCAUG | | | | | | |
| | −23_0-4_bulge-asymmetric_-UGGU | ACUACUUCCACGGCUACUG | | | | | | |
| | −23_1-1_wobble_U-G | AAUUCCUGCCGUGGUCACU | | | | | | |
| | −20_1-1_wobble_U-G | GUCGUACAUUGGCCACUCCC | | | | | | |
| | −16_4-0_bulge-asymmetric_GUAA- | AGU | | | | | | |
| | −6_1-1_mismatch_U-C | | | | | | | |
| | 0_1-1_mismatch_A-C | | | | | | | |
| | 5_1-1_mismatch_U-U | | | | | | | |
| | 9_1-1_mismatch_U-C | | | | | | | |
| | 15_4-4_bulge-symmetric_AAAG- | | | | | | | |
| | GAGC | | | | | | | |
| | 28_6-6_internal_loop- | | | | | | | |
| | symmetric_AGGCCA-AUCUAG | | | | | | | |
| | 40_4-4_bulge-symmetric_GAGU- | | | | | | | |
| | CAGA | | | | | | | |
| 155 | −33_4-4_bulge-symmetric_UUCG- | GCCACACAGACCUCACCCCA | 0.18 | 0.94 | 0.947 | 1.003 | 1.418 | 1.509 |
| | ACAU | CUUUGAAAGUCCUUUCAUU | | | | | | |
| | −18_6-6_internal_loop- | AAUACUUCCACGGCUAAUG | | | | | | |
| | symmetric_UGGUGU-UUGUGU | AAUUCCUUUUUGUGUCACU | | | | | | |
| | 0_1-1_mismatch_A-C | GUCGUACAUUGGCCACUCCC | | | | | | |
| | 5_1-1_mismatch_U-U | AGU | | | | | | |
| | 11_1-1_mismatch_C-U | | | | | | | |
| | 30_6-6_internal_loop- | | | | | | | |
| | symmetric_GCCAAG-ACCCCA | | | | | | | |
| | 40_4-4_bulge-symmetric_GAGU- | | | | | | | |
| | CAGA | | | | | | | |
| 156 | −33_4-4_bulge-symmetric_UUCG- | GCCACACAGACCUCGAACGA | 0.194 | 0.621 | 0.701 | 1.093 | 1.403 | 1.489 |
| | ACAU | CUUUGAAAGUCCUUUCAUG | | | | | | |
| | −20_4-4_bulge-symmetric_UGGU- | AAUAGUUCCACGGCUACCA | | | | | | |
| | UAGU | AUGUCCUUUACUAGUCACU | | | | | | |
| | −6_6-6_internal_loop- | GUCGUACAUUGGCCACUCCC | | | | | | |
| | symmetric_AUUCAU-CCAAUG | AGU | | | | | | |
| | 0_1-1_mismatch_A-C | | | | | | | |
| | 5_2-2_bulge-symmetric_UG-GU | | | | | | | |
| | 30_6-6_internal_loop- | | | | | | | |
| | symmetric_GCCAAG-GAACGA | | | | | | | |
| | 40_4-4_bulge-symmetric_GAGU- | | | | | | | |
| | CAGA | | | | | | | |
| 157 | −33_4-4_bulge-symmetric_UUCG- | GCCACACAGACCUCAGCAAG | 0.133 | 0.525 | 0.568 | 0.966 | 1.205 | 1.261 |
| | ACAU | CUUUGAAAGUCCUUUCAUG | | | | | | |
| | | AAUACAUCCACGGCCGGCU | | | | | | |
| | | AAUUCGAGCUGUAGUCACU | | | | | | |
| | | GUCGUACAUUGGCCACUCCC | | | | | | |
| | | AGU | | | | | | |
| 158 | −33_4-4_bulge-symmetric_UUCG- | GCCACACAGACCUCCUACAA | 0.022 | 0.843 | 0.857 | 0.947 | 1.673 | 1.582 |
| | ACAU | AAUUGAAAGUAAAAUCAUG | | | | | | |
| | −23_0-1_bulge-asymmetric_-U | AAUACCACCACGGCUUAUG | | | | | | |
| | −23_1-1_wobble_U-G | AAUUCCUGUUUCGGUCACU | | | | | | |
| | −22_1-1_mismatch_G-G | GUCGUACAUUGGCCACUCCC | | | | | | |
| | −16_5-4_internal_loop- | AGU | | | | | | |
| | asymmetric_UGUAA-GUUU | | | | | | | |
| | −5_1-1_mismatch_U-U | | | | | | | |
| | 0_1-1_mismatch_A-C | | | | | | | |
| | 4_2-2_bulge-symmetric_AU-CA | | | | | | | |
| | 16_4-4_bulge-symmetric_AAGG- | | | | | | | |
| | AAAA | | | | | | | |
| | 28_6-6_internal_loop- | | | | | | | |
| | symmetric_AGGCCA-ACAAAA | | | | | | | |
| | 40_4-4_bulge-symmetric_GAGU- | | | | | | | |
| | CAGA | | | | | | | |
| 159 | −33_4-4_bulge-symmetric_UUCG- | GCCACACAGAGGGAAAUGG | 0.154 | 0.926 | 0.909 | 0.787 | 1.138 | 1.232 |
| | ACAU | CCUUUGAAAGUCCUUUCAU | | | | | | |
| | −14_10−10_internal_loop- | GAAUACAUCCACGUGCUAC | | | | | | |
| | symmetric_UGGUGUAAAG- | CGAAUUCAUUCUGUAGUCA | | | | | | |
| | AUUCUGUAGU | CUGUCGUACAUUGGCCACU | | | | | | |
| | −6_2-2_bulge-symmetric_AU-CC | CCCAGU | | | | | | |
| | −1_0-1_bulge-asymmetric_-U | | | | | | | |
| | 0_1-1_mismatch_A-C | | | | | | | |
| | 34_10−10_internal_loop- | | | | | | | |
| | symmetric_AGGAGGGAGU- | | | | | | | |
| | CAGAGGGAAA | | | | | | | |
| 160 | −33_4-4_bulge-symmetric_UUCG- | GCCACACAGAAAGAAAUGG | 0.433 | 0.925 | 0.88 | 0.888 | 1.162 | 1.174 |
| | ACAU | CCUUUGAAAGUCCUUUCAU | | | | | | |
| | −23_0-2_bulge-asymmetric_-GU | GAAUAGAUCCACGGCUAAU | | | | | | |
| | −22_1-1_wobble_G-U | GAUCGGUAUUACUAGUCAC | | | | | | |
| | −19_1-1_wobble_G-U | UGUCGUACAUUGGCCACUC | | | | | | |
| | −14_5-5_internal_loop- | CCAGU | | | | | | |
| | symmetric_UAAAG-GGUAU | | | | | | | |
| | −10_2-0_bulge-asymmetric_AU- | | | | | | | |
| | 0_1-1_mismatch_A-C | | | | | | | |
| | 6_1-1_mismatch_G-G | | | | | | | |
| | 34_10−10_internal_loop- | | | | | | | |
| | symmetric_AGGAGGGAGU- | | | | | | | |
| | CAGAAAGAAA | | | | | | | |
| 161 | −33_4-4_bulge-symmetric_UUCG- | GCCACACAGACCUCCUUGAU | 0.033 | 0.706 | 0.691 | 0.92 | 1.477 | 1.428 |
| | ACAU | GGUGGAAAGUCCUUACCUG | | | | | | |
| | −23_0-2_bulge-asymmetric_-GU | AAUACAUCCACGGCUAAUG | | | | | | |
| | −22_1-1_wobble_G-U | AUCUGAGUUACUAGUCACU | | | | | | |
| | −19_1-1_wobble_G-U | GUCGUACAUUGGCCACUCCC | | | | | | |
| | −15_4-4_bulge-symmetric_UAAA- | AGU | | | | | | |
| | GAGU | | | | | | | |
| | −14_1-1_wobble_G-U | | | | | | | |
| | −10_2-0_bulge-asymmetric_AU- | | | | | | | |
| | 0_1-1_mismatch_A-C | | | | | | | |
| | 13 1-1_mismatch_U-C | | | | | | | |
| | 15_1-1_mismatch_A-A | | | | | | | |
| | 26_6-6_internal_loop- | | | | | | | |
| | symmetric_AAAGGC-AUGGUG | | | | | | | |
| | 40_4-4_bulge-symmetric_GAGU- | | | | | | | |
| | CAGA | | | | | | | |
| 162 | −33_4-4_bulge-symmetric_UUCG- | GCCACACAGACCUCCUAAUG | 0.151 | 0.912 | 0.896 | 1.041 | 1.612 | 1.602 |
| | ACAU | AAUUGAAAGUCCUUUCAUG | | | | | | |
| | −20_4-4_bulge-symmetric_UGGU- | UAUACAUCCACGGUUAAUC | | | | | | |
| | UAGU | ACCGACUUUACUAGUCACU | | | | | | |
| | −8_6-6_internal_loop- | GUCGUACAUUGGCCACUCCC | | | | | | |
| | symmetric_GAAUUC-CACCGA | AGU | | | | | | |
| | −3 1-1_wobble_G-U | | | | | | | |
| | 0_1-1_mismatch_A-C | | | | | | | |
| | 10_1-1_mismatch_U-U | | | | | | | |
| | 28_6-6_internal_loop- | | | | | | | |
| | symmetric_AGGCCA-AAUGAA | | | | | | | |
| | 40_4-4_bulge-symmetric_GAGU- | | | | | | | |
| | CAGA | | | | | | | |
| 163 | −33_4-4_bulge-symmetric_UUCG- | GCCACACAGACCUCCUUGGC | 0.043 | 0.897 | 0.872 | 0.928 | 1.55 | 1.492 |
| | ACAU | GGUGCCAAGUCCCGUCAUG | | | | | | |
| | −23_1-0_bulge-asymmetric_U- | AAUACAUCCACGGCUAAUG | | | | | | |
| | −22_1-1_wobble_G-U | AAUUCGGGGGAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_4-5_internal_loop- | | | | | | | |
| | asymmetric_AAAG-GGGGG | | | | | | | |
| | 0_1-1_mismatch_A-C | | | | | | | |
| | 16_2-2_bulge-symmetric_AA-CG | | | | | | | |
| | 24_6-6_internal_loop- | | | | | | | |
| | symmetric_UCAAAG-GGUGCC | | | | | | | |
| | 40_4-4_bulge-symmetric_GAGU- | | | | | | | |
| | CAGA | | | | | | | |
| 164 | −33_4-4_bulge-symmetric_UUCG- | GCCACACAGACCUCAAUUA | 0.078 | 0.928 | 0.931 | 0.974 | 1.312 | 1.324 |
| | ACAU | ACUUUGAAAGUACUUUCAU | | | | | | |
| | −21_3-4_bulge-asymmetric_UGG- | GAUUACAUCCACGAUAAUG | | | | | | |
| | UAGU | AAUUCACAACGUAGUCACU | | | | | | |
| | −20_1-1_wobble_U-G | GUCGUACAUUGGCCACUCCC | | | | | | |
| | −14_4-3_bulge-asymmetric_AAAG- | AGU | | | | | | |
| | ACA | | | | | | | |
| | −2_2-1_bulge-asymmetric_GC-A | | | | | | | |
| | 0_1-1_mismatch_A-C | | | | | | | |
| | 9_1-1_mismatch_U-U | | | | | | | |
| | 19_1-1_mismatch_G-A | | | | | | | |
| | 30_6-6_internal_loop- | | | | | | | |
| | symmetric_GCCAAG-AAUUAA | | | | | | | |
| | 40_4-4_bulge-symmetric_GAGU- | | | | | | | |
| | CAGA | | | | | | | |
| 165 | −33_4-4_bulge-symmetric_UUCG- | GCCACACAGACCUCCUAUCA | 0.04 | 0.581 | 0.652 | 0.974 | 1.41 | 1.457 |
| | ACAU | GGUUGAAAGUCCAUUCAUG | | | | | | |
| | −20_4-4_bulge-symmetric_UGGU- | AAUAUAUCCACGAUAAGCG | | | | | | |
| | UAGU | GAAUUCCUUUACUAGUCAC | | | | | | |
| | −7→0_8-9_internal_loop- | UGUCGUACAUUGGCCACUC | | | | | | |
| | asymmetric_AUUAGCCA- | CCAGU | | | | | | |
| | CGAUAAGCG | | | | | | | |
| | 6_1-1_wobble_G-U | | | | | | | |
| | 17_1-1_mismatch_A-A | | | | | | | |
| | 28_6-6_internal_loop- | | | | | | | |
| | symmetric_AGGCCA-AUCAGG | | | | | | | |
| | 40_4-4_bulge-symmetric_GAGU- | | | | | | | |
| | CAGA | | | | | | | |
| 166 | −33_4-4_bulge-symmetric_UUCG- | GCCACACAGACCUCCUGUUA | 0.03 | 0.599 | 0.572 | 0.916 | 1.411 | 1.411 |
| | ACAU | UAUUGAAAGUGACGACAUG | | | | | | |
| | −18_6-6_internal_loop- | AAUACAUCCACGGCUAAUG | | | | | | |
| | symmetric_UGGUGU-UGAAAU | AAUUCCUUUUGAAAUCACU | | | | | | |
| | 0_1-1_mismatch_A-C | GUCGUACAUUGGCCACUCCC | | | | | | |
| | 15_5-5_internal_loop- | AGU | | | | | | |
| | symmetric_AAAGG-GACGA | | | | | | | |
| | 28_6-6_internal_loop- | | | | | | | |
| | symmetric_AGGCCA-GUUAUA | | | | | | | |
| | 40_4-4_bulge-symmetric_GAGU- | | | | | | | |
| | CAGA | | | | | | | |
| 167 | −33_4-4_bulge-symmetric_UUCG- | GCCACACAGACCUCCUAGAA | 0.061 | 0.908 | 0.884 | 0.931 | 1.517 | 1.511 |
| | ACAU | GAUUGAAAGUCCGUUCAUG | | | | | | |
| | −23_1-0_bulge-asymmetric_U- | AAUACAUCCACGGUUAAUG | | | | | | |
| | −22_1-1_wobble_G-U | AAUUCGAUGGAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_4-5_internal_loop- | | | | | | | |
| | asymmetric_AAAG-GAUGG | | | | | | | |
| | −3_1-1_wobble_G-U | | | | | | | |
| | 0_1-1_mismatch_A-C | | | | | | | |
| | 17_1-1_mismatch_A-G | | | | | | | |
| | 28_6-6_internal_loop- | | | | | | | |
| | symmetric_AGGCCA-AGAAGA | | | | | | | |
| | 40_4-4_bulge-symmetric_GAGU- | | | | | | | |
| | CAGA | | | | | | | |
| 168 | −33_4-4_bulge-symmetric_UUCG- | GCCACACAGACCUCCUUGUU | 0.03 | 0.761 | 0.855 | 0.94 | 1.577 | 1.544 |
| | ACAU | AGCAGAAAGUAUCUUCAUA | | | | | | |
| | −20_6-6_internal_loop- | AAUAGAUCCACGGCUAAUG | | | | | | |
| | symmetric_UGUGGU-UAGAAU | AAUUCCUUUACUAGAAUCU | | | | | | |
| | 0_1-1_mismatch_A-C | GUCGUACAUUGGCCACUCCC | | | | | | |
| | 6_1-1_mismatch_G-G | AGU | | | | | | |
| | 11_1-1_mismatch_C-A | | | | | | | |
| | 17_3-3_bulge-symmetric_AGG-AUC | | | | | | | |
| | 26_6-6_internal_loop- | | | | | | | |
| | symmetric_AAAGGC-UUAGCA | | | | | | | |
| | 40_4-4_bulge-symmetric_GAGU- | | | | | | | |
| | CAGA | | | | | | | |
| 169 | −33_4-4_bulge-symmetric_UUCG- | GCCACACAGACCUCCUACAU | 0.049 | 0.834 | 0.842 | 0.957 | 1.639 | 1.617 |
| | ACAU | UCUUGAAAGUAGUUUCGUG | | | | | | |
| | −20_4-4_bulge-symmetric_UGGU- | AAUACAUCCACGGCUAUUC | | | | | | |
| | UAGU | ACCUCCUUUACUAGUCACU | | | | | | |
| | −6_6-6_internal_loop- | GUCGUACAUUGGCCACUCCC | | | | | | |
| | symmetric_AUUCAU-UUCACC | AGU | | | | | | |
| | 0_1-1_mismatch_A-C | | | | | | | |
| | 13 1-1_wobble_U-G | | | | | | | |
| | 18_2-2_bulge-symmetric_GG-AG | | | | | | | |
| | 28_6-6_internal_loop- | | | | | | | |
| | symmetric_AGGCCA-ACAUUC | | | | | | | |
| | 40_4-4_bulge-symmetric_GAGU- | | | | | | | |
| | CAGA | | | | | | | |
| 170 | −33_4-4_bulge-symmetric_UUCG- | GCCACACAGACCUCCUUGAU | 0.031 | 0.685 | 0.721 | 0.943 | 1.494 | 1.497 |
| | ACAU | GGCGGAAAGUCCUUUCAUG | | | | | | |
| | −23_0-4_bulge-asymmetric_-UGGU | GCACAUCCACGGCUAACAA | | | | | | |
| | −23_1-1_wobble_U-G | UUCCUACCGUGGUCACUGU | | | | | | |
| | −16_4-0_bulge-asymmetric_GUAA- | CGUACAUUGGCCACUCCCAG | | | | | | |
| | −7_2-1_bulge-asymmetric_CA-C | U | | | | | | |
| | 0_1-1_mismatch_A-C | | | | | | | |
| | 8_2-1_bulge-asymmetric_AU-C | | | | | | | |
| | 10_1-1_wobble_U-G | | | | | | | |
| | 26_6-6_internal_loop- | | | | | | | |
| | symmetric_AAAGGC-AUGGCG | | | | | | | |
| | 40_4-4_bulge-symmetric_GAGU- | | | | | | | |
| | CAGA | | | | | | | |
| 171 | −33_4-4_bulge-symmetric_UUCG- | GCCACACAAGUAGGCUUGG | 0.073 | 0.775 | 0.782 | 0.881 | 1.436 | 1.404 |
| | ACAU | CCUUUGAAAGUCCUUUCCC | | | | | | |
| | −20_4-4_bulge-symmetric_UGGU- | UGAAUACCCCACGGCUAAU | | | | | | |
| | UAGU | UUAGAGCUUUACUAGUCAC | | | | | | |
| | −8_6-6_internal_loop- | UGUCGUACAUUGGCCACUC | | | | | | |
| | symmetric_GAAUUC-UUAGAG | CCAGU | | | | | | |
| | 0_1-1_mismatch_A-C | | | | | | | |
| | 4_2-1_bulge-asymmetric_AU-C | | | | | | | |
| | 13_1-2_bulge-asymmetric_U-CC | | | | | | | |
| | 36_8-8_internal_loop- | | | | | | | |
| | symmetric_GAGGGAGU- | | | | | | | |
| | CAAGUAGG | | | | | | | |
| 172 | −33_4-4_bulge-symmetric_UUCG- | GCCACACAGACCAGAGCUGC | 0.049 | 0.932 | 0.89 | 0.849 | 1.287 | 1.242 |
| | ACAU | CUUUGAAAGUCCUUUGUUG | | | | | | |
| | −18_6-6_internal_loop- | AAUAGAUCCACGGCUAAUG | | | | | | |
| | symmetric_UGGUGU-UGGUAU | AAUUCCUUUUGGUAUCACU | | | | | | |
| | 0_1-1_mismatch_A-C | GUCGUACAUUGGCCACUCCC | | | | | | |
| | 6_1-1_mismatch_G-G | AGU | | | | | | |
| | 13_2-2_bulge-symmetric_UG-GU | | | | | | | |
| | 32_6-6_internal_loop- | | | | | | | |
| | symmetric_CAAGGA-AGAGCU | | | | | | | |
| | 40_4-4_bulge-symmetric_GAGU- | | | | | | | |
| | CAGA | | | | | | | |
| 173 | −33_4-4_bulge-symmetric_UUCG- | GCCACAUGUAUAUCCUUGG | 0.235 | 0.916 | 0.892 | 0.739 | 0.799 | 0.818 |
| | ACAU | CCUUUGAAAGUCCAUUCAU | | | | | | |
| | −20_4-4_bulge-symmetric_UGGU- | GAAUACAUCCACGGCUACG | | | | | | |
| | UAGU | UCGGUCCUUUACUAGUCAC | | | | | | |
| | −6_6-6_internal_loop- | UGUCGUACAUUGGCCACUC | | | | | | |
| | symmetric_AUUCAU-CGUCGG | CCAGU | | | | | | |
| | 0_1-1_mismatch_A-C | | | | | | | |
| | 17_1-1_mismatch_A-A | | | | | | | |
| | 38_6-6_internal_loop- | | | | | | | |
| | symmetric_GGGAGU-UGUAUA | | | | | | | |
| 174 | −33_4-4_bulge-symmetric_UUCG- | GCCACACAGACCGGAGUUG | 0.198 | 0.941 | 0.916 | 0.904 | 1.135 | 1.238 |
| | ACAU | CCUUUGAAAGUCCUUUCAU | | | | | | |
| | −18_6-6_internal_loop- | GAAUACACACACGGCAAUG | | | | | | |
| | symmetric_UGGUGU-CUCGUU | AAUUCCUUUCUCGUUCACU | | | | | | |
| | −4_1-0_bulge-asymmetric_A- | GUCGUACAUUGGCCACUCCC | | | | | | |
| | 0_1-1_mismatch_A-C | AGU | | | | | | |
| | 3_2-2_bulge-symmetric_GA-CA | | | | | | | |
| | 32_6-6_internal_loop- | | | | | | | |
| | symmetric_CAAGGA-GGAGUU | | | | | | | |
| | 40_4-4_bulge-symmetric_GAGU- | | | | | | | |
| | CAGA | | | | | | | |
| 175 | −33_4-4_bulge-symmetric_UUCG- | GCCACACAGACCUCCUAUUG | 0.079 | 0.883 | 0.883 | 0.942 | 1.284 | 1.283 |
| | ACAU | ACUUGAAAGUCCAUUCAUG | | | | | | |
| | −20_4-4_bulge-symmetric_UGGU- | AAUACAUCCACGGUUUAUG | | | | | | |
| | UAGU | AAUGGCAUGACUAGUCACU | | | | | | |
| | −12_6-6_internal_loop- | GUCGUACAUUGGCCACUCCC | | | | | | |
| | symmetric_AAAGGA-GGCAUG | AGU | | | | | | |
| | −5_1-1_mismatch_U-U | | | | | | | |
| | −3_1-1_wobble_G-U | | | | | | | |
| | 0_1-1_mismatch_A-C | | | | | | | |
| | 17_1-1_mismatch_A-A | | | | | | | |
| | 28_6-6_internal_loop- | | | | | | | |
| | symmetric_AGGCCA-AUUGAC | | | | | | | |
| | 40_4-4_bulge-symmetric_GAGU- | | | | | | | |
| | CAGA | | | | | | | |
| 176 | −33_4-4_bulge-symmetric_UUCG- | GCCACACAGACCUCCUUGUA | 0.02 | 0.758 | 0.771 | 0.926 | 1.581 | 1.552 |
| | ACAU | UGUGGAAAGUCCUUUGCUG | | | | | | |
| | −20_4-4_bulge-symmetric_UGGU- | AAUACAUCCACGUGUAAUC | | | | | | |
| | UAGU | UCUAACUUUACUAGUCACU | | | | | | |
| | −8_6-6_internal_loop- | GUCGUACAUUGGCCACUCCC | | | | | | |
| | symmetric_GAAUUC-CUCUAA | AGU | | | | | | |
| | −2_2-2_bulge-symmetric_GC-UG | | | | | | | |
| | 0_1-1_mismatch_A-C | | | | | | | |
| | 13_2-2_bulge-symmetric_UG-GC | | | | | | | |
| | 26_6-6_internal_loop- | | | | | | | |
| | symmetric_AAAGGC-UAUGUG | | | | | | | |
| | 40_4-4_bulge-symmetric_GAGU- | | | | | | | |
| | CAGA | | | | | | | |
| 177 | −33_4-4_bulge-symmetric_UUCG- | GCCACACAGACCUCCUUGGC | 0.047 | 0.749 | 0.768 | 0.935 | 1.389 | 1.324 |
| | ACAU | CUGGACUUGUCCUUUCACU | | | | | | |
| | −20_4-4_bulge-symmetric_UGGU- | AAUACAUGCACGGCUAAUA | | | | | | |
| | UAGU | CUAUACUUUACUAGUCACU | | | | | | |
| | −8_6-6_internal_loop- | GUCGUACAUUGGCCACUCCC | | | | | | |
| | symmetric_GAAUUC-ACUAUA | AGU | | | | | | |
| | 0_1-1_mismatch_A-C | | | | | | | |
| | 3_1-1_mismatch_G-G | | | | | | | |
| | 11_2-2_bulge-symmetric_CA-CU | | | | | | | |
| | 22_6-6_internal_loop- | | | | | | | |
| | symmetric_UUUCAA-GGACUU | | | | | | | |
| | 40_4-4_bulge-symmetric_GAGU- | | | | | | | |
| | CAGA | | | | | | | |
| 178 | −33_4-4_bulge-symmetric_UUCG- | GCCACACAGAAUAGAAUGG | 0.166 | 0.571 | 0.562 | 0.859 | 0.895 | 0.997 |
| | ACAU | CCUUUGAAAGUCCUUUCAU | | | | | | |
| | −20_4-4_bulge-symmetric_UGGU- | UAAUACCUCCACAGCUAAC | | | | | | |
| | UAGU | GAUAGAGCUUACUAGUCAC | | | | | | |
| | −10_6-6_internal_loop- | UGUCGUACAUUGGCCACUC | | | | | | |
| | symmetric_AGGAAU-UAGAGC | CCAGU | | | | | | |
| | −7_1-1_mismatch_A-C | | | | | | | |
| | −1→0_2-2_bulge-symmetric_CA-CA | | | | | | | |
| | 5_1-1_mismatch_U-C | | | | | | | |
| | 11_1-1_mismatch_C-U | | | | | | | |
| | 34_10−10_internal_loop- | | | | | | | |
| | symmetric_AGGAGGGAGU- | | | | | | | |
| | CAGAAUAGAA | | | | | | | |
| 179 | −33_4-4_bulge-symmetric_UUCG- | GCCACACAGGGGCACUUGG | 0.058 | 0.705 | 0.706 | 0.659 | 0.58 | 0.578 |
| | ACAU | CCUUUGAAAGUCACUUUCA | | | | | | |
| | −16_8-8_internal_loop- | UAAUACAUACACGGCUCUC | | | | | | |
| | symmetric_UGGUGUAA- | GAAUUCCUCUCUUGGUCAC | | | | | | |
| | CUCUUGGU | UGUCGUACAUUGGCCACUC | | | | | | |
| | −5_3-3_bulge-symmetric_AUU-CUC | CCAGU | | | | | | |
| | 0_1-1_mismatch_A-C | | | | | | | |
| | 3_2-2_bulge-symmetric_GA-UA | | | | | | | |
| | 11_1-0_bulge-asymmetric_C- | | | | | | | |
| | 18_0-1_bulge-asymmetric_-A | | | | | | | |
| | 36_8-8_internal_loop- | | | | | | | |
| | symmetric_GAGGGAGU- | | | | | | | |
| | CAGGGGCA | | | | | | | |
| 180 | −33_4-4_bulge-symmetric_UUCG- | GCCACACAGACCUCCUCAAU | 0.049 | 0.75 | 0.745 | 0.963 | 1.551 | 1.527 |
| | ACAU | GAUUGAAAGUCGGUUCAUG | | | | | | |
| | −23_0-2_bulge-asymmetric_-GU | AAUAGAUCCACGGCUAAUG | | | | | | |
| | −22_1-1_wobble_G-U | ACCGAGAUUACUAGUCACU | | | | | | |
| | −19_1-1_wobble_G-U | GUCGUACAUUGGCCACUCCC | | | | | | |
| | −15_4-5_internal_loop- | AGU | | | | | | |
| | asymmetric_UAAA-GAGAU | | | | | | | |
| | −10_3-0_bulge-asymmetric_AAU- | | | | | | | |
| | 0_1-1_mismatch_A-C | | | | | | | |
| | 6_1-1_mismatch_G-G | | | | | | | |
| | 17_2-2_bulge-symmetric_AG-GG | | | | | | | |
| | 28_6-6_internal_loop- | | | | | | | |
| | symmetric_AGGCCA-CAAUGA | | | | | | | |
| | 40_4-4_bulge-symmetric_GAGU- | | | | | | | |
| | CAGA | | | | | | | |
| 181 | −33_4-4_bulge-symmetric_UUCG- | GCCACACAGACCAAGCGCGC | 0.072 | 0.918 | 0.907 | 0.939 | 1.596 | 1.529 |
| | ACAU | CUUUGAAAGUCCAUUCAUU | | | | | | |
| | −20_4-4_bulge-symmetric_UGGU- | AAUAACUCCACGGCUAAUU | | | | | | |
| | UAGU | UCUUACUUUACUAGUCACU | | | | | | |
| | −8_6-6_internal_loop- | GUCGUACAUUGGCCACUCCC | | | | | | |
| | symmetric_GAAUUC-UUCUUA | AGU | | | | | | |
| | 0_1-1_mismatch_A-C | | | | | | | |
| | 5_2-2_bulge-symmetric_UG-AC | | | | | | | |
| | 11_1-1_mismatch_C-U | | | | | | | |
| | 17_1-1_mismatch_A-A | | | | | | | |
| | 32_6-6_internal_loop- | | | | | | | |
| | symmetric_CAAGGA-AAGCGC | | | | | | | |
| | 40_4-4_bulge-symmetric_GAGU- | | | | | | | |
| | CAGA | | | | | | | |
| 182 | −33_4-4_bulge-symmetric_UUCG- | GCCACACAGACCUCCUAUCA | 0.082 | 0.913 | 0.892 | 0.98 | 1.606 | 1.599 |
| | ACAU | GAUUGAAAGUGCUUUCAUG | | | | | | |
| | −23_0-4_bulge-asymmetric_-UAGU | AAUACAUCCACAGCUAAUG | | | | | | |
| | −23_1-1_wobble_U-G | AAUUCGCAGCGUAGUCACU | | | | | | |
| | −21_1-1_mismatch_G-G | GUCGUACAUUGGCCACUCCC | | | | | | |
| | −18_1-1_wobble_U-G | AGU | | | | | | |
| | −14_4-0_bulge-asymmetric_AAAG- | | | | | | | |
| | −1→0_2-2_bulge-symmetric_CA-CA | | | | | | | |
| | 19_1-1_mismatch_G-G | | | | | | | |
| | 28_6-6_internal_loop- | | | | | | | |
| | symmetric_AGGCCA-AUCAGA | | | | | | | |
| | 40_4-4_bulge-symmetric_GAGU- | | | | | | | |
| | CAGA | | | | | | | |
| 183 | −33_4-4_bulge-symmetric_UUCG- | GCCACACAGACCUCAGCCAG | 0.072 | 0.912 | 0.888 | 0.957 | 1.609 | 1.576 |
| | ACAU | CUUUGAAAGUACUUUCAUG | | | | | | |
| | −22_2-3_bulge-asymmetric_UG-AGU | AAUACAUCCACGGAGUGAA | | | | | | |
| | −21_1-1_wobble_G-U | UUCGACGCAUAGUCACUGU | | | | | | |
| | −18_1-1_wobble_U-G | CGUACAUUGGCCACUCCCAG | | | | | | |
| | −14_4-3_bulge-asymmetric_AAAG- | U | | | | | | |
| | GAC | | | | | | | |
| | −6_1-1_wobble_U-G | | | | | | | |
| | −3_2-0_bulge-asymmetric_AG- | | | | | | | |
| | 0_1-1_mismatch_A-C | | | | | | | |
| | 19_1-1_mismatch_G-A | | | | | | | |
| | 30_6-6_internal_loop- | | | | | | | |
| | symmetric_GCCAAG-AGCCAG | | | | | | | |
| | 40_4-4_bulge-symmetric_GAGU- | | | | | | | |
| | CAGA | | | | | | | |
| 184 | −33_4-4_bulge-symmetric_UUCG- | GCCACACAGACCUCCUAUCA | 0.182 | 0.689 | 0.706 | 1.078 | 1.48 | 1.494 |
| | ACAU | UAUUGAAAGUCCUUUCAUG | | | | | | |
| | −20_4-4_bulge-symmetric_UGGU- | CAUACACCCACGGCAUGUU | | | | | | |
| | UAGU | ACGAGCUUUACUAGUCACU | | | | | | |
| | −4_10−10_internal_loop- | GUCGUACAUUGGCCACUCCC | | | | | | |
| | symmetric_GAAUUCAUUA- | AGU | | | | | | |
| | AUGUUACGAG | | | | | | | |
| | 0_1-1_mismatch_A-C | | | | | | | |
| | 4_1-1_mismatch_A-C | | | | | | | |
| | 10_1-1_mismatch_U-C | | | | | | | |
| | 28_6-6_internal_loop- | | | | | | | |
| | symmetric_AGGCCA-AUCAUA | | | | | | | |
| | 40_4-4_bulge-symmetric_GAGU- | | | | | | | |
| | CAGA | | | | | | | |
| 185 | −33_4-4_bulge-symmetric_UUCG- | GCCACACAGACCUCCUUGGC | 0.078 | 0.635 | 0.714 | 0.968 | 1.42 | 1.44 |
| | ACAU | AAGGUCAAGUCCUUUCAUG | | | | | | |
| | −16_8-8_internal_loop- | AAUACCUUCCACGGCUCUA | | | | | | |
| | symmetric_UGGUGUAA- | GAAUUCCUACUGGAGUCAC | | | | | | |
| | ACUGGAGU | UGUCGUACAUUGGCCACUC | | | | | | |
| | −5_3-3_bulge-symmetric_AUU-CUA | CCAGU | | | | | | |
| | 0_1-1_mismatch_A-C | | | | | | | |
| | 5_1-2_bulge-asymmetric_U-CU | | | | | | | |
| | 24_6-6_internal_loop- | | | | | | | |
| | symmetric_UCAAAG-AAGGUC | | | | | | | |
| | 40_4-4_bulge-symmetric_GAGU- | | | | | | | |
| | CAGA | | | | | | | |
| 186 | −33_4-4_bulge-symmetric_UUCG- | GCCACACAGACCAAGGUCGC | 0.044 | 0.851 | 0.883 | 0.911 | 1.462 | 1.451 |
| | ACAU | CUUUGAAAGUGUUUCAAGA | | | | | | |
| | −20_4-4_bulge-symmetric_UGGU- | AUACCUCCACGGCUACGUA | | | | | | |
| | UAGU | GAUCCUUUACUAGUCACUG | | | | | | |
| | −6_6-6_internal_loop- | UCGUACAUUGGCCACUCCCA | | | | | | |
| | symmetric_AUUCAU-CGUAGA | GU | | | | | | |
| | 0_1-1_mismatch_A-C | | | | | | | |
| | 5_1-1_mismatch_U-C | | | | | | | |
| | 12 1-1_mismatch_A-A | | | | | | | |
| | 18_2-1_bulge-asymmetric_GG-G | | | | | | | |
| | 32_6-6_internal_loop- | | | | | | | |
| | symmetric_CAAGGA-AAGGUC | | | | | | | |
| | 40_4-4_bulge-symmetric_GAGU- | | | | | | | |
| | CAGA | | | | | | | |
| 187 | −33_4-4_bulge-symmetric_UUCG- | GCCACACAAAGACACUUGG | 0.109 | 0.918 | 0.902 | 0.733 | 0.86 | 0.859 |
| | ACAU | CCUUUGAAAGUGCUUGCAU | | | | | | |
| | −23_0-2_bulge-asymmetric_-GU | GAAUACAUCCACGGCCAAU | | | | | | |
| | −22_1-1_wobble_G-U | GACCGAUGUUACUAGUCAC | | | | | | |
| | −19_1-1_wobble_G-U | UGUCGUACAUUGGCCACUC | | | | | | |
| | −15_4-5_internal_loop- | CCAGU | | | | | | |
| | asymmetric_UAAA-GAUGU | | | | | | | |
| | −10_3-0_bulge-asymmetric_AAU- | | | | | | | |
| | −4_1-1_mismatch_A-C | | | | | | | |
| | 0_1-1_mismatch_A-C | | | | | | | |
| | 15_1-1_mismatch_A-G | | | | | | | |
| | 19_1-1_mismatch_G-G | | | | | | | |
| | 36_8-8_internal_loop- | | | | | | | |
| | symmetric_GAGGGAGU- | | | | | | | |
| | CAAAGACA | | | | | | | |
| 188 | −33_4-4_bulge-symmetric_UUCG- | GCCACACAGACCUCCUUGGC | 0.275 | 0.923 | 0.931 | 1.129 | 1.67 | 1.658 |
| | ACAU | AGGCUCAAGUCCUUUCAUG | | | | | | |
| | −20_6-6_internal_loop- | AAUACCCCCACGGCUAAUG | | | | | | |
| | symmetric_UGUGGU-UUUUGU | AAUUCCUUUACUUUUGUCU | | | | | | |
| | 0_1-1_mismatch_A-C | GUCGUACAUUGGCCACUCCC | | | | | | |
| | 4_2-2_bulge-symmetric_AU-CC | AGU | | | | | | |
| | 24_6-6_internal_loop- | | | | | | | |
| | symmetric_UCAAAG-AGGCUC | | | | | | | |
| | 40_4-4_bulge-symmetric_GAGU- | | | | | | | |
| | CAGA | | | | | | | |
| 189 | −33_4-4_bulge-symmetric_UUCG- | GCCACACAGACCUCCUCAAG | 0.032 | 0.919 | 0.883 | 0.929 | 1.566 | 1.586 |
| | ACAU | AAUUGAAAGUCUUUUCAUG | | | | | | |
| | −16_8-8_internal_loop- | ACGACAUCCACGGCUAAUG | | | | | | |
| | symmetric_UGGUGUAA- | AAUUCCUCGGUAAGUCACU | | | | | | |
| | CGGUAAGU | GUCGUACAUUGGCCACUCCC | | | | | | |
| | 0_1-1_mismatch_A-C | AGU | | | | | | |
| | 8_2-2_bulge-symmetric_AU-CG | | | | | | | |
| | 18_1-1_wobble_G-U | | | | | | | |
| | 28_6-6_internal_loop- | | | | | | | |
| | symmetric_AGGCCA-CAAGAA | | | | | | | |
| | 40_4-4_bulge-symmetric_GAGU- | | | | | | | |
| | CAGA | | | | | | | |
| 190 | −33_4-4_bulge-symmetric_UUCG- | GCCACACAGAAACAGAUGG | 0.315 | 0.553 | 0.724 | 0.99 | 0.918 | 1.064 |
| | ACAU | CCUUUGAAAGUCCUUUCAU | | | | | | |
| | −20_4-4_bulge-symmetric_UGGU- | CACUACAUCCACGGCUAAU | | | | | | |
| | UAGU | UGUGCGCUUUACUAGUCAC | | | | | | |
| | −8_6-6_internal_loop- | UGUCGUACAUUGGCCACUC | | | | | | |
| | symmetric_GAAUUC-UGUGCG | CCAGU | | | | | | |
| | 0_1-1_mismatch_A-C | | | | | | | |
| | 9_3-3_bulge-symmetric_UUC-CAC | | | | | | | |
| | 34_10−10_internal_loop- | | | | | | | |
| | symmetric_AGGAGGGAGU- | | | | | | | |
| | CAGAAACAGA | | | | | | | |
| 191 | −33_4-4_bulge-symmetric_UUCG- | GCCACACAGACCUCCUUGUA | 0.085 | 0.926 | 0.925 | 0.916 | 1.678 | 1.622 |
| | ACAU | AAAGGAAAGUCCUUUCAUG | | | | | | |
| | −20_6-6_internal_loop- | AAUACAUCCACGGGUUGAA | | | | | | |
| | symmetric_UGUGGU-UGGGGU | UUCCUUUACUGGGGUCUGU | | | | | | |
| | −3_4-2_bulge-asymmetric_UUAG-GU | CGUACAUUGGCCACUCCCAG | | | | | | |
| | 0_1-1_mismatch_A-C | U | | | | | | |
| | 26_6-6_internal_loop- | | | | | | | |
| | symmetric_AAAGGC-UAAAAG | | | | | | | |
| | 40_4-4_bulge-symmetric_GAGU- | | | | | | | |
| | CAGA | | | | | | | |
| 192 | −33_4-4_bulge-symmetric_UUCG- | GCCACACAGACCUCCUUGGC | 0.047 | 0.913 | 0.912 | 0.936 | 1.467 | 1.435 |
| | ACAU | CUGAACAUGUCUUUCAUGA | | | | | | |
| | −23_0-2_bulge-asymmetric_-GU | AUACCUCCACGGCUAAUGA | | | | | | |
| | −22_1-1_wobble_G-U | AUGCAGUCACUAGUCACUG | | | | | | |
| | −12 7-5_internal_loop- | UCGUACAUUGGCCACUCCCA | | | | | | |
| | asymmetric_UAAAGGA-GCAGU | GU | | | | | | |
| | 0_1-1_mismatch_A-C | | | | | | | |
| | 5_1-1_mismatch_U-C | | | | | | | |
| | 19_1-0_bulge-asymmetric_G- | | | | | | | |
| | 22_6-6_internal_loop- | | | | | | | |
| | symmetric_UUUCAA-GAACAU | | | | | | | |
| | 40_4-4_bulge-symmetric_GAGU- | | | | | | | |
| | CAGA | | | | | | | |
| 193 | −33_4-4_bulge-symmetric_UUCG- | GCCACACAGACCUCCUUGAA | 0.13 | 0.867 | 0.845 | 0.988 | 1.626 | 1.564 |
| | ACAU | GACAGAAAGUUCUUUCAUG | | | | | | |
| | −20_4-4_bulge-symmetric_UGGU- | AAUACAUCCACGGCUAAUU | | | | | | |
| | UAGU | ACGUACUUUACUAGUCACU | | | | | | |
| | −8_6-6_internal_loop- | GUCGUACAUUGGCCACUCCC | | | | | | |
| | symmetric_GAAUUC-UACGUA | AGU | | | | | | |
| | 0_1-1_mismatch_A-C | | | | | | | |
| | 19_1-1_wobble_G-U | | | | | | | |
| | 26_6-6_internal_loop- | | | | | | | |
| | symmetric_AAAGGC-AAGACA | | | | | | | |
| | 40_4-4_bulge-symmetric_GAGU- | | | | | | | |
| | CAGA | | | | | | | |
| 194 | −33_4-4_bulge-symmetric_UUCG- | GCCACACAAGAAGACUUGG | 0.08 | 0.956 | 0.941 | 0.511 | 0.918 | 0.843 |
| | ACAU | CCUUUGAAAGUCAUUUCAU | | | | | | |
| | −18_6-6_internal_loop- | GCAUACAUUCACGGCUAAU | | | | | | |
| | symmetric_UGGUGU-UUUGUU | GAAUUCCUUUUUUGUUCAC | | | | | | |
| | 0_1-1_mismatch_A-C | UGUCGUACAUUGGCCACUC | | | | | | |
| | 3 1-1_wobble_G-U | CCAGU | | | | | | |
| | 10_1-1_mismatch_U-C | | | | | | | |
| | 18_1-1_mismatch_G-A | | | | | | | |
| | 36_8-8_internal_loop- | | | | | | | |
| | symmetric_GAGGGAGU- | | | | | | | |
| | CAAGAAGA | | | | | | | |
| 195 | −18_6-6_internal_loop- | GCCACACAAGAAGACUUGG | 0.092 | 0.95 | 0.922 | 0.45 | 0.969 | 0.827 |
| | symmetric_UGGUGU-UUUGUU | CCUUUGAAAGUCAUUUCAU | | | | | | |
| | 0_1-1_mismatch_A-C | GCAUACAUUCACGGCUAAU | | | | | | |
| | 3 1-1_wobble_G-U | GAAUUCCUUUUUUGUUCAC | | | | | | |
| | 10_1-1_mismatch_U-C | UGUCGUCGAAUGGCCACUC | | | | | | |
| | 18_1-1_mismatch_G-A | CCAGU | | | | | | |
| | 36_8-8_internal_loop- | | | | | | | |
| | symmetric_GAGGGAGU- | | | | | | | |
| | CAAGAAGA | | | | | | | |
| 196 | −33_4-4_bulge-symmetric_UUCG- | GCCACACAGACCUCCUAACA | 0.131 | 0.53 | 0.7 | 1.049 | 1.384 | 1.521 |
| | ACAU | GGUUGAAAGUCCUUUCAUC | | | | | | |
| | −20_4-4_bulge-symmetric_UGGU- | UUUACUUCCACGGCUACCG | | | | | | |
| | UAGU | UAGUCCUUUACUAGUCACU | | | | | | |
| | −6_6-6_internal_loop- | GUCGUACAUUGGCCACUCCC | | | | | | |
| | symmetric_AUUCAU-CCGUAG | AGU | | | | | | |
| | 0_1-1_mismatch_A-C | | | | | | | |
| | 5_1-1_mismatch_U-U | | | | | | | |
| | 9_3-3_bulge-symmetric_UUC-CUU | | | | | | | |
| | 28_6-6_internal_loop- | | | | | | | |
| | symmetric_AGGCCA-AACAGG | | | | | | | |
| | 40_4-4_bulge-symmetric_GAGU- | | | | | | | |
| | CAGA | | | | | | | |
| 197 | −33_4-4_bulge-symmetric_UUCG- | GCCACACAGACCUCCUUGGC | 0.23 | 0.928 | 0.905 | 1.022 | 1.552 | 1.474 |
| | ACAU | AAUAAUAAGUCCUUUCAUG | | | | | | |
| | −20_4-4_bulge-symmetric_UGGU- | AAUACAUCCACGGCUAAUA | | | | | | |
| | UAGU | AAUAGGACGACUAGUCACU | | | | | | |
| | −12_6-6_internal_loop- | GUCGUACAUUGGCCACUCCC | | | | | | |
| | symmetric_AAAGGA-AGGACG | AGU | | | | | | |
| | −8_1-1_mismatch_C-A | | | | | | | |
| | 0_1-1_mismatch_A-C | | | | | | | |
| | 24_6-6_internal_loop- | | | | | | | |
| | symmetric_UCAAAG-AAUAAU | | | | | | | |
| | 40_4-4_bulge-symmetric_GAGU- | | | | | | | |
| | CAGA | | | | | | | |
| 198 | −33_4-4_bulge-symmetric_UUCG- | GCCACACAGAGAGAUAUGG | 0.098 | 0.554 | 0.662 | 0.939 | 1.208 | 1.153 |
| | ACAU | CCUUUGAAAGUCCCUAUUU | | | | | | |
| | −20_4-4_bulge-symmetric_UGGU- | CAUGAUUACGUCCACGGCU | | | | | | |
| | UAGU | AAUCCAGGACUUUACUAGU | | | | | | |
| | −8_6-6_internal_loop- | CACUGUCGUACAUUGGCCA | | | | | | |
| | symmetric_GAAUUC-CCAGGA | CUCCCAGU | | | | | | |
| | 0_1-1_mismatch_A-C | | | | | | | |
| | 5_1-1_wobble_U-G | | | | | | | |
| | 9_1-1_mismatch_U-U | | | | | | | |
| | 17_0-3_bulge-asymmetric_-CUA | | | | | | | |
| | 34_10−10_internal_loop- | | | | | | | |
| | symmetric_AGGAGGGAGU- | | | | | | | |
| | CAGAGAGAUA | | | | | | | |
| 199 | −33_4-4_bulge-symmetric_UUCG- | GCCACACAGACCUCAGCCUA | 0.131 | 0.921 | 0.906 | 1.014 | 1.308 | 1.416 |
| | ACAU | CUUUGAAAGUCCAUUCAUG | | | | | | |
| | −23_0-4_bulge-asymmetric_-UAGU | AAUACAUCCACGGCUUAUG | | | | | | |
| | −23_1-1_wobble_U-G | AAUUCACAGCGUAGUCACU | | | | | | |
| | −21_1-1_mismatch_G-G | GUCGUACAUUGGCCACUCCC | | | | | | |
| | −14_4-0_bulge-asymmetric_AAAG- | AGU | | | | | | |
| | −5_1-1_mismatch_U-U | | | | | | | |
| | 0_1-1_mismatch_A-C | | | | | | | |
| | 17_1-1_mismatch_A-A | | | | | | | |
| | 30_6-6_internal_loop- | | | | | | | |
| | symmetric_GCCAAG-AGCCUA | | | | | | | |
| | 40_4-4_bulge-symmetric_GAGU- | | | | | | | |
| | CAGA | | | | | | | |
| 200 | −33_4-4_bulge-symmetric_UUCG- | GCCACACAGACCUCCUAUUU | 0.053 | 0.624 | 0.625 | 0.945 | 1.428 | 1.423 |
| | ACAU | ACUUGAAAGUCCUGUCAUG | | | | | | |
| | −20_4-4_bulge-symmetric_UGGU- | AAUACAUCCACGGCGGUAA | | | | | | |
| | UAGU | UCCGGGGCUUUACUAGUCA | | | | | | |
| | −8_6-6_internal_loop- | CUGUCGUACAUUGGCCACU | | | | | | |
| | symmetric_GAAUUC-CCGGGG | CCCAGU | | | | | | |
| | −3_0-2_bulge-asymmetric_-GG | | | | | | | |
| | 0_1-1_mismatch_A-C | | | | | | | |
| | 16_1-1_mismatch_A-G | | | | | | | |
| | 28_6-6_internal_loop- | | | | | | | |
| | symmetric_AGGCCA-AUUUAC | | | | | | | |
| | 40_4-4_bulge-symmetric_GAGU- | | | | | | | |
| | CAGA | | | | | | | |
| 201 | −33_4-4_bulge-symmetric_UUCG- | GCCACACAGACCUCCUCCAU | 0.046 | 0.921 | 0.915 | 0.939 | 1.735 | 1.721 |
| | ACAU | UCUUGAAAGUCCUUUUAUG | | | | | | |
| | −18_6-6_internal_loop- | AAUAGAUCCACGGCAAAUG | | | | | | |
| | symmetric_UGGUGU-UAGUGU | AAUUCCUUUUAGUGUCACU | | | | | | |
| | −4_1-1_mismatch_A-A | GUCGUACAUUGGCCACUCCC | | | | | | |
| | 0_1-1_mismatch_A-C | AGU | | | | | | |
| | 6_1-1_mismatch_G-G | | | | | | | |
| | 14_1-1_wobble_G-U | | | | | | | |
| | 28_6-6_internal_loop- | | | | | | | |
| | symmetric_AGGCCA-CCAUUC | | | | | | | |
| | 40_4-4_bulge-symmetric_GAGU- | | | | | | | |
| | CAGA | | | | | | | |
| 202 | −33_4-4_bulge-symmetric_UUCG- | GCCACAUGAAAGUCCUUGG | 0.147 | 0.892 | 0.88 | 0.586 | 0.702 | 0.759 |
| | ACAU | CCUUUGAAAGUCCUUUGAU | | | | | | |
| | −20_4-4_bulge-symmetric_UGGU- | GAAUACAUCCACGGCUACG | | | | | | |
| | UAGU | CGACACAGAUUACUAGUCA | | | | | | |
| | −10_6-6_internal_loop- | CUGUCGUACAUUGGCCACU | | | | | | |
| | symmetric_AGGAAU-CACAGA | CCCAGU | | | | | | |
| | −6_2-3_bulge-asymmetric_AU-CGC | | | | | | | |
| | 0_1-1_mismatch_A-C | | | | | | | |
| | 14_1-1_mismatch_G-G | | | | | | | |
| | 38_6-6_internal_loop- | | | | | | | |
| | symmetric_GGGAGU-UGAAAG | | | | | | | |
| 203 | −33_4-4_bulge-symmetric_UUCG- | GCCACACAGACCUCCUAACA | 0.044 | 0.908 | 0.907 | 0.947 | 1.706 | 1.726 |
| | ACAU | GAUUGAAAGUUAUUCAUGA | | | | | | |
| | −18_6-6_internal_loop- | AUACAUCCACGGCUAAUGA | | | | | | |
| | symmetric_UGGUGU-UGAAAU | AUUCCUUUUGAAAUCACUG | | | | | | |
| | 0_1-1_mismatch_A-C | UCGUACAUUGGCCACUCCCA | | | | | | |
| | 17_2-1_bulge-asymmetric_AG-A | GU | | | | | | |
| | 19_1-1_wobble_G-U | | | | | | | |
| | 28_6-6_internal_loop- | | | | | | | |
| | symmetric_AGGCCA-AACAGA | | | | | | | |
| | 40_4-4_bulge-symmetric_GAGU- | | | | | | | |
| | CAGA | | | | | | | |
| 204 | −33_4-4_bulge-symmetric_UUCG- | GCCACACAGACCUCCUCUCA | 0.224 | 0.933 | 0.935 | 1.114 | 1.707 | 1.694 |
| | ACAU | AGUUGAAAGUCCUUUCAUG | | | | | | |
| | −16_8-8_internal_loop- | AAUAUAUCCACGGCUUAAU | | | | | | |
| | symmetric_UGGUGUAA- | UCCUAAUGAAGUCACUGUC | | | | | | |
| | AAUGAAGU | GUACAUUGGCCACUCCCAG | | | | | | |
| | −5_4-1_bulge-asymmetric_CAUU-U | U | | | | | | |
| | 0_1-1_mismatch_A-C | | | | | | | |
| | 6_1-1_wobble_G-U | | | | | | | |
| | 28_6-6_internal_loop- | | | | | | | |
| | symmetric_AGGCCA-CUCAAG | | | | | | | |
| | 40_4-4_bulge-symmetric_GAGU- | | | | | | | |
| | CAGA | | | | | | | |
| 205 | −33_4-4_bulge-symmetric_UUCG- | GCCACACAGACCUCCUUGAA | 0.087 | 0.936 | 0.911 | 0.944 | 1.67 | 1.556 |
| | ACAU | UAUGGAAAGUCCCUUCAUG | | | | | | |
| | −23_1-0_bulge-asymmetric_U- | AAUAUAUCCACGGCUAAUG | | | | | | |
| | −22_1-1_wobble_G-U | AAUUCGGCUUAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_2-3_bulge-asymmetric_AG-GGC | | | | | | | |
| | 0_1-1_mismatch_A-C | | | | | | | |
| | 6_1-1_wobble_G-U | | | | | | | |
| | 17_1-1_mismatch_A-C | | | | | | | |
| | 26_6-6_internal_loop- | | | | | | | |
| | symmetric_AAAGGC-AAUAUG | | | | | | | |
| | 40_4-4_bulge-symmetric_GAGU- | | | | | | | |
| | CAGA | | | | | | | |
| 206 | −33_4-4_bulge-symmetric_UUCG- | GCCACAUGGUUAUCCUUGG | 0.021 | 0.542 | 0.647 | 0.9 | 1.298 | 1.376 |
| | ACAU | CCUUUGAAAGUCCUGGUGC | | | | | | |
| | −20_4-4_bulge-symmetric_UGGU- | UUGAAUCAAUCCACGGCUA | | | | | | |
| | UAGU | CGACAGUCCUUUACUAGUC | | | | | | |
| | −6_6-6_internal_loop- | ACUGUCGUACAUUGGCCAC | | | | | | |
| | symmetric_AUUCAU-CGACAG | UCCCAGU | | | | | | |
| | 0_1-1_mismatch_A-C | | | | | | | |
| | 6_2-2_bulge-symmetric_GU-CA | | | | | | | |
| | 13 1-1_mismatch_U-U | | | | | | | |
| | 15_2-4_bulge-asymmetric_AA-GGUG | | | | | | | |
| | 38_6-6_internal_loop- | | | | | | | |
| | symmetric_GGGAGU-UGGUUA | | | | | | | |
| 207 | −33_4-4_bulge-symmetric_UUCG- | GCCACACAGACCUCCUUGGC | 0.089 | 0.54 | 0.662 | 0.958 | 1.219 | 1.287 |
| | ACAU | AUUACUAAGUCCUUUCAUG | | | | | | |
| | −23_0-6_internal_loop-asymmetric_- | AUCACAUCCACGGCUAAAU | | | | | | |
| | ACUAGU | GCAUCGACUAGUCACUGUC | | | | | | |
| | −23_1-1_wobble_U-G | GUACAUUGGCCACUCCCAG | | | | | | |
| | −21_1-1_wobble_G-U | U | | | | | | |
| | −18_1-1_wobble_U-G | | | | | | | |
| | −7_10-1_internal_loop- | | | | | | | |
| | asymmetric_AAGGAAUUCA-A | | | | | | | |
| | 0_1-1_mismatch_A-C | | | | | | | |
| | 8_2-2_bulge-symmetric_AU-UC | | | | | | | |
| | 24_6-6_internal_loop- | | | | | | | |
| | symmetric_UCAAAG-AUUACU | | | | | | | |
| | 40_4-4_bulge-symmetric_GAGU- | | | | | | | |
| | CAGA | | | | | | | |
| 208 | −33_4-4_bulge-symmetric_UUCG- | GCCACACAAAGGGACUUGG | 0.061 | 0.886 | 0.862 | 0.608 | 0.85 | 0.783 |
| | ACAU | CCUUUGAAAGUCCUUACAU | | | | | | |
| | −20_4-4_bulge-symmetric_UGGU- | GAAUUCAUCCACGGCUAAU | | | | | | |
| | UAGU | UCUUGGCUUUACUAGUCAC | | | | | | |
| | −8_6-6_internal_loop- | UGUCGUACAUUGGCCACUC | | | | | | |
| | symmetric_GAAUUC-UCUUGG | CCAGU | | | | | | |
| | 0_1-1_mismatch_A-C | | | | | | | |
| | 7_1-1_mismatch_U-U | | | | | | | |
| | 15_1-1_mismatch_A-A | | | | | | | |
| | 36_8-8_internal_loop- | | | | | | | |
| | symmetric_GAGGGAGU- | | | | | | | |
| | CAAAGGGA | | | | | | | |
| 209 | −33_4-4_bulge-symmetric_UUCG- | GCCACACAUAUAUCCUUGG | 0.121 | 0.52 | 0.725 | 0.8 | 0.71 | 0.871 |
| | ACAU | CCUUUGAAAGUCCUGUCAU | | | | | | |
| | −16_8-8_internal_loop- | GAGUAGGAUCCACGGCUUC | | | | | | |
| | symmetric_UGGUGUAA- | AUAAUUCCUCAGAGAGUCA | | | | | | |
| | CAGAGAGU | CUGUCGUACAUUGGCCACU | | | | | | |
| | −5_4-4_bulge-symmetric_CAUU- | CCCAGU | | | | | | |
| | UCAU | | | | | | | |
| | 0_1-1_mismatch_A-C | | | | | | | |
| | 6_1-2_bulge-asymmetric_G-GG | | | | | | | |
| | 9_1-1_wobble_U-G | | | | | | | |
| | 16_1-1_mismatch_A-G | | | | | | | |
| | 38_6-6_internal_loop- | | | | | | | |
| | symmetric_GGGAGU-CAUAUA | | | | | | | |
| 210 | −33_4-4_bulge-symmetric_UUCG- | GCCACACAGACCUCCUCAUA | 0.021 | 0.616 | 0.672 | 0.938 | 1.46 | 1.48 |
| | ACAU | AGUUGAAAGUCCUUUCAAA | | | | | | |
| | −22_2-5_internal_loop- | AAUAGCCCCACGGCUAUUG | | | | | | |
| | asymmetric_UG-GUAGU | AAUUCGGCGCGUAGUCACU | | | | | | |
| | −20_1-1_wobble_U-G | GUCGUACAUUGGCCACUCCC | | | | | | |
| | −18_1-1_wobble_U-G | AGU | | | | | | |
| | −14_4-1_bulge-asymmetric_AAAG-G | | | | | | | |
| | −6_1-1_mismatch_U-U | | | | | | | |
| | 0_1-1_mismatch_A-C | | | | | | | |
| | 4_3-3_bulge-symmetric_AUG-GCC | | | | | | | |
| | 11_2-2_bulge-symmetric_CA-AA | | | | | | | |
| | 28_6-6_internal_loop- | | | | | | | |
| | symmetric_AGGCCA-CAUAAG | | | | | | | |
| | 40_4-4_bulge-symmetric_GAGU- | | | | | | | |
| | CAGA | | | | | | | |
| 211 | −33_4-4_bulge-symmetric_UUCG- | GCCACACAGACCUCCUUGGC | 0.052 | 0.611 | 0.59 | 0.923 | 1.372 | 1.301 |
| | ACAU | CUGUUCAUGUCUUUUCAUG | | | | | | |
| | −23_0-2_bulge-asymmetric_-GU | AAUACAUCCACGGGUAAUG | | | | | | |
| | −22_1-1_wobble_G-U | AUCGGGCUUACUAGUCACU | | | | | | |
| | −19_1-1_wobble_G-U | GUCGUACAUUGGCCACUCCC | | | | | | |
| | −14_5-5_internal_loop- | AGU | | | | | | |
| | symmetric_UAAAG-GGGCU | | | | | | | |
| | −10_2-0_bulge-asymmetric_AU- | | | | | | | |
| | −3_1-1_mismatch_G-G | | | | | | | |
| | 0_1-1_mismatch_A-C | | | | | | | |
| | 18_1-1_wobble_G-U | | | | | | | |
| | 22_6-6_internal_loop- | | | | | | | |
| | symmetric_UUUCAA-GUUCAU | | | | | | | |
| | 40_4-4_bulge-symmetric_GAGU- | | | | | | | |
| | CAGA | | | | | | | |
| 212 | −33_4-4_bulge-symmetric_UUCG- | GCCACAUGGUAGUCCUUGG | 0.177 | 0.922 | 0.924 | 0.476 | 0.715 | 0.738 |
| | ACAU | CCUUUGAAAGUCCUUUCAU | | | | | | |
| | −18_6-6_internal_loop- | GAAUACAUUCACGGCUCAU | | | | | | |
| | symmetric_UGGUGU-UUUGAU | GAAUUCCUUUUUUGAUCAC | | | | | | |
| | −5_1-1_mismatch_U-C | UGUCGUACAUUGGCCACUC | | | | | | |
| | 0_1-1_mismatch_A-C | CCAGU | | | | | | |
| | 3_1-1_wobble_G-U | | | | | | | |
| | 38_6-6_internal_loop- | | | | | | | |
| | symmetric_GGGAGU-UGGUAG | | | | | | | |
| 213 | −33_4-4_bulge-symmetric_UUCG- | GCCACACAGACCUCCUGACA | 0.023 | 0.571 | 0.667 | 0.94 | 1.381 | 1.453 |
| | ACAU | GAUUGAAAGUGAGUUCAUG | | | | | | |
| | −21_3-5_internal_loop- | AUACCAUCCACGGCUUUCG | | | | | | |
| | asymmetric_UGG-AUAGU | AAUUCCUGCAAUAGUCACU | | | | | | |
| | −18_1-1_wobble_U-G | GUCGUACAUUGGCCACUCCC | | | | | | |
| | −16_2-0_bulge-asymmetric_AA- | AGU | | | | | | |
| | −5_3-3_bulge-symmetric_AUU-UUC | | | | | | | |
| | 0_1-1_mismatch_A-C | | | | | | | |
| | 7_3-3_bulge-symmetric_UAU-UAC | | | | | | | |
| | 17_3-3_bulge-symmetric_AGG-GAG | | | | | | | |
| | 28_6-6_internal_loop- | | | | | | | |
| | symmetric_AGGCCA-GACAGA | | | | | | | |
| | 40_4-4_bulge-symmetric_GAGU- | | | | | | | |
| | CAGA | | | | | | | |
| 214 | −33_4-4_bulge-symmetric_UUCG- | GCCACACAGACCUCCUGUUU | 0.032 | 0.733 | 0.762 | 0.924 | 1.516 | 1.519 |
| | ACAU | UAUUGAAAGUCCUUGCAUG | | | | | | |
| | −20_4-4_bulge-symmetric_UGGU- | GAUACUCCACGGCUACGAU | | | | | | |
| | UAGU | AGUCCUUUACUAGUCACUG | | | | | | |
| | −6_6-6_internal_loop- | UCGUACAUUGGCCACUCCCA | | | | | | |
| | symmetric_AUUCAU-CGAUAG | GU | | | | | | |
| | 0_1-1_mismatch_A-C | | | | | | | |
| | 5_1-0_bulge-asymmetric_U- | | | | | | | |
| | 10_1-1_wobble_U-G | | | | | | | |
| | 15_1-1_mismatch_A-G | | | | | | | |
| | 28_6-6_internal_loop- | | | | | | | |
| | symmetric_AGGCCA-GUUUUA | | | | | | | |
| | 40_4-4_bulge-symmetric_GAGU- | | | | | | | |
| | CAGA | | | | | | | |
| 215 | −33_4-4_bulge-symmetric_UUCG- | GCCACACAGGAGCACUUGG | 0.171 | 0.945 | 0.946 | 0.618 | 0.771 | 0.748 |
| | ACAU | CCUUUGAAAGUCCGUUCAU | | | | | | |
| | −14_10−10_internal_loop- | GAAUACAUCCACGGCUAUU | | | | | | |
| | symmetric_UGGUGUAAAG- | GAAUUCGACAUGUAGUCAC | | | | | | |
| | GACAUGUAGU | UGUCGUACAUUGGCCACUC | | | | | | |
| | −6_1-1_mismatch_U-U | CCAGU | | | | | | |
| | 0_1-1_mismatch_A-C | | | | | | | |
| | 17_1-1_mismatch_A-G | | | | | | | |
| | 36_8-8_internal_loop- | | | | | | | |
| | symmetric_GAGGGAGU- | | | | | | | |
| | CAGGAGCA | | | | | | | |
| 216 | −33_4-4_bulge-symmetric_UUCG- | GCCACACAGACCAGGUAUG | 0.214 | 0.848 | 0.772 | 0.918 | 1.234 | 1.324 |
| | ACAU | CCUUUGAAAGUCCUUUCAU | | | | | | |
| | −14_10−10_internal_loop- | GAAUACAUCCACGGUUAAA | | | | | | |
| | symmetric_UGGUGUAAAG- | AAUUCGGUGUGUAGUCACU | | | | | | |
| | GGUGUGUAGU | GUCGUACAUUGGCCACUCCC | | | | | | |
| | −7_2-1_bulge-asymmetric_CA-A | AGU | | | | | | |
| | −3_1-1_wobble_G-U | | | | | | | |
| | 0_1-1_mismatch_A-C | | | | | | | |
| | 32_6-6_internal_loop- | | | | | | | |
| | symmetric_CAAGGA-AGGUAU | | | | | | | |
| | 40_4-4_bulge-symmetric_GAGU- | | | | | | | |
| | CAGA | | | | | | | |
| 217 | −33_4-4_bulge-symmetric_UUCG- | GCCACACAGACCUCCUCCAA | 0.091 | 0.968 | 0.97 | 0.994 | 1.791 | 1.81 |
| | ACAU | GGUUGAAAGUCCUAUCAUG | | | | | | |
| | −20_4-4_bulge-symmetric_UGGU- | AAUACAUCCACGGCUAAUU | | | | | | |
| | UAGU | UACGGCUUUACUAGUCACU | | | | | | |
| | −8_6-6_internal_loop- | GUCGUACAUUGGCCACUCCC | | | | | | |
| | symmetric_GAAUUC-UUACGG | AGU | | | | | | |
| | 0_1-1_mismatch_A-C | | | | | | | |
| | 16_1-1_mismatch_A-A | | | | | | | |
| | 27_3-0_bulge-asymmetric_AAG- | | | | | | | |
| | 30_1-1_wobble_G-U | | | | | | | |
| | 33_2-2_bulge-symmetric_AA-AA | | | | | | | |
| | 40_1-1_wobble_G-U | | | | | | | |
| | 41_1-1_mismatch_A-C | | | | | | | |
| | 43_0-3_bulge-asymmetric_-CAG | | | | | | | |
| 218 | −33_4-4_bulge-symmetric_UUCG- | GCCACACAAGAAGGCUUGG | 0.047 | 0.941 | 0.912 | 0.645 | 0.922 | 0.932 |
| | ACAU | CCUUUGAAAGUCCUUUGUU | | | | | | |
| | −22_2-5_internal_loop- | GAAUACAUCCACGGCUAAU | | | | | | |
| | asymmetric_UG-GUAGU | GAAUUCGGCGCGUAGUCAC | | | | | | |
| | −20_1-1_wobble_U-G | UGUCGUACAUUGGCCACUC | | | | | | |
| | −18_1-1_wobble_U-G | CCAGU | | | | | | |
| | −14_4-1_bulge-asymmetric_AAAG-G | | | | | | | |
| | 0_1-1_mismatch_A-C | | | | | | | |
| | 13_2-2_bulge-symmetric_UG-GU | | | | | | | |
| | 36_8-8_internal_loop- | | | | | | | |
| | symmetric_GAGGGAGU- | | | | | | | |
| | CAAGAAGG | | | | | | | |
| 219 | −33_4-4_bulge-symmetric_UUCG- | GCCACACAGACCUCCUUGUA | 0.035 | 0.836 | 0.886 | 0.952 | 1.664 | 1.708 |
| | ACAU | GACGGAAAGUGGUUUCAUU | | | | | | |
| | −20_4-4_bulge-symmetric_UGGU- | CAUAGAUCCACGGCUACGA | | | | | | |
| | UAGU | CGCUCCUUUACUAGUCACU | | | | | | |
| | −6_6-6_internal_loop- | GUCGUACAUUGGCCACUCCC | | | | | | |
| | symmetric_AUUCAU-CGACGC | AGU | | | | | | |
| | 0_1-1_mismatch_A-C | | | | | | | |
| | 6_1-1_mismatch_G-G | | | | | | | |
| | 10_2-2_bulge-symmetric_UC-UC | | | | | | | |
| | 18_2-2_bulge-symmetric_GG-GG | | | | | | | |
| | 26_6-6_internal_loop- | | | | | | | |
| | symmetric_AAAGGC-UAGACG | | | | | | | |
| | 40_4-4_bulge-symmetric_GAGU- | | | | | | | |
| | CAGA | | | | | | | |
| 220 | −33_4-4_bulge-symmetric_UUCG- | GCCACACAAAUAAACUUGG | 0.187 | 0.949 | 0.943 | 0.529 | 0.783 | 0.697 |
| | ACAU | CCUUUGAAAGUCCUUUCAU | | | | | | |
| | −20_4-4_bulge-symmetric_UGGU- | GAAUAUAUCCACGGCUAAU | | | | | | |
| | UAGU | GAAUGCGAUGACUAGUCAC | | | | | | |
| | −12 6-6_internal_loop- | UGUCGUACAUUGGCCACUC | | | | | | |
| | symmetric_AAAGGA-GCGAUG | CCAGU | | | | | | |
| | 0_1-1_mismatch_A-C | | | | | | | |
| | 6_1-1_wobble_G-U | | | | | | | |
| | 36_8-8_internal_loop- | | | | | | | |
| | symmetric_GAGGGAGU- | | | | | | | |
| | CAAAUAAA | | | | | | | |
| 221 | −33_4-4_bulge-symmetric_UUCG- | GCCACACAGACCUCCUUGUA | 0.25 | 0.869 | 0.869 | 1.098 | 1.528 | 1.502 |
| | ACAU | AGUAGAAAGUCCUUUCAUG | | | | | | |
| | −20_4-4_bulge-symmetric_UGGU- | AAUACCACCACGGCCAAUG | | | | | | |
| | UAGU | ACACGACUUACUAGUCACU | | | | | | |
| | −10_6-6_internal_loop- | GUCGUACAUUGGCCACUCCC | | | | | | |
| | symmetric_AGGAAU-CACGAC | AGU | | | | | | |
| | −4_1-1_mismatch_A-C | | | | | | | |
| | 0_1-1_mismatch_A-C | | | | | | | |
| | 4_2-2_bulge-symmetric_AU-CA | | | | | | | |
| | 26_6-6_internal_loop- | | | | | | | |
| | symmetric_AAAGGC-UAAGUA | | | | | | | |
| | 40_4-4_bulge-symmetric_GAGU- | | | | | | | |
| | CAGA | | | | | | | |
| 222 | −33_4-4_bulge-symmetric_UUCG- | GCCACACAGACCUCCUAGAA | 0.155 | 0.863 | 0.848 | 0.99 | 1.369 | 1.357 |
| | ACAU | UCUUGAAAGUCCUUUCAUG | | | | | | |
| | −22_2-3_bulge-asymmetric_UG-AGU | AAUAUAUCCACGGCUAAUU | | | | | | |
| | −21_1-1_wobble_G-U | AAUUCGAGUCAUAGUCACU | | | | | | |
| | −14_5-4_internal_loop- | GUCGUACAUUGGCCACUCCC | | | | | | |
| | asymmetric_UAAAG-GAGU | AGU | | | | | | |
| | −8_1-1_mismatch_C-U | | | | | | | |
| | 0_1-1_mismatch_A-C | | | | | | | |
| | 6_1-1_wobble_G-U | | | | | | | |
| | 28_6-6_internal_loop- | | | | | | | |
| | symmetric_AGGCCA-AGAAUC | | | | | | | |
| | 40_4-4_bulge-symmetric_GAGU- | | | | | | | |
| | CAGA | | | | | | | |
| 223 | −33_4-4_bulge-symmetric_UUCG- | GCCACACAGACCUCCUUGGC | 0.44 | 0.925 | 0.921 | 1.218 | 1.684 | 1.581 |
| | ACAU | GGGGUUAAGUCCUUUCAUG | | | | | | |
| | −20_4-4_bulge-symmetric_UGGU- | AAUACACCCACGGCUAAUCC | | | | | | |
| | UAGU | UUGGCUUUACUAGUCACUG | | | | | | |
| | −8_6-6_internal_loop- | UCGUACAUUGGCCACUCCCA | | | | | | |
| | symmetric_GAAUUC-CCUUGG | GU | | | | | | |
| | 0_1-1_mismatch_A-C | | | | | | | |
| | 4_1-1_mismatch_A-C | | | | | | | |
| | 24_6-6_internal_loop- | | | | | | | |
| | symmetric_UCAAAG-GGGGUU | | | | | | | |
| | 40_4-4_bulge-symmetric_GAGU- | | | | | | | |
| | CAGA | | | | | | | |
| 224 | −33_4-4_bulge-symmetric_UUCG- | GCCACACAGACCUCCUGUAA | 0.285 | 0.923 | 0.897 | 1.101 | 1.572 | 1.546 |
| | ACAU | UGUUGAAAGUCCUUUCAUG | | | | | | |
| | −23_1-0_bulge-asymmetric_U- | AAUACAUCUACGGCUAACG | | | | | | |
| | −22_1-1_wobble_G-U | AAUUCGGGCUAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_3-4_bulge-asymmetric_AAG- | | | | | | | |
| | GGGC | | | | | | | |
| | −7_1-1_mismatch_A-C | | | | | | | |
| | 0_1-1_mismatch_A-C | | | | | | | |
| | 2 1-1_wobble_G-U | | | | | | | |
| | 28_6-6_internal_loop- | | | | | | | |
| | symmetric_AGGCCA-GUAAUG | | | | | | | |
| | 40_4-4_bulge-symmetric_GAGU- | | | | | | | |
| | CAGA | | | | | | | |
| 225 | −33_4-4_bulge-symmetric_UUCG- | GCCACACAGACCUCCUCUCA | 0.251 | 0.858 | 0.871 | 1.117 | 1.639 | 1.619 |
| | ACAU | GGUUGAAAGUCCUUUCAUG | | | | | | |
| | −22_2-9_internal_loop- | GAUACAUCCACGGCUUCCG | | | | | | |
| | asymmetric_UG-AUUACUAGU | AUGCAUAUUACUAGUCACU | | | | | | |
| | −21_1-1_wobble_G-U | GUCGUACAUUGGCCACUCCC | | | | | | |
| | −18_1-1_wobble_U-G | AGU | | | | | | |
| | −15_2-2_bulge-symmetric_AA-GA | | | | | | | |
| | −5_7-0_internal_loop- | | | | | | | |
| | asymmetric_AUUCAUU- | | | | | | | |
| | 0_1-1_mismatch_A-C | | | | | | | |
| | 10_1-1_wobble_U-G | | | | | | | |
| | 28_6-6_internal_loop- | | | | | | | |
| | symmetric_AGGCCA-CUCAGG | | | | | | | |
| | 40_4-4_bulge-symmetric_GAGU- | | | | | | | |
| | CAGA | | | | | | | |
| 226 | −33_4-4_bulge-symmetric_UUCG- | GCCACACAGACCUCCUGAAA | 0.171 | 0.812 | 0.78 | 1.017 | 1.497 | 1.487 |
| | ACAU | UGUUGAAAGUCCUUUCAUG | | | | | | |
| | −20_4-4_bulge-symmetric_UGGU- | AAUACAUCCACGGGUAAUU | | | | | | |
| | UAGU | AUACGCUUUACUAGUCACU | | | | | | |
| | −8_6-6_internal_loop- | GUCGUACAUUGGCCACUCCC | | | | | | |
| | symmetric_GAAUUC-UAUACG | AGU | | | | | | |
| | −3_1-1_mismatch_G-G | | | | | | | |
| | 0_1-1_mismatch_A-C | | | | | | | |
| | 28_6-6_internal_loop- | | | | | | | |
| | symmetric_AGGCCA-GAAAUG | | | | | | | |
| | 40_4-4_bulge-symmetric_GAGU- | | | | | | | |
| | CAGA | | | | | | | |
| 227 | −33_4-4_bulge-symmetric_UUCG- | GCCACACAGACCUCCUUGUA | 0.156 | 0.709 | 0.792 | 1.069 | 1.439 | 1.547 |
| | ACAU | GACCGAAAGUCCUUUCAUG | | | | | | |
| | −20_4-4_bulge-symmetric_UGGU- | AAUAUCAUCCAUAGCUAAU | | | | | | |
| | UAGU | UGCACACUUUACUAGUCAC | | | | | | |
| | −8_6-6_internal_loop- | UGUCGUACAUUGGCCACUC | | | | | | |
| | symmetric_GAAUUC-UGCACA | CCAGU | | | | | | |
| | −1_1-1_mismatch_C-A | | | | | | | |
| | 6_0-1_bulge-asymmetric_-U | | | | | | | |
| | 26_6-6_internal_loop- | | | | | | | |
| | symmetric_AAAGGC-UAGACC | | | | | | | |
| | 40_4-4_bulge-symmetric_GAGU- | | | | | | | |
| | CAGA | | | | | | | |
| 228 | −33_4-4_bulge-symmetric_UUCG- | GCCACACAGACCUCCUCUAU | 0.219 | 0.539 | 0.667 | 1.128 | 1.387 | 1.453 |
| | ACAU | ACUUGAAAGUCCUUUCAUG | | | | | | |
| | −20_4-4_bulge-symmetric_UGGU- | AAUAGAUCCACGGUUACCU | | | | | | |
| | UAGU | UGUGAAUUCCUUUACUAGU | | | | | | |
| | −6_1-1_wobble_U-G | CACUGUCGUACAUUGGCCA | | | | | | |
| | −5_0-4_bulge-asymmetric_-CCUU | CUCCCAGU | | | | | | |
| | −3 1-1_wobble_G-U | | | | | | | |
| | 0_1-1_mismatch_A-C | | | | | | | |
| | 6_1-1_mismatch_G-G | | | | | | | |
| | 28_6-6_internal_loop- | | | | | | | |
| | symmetric_AGGCCA-CUAUAC | | | | | | | |
| | 40_4-4_bulge-symmetric_GAGU- | | | | | | | |
| | CAGA | | | | | | | |
| 229 | −33_4-4_bulge-symmetric_UUCG- | GCCACACAGACCUCGCAAUG | 0.309 | 0.887 | 0.897 | 1.151 | 1.594 | 1.615 |
| | ACAU | CUUUGAAAGUCCUUUCAUG | | | | | | |
| | −20_4-4_bulge-symmetric_UGGU- | AAUAAAUCCACGGCUAAUG | | | | | | |
| | UAGU | AAUGAGACGACUAGUCACU | | | | | | |
| | −12_6-6_internal_loop- | GUCGUACAUUGGCCACUCCC | | | | | | |
| | symmetric_AAAGGA-GAGACG | AGU | | | | | | |
| | 0_1-1_mismatch_A-C | | | | | | | |
| | 6_1-1_mismatch_G-A | | | | | | | |
| | 30_6-6_internal_loop- | | | | | | | |
| | symmetric_GCCAAG-GCAAUG | | | | | | | |
| | 40_4-4_bulge-symmetric_GAGU- | | | | | | | |
| | CAGA | | | | | | | |
| 230 | −33_4-4_bulge-symmetric_UUCG- | GCCACACAGACCUCCUAUCA | 0.059 | 0.838 | 0.802 | 0.924 | 1.505 | 1.478 |
| | ACAU | AGUUGAAAGUCCUUUCAGC | | | | | | |
| | −18_6-6_internal_loop- | AUACAUCCACGGCUAAUGA | | | | | | |
| | symmetric_UGGUGU-UGUUUU | AUUCCUUUUGUUUUCACUG | | | | | | |
| | 0_1-1_mismatch_A-C | UCGUACAUUGGCCACUCCCA | | | | | | |
| | 10_1-1_mismatch_U-C | GU | | | | | | |
| | 12_1-0_bulge-asymmetric_A- | | | | | | | |
| | 28_6-6_internal_loop- | | | | | | | |
| | symmetric_AGGCCA-AUCAAG | | | | | | | |
| | 40_4-4_bulge-symmetric_GAGU- | | | | | | | |
| | CAGA | | | | | | | |
| 231 | −33_4-4_bulge-symmetric_UUCG- | GCCACACAGACCUCCUUGGC | 0.221 | 0.778 | 0.783 | 1.084 | 1.511 | 1.523 |
| | ACAU | GGAGCCAAGUCCUUUCAUG | | | | | | |
| | −20_4-4_bulge-symmetric_UGGU- | AAUACAACCACGGCGACGG | | | | | | |
| | UAGU | ACCAGGGUUACUAGUCACU | | | | | | |
| | −10_6-6_internal_loop- | GUCGUACAUUGGCCACUCCC | | | | | | |
| | symmetric_AGGAAU-CCAGGG | AGU | | | | | | |
| | −4_4-4_bulge-symmetric_AUUA- | | | | | | | |
| | GACG | | | | | | | |
| | 0_1-1_mismatch_A-C | | | | | | | |
| | 4_1-1_mismatch_A-A | | | | | | | |
| | 24_6-6_internal_loop- | | | | | | | |
| | symmetric_UCAAAG-GGAGCC | | | | | | | |
| | 40_4-4_bulge-symmetric_GAGU- | | | | | | | |
| | CAGA | | | | | | | |
| 232 | −33_4-4_bulge-symmetric_UUCG- | GCCACACAGACCAAAAGCGC | 0.207 | 0.722 | 0.803 | 1.048 | 1.339 | 1.398 |
| | ACAU | CUUUGAAAGUCCUUUCAUG | | | | | | |
| | −23_0-2_bulge-asymmetric_-GU | GAUCGAUCCACGGCUUCUG | | | | | | |
| | −22_1-1_wobble_G-U | ACCAUGAUUACUAGUCACU | | | | | | |
| | −19_1-1_wobble_G-U | GUCGUACAUUGGCCACUCCC | | | | | | |
| | −15_4-5_internal_loop- | AGU | | | | | | |
| | asymmetric_UAAA-AUGAU | | | | | | | |
| | −10_3-0_bulge-asymmetric_AAU- | | | | | | | |
| | −5_2-2_bulge-symmetric_UU-UC | | | | | | | |
| | 0_1-1_mismatch_A-C | | | | | | | |
| | 6_2-2_bulge-symmetric_GU-CG | | | | | | | |
| | 10_1-1_wobble_U-G | | | | | | | |
| | 32_6-6_internal_loop- | | | | | | | |
| | symmetric_CAAGGA-AAAAGC | | | | | | | |
| | 40_4-4_bulge-symmetric_GAGU- | | | | | | | |
| | CAGA | | | | | | | |
| 233 | −33_4-4_bulge-symmetric_UUCG- | GCCACACAGACCUCCUGAUU | 0.29 | 0.919 | 0.922 | 1.096 | 1.619 | 1.641 |
| | ACAU | AAUUGAAAGUCCUUUCAUG | | | | | | |
| | −14_10−10_internal_loop- | AAUACAUCCACGGCUAUAU | | | | | | |
| | symmetric_UGGUGUAAAG- | AAUUCAGUAUGUAGUCACU | | | | | | |
| | AGUAUGUAGU | GUCGUACAUUGGCCACUCCC | | | | | | |
| | −6_3-3_bulge-symmetric_CAU-UAU | AGU | | | | | | |
| | 0_1-1_mismatch_A-C | | | | | | | |
| | 28_6-6_internal_loop- | | | | | | | |
| | symmetric_AGGCCA-GAUUAA | | | | | | | |
| | 40_4-4_bulge-symmetric_GAGU- | | | | | | | |
| | CAGA | | | | | | | |
| 234 | −33_4-4_bulge-symmetric_UUCG- | GCCACACAGACCUCCUCUUA | 0.077 | 0.688 | 0.716 | 0.989 | 1.49 | 1.502 |
| | ACAU | UAUUGAAAGUCCUUUCAUG | | | | | | |
| | −20_4-4_bulge-symmetric_UGGU- | AAUAUAUCGACGGCUACGA | | | | | | |
| | UAGU | CAAUCCUUUACUAGUCACU | | | | | | |
| | −6_6-6_internal_loop- | GUCGUACAUUGGCCACUCCC | | | | | | |
| | symmetric_AUUCAU-CGACAA | AGU | | | | | | |
| | 0_1-1_mismatch_A-C | | | | | | | |
| | 2_1-1_mismatch_G-G | | | | | | | |
| | 6_1-1_wobble_G-U | | | | | | | |
| | 28_6-6_internal_loop- | | | | | | | |
| | symmetric_AGGCCA-CUUAUA | | | | | | | |
| | 40_4-4_bulge-symmetric_GAGU- | | | | | | | |
| | CAGA | | | | | | | |
| 235 | −33_4-4_bulge-symmetric_UUCG- | GCCACACAAGGACACUUGG | 0.203 | 0.921 | 0.904 | 0.714 | 0.727 | 0.832 |
| | ACAU | CCUUUGAAAGUCCUUUCAU | | | | | | |
| | −23_1-0_bulge-asymmetric_U- | GAAUACAUUCACGACUUUU | | | | | | |
| | −22_1-1_wobble_G-U | GAAUUCGAAGUAUAGUCAC | | | | | | |
| | −21_1-1_mismatch_G-G | UGUCGUACAUUGGCCACUC | | | | | | |
| | −19_1-1_wobble_G-U | CCAGU | | | | | | |
| | −14_3-4_bulge-asymmetric_AAG- | | | | | | | |
| | GAAG | | | | | | | |
| | −5_2-2_bulge-symmetric_UU-UU | | | | | | | |
| | −2 1-1_mismatch_C-A | | | | | | | |
| | 0_1-1_mismatch_A-C | | | | | | | |
| | 3_1-1_wobble_G-U | | | | | | | |
| | 36_8-8_internal_loop- | | | | | | | |
| | symmetric_GAGGGAGU- | | | | | | | |
| | CAAGGACA | | | | | | | |
| 236 | −33_4-4_bulge-symmetric_UUCG- | GCCACACAGACCUCCUUGAA | 0.028 | 0.57 | 0.619 | 0.928 | 1.412 | 1.408 |
| | ACAU | UCAAGAAAGUCGGUUCAUG | | | | | | |
| | −20_4-4_bulge-symmetric_UGGU- | AAUACUCCCACGGCUAAUU | | | | | | |
| | UAGU | AGCGACUUUACUAGUCACU | | | | | | |
| | −8_6-6_internal_loop- | GUCGUACAUUGGCCACUCCC | | | | | | |
| | symmetric_GAAUUC-UAGCGA | AGU | | | | | | |
| | 0_1-1_mismatch_A-C | | | | | | | |
| | 4_2-2_bulge-symmetric_AU-UC | | | | | | | |
| | 17_2-2_bulge-symmetric_AG-GG | | | | | | | |
| | 26_6-6_internal_loop- | | | | | | | |
| | symmetric_AAAGGC-AAUCAA | | | | | | | |
| | 40_4-4_bulge-symmetric_GAGU- | | | | | | | |
| | CAGA | | | | | | | |
| 237 | −33_4-4_bulge-symmetric_UUCG- | GCCACACAGACCUCCUGUUA | 0.113 | 0.91 | 0.876 | 0.977 | 1.604 | 1.571 |
| | ACAU | UGUUGAAAGUCCUUUCAUG | | | | | | |
| | −16_8-8_internal_loop- | AAUACAUCGACGGCUAAUG | | | | | | |
| | symmetric_UGGUGUAA- | AAUUCCUCCUGUGGUCACU | | | | | | |
| | CCUGUGGU | GUCGUACAUUGGCCACUCCC | | | | | | |
| | 0_1-1_mismatch_A-C | AGU | | | | | | |
| | 2_1-1_mismatch_G-G | | | | | | | |
| | 28_6-6_internal_loop- | | | | | | | |
| | symmetric_AGGCCA-GUUAUG | | | | | | | |
| | 40_4-4_bulge-symmetric_GAGU- | | | | | | | |
| | CAGA | | | | | | | |
| 238 | −33_4-4_bulge-symmetric_UUCG- | GCCACACAGACCUCCUUGUA | 0.037 | 0.798 | 0.772 | 0.919 | 1.509 | 1.408 |
| | ACAU | GGGGGAAAGUCCUUUCCGG | | | | | | |
| | −23_0-1_bulge-asymmetric_-U | CAUGCAUCCACGGCUAGUG | | | | | | |
| | 3_1-1_wobble_U-G | AAUUCCUGGUAUGGUCACU | | | | | | |
| | −22_1-1_mismatch_G-G | GUCGUACAUUGGCCACUCCC | | | | | | |
| | −21_1-1_wobble_G-U | AGU | | | | | | |
| | −19_1-1_wobble_G-U | | | | | | | |
| | −16_3-2_bulge-asymmetric_UAA-GG | | | | | | | |
| | −6_1-1_wobble_U-G | | | | | | | |
| | 0_1-1_mismatch_A-C | | | | | | | |
| | 7_1-1_wobble_U-G | | | | | | | |
| | 10_1-1_mismatch_U-C | | | | | | | |
| | 12_2-2_bulge-symmetric_AU-CG | | | | | | | |
| | 26_6-6_internal_loop- | | | | | | | |
| | symmetric_AAAGGC-UAGGGG | | | | | | | |
| | 40_4-4_bulge-symmetric_GAGU- | | | | | | | |
| | CAGA | | | | | | | |
| 239 | −33_4-4_bulge-symmetric_UUCG- | GCCACACAGACCUCCUUGUA | 0.033 | 0.911 | 0.881 | 0.905 | 1.673 | 1.607 |
| | ACAU | GUAGGAAAGUCGUUUCCAU | | | | | | |
| | −18_6-6_internal_loop- | GAAUACAUCCACGGCUAAU | | | | | | |
| | symmetric_UGGUGU-UUCGUU | GAAUUCCUUUUUCGUUCAC | | | | | | |
| | 0_1-1_mismatch_A-C | UGUCGUACAUUGGCCACUC | | | | | | |
| | 14_0-1_bulge-asymmetric_-C | CCAGU | | | | | | |
| | 18_1-1_mismatch_G-G | | | | | | | |
| | 26_6-6_internal_loop- | | | | | | | |
| | symmetric_AAAGGC-UAGUAG | | | | | | | |
| | 40_4-4_bulge-symmetric_GAGU- | | | | | | | |
| | CAGA | | | | | | | |
| 240 | −33_4-4_bulge-symmetric_UUCG- | GCCACACAGACCUCCUUGAA | 0.07 | 0.924 | 0.891 | 0.931 | 1.654 | 1.614 |
| | ACAU | AAUGGAAAGUCCUGUCAUU | | | | | | |
| | −20_4-4_bulge-symmetric_UGGU- | AAUACAUCCACGGCUAAUU | | | | | | |
| | UAGU | UGGGACUUUACUAGUCACU | | | | | | |
| | −8_6-6_internal_loop- | GUCGUACAUUGGCCACUCCC | | | | | | |
| | symmetric_GAAUUC-UUGGGA | AGU | | | | | | |
| | 0_1-1_mismatch_A-C | | | | | | | |
| | 11_1-1_mismatch_C-U | | | | | | | |
| | 16_1-1_mismatch_A-G | | | | | | | |
| | 26_6-6_internal_loop- | | | | | | | |
| | symmetric_AAAGGC-AAAAUG | | | | | | | |
| | 40_4-4_bulge-symmetric_GAGU- | | | | | | | |
| | CAGA | | | | | | | |
| 241 | −33_4-4_bulge-symmetric_UUCG- | GCCACACAGACCUCCUCACG | 0.038 | 0.515 | 0.569 | 0.913 | 1.285 | 1.285 |
| | ACAU | AAUUGAAAGUCCUUCUUGA | | | | | | |
| | −23_0-1_bulge-asymmetric_-U | AUACAUCCACGGCUACAGA | | | | | | |
| | −23_1-1_wobble_U-G | AUUCCUACAAUGGUCACUG | | | | | | |
| | −22_1-1_mismatch_G-G | UCGUACAUUGGCCACUCCCA | | | | | | |
| | −21_1-1_wobble_G-U | GU | | | | | | |
| | −16_4-3_bulge-asymmetric_GUAA- | | | | | | | |
| | ACA | | | | | | | |
| | −6_2-2_bulge-symmetric_AU-CA | | | | | | | |
| | 0_1-1_mismatch_A-C | | | | | | | |
| | 13_1-1_mismatch_U-U | | | | | | | |
| | 17_1-0_bulge-asymmetric_A- | | | | | | | |
| | 28_6-6_internal_loop- | | | | | | | |
| | symmetric_AGGCCA-CACGAA | | | | | | | |
| | 40_4-4_bulge-symmetric_GAGU- | | | | | | | |
| | CAGA | | | | | | | |
| 242 | −33_4-4_bulge-symmetric_UUCG- | GCCACACAGACCUCCUUGGC | 0.033 | 0.848 | 0.867 | 0.932 | 1.584 | 1.586 |
| | ACAU | GAUGCUAAGUAGGUUCAUG | | | | | | |
| | −22_2-3_bulge-asymmetric_UG-AGU | AAUACGUCCACGGCAAUGG | | | | | | |
| | −21_1-1_wobble_G-U | AAUUCGGGUCAUAGUCACU | | | | | | |
| | −15_4-4_bulge-symmetric_UAAA- | GUCGUACAUUGGCCACUCCC | | | | | | |
| | GGGU | AGU | | | | | | |
| | −13_1-1_wobble_G-U | | | | | | | |
| | −11_1-1_mismatch_A-A | | | | | | | |
| | −9_1-1_wobble_U-G | | | | | | | |
| | −4_1-0_bulge-asymmetric_A- | | | | | | | |
| | 0_1-1_mismatch_A-C | | | | | | | |
| | 5_1-1_wobble_U-G | | | | | | | |
| | 17_3-3_bulge-symmetric_AGG-AGG | | | | | | | |
| | 24_6-6_internal_loop- | | | | | | | |
| | symmetric_UCAAAG-GAUGCU | | | | | | | |
| | 40_4-4_bulge-symmetric_GAGU- | | | | | | | |
| | CAGA | | | | | | | |
| 243 | −33_4-4_bulge-symmetric_UUCG- | GCCACACAAGGGCGCUUGG | 0.116 | 0.944 | 0.928 | 0.718 | 0.804 | 0.827 |
| | ACAU | CCUUUGAAAGUCCUUGCAU | | | | | | |
| | −20_4-4_bulge-symmetric_UGGU- | GAAUACACCCACGGCUACG | | | | | | |
| | UAGU | ACUGUCCUUUACUAGUCAC | | | | | | |
| | −6_6-6_internal_loop- | UGUCGUACAUUGGCCACUC | | | | | | |
| | symmetric_AUUCAU-CGACUG | CCAGU | | | | | | |
| | 0_1-1_mismatch_A-C | | | | | | | |
| | 4_1-1_mismatch_A-C | | | | | | | |
| | 15_1-1_mismatch_A-G | | | | | | | |
| | 36_8-8_internal_loop- | | | | | | | |
| | symmetric_GAGGGAGU- | | | | | | | |
| | CAAGGGCG | | | | | | | |
| 244 | −33_4-4_bulge-symmetric_UUCG- | GCCACACAGGAAAGCUUGG | 0.44 | 0.926 | 0.917 | 0.833 | 0.703 | 0.772 |
| | ACAU | CCUUUGAAAGUCCUUUCAU | | | | | | |
| | −20_4-4_bulge-symmetric_UGGU- | GAAUACCGCCACGGCUAAU | | | | | | |
| | UAGU | UUGAGACUUUACUAGUCAC | | | | | | |
| | −8_6-6_internal_loop- | UGUCGUACAUUGGCCACUC | | | | | | |
| | symmetric_GAAUUC-UUGAGA | CCAGU | | | | | | |
| | 0_1-1_mismatch_A-C | | | | | | | |
| | 4_2-2_bulge-symmetric_AU-CG | | | | | | | |
| | 36_8-8_internal_loop- | | | | | | | |
| | symmetric_GAGGGAGU- | | | | | | | |
| | CAGGAAAG | | | | | | | |
| 245 | −33_4-4_bulge-symmetric_UUCG- | GCCACACAGACCUCCUCAUU | 0.182 | 0.92 | 0.929 | 1.015 | 1.623 | 1.583 |
| | ACAU | UGUUGAAAGUCCUCUCAUG | | | | | | |
| | −20_4-4_bulge-symmetric_UGGU- | AAUACAUCCACGGCUAAUC | | | | | | |
| | UAGU | AUUGGCUUUACUAGUCACU | | | | | | |
| | −8_6-6_internal_loop- | GUCGUACAUUGGCCACUCCC | | | | | | |
| | symmetric_GAAUUC-CAUUGG | AGU | | | | | | |
| | 0_1-1_mismatch_A-C | | | | | | | |
| | 16_1-1_mismatch_A-C | | | | | | | |
| | 28_6-6_internal_loop- | | | | | | | |
| | symmetric_AGGCCA-CAUUUG | | | | | | | |
| | 40_4-4_bulge-symmetric_GAGU- | | | | | | | |
| | CAGA | | | | | | | |
| 246 | −33_4-4_bulge-symmetric_UUCG- | GCCACACAGACCUCAAGCAG | 0.305 | 0.896 | 0.879 | 1.142 | 1.459 | 1.448 |
| | ACAU | CUUUGAAAGUCCUUUCAUG | | | | | | |
| | −20_4-4_bulge-symmetric_UGGU- | AAUACCUCCACGGCUAAUCC | | | | | | |
| | UAGU | AAGGCUUUACUAGUCACUG | | | | | | |
| | −8_6-6_internal_loop- | UCGUACAUUGGCCACUCCCA | | | | | | |
| | symmetric_GAAUUC-CCAAGG | GU | | | | | | |
| | 0_1-1_mismatch_A-C | | | | | | | |
| | 5_1-1_mismatch_U-C | | | | | | | |
| | 30_6-6_internal_loop- | | | | | | | |
| | symmetric_GCCAAG-AAGCAG | | | | | | | |
| | 40_4-4_bulge-symmetric_GAGU- | | | | | | | |
| | CAGA | | | | | | | |
| 247 | −33_4-4_bulge-symmetric_UUCG- | GCCACACAGACCUCCUUGUA | 0.034 | 0.915 | 0.911 | 0.929 | 1.686 | 1.661 |
| | ACAU | ACACGAAAGUCCUUUUAUG | | | | | | |
| | −23_0-1_bulge-asymmetric_-U | AAUAUAUCCACGGCUAUAU | | | | | | |
| | −23_1-1_wobble_U-G | AAUUCCUAAAAUGGUCACU | | | | | | |
| | −22_1-1_mismatch_G-G | GUCGUACAUUGGCCACUCCC | | | | | | |
| | −21_1-1_wobble_G-U | AGU | | | | | | |
| | −16_4-3_bulge-asymmetric_GUAA- | | | | | | | |
| | AAA | | | | | | | |
| | −6_3-3_bulge-symmetric_CAU-UAU | | | | | | | |
| | 0_1-1_mismatch_A-C | | | | | | | |
| | 6_1-1_wobble_G-U | | | | | | | |
| | 14_1-1_wobble_G-U | | | | | | | |
| | 26_6-6_internal_loop- | | | | | | | |
| | symmetric_AAAGGC-UAACAC | | | | | | | |
| | 40_4-4_bulge-symmetric_GAGU- | | | | | | | |
| | CAGA | | | | | | | |
| 248 | −33_4-4_bulge-symmetric_UUCG- | GCCACAUGGAAGUCCUUGG | 0.174 | 0.919 | 0.939 | 0.615 | 1.187 | 1.061 |
| | ACAU | CCUUUGAAAGUCCUUUCAU | | | | | | |
| | −20_4-4_bulge-symmetric_UGGU- | GAAUCGAUCCACGGCUAAU | | | | | | |
| | UAGU | CUACGGCUUUACUAGUCAC | | | | | | |
| | −8_6-6_internal_loop- | UGUCGUACAUUGGCCACUC | | | | | | |
| | symmetric_GAAUUC-CUACGG | CCAGU | | | | | | |
| | 0_1-1_mismatch_A-C | | | | | | | |
| | 6_2-2_bulge-symmetric_GU-CG | | | | | | | |
| | 38_6-6_internal_loop- | | | | | | | |
| | symmetric_GGGAGU-UGGAAG | | | | | | | |
| 249 | −33_4-4_bulge-symmetric_UUCG- | GCCACACAGACCGAGCGUGC | 0.33 | 0.95 | 0.927 | 0.913 | 0.839 | 0.914 |
| | ACAU | CUUUGAAAGUCCUUUCAUG | | | | | | |
| | −22_2-5_internal_loop- | AAUACAACCACGGCUAAUG | | | | | | |
| | asymmetric_UG-GUAGU | AAUUCGACACGUAGUCACU | | | | | | |
| | −14_4-1_bulge-asymmetric_AAAG-G | GUCGUACAUUGGCCACUCCC | | | | | | |
| | 0_1-1_mismatch_A-C | AGU | | | | | | |
| | 4_1-1_mismatch_A-A | | | | | | | |
| | 32_6-6_internal_loop- | | | | | | | |
| | symmetric_CAAGGA-GAGCGU | | | | | | | |
| | 40_4-4_bulge-symmetric_GAGU- | | | | | | | |
| | CAGA | | | | | | | |
| 250 | −33_4-4_bulge-symmetric_UUCG- | GCCACACAGAGAUAAAUGG | 0.162 | 0.938 | 0.905 | 0.743 | 1.155 | 1.127 |
| | ACAU | CCUUUGAAAGUGGUUUCAU | | | | | | |
| | −23_1-0_bulge-asymmetric_U- | GAAUACAUCCACGGCUAAU | | | | | | |
| | −22_1-1_wobble_G-U | GAAUUCAAACGAUAGUCAC | | | | | | |
| | −21_1-1_mismatch_G-G | UGUCGUACAUUGGCCACUC | | | | | | |
| | −19_1-1_wobble_G-U | CCAGU | | | | | | |
| | −14_4-5_internal_loop- | | | | | | | |
| | asymmetric_AAAG-AAACG | | | | | | | |
| | 0_1-1_mismatch_A-C | | | | | | | |
| | 18_2-2_bulge-symmetric_GG-GG | | | | | | | |
| | 34_10−10_internal_loop- | | | | | | | |
| | symmetric_AGGAGGGAGU- | | | | | | | |
| | CAGAGAUAAA | | | | | | | |
| 251 | −33_4-4_bulge-symmetric_UUCG- | GCCACACAGACCUCCUAAUU | 0.041 | 0.947 | 0.934 | 0.952 | 1.703 | 1.646 |
| | ACAU | UGUUGAAAGUCACUUCAUG | | | | | | |
| | −23_0-1_bulge-asymmetric_-U | AAUACCUCCACGGCUAAUU | | | | | | |
| | −23_1-1_wobble_U-G | AAUUCCUAGGAUGGUCACU | | | | | | |
| | −22_1-1_mismatch_G-G | GUCGUACAUUGGCCACUCCC | | | | | | |
| | −21_1-1_wobble_G-U | AGU | | | | | | |
| | −19_1-1_mismatch_G-G | | | | | | | |
| | −18_1-1_wobble_U-G | | | | | | | |
| | −16_2-1_bulge-asymmetric_AA-A | | | | | | | |
| | −8_1-1_mismatch_C-U | | | | | | | |
| | 0_1-1_mismatch_A-C | | | | | | | |
| | 5_1-1_mismatch_U-C | | | | | | | |
| | 17_2-2_bulge-symmetric_AG-AC | | | | | | | |
| | 28_6-6_internal_loop- | | | | | | | |
| | symmetric_AGGCCA-AAUUUG | | | | | | | |
| | 40_4-4_bulge-symmetric_GAGU- | | | | | | | |
| | CAGA | | | | | | | |
| 252 | −33_4-4_bulge-symmetric_UUCG- | GCCACACAGACCUCCUUGAA | 0.062 | 0.927 | 0.91 | 0.912 | 1.657 | 1.633 |
| | ACAU | AGCAGAAAGUCCUUUGAUG | | | | | | |
| | −23_0-2_bulge-asymmetric_-GU | AAUACAUCCACGGGUAAUA | | | | | | |
| | −22_1-1_wobble_G-U | AAUGUGCGAACUAGUCACU | | | | | | |
| | −12_8-6_internal_loop- | GUCGUACAUUGGCCACUCCC | | | | | | |
| | asymmetric_GUAAAGGA-GUGCGA | AGU | | | | | | |
| | −8_1-1_mismatch_C-A | | | | | | | |
| | −3_1-1_mismatch_G-G | | | | | | | |
| | 0_1-1_mismatch_A-C | | | | | | | |
| | 14_1-1_mismatch_G-G | | | | | | | |
| | 26_6-6_internal_loop- | | | | | | | |
| | symmetric_AAAGGC-AAAGCA | | | | | | | |
| | 40_4-4_bulge-symmetric_GAGU- | | | | | | | |
| | CAGA | | | | | | | |
| 253 | −33_4-4_bulge-symmetric_UUCG- | GCCACACAGACCUCCUCUCG | 0.087 | 0.589 | 0.629 | 0.992 | 1.407 | 1.428 |
| | ACAU | AAUUGAAAGUCCUUUCAUG | | | | | | |
| | −16_8-8_internal_loop- | AAUACGCCACGGCUCGGAA | | | | | | |
| | symmetric_UGGUGUAA- | UUCCUGAUUUGGUCACUGU | | | | | | |
| | GAUUUGGU | CGUACAUUGGCCACUCCCAG | | | | | | |
| | −5_3-2_bulge-asymmetric_AUU-CG | U | | | | | | |
| | 0_1-1_mismatch_A-C | | | | | | | |
| | 4_1-0_bulge-asymmetric_A- | | | | | | | |
| | 5_1-1_wobble_U-G | | | | | | | |
| | 28_6-6_internal_loop- | | | | | | | |
| | symmetric_AGGCCA-CUCGAA | | | | | | | |
| | 40_4-4_bulge-symmetric_GAGU- | | | | | | | |
| | CAGA | | | | | | | |
| 254 | −33_4-4_bulge-symmetric_UUCG- | GCCACACAGACCUCCUGUUU | 0.065 | 0.566 | 0.573 | 0.929 | 1.328 | 1.317 |
| | ACAU | ACUUGAAAGUCCUCUCAUG | | | | | | |
| | −20_4-4_bulge-symmetric_UGGU- | AAUACAUCCACGGUAAUUC | | | | | | |
| | UAGU | AAGGCUUUACUAGUCACUG | | | | | | |
| | −8_6-6_internal_loop- | UCGUACAUUGGCCACUCCCA | | | | | | |
| | symmetric_GAAUUC-UCAAGG | GU | | | | | | |
| | −3_1-0_bulge-asymmetric_G- | | | | | | | |
| | 0_1-1_mismatch_A-C | | | | | | | |
| | 16_1-1_mismatch_A-C | | | | | | | |
| | 28_6-6_internal_loop- | | | | | | | |
| | symmetric_AGGCCA-GUUUAC | | | | | | | |
| | 40_4-4_bulge-symmetric_GAGU- | | | | | | | |
| | CAGA | | | | | | | |
| 255 | −33_4-4_bulge-symmetric_UUCG- | GCCACACAAAAAGGCUUGG | 0.1 | 0.862 | 0.844 | 0.513 | 0.624 | 0.662 |
| | ACAU | CCUUUGAAAGUCCUGUCAU | | | | | | |
| | −22_2-3_bulge-asymmetric_UG-AGU | AAAUACAUCCACGGCUAAU | | | | | | |
| | −21_1-1_wobble_G-U | UAAUUCGGGUCAUAGUCAC | | | | | | |
| | −14_5-4_internal_loop- | UGUCGUACAUUGGCCACUC | | | | | | |
| | asymmetric_UAAAG-GGGU | CCAGU | | | | | | |
| | −8_1-1_mismatch_C-U | | | | | | | |
| | 0_1-1_mismatch_A-C | | | | | | | |
| | 11_1-1_mismatch_C-A | | | | | | | |
| | 16_1-1_mismatch_A-G | | | | | | | |
| | 36_8-8_internal_loop- | | | | | | | |
| | symmetric_GAGGGAGU- | | | | | | | |
| | CAAAAAGG | | | | | | | |
| 256 | −33_4-4_bulge-symmetric_UUCG- | GCCACACAGACCUCCUCAUA | 0.048 | 0.77 | 0.832 | 0.952 | 1.525 | 1.511 |
| | ACAU | UGUUGAAAGUCCUUUCUUG | | | | | | |
| | −16_8-8_internal_loop- | AAUACCAUCCACGGCUAAA | | | | | | |
| | symmetric_UGGUGUAA- | AAUUCCUCUCGUGGUCACU | | | | | | |
| | CUCGUGGU | GUCGUACAUUGGCCACUCCC | | | | | | |
| | −7_2-1_bulge-asymmetric_CA-A | AGU | | | | | | |
| | 0_1-1_mismatch_A-C | | | | | | | |
| | 6_0-1_bulge-asymmetric_-C | | | | | | | |
| | 13_1-1_mismatch_U-U | | | | | | | |
| | 28_6-6_internal_loop- | | | | | | | |
| | symmetric_AGGCCA-CAUAUG | | | | | | | |
| | 40_4-4_bulge-symmetric_GAGU- | | | | | | | |
| | CAGA | | | | | | | |
| 257 | −33_4-4_bulge-symmetric_UUCG- | GCCACACAGACCUCCUUGGC | 0.272 | 0.928 | 0.933 | 1.073 | 1.482 | 1.439 |
| | ACAU | GGUGCUAAGUCCUUUCAUC | | | | | | |
| | −23_0-2_bulge-asymmetric_-GU | AAUACAUCCACGGCUAAUG | | | | | | |
| | −22_1-1_wobble_G-U | AAUAGACAAACUAGUCACU | | | | | | |
| | −12 8-6_internal_loop- | GUCGUACAUUGGCCACUCCC | | | | | | |
| | asymmetric_GUAAAGGA-AGACAA | AGU | | | | | | |
| | 0_1-1_mismatch_A-C | | | | | | | |
| | 11_1-1_mismatch_C-C | | | | | | | |
| | 24_6-6_internal_loop- | | | | | | | |
| | symmetric_UCAAAG-GGUGCU | | | | | | | |
| | 40_4-4_bulge-symmetric_GAGU- | | | | | | | |
| | CAGA | | | | | | | |
| 258 | −33_4-4_bulge-symmetric_UUCG- | GCCACACAGACCUCCUUGGC | 0.116 | 0.919 | 0.924 | 1.002 | 1.664 | 1.628 |
| | ACAU | GAAAACAAGUCCUUUCAUG | | | | | | |
| | −23_1-0_bulge-asymmetric_U- | AAUAUUUCCACGGCUAAUG | | | | | | |
| | −22_1-1_wobble_G-U | AAUUCGCCGGAUAGUCACU | | | | | | |
| | −21_1-1_mismatch_G-G | GUCGUACAUUGGCCACUCCC | | | | | | |
| | −19_1-1_wobble_G-U | AGU | | | | | | |
| | −14_4-5_internal_loop- | | | | | | | |
| | asymmetric_AAAG-GCCGG | | | | | | | |
| | 0_1-1_mismatch_A-C | | | | | | | |
| | 5_1-1_mismatch_U-U | | | | | | | |
| | 6_1-1_wobble_G-U | | | | | | | |
| | 24_6-6_internal_loop- | | | | | | | |
| | symmetric_UCAAAG-GAAAAC | | | | | | | |
| | 40_4-4_bulge-symmetric_GAGU- | | | | | | | |
| | CAGA | | | | | | | |
| 259 | −33_4-4_bulge-symmetric_UUCG- | GCCACACAGACCUCCUCGAG | 0.132 | 0.921 | 0.913 | 0.995 | 1.486 | 1.442 |
| | ACAU | AGUUGAAAGUCCUCUCAUG | | | | | | |
| | −23_0-2_bulge-asymmetric_-GU | AAUAGAUCCACGGCUAAUG | | | | | | |
| | −22_1-1_wobble_G-U | AAUGUGGGCACUAGUCACU | | | | | | |
| | −18_1-1_wobble_U-G | GUCGUACAUUGGCCACUCCC | | | | | | |
| | −12 6-4_internal_loop- | AGU | | | | | | |
| | asymmetric_AAAGGA-GUGG | | | | | | | |
| | 0_1-1_mismatch_A-C | | | | | | | |
| | 6_1-1_mismatch_G-G | | | | | | | |
| | 16_1-1_mismatch_A-C | | | | | | | |
| | 28_6-6_internal_loop- | | | | | | | |
| | symmetric_AGGCCA-CGAGAG | | | | | | | |
| | 40_4-4_bulge-symmetric_GAGU- | | | | | | | |
| | CAGA | | | | | | | |
| 260 | −33_4-4_bulge-symmetric_UUCG- | GCCACACAGAGACAACUGG | 0.168 | 0.834 | 0.813 | 0.622 | 0.689 | 0.736 |
| | ACAU | CCUUUGAAAGUCCUUUCAU | | | | | | |
| | −23_0-2_bulge-asymmetric_-GU | AAAUACAUCCACGGUUAAU | | | | | | |
| | −22_1-1_wobble_G-U | GAAUGUGGGCACUAGUCAC | | | | | | |
| | −18_1-1_wobble_U-G | UGUCGUACAUUGGCCACUC | | | | | | |
| | −12 6-4_internal_loop- | CCAGU | | | | | | |
| | asymmetric_AAAGGA-GUGG | | | | | | | |
| | −3 1-1_wobble_G-U | | | | | | | |
| | 0_1-1_mismatch_A-C | | | | | | | |
| | 11_1-1_mismatch_C-A | | | | | | | |
| | 34_1-0_bulge-asymmetric_A- | | | | | | | |
| | 36_2-2_bulge-symmetric_GA-AA | | | | | | | |
| | 39_5-6_internal_loop- | | | | | | | |
| | asymmetric_GGAGU-CAGAGA | | | | | | | |
| 261 | −33_4-4_bulge-symmetric_UUCG- | GCCACACAGACCUCCUUGAU | 0.255 | 0.894 | 0.882 | 1.115 | 1.681 | 1.61 |
| | ACAU | GACAGAAAGUCCUUUCAUG | | | | | | |
| | −23_0-2_bulge-asymmetric_-GU | AAUGCAGCCACGGCUAAUG | | | | | | |
| | −22_1-1_wobble_G-U | ACGCUUGUUACUAGUCACU | | | | | | |
| | −19_1-1_wobble_G-U | GUCGUACAUUGGCCACUCCC | | | | | | |
| | −17_2-2_bulge-symmetric_UA-GU | AGU | | | | | | |
| | −10_4-2_bulge-asymmetric_GAAU- | | | | | | | |
| | CG | | | | | | | |
| | 0_1-1_mismatch_A-C | | | | | | | |
| | 4_1-1_mismatch_A-G | | | | | | | |
| | 7_1-1_wobble_U-G | | | | | | | |
| | 26_6-6_internal_loop- | | | | | | | |
| | symmetric_AAAGGC-AUGACA | | | | | | | |
| | 40_4-4_bulge-symmetric_GAGU- | | | | | | | |
| | CAGA | | | | | | | |
| 262 | −33_4-4_bulge-symmetric_UUCG- | GCCACAUAGGGAUCCUUGG | 0.403 | 0.927 | 0.929 | 0.542 | 0.701 | 0.726 |
| | ACAU | CCUUUGAAAGUCCUUUCAU | | | | | | |
| | −18_6-6_internal_loop- | GAAUACAUCCACGGCGAAU | | | | | | |
| | symmetric_UGGUGU-UGUUAU | GAAUUCCUUUUGUUAUCAC | | | | | | |
| | −4_1-1_mismatch_A-G | UGUCGUACAUUGGCCACUC | | | | | | |
| | 0_1-1_mismatch_A-C | CCAGU | | | | | | |
| | 38_6-6_internal_loop- | | | | | | | |
| | symmetric_GGGAGU-UAGGGA | | | | | | | |
| 263 | −33_4-4_bulge-symmetric_UUCG- | GCCACACAGACCUCCUAUUG | 0.11 | 0.748 | 0.764 | 0.997 | 1.503 | 1.494 |
| | ACAU | AAUUGAAAGUCCUUUCAUG | | | | | | |
| | −23_0-4_bulge-asymmetric_-UAGU | AAUAGGAUCCACGGAUACU | | | | | | |
| | −23_1-1_wobble_U-G | GAAUUCGCCCCGUAGUCAC | | | | | | |
| | −20_1-1_mismatch_U-C | UGUCGUACAUUGGCCACUC | | | | | | |
| | −18_1-1_wobble_U-G | CCAGU | | | | | | |
| | −14_4-0_bulge-asymmetric_AAAG- | | | | | | | |
| | −6_1-1_mismatch_U-C | | | | | | | |
| | −3 1-1_mismatch_G-A | | | | | | | |
| | 0_1-1_mismatch_A-C | | | | | | | |
| | 6_1-2_bulge-asymmetric_G-GG | | | | | | | |
| | 28_6-6_internal_loop- | | | | | | | |
| | symmetric_AGGCCA-AUUGAA | | | | | | | |
| | 40_4-4_bulge-symmetric_GAGU- | | | | | | | |
| | CAGA | | | | | | | |
| 264 | −33_4-4_bulge-symmetric_UUCG- | GCCACACAGACCUCAGCCUA | 0.33 | 0.93 | 0.916 | 1.196 | 1.435 | 1.472 |
| | ACAU | CUUUGAAAGUCCUUUCAUC | | | | | | |
| | −20_4-4_bulge-symmetric_UGGU- | AAUACCACCACGGCUAAUG | | | | | | |
| | UAGU | ACAGUACUUACUAGUCACU | | | | | | |
| | −10_6-6_internal_loop- | GUCGUACAUUGGCCACUCCC | | | | | | |
| | symmetric_AGGAAU-CAGUAC | AGU | | | | | | |
| | 0_1-1_mismatch_A-C | | | | | | | |
| | 4_2-2_bulge-symmetric_AU-CA | | | | | | | |
| | 11_1-1_mismatch_C-C | | | | | | | |
| | 30_6-6_internal_loop- | | | | | | | |
| | symmetric_GCCAAG-AGCCUA | | | | | | | |
| | 40_4-4_bulge-symmetric_GAGU- | | | | | | | |
| | CAGA | | | | | | | |
| 265 | −33_4-4_bulge-symmetric_UUCG- | GCCACACAGACCUCCUUGGC | 0.262 | 0.847 | 0.888 | 1.131 | 1.584 | 1.621 |
| | ACAU | GACGAUAAGUCCUUUCAUG | | | | | | |
| | −23_0-2_bulge-asymmetric_-GU | AAUACAGCCACGGCUAUCA | | | | | | |
| | −22_1-1_wobble_G-U | UGAAUAUAGGCACUAGUCA | | | | | | |
| | −18_1-1_wobble_U-G | CUGUCGUACAUUGGCCACU | | | | | | |
| | −12 6-4_internal_loop- | CCCAGU | | | | | | |
| | asymmetric_AAAGGA-AUAG | | | | | | | |
| | −5_0-2_bulge-asymmetric_-UC | | | | | | | |
| | 0_1-1_mismatch_A-C | | | | | | | |
| | 4_1-1_mismatch_A-G | | | | | | | |
| | 24_6-6_internal_loop- | | | | | | | |
| | symmetric_UCAAAG-GACGAU | | | | | | | |
| | 40_4-4_bulge-symmetric_GAGU- | | | | | | | |
| | CAGA | | | | | | | |
| 266 | −33_4-4_bulge-symmetric_UUCG- | GCCACACAGAGUAGCUUGG | 0.382 | 0.666 | 0.706 | 0.871 | 0.77 | 0.842 |
| | ACAU | CCUUUGAAAGUCCUUUCAU | | | | | | |
| | −20_4-4_bulge-symmetric_UGGU- | GAAUAGAGCCACGGCUAAU | | | | | | |
| | UAGU | UUCGCACUUUACUAGUCAC | | | | | | |
| | −8_6-6_internal_loop- | UGUCGUACAUUGGCCACUC | | | | | | |
| | symmetric_GAAUUC-UUCGCA | CCAGU | | | | | | |
| | 0_1-1_mismatch_A-C | | | | | | | |
| | 4_1-1_mismatch_A-G | | | | | | | |
| | 6_1-1_mismatch_G-G | | | | | | | |
| | 36_8-8_internal_loop- | | | | | | | |
| | symmetric_GAGGGAGU- | | | | | | | |
| | CAGAGUAG | | | | | | | |
| 267 | −33_4-4_bulge-symmetric_UUCG- | GCCACACAGACCUCCUUGUA | 0.023 | 0.927 | 0.923 | 0.91 | 1.696 | 1.697 |
| | ACAU | GGGGGAAAGUCCUUGAAUG | | | | | | |
| | −20_6-6_internal_loop- | AAUACAUCCACGGCUAAUG | | | | | | |
| | symmetric_UGUGGU-UGUGUU | AAUUCCUUUACUGUGUUCU | | | | | | |
| | 0_1-1_mismatch_A-C | GUCGUACAUUGGCCACUCCC | | | | | | |
| | 14_2-2_bulge-symmetric_GA-GA | AGU | | | | | | |
| | 26_6-6_internal_loop- | | | | | | | |
| | symmetric_AAAGGC-UAGGGG | | | | | | | |
| | 40_4-4_bulge-symmetric_GAGU- | | | | | | | |
| | CAGA | | | | | | | |
| 268 | −33_4-4_bulge-symmetric_UUCG- | GCCACACAGACCUCCUUGUA | 0.025 | 0.553 | 0.594 | 0.933 | 1.236 | 1.187 |
| | ACAU | UGGGGAAAGUCCCUUGAUG | | | | | | |
| | −20_4-4_bulge-symmetric_UGGU- | ACACAUCCACGGCUAAUGA | | | | | | |
| | UAGU | CGCGGAUUACUAGUCACUG | | | | | | |
| | −10_6-6_internal_loop- | UCGUACAUUGGCCACUCCCA | | | | | | |
| | symmetric_AGGAAU-CGCGGA | GU | | | | | | |
| | 0_1-1_mismatch_A-C | | | | | | | |
| | 8_2-1_bulge-asymmetric_AU-C | | | | | | | |
| | 14_1-1_mismatch_G-G | | | | | | | |
| | 17_1-1_mismatch_A-C | | | | | | | |
| | 26_6-6_internal_loop- | | | | | | | |
| | symmetric_AAAGGC-UAUGGG | | | | | | | |
| | 40_4-4_bulge-symmetric_GAGU- | | | | | | | |
| | CAGA | | | | | | | |
| 269 | −33_4-4_bulge-symmetric_UUCG- | GCCACACAGACCUCCUACUG | 0.034 | 0.752 | 0.783 | 0.939 | 1.404 | 1.366 |
| | ACAU | ACUUGAAAGUCAGUUUCAU | | | | | | |
| | −20_4-4_bulge-symmetric_UGGU- | GAAUACAUCCACGGCUAUC | | | | | | |
| | UAGU | UAGCUCCUUUACUAGUCAC | | | | | | |
| | −6_6-6_internal_loop- | UGUCGUACAUUGGCCACUC | | | | | | |
| | symmetric_AUUCAU-UCUAGC | CCAGU | | | | | | |
| | 0_1-1_mismatch_A-C | | | | | | | |
| | 18_1-2_bulge-asymmetric_G-AG | | | | | | | |
| | 28_6-6_internal_loop- | | | | | | | |
| | symmetric_AGGCCA-ACUGAC | | | | | | | |
| | 40_4-4_bulge-symmetric_GAGU- | | | | | | | |
| | CAGA | | | | | | | |
| 270 | −33_4-4_bulge-symmetric_UUCG- | GCCACACAGACCUCCUUGUU | 0.047 | 0.76 | 0.742 | 0.949 | 1.587 | 1.526 |
| | ACAU | GAUGGAAAGUCCUUUCAUG | | | | | | |
| | −20_4-4_bulge-symmetric_UGGU- | AAUACCCCACGGCUAAUAC | | | | | | |
| | UAGU | AGGGCUUUACUAGUCACUG | | | | | | |
| | −8_6-6_internal_loop- | UCGUACAUUGGCCACUCCCA | | | | | | |
| | symmetric_GAAUUC-ACAGGG | GU | | | | | | |
| | 0_1-1_mismatch_A-C | | | | | | | |
| | 4_2-1_bulge-asymmetric_AU-C | | | | | | | |
| | 26_6-6_internal_loop- | | | | | | | |
| | symmetric_AAAGGC-UUGAUG | | | | | | | |
| | 40_4-4_bulge-symmetric_GAGU- | | | | | | | |
| | CAGA | | | | | | | |
| 271 | −33_4-4_bulge-symmetric_UUCG- | GCCACACAGACCUCCUUGUU | 0.296 | 0.959 | 0.942 | 1.135 | 1.726 | 1.664 |
| | ACAU | GAAGGAAAGUCCUUUCAUG | | | | | | |
| | −18_6-6_internal_loop- | AAUACUUCCACGGCUAAUG | | | | | | |
| | symmetric_UGGUGU-UUUGUU | AAUUCCUUUUUUGUUCACU | | | | | | |
| | 0_1-1_mismatch_A-C | GUCGUACAUUGGCCACUCCC | | | | | | |
| | 5_1-1_mismatch_U-U | AGU | | | | | | |
| | 26_6-6_internal_loop- | | | | | | | |
| | symmetric_AAAGGC-UUGAAG | | | | | | | |
| | 40_4-4_bulge-symmetric_GAGU- | | | | | | | |
| | CAGA | | | | | | | |
| 272 | −33_4-4_bulge-symmetric_UUCG- | GCCACACAGACCUCGAUCCG | 0.221 | 0.95 | 0.928 | 1.071 | 1.218 | 1.302 |
| | ACAU | CUUUGAAAGUCCUUUCAUG | | | | | | |
| | −23_0-1_bulge-asymmetric_-U | UAUACAUCCACGGCUAAUG | | | | | | |
| | −23_1-1_wobble_U-G | AAUUCCUCAUUCGGUCACU | | | | | | |
| | −22_1-1_mismatch_G-G | GUCGUACAUUGGCCACUCCC | | | | | | |
| | −20_1-1_mismatch_U-U | AGU | | | | | | |
| | −19_1-1_wobble_G-U | | | | | | | |
| | −16_2-1_bulge-asymmetric_AA-C | | | | | | | |
| | 0_1-1_mismatch_A-C | | | | | | | |
| | 10_1-1_mismatch_U-U | | | | | | | |
| | 30_6-6_internal_loop- | | | | | | | |
| | symmetric_GCCAAG-GAUCCG | | | | | | | |
| | 40_4-4_bulge-symmetric_GAGU- | | | | | | | |
| | CAGA | | | | | | | |
| 273 | −33_4-4_bulge-symmetric_UUCG- | GCCACACAGACCUCCUUGUA | 0.052 | 0.862 | 0.833 | 0.887 | 1.604 | 1.588 |
| | ACAU | GGACGAAAGUACUUUCAUG | | | | | | |
| | −20_6-6_internal_loop- | AAUACAUCCAUAGAUGCUG | | | | | | |
| | symmetric_UGUGGU-UAGAAU | AAUUCCUUUACUAGAAUCU | | | | | | |
| | −6_1-1_mismatch_U-C | GUCGUACAUUGGCCACUCCC | | | | | | |
| | −5_1-1_wobble_U-G | AGU | | | | | | |
| | −3_1-1_mismatch_G-A | | | | | | | |
| | −1_1-1_mismatch_C-A | | | | | | | |
| | 19_1-1_mismatch_G-A | | | | | | | |
| | 26_6-6_internal_loop- | | | | | | | |
| | symmetric_AAAGGC-UAGGAC | | | | | | | |
| | 40_4-4_bulge-symmetric_GAGU- | | | | | | | |
| | CAGA | | | | | | | |
| 274 | −33_4-4_bulge-symmetric_UUCG- | GCCACACAGACCUCGCAAUA | 0.284 | 0.527 | 0.635 | 1.151 | 1.136 | 1.32 |
| | ACAU | CUUUGAAAGUCCUUUCAUG | | | | | | |
| | −23_0-2_bulge-asymmetric_-GU | AAUACAGCCACGGCAAAUU | | | | | | |
| | −22_1-1_wobble_G-U | CCUGGCUUUACUAGUCACU | | | | | | |
| | −19_1-1_wobble_G-U | GUCGUACAUUGGCCACUCCC | | | | | | |
| | −16_3-5_internal_loop- | AGU | | | | | | |
| | asymmetric_UAA-GGCUU | | | | | | | |
| | −4_5-1_internal_loop- | | | | | | | |
| | asymmetric_CAUUA-A | | | | | | | |
| | 0_1-1_mismatch_A-C | | | | | | | |
| | 4_1-1_mismatch_A-G | | | | | | | |
| | 30_6-6_internal_loop- | | | | | | | |
| | symmetric_GCCAAG-GCAAUA | | | | | | | |
| | 40_4-4_bulge-symmetric_GAGU- | | | | | | | |
| | CAGA | | | | | | | |
| 275 | −33_4-4_bulge-symmetric_UUCG- | GCCACAUAGGGGUCCUUGG | 0.116 | 0.92 | 0.929 | 0.604 | 0.796 | 0.786 |
| | ACAU | CCUUUGAAAGUCUUUUCAU | | | | | | |
| | −20_4-4_bulge-symmetric_UGGU- | GAAUUCAUCCACGGCUACG | | | | | | |
| | UAGU | CUAGUCCUUUACUAGUCAC | | | | | | |
| | −6_6-6_internal_loop- | UGUCGUACAUUGGCCACUC | | | | | | |
| | symmetric_AUUCAU-CGCUAG | CCAGU | | | | | | |
| | 0_1-1_mismatch_A-C | | | | | | | |
| | 7_1-1_mismatch_U-U | | | | | | | |
| | 18_1-1_wobble_G-U | | | | | | | |
| | 38_6-6_internal_loop- | | | | | | | |
| | symmetric_GGGAGU-UAGGGG | | | | | | | |
| 276 | −33_4-4_bulge-symmetric_UUCG- | GCCACACAGACCAGGUAUG | 0.065 | 0.584 | 0.629 | 0.971 | 1.28 | 1.326 |
| | ACAU | CCUUUGAAAGUCAAUUCAU | | | | | | |
| | −20_4-4_bulge-symmetric_UGGU- | GAAUACUUCCACGACUAUA | | | | | | |
| | UAGU | AAACUCCUUUACUAGUCAC | | | | | | |
| | −6_6-6_internal_loop- | UGUCGUACAUUGGCCACUC | | | | | | |
| | symmetric_AUUCAU-UAAAAC | CCAGU | | | | | | |
| | −2_1-1_mismatch_C-A | | | | | | | |
| | 0_1-1_mismatch_A-C | | | | | | | |
| | 5_1-1_mismatch_U-U | | | | | | | |
| | 17_2-2_bulge-symmetric_AG-AA | | | | | | | |
| | 32_6-6_internal_loop- | | | | | | | |
| | symmetric_CAAGGA-AGGUAU | | | | | | | |
| | 40_4-4_bulge-symmetric_GAGU- | | | | | | | |
| | CAGA | | | | | | | |
| 277 | −33_4-4_bulge-symmetric_UUCG- | GCCACACAGACCUCCUUGAU | 0.379 | 0.913 | 0.908 | 1.219 | 1.72 | 1.652 |
| | ACAU | AGGCGAAAGUCCUUUCAUG | | | | | | |
| | −20_4-4_bulge-symmetric_UGGU- | AAUAACUCCACGGCUAUCA | | | | | | |
| | UAGU | UACUCCUUUACUAGUCACU | | | | | | |
| | −6_6-6_internal_loop- | GUCGUACAUUGGCCACUCCC | | | | | | |
| | symmetric_AUUCAU-UCAUAC | AGU | | | | | | |
| | 0_1-1_mismatch_A-C | | | | | | | |
| | 5_2-2_bulge-symmetric_UG-AC | | | | | | | |
| | 26_6-6_internal_loop- | | | | | | | |
| | symmetric_AAAGGC-AUAGGC | | | | | | | |
| | 40_4-4_bulge-symmetric_GAGU- | | | | | | | |
| | CAGA | | | | | | | |
| 278 | −33_4-4_bulge-symmetric_UUCG- | GCCACACAGACCUCCUUGUA | 0.034 | 0.886 | 0.877 | 0.916 | 1.639 | 1.631 |
| | ACAU | UGAGGAAAGUCCUUUCCAG | | | | | | |
| | −18_6-6_internal_loop- | AAUACAUCCACGGCUAAUG | | | | | | |
| | symmetric_UGGUGU-UAGUGU | AAUUCCUUUUAGUGUCACU | | | | | | |
| | 0_1-1_mismatch_A-C | GUCGUACAUUGGCCACUCCC | | | | | | |
| | 12_2-2_bulge-symmetric_AU-CA | AGU | | | | | | |
| | 26_6-6_internal_loop- | | | | | | | |
| | symmetric_AAAGGC-UAUGAG | | | | | | | |
| | 40_4-4_bulge-symmetric_GAGU- | | | | | | | |
| | CAGA | | | | | | | |
| 279 | −33_4-4_bulge-symmetric_UUCG- | GCCACACAGACCUCCUGACU | 0.122 | 0.771 | 0.771 | 0.838 | 0.915 | 0.925 |
| | ACAU | AAUUGAAAGUCCUUUCAUG | | | | | | |
| | −20_4-4_bulge-symmetric_UGGU- | AAUACACGCACGGCUCGUG | | | | | | |
| | UAGU | ACACGGAUUACUAGUCACU | | | | | | |
| | −10_6-6_internal_loop- | GUCGUACAUUGGCCACUCCC | | | | | | |
| | symmetric_AGGAAU-CACGGA | AGU | | | | | | |
| | −6_1-1_wobble_U-G | | | | | | | |
| | −5_1-1_mismatch_U-C | | | | | | | |
| | 0_1-1_mismatch_A-C | | | | | | | |
| | 3_2-2_bulge-symmetric_GA-CG | | | | | | | |
| | 28_6-6_internal_loop- | | | | | | | |
| | symmetric_AGGCCA-GACUAA | | | | | | | |
| | 40_4-4_bulge-symmetric_GAGU- | | | | | | | |
| | CAGA | | | | | | | |
| 280 | −33_4-4_bulge-symmetric_UUCG- | GCCACACAGACCUCCUGAUA | 0.158 | 0.75 | 0.756 | 1.007 | 1.405 | 1.431 |
| | ACAU | GAUUGAAAGUCCUUUCAUG | | | | | | |
| | −20_4-4_bulge-symmetric_UGGU- | CCUACAUCCACGGCUAAUA | | | | | | |
| | UAGU | UGCGGCUUUACUAGUCACU | | | | | | |
| | −8_6-6_internal_loop- | GUCGUACAUUGGCCACUCCC | | | | | | |
| | symmetric_GAAUUC-AUGCGG | AGU | | | | | | |
| | 0_1-1_mismatch_A-C | | | | | | | |
| | 9_2-2_bulge-symmetric_UU-CC | | | | | | | |
| | 28_6-6_internal_loop- | | | | | | | |
| | symmetric_AGGCCA-GAUAGA | | | | | | | |
| | 40_4-4_bulge-symmetric_GAGU- | | | | | | | |
| | CAGA | | | | | | | |
| 281 | −33_4-4_bulge-symmetric_UUCG- | GCCACACAGACCUCCUUGUA | 0.072 | 0.781 | 0.835 | 1.004 | 1.615 | 1.638 |
| | ACAU | AGCGGAAAGUCCCUUCAUG | | | | | | |
| | −23_0-1_bulge-asymmetric_-U | AAUAAGAUCCACGGCUUUA | | | | | | |
| | −23_1-1_wobble_U-G | AAAUUCCUCAUUCGGUCAC | | | | | | |
| | −22_1-1_mismatch_G-G | UGUCGUACAUUGGCCACUC | | | | | | |
| | −20_1-1_mismatch_U-U | CCAGU | | | | | | |
| | −19_1-1_wobble_G-U | | | | | | | |
| | −16_2-1_bulge-asymmetric_AA-C | | | | | | | |
| | −5_4-4_bulge-symmetric_CAUU- | | | | | | | |
| | UUAA | | | | | | | |
| | 0_1-1_mismatch_A-C | | | | | | | |
| | 6_1-2_bulge-asymmetric_G-AG | | | | | | | |
| | 17_1-1_mismatch_A-C | | | | | | | |
| | 26_6-6_internal_loop- | | | | | | | |
| | symmetric_AAAGGC-UAAGCG | | | | | | | |
| | 40_4-4_bulge-symmetric_GAGU- | | | | | | | |
| | CAGA | | | | | | | |
| 282 | −33_4-4_bulge-symmetric_UUCG- | GCCACACAGACCGGGUACGC | 0.03 | 0.831 | 0.764 | 0.93 | 1.672 | 1.538 |
| | ACAU | CUUUGAAAGUAUCAUGAAU | | | | | | |
| | −18_6-6_internal_loop- | ACAUCCACGGCUAACGAAU | | | | | | |
| | symmetric_UGGUGU-UGUUUU | UCCUUUUGUUUUCACUGUC | | | | | | |
| | −7_1-1_mismatch_A-C | GUACAUUGGCCACUCCCAG | | | | | | |
| | 0_1-1_mismatch_A-C | U | | | | | | |
| | 16_4-1_bulge-asymmetric_AAGG-A | | | | | | | |
| | 32_6-6_internal_loop- | | | | | | | |
| | symmetric_CAAGGA-GGGUAC | | | | | | | |
| | 40_4-4_bulge-symmetric_GAGU- | | | | | | | |
| | CAGA | | | | | | | |
| 283 | −33_4-4_bulge-symmetric_UUCG- | GCCACACAGACCUCCUAAUG | 0.131 | 0.934 | 0.91 | 1.014 | 1.494 | 1.519 |
| | ACAU | AGUUGAAAGUUCUUUCAUG | | | | | | |
| | −23_0-2_bulge-asymmetric_-GU | AAUACAUCCACGACUAAUG | | | | | | |
| | −23_1-1_wobble_U-G | AAUUCCUAUCCCGGUCACU | | | | | | |
| | −20_1-0_bulge-asymmetric_U- | GUCGUACAUUGGCCACUCCC | | | | | | |
| | −16_3-2_bulge-asymmetric_UAA-AU | AGU | | | | | | |
| | −2_1-1_mismatch_C-A | | | | | | | |
| | 0_1-1_mismatch_A-C | | | | | | | |
| | 19_1-1_wobble_G-U | | | | | | | |
| | 28_6-6_internal_loop- | | | | | | | |
| | symmetric_AGGCCA-AAUGAG | | | | | | | |
| | 40_4-4_bulge-symmetric_GAGU- | | | | | | | |
| | CAGA | | | | | | | |
| 284 | −33_4-4_bulge-symmetric_UUCG- | GCCACACAGACGAGGAACCC | 0.459 | 0.893 | 0.888 | 1.198 | 1.442 | 1.533 |
| | ACAU | CUUUGAAAGUCCUUUCAUG | | | | | | |
| | −20_4-4_bulge-symmetric_UGGU- | AAUACAUCCACGGCUAAAC | | | | | | |
| | UAGU | UUCUCCUUUACUAGUCACU | | | | | | |
| | −7_5-5_internal_loop- | GUCGUACAUUGGCCACUCCC | | | | | | |
| | symmetric_AUUCA-ACUUC | AGU | | | | | | |
| | 0_1-1_mismatch_A-C | | | | | | | |
| | 31_8-8_internal_loop- | | | | | | | |
| | symmetric_CCAAGGAG- | | | | | | | |
| | GAGGAACC | | | | | | | |
| | 40_4-4_bulge-symmetric_GAGU- | | | | | | | |
| | CAGA | | | | | | | |
| 285 | −33_4-4_bulge-symmetric_UUCG- | GCCACACAGACCUCCUAACA | 0.415 | 0.957 | 0.956 | 1.211 | 1.729 | 1.716 |
| | ACAU | GGUUGAAAGUCCUUUCAUG | | | | | | |
| | −20_4-4_bulge-symmetric_UGGU- | AAUACAUCCACGGCUAAUU | | | | | | |
| | UAGU | UGCUACUUUACUAGUCACU | | | | | | |
| | −8_6-6_internal_loop- | GUCGUACAUUGGCCACUCCC | | | | | | |
| | symmetric_GAAUUC-UUGCUA | AGU | | | | | | |
| | 0_1-1_mismatch_A-C | | | | | | | |
| | 28_6-6_internal_loop- | | | | | | | |
| | symmetric_AGGCCA-AACAGG | | | | | | | |
| | 40_4-4_bulge-symmetric_GAGU- | | | | | | | |
| | CAGA | | | | | | | |
| 286 | −33_4-4_bulge-symmetric_UUCG- | GCCACACAGACCUCCUAAAA | 0.046 | 0.931 | 0.909 | 0.934 | 1.701 | 1.638 |
| | ACAU | UGUUGAAAGUGCUUUCAGG | | | | | | |
| | −18_6-6_internal_loop- | AAUACAUCCACGGCUAAUG | | | | | | |
| | symmetric_UGGUGU-UUGUUU | AAUUCCUUUUUGUUUCACU | | | | | | |
| | 0_1-1_mismatch_A-C | GUCGUACAUUGGCCACUCCC | | | | | | |
| | 12_1-1_mismatch_A-G | AGU | | | | | | |
| | 19_1-1_mismatch_G-G | | | | | | | |
| | 28_6-6_internal_loop- | | | | | | | |
| | symmetric_AGGCCA-AAAAUG | | | | | | | |
| | 40_4-4_bulge-symmetric_GAGU- | | | | | | | |
| | CAGA | | | | | | | |
| 287 | −33_4-4_bulge-symmetric_UUCG- | GCCACACAGACCGAACAUGC | 0.138 | 0.933 | 0.935 | 0.735 | 0.835 | 0.885 |
| | ACAU | CUUUGAAAGUCCUUUGAUG | | | | | | |
| | −14_10−10_internal_loop- | CAUACAUCCACGGCUAAUG | | | | | | |
| | symmetric_UGGUGUAAAG- | AAUUCGAUAUGUAGUCACU | | | | | | |
| | GAUAUGUAGU | GUCGUACAUUGGCCACUCCC | | | | | | |
| | 0_1-1_mismatch_A-C | AGU | | | | | | |
| | 10_1-1_mismatch_U-C | | | | | | | |
| | 14_1-1_mismatch_G-G | | | | | | | |
| | 32_6-6_internal_loop- | | | | | | | |
| | symmetric_CAAGGA-GAACAU | | | | | | | |
| | 40_4-4_bulge-symmetric_GAGU- | | | | | | | |
| | CAGA | | | | | | | |
| 288 | −33_4-4_bulge-symmetric_UUCG- | GCCACACAGACCAUUAGCGC | 0.147 | 0.919 | 0.917 | 0.89 | 1.006 | 1.042 |
| | ACAU | CUUUGAAAGUCCUUUGAUG | | | | | | |
| | −20_4-4_bulge-symmetric_UGGU- | CAUACAUCCACGGCUAUUU | | | | | | |
| | UAGU | AGGUCCUUUACUAGUCACU | | | | | | |
| | −6_6-6_internal_loop- | GUCGUACAUUGGCCACUCCC | | | | | | |
| | symmetric_AUUCAU-UUUAGG | AGU | | | | | | |
| | 0_1-1_mismatch_A-C | | | | | | | |
| | 10_1-1_mismatch_U-C | | | | | | | |
| | 14_1-1_mismatch_G-G | | | | | | | |
| | 32_6-6_internal_loop- | | | | | | | |
| | symmetric_CAAGGA-AUUAGC | | | | | | | |
| | 40_4-4_bulge-symmetric_GAGU- | | | | | | | |
| | CAGA | | | | | | | |
| 289 | −33_4-4_bulge-symmetric_UUCG- | GCCACACAAAAAAACUUGG | 0.172 | 0.918 | 0.914 | 0.693 | 0.787 | 0.828 |
| | ACAU | CCUUUGAAAGUCCUUUGAU | | | | | | |
| | −20_4-4_bulge-symmetric_UGGU- | GCAUACAUCCACGGCUACA | | | | | | |
| | UAGU | AGUGUCCUUUACUAGUCAC | | | | | | |
| | −6_6-6_internal_loop- | UGUCGUACAUUGGCCACUC | | | | | | |
| | symmetric_AUUCAU-CAAGUG | CCAGU | | | | | | |
| | 0_1-1_mismatch_A-C | | | | | | | |
| | 10_1-1_mismatch_U-C | | | | | | | |
| | 14_1-1_mismatch_G-G | | | | | | | |
| | 36_8-8_internal_loop- | | | | | | | |
| | symmetric_GAGGGAGU- | | | | | | | |
| | CAAAAAAA | | | | | | | |
| 290 | −33_4-4_bulge-symmetric_UUCG- | GCCACACAGACCUCCUGAUA | 0.09 | 0.849 | 0.865 | 0.978 | 1.324 | 1.382 |
| | ACAU | ACUUGAAAGUCCUUUGAUG | | | | | | |
| | −20_4-4_bulge-symmetric_UGGU- | CAUACAUCCACGGCUAAUC | | | | | | |
| | UAGU | UGGGGCUUUACUAGUCACU | | | | | | |
| | −8_6-6_internal_loop- | GUCGUACAUUGGCCACUCCC | | | | | | |
| | symmetric_GAAUUC-CUGGGG | AGU | | | | | | |
| | 0_1-1_mismatch_A-C | | | | | | | |
| | 10_1-1_mismatch_U-C | | | | | | | |
| | 14_1-1_mismatch_G-G | | | | | | | |
| | 28_6-6_internal_loop- | | | | | | | |
| | symmetric_AGGCCA-GAUAAC | | | | | | | |
| | 40_4-4_bulge-symmetric_GAGU- | | | | | | | |
| | CAGA | | | | | | | |
| 291 | −33_4-4_bulge-symmetric_UUCG- | GCCACACAGUAUUACUUGG | 0.189 | 0.928 | 0.924 | 0.615 | 0.777 | 0.823 |
| | ACAU | CCUUUGAAAGUCCUUUGAU | | | | | | |
| | −20_4-4_bulge-symmetric_UGGU- | GCAUACAUCCACGGCUAAU | | | | | | |
| | UAGU | UAGCAGCUUUACUAGUCAC | | | | | | |
| | −8_6-6_internal_loop- | UGUCGUACAUUGGCCACUC | | | | | | |
| | symmetric_GAAUUC-UAGCAG | CCAGU | | | | | | |
| | 0_1-1_mismatch_A-C | | | | | | | |
| | 10_1-1_mismatch_U-C | | | | | | | |
| | 14_1-1_mismatch_G-G | | | | | | | |
| | 36_8-8_internal_loop- | | | | | | | |
| | symmetric_GAGGGAGU- | | | | | | | |
| | CAGUAUUA | | | | | | | |
| 292 | −33_4-4_bulge-symmetric_UUCG- | GCCACAUACAAGUCCUUGG | 0.238 | 0.937 | 0.933 | 0.614 | 0.737 | 0.791 |
| | ACAU | CCUUUGAAAGUCCUUUGAU | | | | | | |
| | −20_4-4_bulge-symmetric_UGGU- | GCAUACAUCCACGGCUAAU | | | | | | |
| | UAGU | UUAGUACUUUACUAGUCAC | | | | | | |
| | −8_6-6_internal_loop- | UGUCGUACAUUGGCCACUC | | | | | | |
| | symmetric_GAAUUC-UUAGUA | CCAGU | | | | | | |
| | 0_1-1_mismatch_A-C | | | | | | | |
| | 10_1-1_mismatch_U-C | | | | | | | |
| | 14_1-1_mismatch_G-G | | | | | | | |
| | 38_6-6_internal_loop- | | | | | | | |
| | symmetric_GGGAGU-UACAAG | | | | | | | |
| 293 | −8_6-6_internal_loop- | GCCACAACUCCCUCGAACGA | 0.359 | 0.834 | 0.744 | 1.075 | 1.236 | 1.183 |
| | symmetric_GAAUUC-UAUAAG | CUUUGAAAGUCCUUUCAUG | | | | | | |
| | 0_1-1_mismatch_A-C | AAUAGAUCCACGGCUAAUU | | | | | | |
| | 6_1-1_mismatch_G-G | AUAAGCUUUACACCACACU | | | | | | |
| | 30_6-6_internal_loop- | GUCGUCGAAUGGCCACUCCC | | | | | | |
| | symmetric_GCCAAG-GAACGA | AGU | | | | | | |
| 294 | −33_4-4_bulge-symmetric_UUCG- | GCCACAACUCCCUCGAACGA | 0.341 | 0.782 | 0.743 | 1.075 | 1.206 | 1.326 |
| | ACAU | CUUUGAAAGUCCUUUCAUG | | | | | | |
| | −20_4-4_bulge-symmetric_UGGU- | AAUAGAUCCACGGCUAAUU | | | | | | |
| | UAGU | AUAAGCUUUACUAGUCACU | | | | | | |
| | −8_6-6_internal_loop- | GUCGUACAUUGGCCACUCCC | | | | | | |
| | symmetric_GAAUUC-UAUAAG | AGU | | | | | | |
| | 0_1-1_mismatch_A-C | | | | | | | |
| | 6_1-1_mismatch_G-G | | | | | | | |
| | 30_6-6_internal_loop- | | | | | | | |
| | symmetric_GCCAAG-GAACGA | | | | | | | |
| 295 | −6_6-6_internal_loop- | GCCACAACUCCCUCCUUGGC | 0.288 | 0.778 | 0.713 | 0.938 | 1.218 | 1.198 |
| | symmetric_AUUCAU-UGCCUG | AAGUCCAAGUCCUUUCAUG | | | | | | |
| | 0_1-1_mismatch_A-C | AAUAGAUCCACGGCUAUGC | | | | | | |
| | 6_1-1_mismatch_G-G | CUGUCCUUUACACCACACUG | | | | | | |
| | 24_6-6_internal_loop- | UCGUCGAAUGGCCACUCCCA | | | | | | |
| | symmetric_UCAAAG-AAGUCC | GU | | | | | | |
| 296 | −33_4-4_bulge-symmetric_UUCG- | GCCACACAGACCUCCUUGGC | 0.27 | 0.917 | 0.911 | 1.136 | 1.65 | 1.595 |
| | ACAU | AAGUCCAAGUCCUUUCAUG | | | | | | |
| | −20_4-4_bulge-symmetric_UGGU- | AAUAGAUCCACGGCUAUGC | | | | | | |
| | UAGU | CUGUCCUUUACUAGUCACU | | | | | | |
| | −6_6-6_internal_loop- | GUCGUACAUUGGCCACUCCC | | | | | | |
| | symmetric_AUUCAU-UGCCUG | AGU | | | | | | |
| | 0_1-1_mismatch_A-C | | | | | | | |
| | 6_1-1_mismatch_G-G | | | | | | | |
| | 24_6-6_internal_loop- | | | | | | | |
| | symmetric_UCAAAG-AAGUCC | | | | | | | |
| | 40_4-4_bulge-symmetric_GAGU- | | | | | | | |
| | CAGA | | | | | | | |
| 297 | −6_6-6_internal_loop- | GCCACACAGACCUCCUUGGC | 0.475 | 0.881 | 0.864 | 1.177 | 1.57 | 1.424 |
| | symmetric_AUUCAU-UGCCUG | AAGUCCAAGUCCUUUCAUG | | | | | | |
| | 0_1-1_mismatch_A-C | AAUAGAUCCACGGCUAUGC | | | | | | |
| | 6_1-1_mismatch_G-G | CUGUCCUUUACACCACACUG | | | | | | |
| | 24_6-6_internal_loop- | UCGUCGAAUGGCCACUCCCA | | | | | | |
| | symmetric_UCAAAG-AAGUCC | GU | | | | | | |
| | 40_4-4_bulge-symmetric_GAGU- | | | | | | | |
| | CAGA | | | | | | | |
| 298 | −33_4-4_bulge-symmetric_UUCG- | GCCACAACUCCCUCCUUGGC | 0.302 | 0.783 | 0.806 | 0.955 | 1.235 | 1.28 |
| | ACAU | AAGUCCAAGUCCUUUCAUG | | | | | | |
| | −20_4-4_bulge-symmetric_UGGU- | AAUAGAUCCACGGCUAUGC | | | | | | |
| | UAGU | CUGUCCUUUACUAGUCACU | | | | | | |
| | −6_6-6_internal_loop- | GUCGUACAUUGGCCACUCCC | | | | | | |
| | symmetric_AUUCAU-UGCCUG | AGU | | | | | | |
| | 0_1-1_mismatch_A-C | | | | | | | |
| | 6_1-1_mismatch_G-G | | | | | | | |
| | 24_6-6_internal_loop- | | | | | | | |
| | symmetric_UCAAAG-AAGUCC | | | | | | | |
| 299 | −12_6-6_internal_loop- | GCCACAACUCCCUCCUUGAA | 0.304 | 0.773 | 0.734 | 1.017 | 1.3 | 1.195 |
| | symmetric_AAAGGA-GGUUUG | AGCAGAAAGUCCUUUCGUG | | | | | | |
| | −4_3-2_bulge-asymmetric_UUA-AC | AAUACAUCCACGGCACUGA | | | | | | |
| | 0_1-1_mismatch_A-C | AUGGUUUGACACCACACUG | | | | | | |
| | 13_1-1_wobble_U-G | UCGUCGAAUGGCCACUCCCA | | | | | | |
| | 26_6-6_internal_loop- | GU | | | | | | |
| | symmetric_AAAGGC-AAAGCA | | | | | | | |
| 300 | −33_4-4_bulge-symmetric_UUCG- | GCCACACAGACCUCCUUGAA | 0.217 | 0.862 | 0.873 | 1.086 | 1.612 | 1.596 |
| | ACAU | AGCAGAAAGUCCUUUCGUG | | | | | | |
| | −23_0-2_bulge-asymmetric_-GU | AAUACAUCCACGGCACUGA | | | | | | |
| | −22_1-1_wobble_G-U | AUGGUUUGACUAGUCACUG | | | | | | |
| | −19_1-0_bulge-asymmetric_G- | UCGUACAUUGGCCACUCCCA | | | | | | |
| | −18_1-1_wobble_U-G | GU | | | | | | |
| | −12_3-2_bulge-asymmetric_GGA-GG | | | | | | | |
| | −4_3-2_bulge-asymmetric_UUA-AC | | | | | | | |
| | 0_1-1_mismatch_A-C | | | | | | | |
| | 13 1-1_wobble_U-G | | | | | | | |
| | 26_6-6_internal_loop- | | | | | | | |
| | symmetric_AAAGGC-AAAGCA | | | | | | | |
| | 40_4-4_bulge-symmetric_GAGU- | | | | | | | |
| | CAGA | | | | | | | |
| 301 | −12_6-6_internal_loop- | GCCACACAGACCUCCUUGAA | 0.639 | 0.922 | 0.873 | 1.391 | 1.641 | 1.649 |
| | symmetric_AAAGGA-GGUUUG | AGCAGAAAGUCCUUUCGUG | | | | | | |
| | −4_3-2_bulge-asymmetric_UUA-AC | AAUACAUCCACGGCACUGA | | | | | | |
| | 0_1-1_mismatch_A-C | AUGGUUUGACACCACACUG | | | | | | |
| | 13_1-1_wobble_U-G | UCGUCGAAUGGCCACUCCCA | | | | | | |
| | 26_6-6_internal_loop- | GU | | | | | | |
| | symmetric_AAAGGC-AAAGCA | | | | | | | |
| | 40_4-4_bulge-symmetric_GAGU- | | | | | | | |
| | CAGA | | | | | | | |
| 302 | −33_4-4_bulge-symmetric_UUCG- | GCCACAACUCCCUCCUUGAA | 0.182 | 0.772 | 0.718 | 0.9 | 1.262 | 1.18 |
| | ACAU | AGCAGAAAGUCCUUUCGUG | | | | | | |
| | −23_0-2_bulge-asymmetric_-GU | AAUACAUCCACGGCACUGA | | | | | | |
| | −22_1-1_wobble_G-U | AUGGUUUGACUAGUCACUG | | | | | | |
| | −19_1-0_bulge-asymmetric_G- | UCGUACAUUGGCCACUCCCA | | | | | | |
| | −18_1-1_wobble_U-G | GU | | | | | | |
| | −12_3-2_bulge-asymmetric_GGA-GG | | | | | | | |
| | −4_3-2_bulge-asymmetric_UUA-AC | | | | | | | |
| | 0_1-1_mismatch_A-C | | | | | | | |
| | 13 1-1_wobble_U-G | | | | | | | |
| | 26_6-6_internal_loop- | | | | | | | |
| | symmetric_AAAGGC-AAAGCA | | | | | | | |
| 303 | −18_6-6_internal_loop- | GCCACAACUCCCUCGCGCAA | 0.368 | 0.806 | 0.803 | 1.136 | 1.286 | 1.315 |
| | symmetric_UGGUGU-UGGAGU | CUUUGAAAGUCCUUUCAUG | | | | | | |
| | −6_3-3_bulge-symmetric_CAU-CAU | CAUAGAUCCACGGCUACAU | | | | | | |
| | 0_1-1_mismatch_A-C | AAUUCCUUUUGGAGUCACU | | | | | | |
| | 6_1-1_mismatch_G-G | GUCGUCGAAUGGCCACUCCC | | | | | | |
| | 10_1-1_mismatch_U-C | AGU | | | | | | |
| | 30_6-6_internal_loop- | | | | | | | |
| | symmetric_GCCAAG-GCGCAA | | | | | | | |
| 304 | −18_6-6_internal_loop- | GCCACACAGACCUCGCGCAA | 0.459 | 0.895 | 0.872 | 1.247 | 1.496 | 1.552 |
| | symmetric_UGGUGU-UGGAGU | CUUUGAAAGUCCUUUCAUG | | | | | | |
| | −6_3-3_bulge-symmetric_CAU-CAU | CAUAGAUCCACGGCUACAU | | | | | | |
| | 0_1-1_mismatch_A-C | AAUUCCUUUUGGAGUCACU | | | | | | |
| | 6_1-1_mismatch_G-G | GUCGUCGAAUGGCCACUCCC | | | | | | |
| | 10_1-1_mismatch_U-C | AGU | | | | | | |
| | 30_6-6_internal_loop- | | | | | | | |
| | symmetric_GCCAAG-GCGCAA | | | | | | | |
| | 40_4-4_bulge-symmetric_GAGU- | | | | | | | |
| | CAGA | | | | | | | |
| 305 | −33_4-4_bulge-symmetric_UUCG- | GCCACAACUCCCUCGCGCAA | 0.29 | 0.816 | 0.843 | 1.037 | 1.368 | 1.36 |
| | ACAU | CUUUGAAAGUCCUUUCAUG | | | | | | |
| | −18_6-6_internal_loop- | CAUAGAUCCACGGCUACAU | | | | | | |
| | symmetric_UGGUGU-UGGAGU | AAUUCCUUUUGGAGUCACU | | | | | | |
| | −6_3-3_bulge-symmetric_CAU-CAU | GUCGUACAUUGGCCACUCCC | | | | | | |
| | 0_1-1_mismatch_A-C | AGU | | | | | | |
| | 6_1-1_mismatch_G-G | | | | | | | |
| | 10_1-1_mismatch_U-C | | | | | | | |
| | 30_6-6_internal_loop- | | | | | | | |
| | symmetric_GCCAAG-GCGCAA | | | | | | | |
| 306 | −10_6-6_internal_loop- | GCCACAACUCCCUCAGCCUA | 0.395 | 0.757 | 0.676 | 1.043 | 1.165 | 1.189 |
| | symmetric_AGGAAU-CAGUAC | CUUUGAAAGUCCUUUCAUC | | | | | | |
| | 0_1-1_mismatch_A-C | AAUACCACCACGGCUAAUG | | | | | | |
| | 4_2-2_bulge-symmetric_AU-CA | ACAGUACUUACACCACACU | | | | | | |
| | 11_1-1_mismatch_C-C | GUCGUCGAAUGGCCACUCCC | | | | | | |
| | 30_6-6_internal_loop- | AGU | | | | | | |
| | symmetric_GCCAAG-AGCCUA | | | | | | | |
| 307 | −10_6-6_internal_loop- | GCCACACAGACCUCAGCCUA | 0.712 | 0.927 | 0.884 | 1.405 | 1.283 | 1.438 |
| | symmetric_AGGAAU-CAGUAC | CUUUGAAAGUCCUUUCAUC | | | | | | |
| | 0_1-1_mismatch_A-C | AAUACCACCACGGCUAAUG | | | | | | |
| | 4_2-2_bulge-symmetric_AU-CA | ACAGUACUUACACCACACU | | | | | | |
| | 11_1-1_mismatch_C-C | GUCGUCGAAUGGCCACUCCC | | | | | | |
| | 30_6-6_internal_loop- | AGU | | | | | | |
| | symmetric_GCCAAG-AGCCUA | | | | | | | |
| | 40_4-4_bulge-symmetric_GAGU- | | | | | | | |
| | CAGA | | | | | | | |
| 308 | −33_4-4_bulge-symmetric_UUCG- | GCCACAACUCCCUCAGCCUA | 0.302 | 0.792 | 0.804 | 1.099 | 1.208 | 1.317 |
| | ACAU | CUUUGAAAGUCCUUUCAUC | | | | | | |
| | −20_4-4_bulge-symmetric_UGGU- | AAUACCACCACGGCUAAUG | | | | | | |
| | UAGU | ACAGUACUUACUAGUCACU | | | | | | |
| | −10_6-6_internal_loop- | GUCGUACAUUGGCCACUCCC | | | | | | |
| | symmetric_AGGAAU-CAGUAC | AGU | | | | | | |
| | 0_1-1_mismatch_A-C | | | | | | | |
| | 4_2-2_bulge-symmetric_AU-CA | | | | | | | |
| | 11_1-1_mismatch_C-C | | | | | | | |
| | 30_6-6_internal_loop- | | | | | | | |
| | symmetric_GCCAAG-AGCCUA | | | | | | | |
| 309 | −14_6-6_internal_loop- | GCCACAACUCCCUCGGACAA | 0.465 | 0.751 | 0.744 | 1.127 | 1.166 | 1.25 |
| | symmetric_GUAAAG-GAACCG | CUUUGAAAGUCCUUUCAUG | | | | | | |
| | −5_4-4_bulge-symmetric_CAUU- | AAUACAACCACGGCUCAAC | | | | | | |
| | CAAC | AAUUCGAACCGACCACACU | | | | | | |
| | 0_1-1_mismatch_A-C | GUCGUCGAAUGGCCACUCCC | | | | | | |
| | 4_1-1_mismatch_A-A | AGU | | | | | | |
| | 30_6-6_internal_loop- | | | | | | | |
| | symmetric_GCCAAG-GGACAA | | | | | | | |
| 310 | −14_6-6_internal_loop- | GCCACACAGACCUCGGACAA | 0.613 | 0.78 | 0.769 | 1.353 | 1.317 | 1.381 |
| | symmetric_GUAAAG-GAACCG | CUUUGAAAGUCCUUUCAUG | | | | | | |
| | −5_4-4_bulge-symmetric_CAUU- | AAUACAACCACGGCUCAAC | | | | | | |
| | CAAC | AAUUCGAACCGACCACACU | | | | | | |
| | 0_1-1_mismatch_A-C | GUCGUCGAAUGGCCACUCCC | | | | | | |
| | 4_1-1_mismatch_A-A | AGU | | | | | | |
| | 30_6-6_internal_loop- | | | | | | | |
| | symmetric_GCCAAG-GGACAA | | | | | | | |
| | 40_4-4_bulge-symmetric_GAGU- | | | | | | | |
| | CAGA | | | | | | | |
| 311 | −33_4-4_bulge-symmetric_UUCG- | GCCACAACUCCCUCGGACAA | 0.353 | 0.7 | 0.782 | 1.111 | 1.209 | 1.288 |
| | ACAU | CUUUGAAAGUCCUUUCAUG | | | | | | |
| | −23_0-4_bulge-asymmetric_-UAGU | AAUACAACCACGGCUCAAC | | | | | | |
| | −23_1-1_wobble_U-G | AAUUCGAACCGUAGUCACU | | | | | | |
| | −14_6-2_internal_loop- | GUCGUACAUUGGCCACUCCC | | | | | | |
| | asymmetric_GUAAAG-GA | AGU | | | | | | |
| | −5_4-4_bulge-symmetric_CAUU- | | | | | | | |
| | CAAC | | | | | | | |
| | 0_1-1_mismatch_A-C | | | | | | | |
| | 4_1-1_mismatch_A-A | | | | | | | |
| | 30_6-6_internal_loop- | | | | | | | |
| | symmetric_GCCAAG-GGACAA | | | | | | | |
| 312 | −14_6-6_internal_loop- | GCCACAACUCCCUCCUUGAU | 0.268 | 0.749 | 0.766 | 0.947 | 1.183 | 1.222 |
| | symmetric_GUAAAG-GAUCCA | GGCGGAAAGUCCUUUCAUG | | | | | | |
| | −7_1-1_mismatch_A-A | AAUACAUCCACGGCUAAAG | | | | | | |
| | 0_1-1_mismatch_A-C | AAUUCGAUCCAACCACACU | | | | | | |
| | 26_6-6_internal_loop- | GUCGUCGAAUGGCCACUCCC | | | | | | |
| | symmetric_AAAGGC-AUGGCG | AGU | | | | | | |
| 313 | −14_6-6_internal_loop- | GCCACACAGACCUCCUUGAU | 0.398 | 0.895 | 0.871 | 1.122 | 1.534 | 1.463 |
| | symmetric_GUAAAG-GAUCCA | GGCGGAAAGUCCUUUCAUG | | | | | | |
| | −7_1-1_mismatch_A-A | AAUACAUCCACGGCUAAAG | | | | | | |
| | 0_1-1_mismatch_A-C | AAUUCGAUCCAACCACACU | | | | | | |
| | 26_6-6_internal_loop- | GUCGUCGAAUGGCCACUCCC | | | | | | |
| | symmetric_AAAGGC-AUGGCG | AGU | | | | | | |
| | 40_4-4_bulge-symmetric_GAGU- | | | | | | | |
| | CAGA | | | | | | | |
| 314 | −33_4-4_bulge-symmetric_UUCG- | GCCACAACUCCCUCCUUGAU | 0.246 | 0.809 | 0.773 | 0.969 | 1.228 | 1.288 |
| | ACAU | GGCGGAAAGUCCUUUCAUG | | | | | | |
| | −22_2-3_bulge-asymmetric_UG-AGU | AAUACAUCCACGGCUAAAG | | | | | | |
| | −21_1-1_wobble_G-U | AAUUCGAUCCAUAGUCACU | | | | | | |
| | −14_5-4_internal_loop- | GUCGUACAUUGGCCACUCCC | | | | | | |
| | asymmetric_UAAAG-GAUC | AGU | | | | | | |
| | −7_1-1_mismatch_A-A | | | | | | | |
| | 0_1-1_mismatch_A-C | | | | | | | |
| | 26_6-6_internal_loop- | | | | | | | |
| | symmetric_AAAGGC-AUGGCG | | | | | | | |
| 315 | −6_6-6_internal_loop- | GCCACAACUCCCUCGACUAA | 0.331 | 0.817 | 0.824 | 1.013 | 1.334 | 1.325 |
| | symmetric_AUUCAU-CUAGUG | CUUUGAAAGUCCUUUCAUG | | | | | | |
| | 0_1-1_mismatch_A-C | AAUAGCUCCACGGCUACUA | | | | | | |
| | 5_2-2_bulge-symmetric_UG-GC | GUGUCCUUUACACCACACU | | | | | | |
| | 30_6-6_internal_loop- | GUCGUCGAAUGGCCACUCCC | | | | | | |
| | symmetric_GCCAAG-GACUAA | AGU | | | | | | |
| 316 | −6_6-6_internal_loop- | GCCACACAGACCUCGACUAA | 0.633 | 0.919 | 0.917 | 1.365 | 1.65 | 1.631 |
| | symmetric_AUUCAU-CUAGUG | CUUUGAAAGUCCUUUCAUG | | | | | | |
| | 0_1-1_mismatch_A-C | AAUAGCUCCACGGCUACUA | | | | | | |
| | 5_2-2_bulge-symmetric_UG-GC | GUGUCCUUUACACCACACU | | | | | | |
| | 30_6-6_internal_loop- | GUCGUCGAAUGGCCACUCCC | | | | | | |
| | symmetric_GCCAAG-GACUAA | AGU | | | | | | |
| | 40_4-4_bulge-symmetric_GAGU- | | | | | | | |
| | CAGA | | | | | | | |
| 317 | −33_4-4_bulge-symmetric_UUCG- | GCCACAACUCCCUCGACUAA | 0.276 | 0.798 | 0.808 | 1.043 | 1.31 | 1.299 |
| | ACAU | CUUUGAAAGUCCUUUCAUG | | | | | | |
| | −20_4-4_bulge-symmetric_UGGU- | AAUAGCUCCACGGCUACUA | | | | | | |
| | UAGU | GUGUCCUUUACUAGUCACU | | | | | | |
| | −6_6-6_internal_loop- | GUCGUACAUUGGCCACUCCC | | | | | | |
| | symmetric_AUUCAU-CUAGUG | AGU | | | | | | |
| | 0_1-1_mismatch_A-C | | | | | | | |
| | 5_2-2_bulge-symmetric_UG-GC | | | | | | | |
| | 30_6-6_internal_loop- | | | | | | | |
| | symmetric_GCCAAG-GACUAA | | | | | | | |
| 318 | −8_6-6_internal_loop- | GCCACAACUCCCUCCUAUAG | 0.381 | 0.759 | 0.749 | 1.048 | 1.145 | 1.165 |
| | symmetric_GAAUUC-CGCCGA | AGUUGAAAGUCCUUUCAUG | | | | | | |
| | 0_1-1_mismatch_A-C | AAUACAUCUACGGCUAAUC | | | | | | |
| | 2 1-1_wobble_G-U | GCCGACUUUACACCACACUG | | | | | | |
| | 28_6-6_internal_loop- | UCGUCGAAUGGCCACUCCCA | | | | | | |
| | symmetric_AGGCCA-AUAGAG | GU | | | | | | |
| 319 | −16_6-6_internal_loop- | GCCACAACUCCCUCCUCUCA | 0.399 | 0.812 | 0.81 | 0.999 | 1.185 | 1.338 |
| | symmetric_GUGUAA-AAUGAA | AGUUGAAAGUCCUUUCAUG | | | | | | |
| | −5_4-1_bulge-asymmetric_CAUU-U | AAUAUAUCCACGGCUUAAU | | | | | | |
| | 0_1-1_mismatch_A-C | UCCUAAUGAACACACUGUC | | | | | | |
| | 6_1-1_wobble_G-U | GUCGAAUGGCCACUCCCAG | | | | | | |
| | 28_6-6_internal_loop- | U | | | | | | |
| | symmetric_AGGCCA-CUCAAG | | | | | | | |
| 320 | −6_6-6_internal_loop- | GCCACAACUCCCUCGCAUCA | 0.243 | 0.822 | 0.771 | 0.97 | 1.234 | 1.231 |
| | symmetric_AUUCAU-CGUCGC | CUUUGAAAGUACUUUCAUU | | | | | | |
| | 0_1-1_mismatch_A-C | AAUACAUCCACGGCUACGU | | | | | | |
| | 11_1-1_mismatch_C-U | CGCUCCUUUACACCACACUG | | | | | | |
| | 19_1-1_mismatch_G-A | UCGUCGAAUGGCCACUCCCA | | | | | | |
| | 30_6-6_internal_loop- | GU | | | | | | |
| | symmetric_GCCAAG-GCAUCA | | | | | | | |
| 321 | −16_6-6_internal_loop- | GCCACAACUCCCUCAAAACA | 0.24 | 0.834 | 0.682 | 0.946 | 1.215 | 1.366 |
| | symmetric_GUGUAA-GAGAUG | CUUUGAAAGUCCUUUCAUG | | | | | | |
| | −4_1-0_bulge-asymmetric_A- | AAUGUCUCCACGGCAAUGA | | | | | | |
| | 0_1-1_mismatch_A-C | AUUCCUGAGAUGCACACUG | | | | | | |
| | 5_2-2_bulge-symmetric_UG-UC | UCGUCGAAUGGCCACUCCCA | | | | | | |
| | 7_1-1_wobble_U-G | GU | | | | | | |
| | 30_6-6_internal_loop- | | | | | | | |
| | symmetric_GCCAAG-AAAACA | | | | | | | |
| 322 | −33_4-4_bulge-symmetric_UUCG- | GCCACACAGACCUCAAAACA | 0.128 | 0.931 | 0.928 | 0.987 | 1.509 | 1.598 |
| | ACAU | CUUUGAAAGUCCUUUCAUG | | | | | | |
| | −23_0-1_bulge-asymmetric_-U | AAUGUCUCCACGGCAAUGA | | | | | | |
| | −23_1-1_wobble_U-G | AUUCCUGAGAUGGUCACUG | | | | | | |
| | −22_1-1_mismatch_G-G | UCGUACAUUGGCCACUCCCA | | | | | | |
| | −21_1-1_wobble_G-U | GU | | | | | | |
| | −19_1-1_mismatch_G-G | | | | | | | |
| | −16_2-1_bulge-asymmetric_AA-G | | | | | | | |
| | −4_1-0_bulge-asymmetric_A- | | | | | | | |
| | 0_1-1_mismatch_A-C | | | | | | | |
| | 5_2-2_bulge-symmetric_UG-UC | | | | | | | |
| | 7_1-1_wobble_U-G | | | | | | | |
| | 30_6-6_internal_loop- | | | | | | | |
| | symmetric_GCCAAG-AAAACA | | | | | | | |
| | 40_4-4_bulge-symmetric_GAGU- | | | | | | | |
| | CAGA | | | | | | | |
| 323 | −12_6-6_internal_loop- | GCCACAACUCCCUCCUUGAU | 0.272 | 0.774 | 0.747 | 0.961 | 1.164 | 1.281 |
| | symmetric_AAAGGA-AGUAAA | AAUCGAAAGUCCUUUCAUG | | | | | | |
| | −2_2-1_bulge-asymmetric_GC-A | AAUACAUCCACGAUAAUGA | | | | | | |
| | 0_1-1_mismatch_A-C | AUAGUAAAACACCACACUG | | | | | | |
| | 26_6-6_internal_loop- | UCGUCGAAUGGCCACUCCCA | | | | | | |
| | symmetric_AAAGGC-AUAAUC | GU | | | | | | |
| 324 | −33_4-4_bulge-symmetric_UUCG- | GCCACACAGACCUCCUUGAU | 0.313 | 0.925 | 0.913 | 1.069 | 1.615 | 1.53 |
| | ACAU | AAUCGAAAGUCCUUUCAUG | | | | | | |
| | −23_0-2_bulge-asymmetric_-GU | AAUACAUCCACGAUAAUGA | | | | | | |
| | −22_1-1_wobble_G-U | AUAGUAAAACUAGUCACUG | | | | | | |
| | −12 8-6_internal loop- | UCGUACAUUGGCCACUCCCA | | | | | | |
| | asymmetric_GUAAAGGA-AGUAAA | GU | | | | | | |
| | −2_2-1_bulge-asymmetric_GC-A | | | | | | | |
| | 0_1-1_mismatch_A-C | | | | | | | |
| | 26_6-6_internal_loop- | | | | | | | |
| | symmetric_AAAGGC-AUAAUC | | | | | | | |
| | 40_4-4_bulge-symmetric_GAGU- | | | | | | | |
| | CAGA | | | | | | | |
| 325 | −6_2-0_bulge-asymmetric_AU- | GCCACAACUCCCUCACACUG | 0.269 | 0.803 | 0.821 | 0.956 | 1.334 | 1.213 |
| | 0_1-1_mismatch_A-C | CUUUGAAAGUCCUUUCAGG | | | | | | |
| | 5_1-1_mismatch_U-C | AAUACCUCCACGGCUAGAA | | | | | | |
| | 12_1-1_mismatch_A-G | UUCCUUUACACCACACUGUC | | | | | | |
| | 30_6-6_internal_loop- | GUCGAAUGGCCACUCCCAG | | | | | | |
| | symmetric_GCCAAG-ACACUG | U | | | | | | |
| 326 | −33_4-4_bulge-symmetric_UUCG- | GCCACACAGACCUCACACUG | 0.079 | 0.88 | 0.887 | 0.946 | 1.597 | 1.608 |
| | ACAU | CUUUGAAAGUCCUUUCAGG | | | | | | |
| | −20_4-4_bulge-symmetric_UGGU- | AAUACCUCCACGGCUAGAA | | | | | | |
| | UAGU | UUCCUUUACUAGUCACUGU | | | | | | |
| | −6_2-0_bulge-asymmetric_AU- | CGUACAUUGGCCACUCCCAG | | | | | | |
| | 0_1-1_mismatch_A-C | U | | | | | | |
| | 5_1-1_mismatch_U-C | | | | | | | |
| | 12_1-1_mismatch_A-G | | | | | | | |
| | 30_6-6_internal_loop- | | | | | | | |
| | symmetric_GCCAAG-ACACUG | | | | | | | |
| | 40_4-4_bulge-symmetric_GAGU- | | | | | | | |
| | CAGA | | | | | | | |
| 327 | −8_6-6_internal_loop- | GCCACAACUCCCUCCUUGAU | 0.254 | 0.784 | 0.77 | 0.898 | 1.212 | 1.225 |
| | symmetric_GAAUUC-AAAACA | AGUGGAAAGUCCUUUCAUG | | | | | | |
| | −6_1-1_wobble_U-G | AUUACAUCCACGGCUAGUA | | | | | | |
| | 0_1-1_mismatch_A-C | AAACACUUUACACCACACU | | | | | | |
| | 9_1-1_mismatch_U-U | GUCGUCGAAUGGCCACUCCC | | | | | | |
| | 26_6-6_internal_loop- | AGU | | | | | | |
| | symmetric_AAAGGC-AUAGUG | | | | | | | |
| 328 | −33_4-4_bulge-symmetric_UUCG- | GCCACACAGACCUCCUUGAU | 0.256 | 0.901 | 0.902 | 1.092 | 1.609 | 1.536 |
| | ACAU | AGUGGAAAGUCCUUUCAUG | | | | | | |
| | −20_4-4_bulge-symmetric_UGGU- | AUUACAUCCACGGCUAGUA | | | | | | |
| | UAGU | AAACACUUUACUAGUCACU | | | | | | |
| | −8_6-6_internal_loop- | GUCGUACAUUGGCCACUCCC | | | | | | |
| | symmetric_GAAUUC-AAAACA | AGU | | | | | | |
| | −6_1-1_wobble_U-G | | | | | | | |
| | 0_1-1_mismatch_A-C | | | | | | | |
| | 9_1-1_mismatch_U-U | | | | | | | |
| | 26_6-6_internal_loop- | | | | | | | |
| | symmetric_AAAGGC-AUAGUG | | | | | | | |
| | 40_4-4_bulge-symmetric_GAGU- | | | | | | | |
| | CAGA | | | | | | | |
| 329 | −10_6-6_internal_loop- | GCCACAACUCCCUCCUAGUA | 0.31 | 0.741 | 0.734 | 0.99 | 1.234 | 1.218 |
| | symmetric_AGGAAU-UCUGAG | GGUUGAAAGUCCUUUCAUU | | | | | | |
| | 0_1-1_mismatch_A-C | AAUAACUCCACGGCUAAUG | | | | | | |
| | 5_2-2_bulge-symmetric_UG-AC | AUCUGAGUUACACCACACU | | | | | | |
| | 11_1-1_mismatch_C-U | GUCGUCGAAUGGCCACUCCC | | | | | | |
| | 28_6-6_internal_loop- | AGU | | | | | | |
| | symmetric_AGGCCA-AGUAGG | | | | | | | |
| 330 | −18_6-6_internal_loop- | GCCACAACUCCCUCCUUGGC | 0.322 | 0.814 | 0.769 | 0.937 | 1.235 | 1.283 |
| | symmetric_UGGUGU-UGAAGU | AACGCUAAGUCCUUUCAUG | | | | | | |
| | −5_1-1_mismatch_U-C | AAUACAUCCACGGCUCAUG | | | | | | |
| | 0_1-1_mismatch_A-C | AAUUCCUUUUGAAGUCACU | | | | | | |
| | 24_6-6_internal_loop- | GUCGUCGAAUGGCCACUCCC | | | | | | |
| | symmetric_UCAAAG-AACGCU | AGU | | | | | | |
| 331 | −33_4-4_bulge-symmetric_UUCG- | GCCACACAGACCUCCUUGGC | 0.283 | 0.914 | 0.911 | 1.064 | 1.58 | 1.513 |
| | ACAU | AACGCUAAGUCCUUUCAUG | | | | | | |
| | −18_6-6_internal_loop- | AAUACAUCCACGGCUCAUG | | | | | | |
| | symmetric_UGGUGU-UGAAGU | AAUUCCUUUUGAAGUCACU | | | | | | |
| | −5_1-1_mismatch_U-C | GUCGUACAUUGGCCACUCCC | | | | | | |
| | 0_1-1_mismatch_A-C | AGU | | | | | | |
| | 24_6-6_internal_loop- | | | | | | | |
| | symmetric_UCAAAG-AACGCU | | | | | | | |
| | 40_4-4_bulge-symmetric_GAGU- | | | | | | | |
| | CAGA | | | | | | | |
| 332 | −14_6-6_internal_loop- | GCCACAACUCCCUCCUUGGC | 0.284 | 0.749 | 0.728 | 0.916 | 1.26 | 1.231 |
| | symmetric_GUAAAG-GACCUG | AACGCUAAGUCCUUUCAUG | | | | | | |
| | −5_1-1_mismatch_U-C | AAUACAUCCACGGCUCAUG | | | | | | |
| | 0_1-1_mismatch_A-C | AAUUCGACCUGACCACACU | | | | | | |
| | 24_6-6_internal_loop- | GUCGUCGAAUGGCCACUCCC | | | | | | |
| | symmetric_UCAAAG-AACGCU | AGU | | | | | | |
| 333 | −33_4-4_bulge-symmetric_UUCG- | GCCACACAGACCUCCUUGGC | 0.26 | 0.92 | 0.938 | 1.071 | 1.599 | 1.624 |
| | ACAU | AACGCUAAGUCCUUUCAUG | | | | | | |
| | −14_10−10_internal_loop- | AAUACAUCCACGGCUCAUG | | | | | | |
| | symmetric_UGGUGUAAAG- | AAUUCGACCUGUAGUCACU | | | | | | |
| | GACCUGUAGU | GUCGUACAUUGGCCACUCCC | | | | | | |
| | −5_1-1_mismatch_U-C | AGU | | | | | | |
| | 0_1-1_mismatch_A-C | | | | | | | |
| | 24_6-6_internal_loop- | | | | | | | |
| | symmetric_UCAAAG-AACGCU | | | | | | | |
| | 40_4-4_bulge-symmetric_GAGU- | | | | | | | |
| | CAGA | | | | | | | |
| 334 | −16_6-6_internal_loop- | GCCACAACUCCCUCCUACAU | 0.247 | 0.741 | 0.65 | 1.002 | 1.208 | 1.101 |
| | symmetric_GUGUAA-AUUUUA | UAUUGAAAGUCCUUUCAUG | | | | | | |
| | −4_2-0_bulge-asymmetric_UA- | AAUCCAUCCACGGCAUGAA | | | | | | |
| | 0_1-1_mismatch_A-C | UUCCUAUUUUACACACUGU | | | | | | |
| | 7_1-1_mismatch_U-C | CGUCGAAUGGCCACUCCCAG | | | | | | |
| | 28_6-6_internal_loop- | U | | | | | | |
| | symmetric_AGGCCA-ACAUUA | | | | | | | |
| 335 | −33_4-4_bulge-symmetric_UUCG- | GCCACACAGACCUCCUACAU | 0.121 | 0.94 | 0.92 | 0.992 | 1.693 | 1.643 |
| | ACAU | UAUUGAAAGUCCUUUCAUG | | | | | | |
| | −16_8-8_internal_loop- | AAUCCAUCCACGGCAUGAA | | | | | | |
| | symmetric_UGGUGUAA- | UUCCUAUUUUAGUCACUGU | | | | | | |
| | AUUUUAGU | CGUACAUUGGCCACUCCCAG | | | | | | |
| | −4_2-0_bulge-asymmetric_UA- | U | | | | | | |
| | 0_1-1_mismatch_A-C | | | | | | | |
| | 7_1-1_mismatch_U-C | | | | | | | |
| | 28_6-6_internal_loop- | | | | | | | |
| | symmetric_AGGCCA-ACAUUA | | | | | | | |
| | 40_4-4_bulge-symmetric_GAGU- | | | | | | | |
| | CAGA | | | | | | | |
| 336 | −6_6-6_internal_loop- | GCCACAACUCCCGAAGCCGC | 0.367 | 0.799 | 0.785 | 1.032 | 1.225 | 1.122 |
| | symmetric_AUUCAU-CCGCCC | CUUUGAAAGUCCUUUCAUG | | | | | | |
| | 0_1-1_mismatch_A-C | AAUAGAUCCACGGCUACCG | | | | | | |
| | 6_1-1_mismatch_G-G | CCCUCCUUUACACCACACUG | | | | | | |
| | 32_6-6_internal_loop- | UCGUCGAAUGGCCACUCCCA | | | | | | |
| | symmetric_CAAGGA-GAAGCC | GU | | | | | | |
| 337 | −33_4-4_bulge-symmetric_UUCG- | GCCACACAGACCGAAGCCGC | 0.408 | 0.921 | 0.915 | 1.18 | 1.442 | 1.532 |
| | ACAU | CUUUGAAAGUCCUUUCAUG | | | | | | |
| | −20_4-4_bulge-symmetric_UGGU- | AAUAGAUCCACGGCUACCG | | | | | | |
| | UAGU | CCCUCCUUUACUAGUCACUG | | | | | | |
| | −6_6-6_internal_loop- | UCGUACAUUGGCCACUCCCA | | | | | | |
| | symmetric_AUUCAU-CCGCCC | GU | | | | | | |
| | 0_1-1_mismatch_A-C | | | | | | | |
| | 6_1-1_mismatch_G-G | | | | | | | |
| | 32_6-6_internal_loop- | | | | | | | |
| | symmetric_CAAGGA-GAAGCC | | | | | | | |
| | 40_4-4_bulge-symmetric_GAGU- | | | | | | | |
| | CAGA | | | | | | | |
| 338 | −14_6-6_internal_loop- | GCCACAACUCCCUCGACACG | 0.315 | 0.814 | 0.764 | 1.035 | 1.244 | 1.315 |
| | symmetric_GUAAAG-GGAAUG | CUUUGAAAGUCCUUUCAUG | | | | | | |
| | −5_2-0_bulge-asymmetric_UU- | AAUACAUCCACGGCUUGAA | | | | | | |
| | 0_1-1_mismatch_A-C | UUCGGAAUGACCACACUGU | | | | | | |
| | 30_6-6_internal_loop- | CGUCGAAUGGCCACUCCCAG | | | | | | |
| | symmetric_GCCAAG-GACACG | U | | | | | | |
| 339 | −33_4-4_bulge-symmetric_UUCG- | GCCACACAGACCUCGACACG | 0.309 | 0.908 | 0.884 | 1.055 | 1.548 | 1.515 |
| | ACAU | CUUUGAAAGUCCUUUCAUG | | | | | | |
| | −14_10−10_internal_loop- | AAUACAUCCACGGCUUGAA | | | | | | |
| | symmetric_UGGUGUAAAG- | UUCGGAAUGUAGUCACUGU | | | | | | |
| | GGAAUGUAGU | CGUACAUUGGCCACUCCCAG | | | | | | |
| | −5_2-0_bulge-asymmetric_UU- | U | | | | | | |
| | 0_1-1_mismatch_A-C | | | | | | | |
| | 30_6-6_internal_loop- | | | | | | | |
| | symmetric_GCCAAG-GACACG | | | | | | | |
| | 40_4-4_bulge-symmetric_GAGU- | | | | | | | |
| | CAGA | | | | | | | |
| 340 | −8_6-6_internal_loop- | GCCACAACUCCCUCCUUGGC | 0.273 | 0.806 | 0.677 | 0.895 | 1.18 | 1.198 |
| | symmetric_GAAUUC-CCUUGG | GGGGUUAAGUCCUUUCAUG | | | | | | |
| | 0_1-1_mismatch_A-C | AAUACACCCACGGCUAAUCC | | | | | | |
| | 4_1-1_mismatch_A-C | UUGGCUUUACACCACACUG | | | | | | |
| | 24_6-6_internal_loop- | UCGUCGAAUGGCCACUCCCA | | | | | | |
| | symmetric_UCAAAG-GGGGUU | GU | | | | | | |
| 341 | −16_6-6_internal_loop- | GCCACAACUCCCUCCUUGUA | 0.264 | 0.792 | 0.739 | 0.943 | 1.224 | 1.3 |
| | symmetric_GUGUAA-CCUCUA | GGAGGAAAGUCCUUUCAUU | | | | | | |
| | −7_0-1_bulge-asymmetric_-G | AAUACAUCCACGGCUAAUG | | | | | | |
| | 0_1-1_mismatch_A-C | GAAUUCCUCCUCUACACACU | | | | | | |
| | 11_1-1_mismatch_C-U | GUCGUCGAAUGGCCACUCCC | | | | | | |
| | 26_6-6_internal_loop- | AGU | | | | | | |
| | symmetric_AAAGGC-UAGGAG | | | | | | | |
| 342 | −33_4-4_bulge-symmetric_UUCG- | GCCACACAGACCUCCUUGUA | 0.211 | 0.941 | 0.928 | 1.073 | 1.613 | 1.543 |
| | ACAU | GGAGGAAAGUCCUUUCAUU | | | | | | |
| | −23_0-2_bulge-asymmetric_-GU | AAUACAUCCACGGCUAAUG | | | | | | |
| | −22_1-1_wobble_G-U | GAAUUCCUCCUCUAGUCAC | | | | | | |
| | −20_1-1_mismatch_U-U | UGUCGUACAUUGGCCACUC | | | | | | |
| | −16_3-1_bulge-asymmetric_UAA-C | CCAGU | | | | | | |
| | −7_0-1_bulge-asymmetric_-G | | | | | | | |
| | 0_1-1_mismatch_A-C | | | | | | | |
| | 11_1-1_mismatch_C-U | | | | | | | |
| | 26_6-6_internal_loop- | | | | | | | |
| | symmetric_AAAGGC-UAGGAG | | | | | | | |
| | 40_4-4_bulge-symmetric_GAGU- | | | | | | | |
| | CAGA | | | | | | | |
| 343 | −10_6-6_internal_loop- | GCCACAACUCCCUCCUUGUA | 0.34 | 0.777 | 0.737 | 0.926 | 1.183 | 1.175 |
| | symmetric_AGGAAU-CGAAGA | ACGCGAAAGUCCUUUCAUG | | | | | | |
| | −5_1-1_mismatch_U-C | AAUACAUCCACGGCUCAUG | | | | | | |
| | 0_1-1_mismatch_A-C | ACGAAGAUUACACCACACU | | | | | | |
| | 26_6-6_internal_loop- | GUCGUCGAAUGGCCACUCCC | | | | | | |
| | symmetric_AAAGGC-UAACGC | AGU | | | | | | |
| 344 | −33_4-4_bulge-symmetric_UUCG- | GCCACACAGACCUCCUUGUA | 0.306 | 0.886 | 0.887 | 1.097 | 1.499 | 1.415 |
| | ACAU | ACGCGAAAGUCCUUUCAUG | | | | | | |
| | −20_4-4_bulge-symmetric_UGGU- | AAUACAUCCACGGCUCAUG | | | | | | |
| | UAGU | ACGAAGAUUACUAGUCACU | | | | | | |
| | −10_6-6_internal_loop- | GUCGUACAUUGGCCACUCCC | | | | | | |
| | symmetric_AGGAAU-CGAAGA | AGU | | | | | | |
| | −5_1-1_mismatch_U-C | | | | | | | |
| | 0_1-1_mismatch_A-C | | | | | | | |
| | 26_6-6_internal_loop- | | | | | | | |
| | symmetric_AAAGGC-UAACGC | | | | | | | |
| | 40_4-4_bulge-symmetric_GAGU- | | | | | | | |
| | CAGA | | | | | | | |
| 345 | −6_8-8_internal_loop- | GCCACAACUCCCUCCUUGGC | 0.263 | 0.789 | 0.783 | 0.921 | 1.341 | 1.226 |
| | symmetric_GAAUUCAU- | GCCAGUAAGUCCUUUCAUG | | | | | | |
| | CGACAGGA | AAUGAAUCCACGGCUACGA | | | | | | |
| | 0_1-1_mismatch_A-C | CAGGACUUUACACCACACU | | | | | | |
| | 6_1-1_mismatch_G-A | GUCGUCGAAUGGCCACUCCC | | | | | | |
| | 7_1-1_wobble_U-G | AGU | | | | | | |
| | 24_6-6_internal_loop- | | | | | | | |
| | symmetric_UCAAAG-GCCAGU | | | | | | | |
| 346 | −33_4-4_bulge-symmetric_UUCG- | GCCACACAGACCUCCUUGGC | 0.07 | 0.869 | 0.879 | 0.942 | 1.604 | 1.571 |
| | ACAU | GCCAGUAAGUCCUUUCAUG | | | | | | |
| | −20_4-4_bulge-symmetric_UGGU- | AAUGAAUCCACGGCUACGA | | | | | | |
| | UAGU | CAGGACUUUACUAGUCACU | | | | | | |
| | −6_8-8_internal_loop- | GUCGUACAUUGGCCACUCCC | | | | | | |
| | symmetric_GAAUUCAU- | AGU | | | | | | |
| | CGACAGGA | | | | | | | |
| | 0_1-1_mismatch_A-C | | | | | | | |
| | 6_1-1_mismatch_G-A | | | | | | | |
| | 7_1-1_wobble_U-G | | | | | | | |
| | 24_6-6_internal loop- | | | | | | | |
| | symmetric_UCAAAG-GCCAGU | | | | | | | |
| | 40_4-4_bulge-symmetric_GAGU- | | | | | | | |
| | CAGA | | | | | | | |
| 347 | −10_6-6_internal_loop- | GCCACAACUCCCUCCUAUCA | 0.351 | 0.803 | 0.809 | 1.029 | 1.227 | 1.302 |
| | symmetric_AGGAAU-CGCAGA | ACUUGAAAGUCCUUUCAUG | | | | | | |
| | 0_1-1_mismatch_A-C | CAUACAUCCACGGCUAAUG | | | | | | |
| | 10_1-1_mismatch_U-C | ACGCAGAUUACACCACACU | | | | | | |
| | 28_6-6_internal_loop- | GUCGUCGAAUGGCCACUCCC | | | | | | |
| | symmetric_AGGCCA-AUCAAC | AGU | | | | | | |
| 348 | −33_4-4_bulge-symmetric_UUCG- | GCCACACAGACCUCCUAUCA | 0.259 | 0.886 | 0.886 | 1.121 | 1.502 | 1.536 |
| | ACAU | ACUUGAAAGUCCUUUCAUG | | | | | | |
| | −20_4-4_bulge-symmetric_UGGU- | CAUACAUCCACGGCUAAUG | | | | | | |
| | UAGU | ACGCAGAUUACUAGUCACU | | | | | | |
| | −10_6-6_internal_loop- | GUCGUACAUUGGCCACUCCC | | | | | | |
| | symmetric_AGGAAU-CGCAGA | AGU | | | | | | |
| | 0_1-1_mismatch_A-C | | | | | | | |
| | 10_1-1_mismatch_U-C | | | | | | | |
| | 28_6-6_internal_loop- | | | | | | | |
| | symmetric_AGGCCA-AUCAAC | | | | | | | |
| | 40_4-4_bulge-symmetric_GAGU- | | | | | | | |
| | CAGA | | | | | | | |
| 349 | −14_6-6_internal_loop- | GCCACAACUCCCUCAGCCAG | 0.246 | 0.83 | 0.823 | 1.062 | 1.396 | 1.303 |
| | symmetric_GUAAAG-GACGCA | CUUUGAAAGUACUUUCAUG | | | | | | |
| | −6_1-1_wobble_U-G | AAUACAUCCACGGAGUGAA | | | | | | |
| | −3_2-0_bulge-asymmetric_AG- | UUCGACGCAACCACACUGUC | | | | | | |
| | 0_1-1_mismatch_A-C | GUCGAAUGGCCACUCCCAG | | | | | | |
| | 19_1-1_mismatch_G-A | U | | | | | | |
| | 30_6-6_internal_loop- | | | | | | | |
| | symmetric_GCCAAG-AGCCAG | | | | | | | |
| 350 | −6_6-6_internal_loop- | GCCACAACUCCCAGGAUCGC | 0.195 | 0.889 | 0.839 | 0.921 | 1.436 | 1.309 |
| | symmetric_AUUCAU-CGACAG | CUUUGAAAGUCCUUCCCUG | | | | | | |
| | 0_1-1_mismatch_A-C | AAUACAUCCACGGCUACGA | | | | | | |
| | 13_1-1_mismatch_U-C | CAGUCCUUUACACCACACUG | | | | | | |
| | 15_1-1_mismatch_A-C | UCGUCGAAUGGCCACUCCCA | | | | | | |
| | 32_6-6_internal_loop- | GU | | | | | | |
| | symmetric_CAAGGA-AGGAUC | | | | | | | |
| 351 | 0_1-1_mismatch_A-C | GCCACAACUCCCUCCUCUUU | 0.395 | 0.817 | 0.78 | 1.07 | 1.346 | 1.331 |
| | 6_1-1_mismatch_G-G | ACUUGAAAGUCCUUUCAUG | | | | | | |
| | 28_6-6_internal_loop- | AAUAGAUCCACGGCUAAUG | | | | | | |
| | symmetric_AGGCCA-CUUUAC | AAUUCCUUUACACCACACU | | | | | | |
| | | GUCGUCGAAUGGCCACUCCC | | | | | | |
| | | AGU | | | | | | |
| 352 | −33_4-4_bulge-symmetric_UUCG- | GCCACACAGACCUCCUCUUU | 0.495 | 0.9 | 0.89 | 1.247 | 1.556 | 1.54 |
| | ACAU | ACUUGAAAGUCCUUUCAUG | | | | | | |
| | −20_4-4_bulge-symmetric_UGGU- | AAUAGAUCCACGGCUAAUG | | | | | | |
| | UAGU | AAUUCCUUUACUAGUCACU | | | | | | |
| | 0_1-1_mismatch_A-C | GUCGUACAUUGGCCACUCCC | | | | | | |
| | 6_1-1_mismatch_G-G | AGU | | | | | | |
| | 28_6-6_internal_loop- | | | | | | | |
| | symmetric_AGGCCA-CUUUAC | | | | | | | |
| | 40_4-4_bulge-symmetric_GAGU- | | | | | | | |
| | CAGA | | | | | | | |
| 353 | −10_6-6_internal_loop- | GCCACAACUCCCUCCUCUCG | 0.303 | 0.798 | 0.781 | 1.006 | 1.222 | 1.183 |
| | symmetric_AGGAAU-UAGAGC | AGUUGAAAGUCCUUUCAUG | | | | | | |
| | −6_2-2_bulge-symmetric_AU-CG | AAUACAUCCACGGCUACGG | | | | | | |
| | 0_1-1_mismatch_A-C | AUAGAGCUUACACCACACU | | | | | | |
| | 28_6-6_internal_loop- | GUCGUCGAAUGGCCACUCCC | | | | | | |
| | symmetric_AGGCCA-CUCGAG | AGU | | | | | | |
| 354 | −33_4-4_bulge-symmetric_UUCG- | GCCACACAGACCUCCUCUCG | 0.246 | 0.838 | 0.831 | 1.047 | 1.493 | 1.473 |
| | ACAU | AGUUGAAAGUCCUUUCAUG | | | | | | |
| | −20_4-4_bulge-symmetric_UGGU- | AAUACAUCCACGGCUACGG | | | | | | |
| | UAGU | AUAGAGCUUACUAGUCACU | | | | | | |
| | −10_6-6_internal_loop- | GUCGUACAUUGGCCACUCCC | | | | | | |
| | symmetric_AGGAAU-UAGAGC | AGU | | | | | | |
| | −6_2-2_bulge-symmetric_AU-CG | | | | | | | |
| | 0_1-1_mismatch_A-C | | | | | | | |
| | 28_6-6_internal_loop- | | | | | | | |
| | symmetric_AGGCCA-CUCGAG | | | | | | | |
| | 40_4-4_bulge-symmetric_GAGU- | | | | | | | |
| | CAGA | | | | | | | |
| 355 | −18_6-6_internal_loop- | GCCACAACUCCCUCCUCUCA | 0.115 | 0.819 | 0.831 | 0.936 | 1.259 | 1.278 |
| | symmetric_UGGUGU-UGUAAU | UCUUGAAAGUCGACUUUCA | | | | | | |
| | −3_2-0_bulge-asymmetric_AG- | UGAAUACAUCCACGGAAUG | | | | | | |
| | 0_1-1_mismatch_A-C | AAUUCCUUUUGUAAUCACU | | | | | | |
| | 18_0-2_bulge-asymmetric_-GA | GUCGUCGAAUGGCCACUCCC | | | | | | |
| | 28_6-6_internal_loop- | AGU | | | | | | |
| | symmetric_AGGCCA-CUCAUC | | | | | | | |
| 356 | −6_6-6_internal_loop- | GCCACAACUCCCUCCUUGAU | 0.285 | 0.748 | 0.751 | 0.964 | 1.192 | 1.153 |
| | symmetric_AUUCAU-UCAUAC | AGGCGAAAGUCCUUUCAUG | | | | | | |
| | 0_1-1_mismatch_A-C | AAUAACUCCACGGCUAUCA | | | | | | |
| | 5_2-2_bulge-symmetric_UG-AC | UACUCCUUUACACCACACUG | | | | | | |
| | 26_6-6_internal_loop- | UCGUCGAAUGGCCACUCCCA | | | | | | |
| | symmetric_AAAGGC-AUAGGC | GU | | | | | | |
| 357 | −8_6-6_internal_loop- | GCCACAACUCCCUCCUCUAA | 0.396 | 0.808 | 0.826 | 1.089 | 1.234 | 1.366 |
| | symmetric_GAAUUC-AACUGG | AGUUGAAAGUCCUUUCAUG | | | | | | |
| | −2_2-1_bulge-asymmetric_GC-A | AAUACAUCCACGAUAAUAA | | | | | | |
| | 0_1-1_mismatch_A-C | CUGGCUUUACACCACACUG | | | | | | |
| | 28_6-6_internal_loop- | UCGUCGAAUGGCCACUCCCA | | | | | | |
| | symmetric_AGGCCA-CUAAAG | GU | | | | | | |
| 358 | −33_4-4_bulge-symmetric_UUCG- | GCCACACAGACCUCCUCUAA | 0.349 | 0.909 | 0.883 | 1.166 | 1.632 | 1.591 |
| | ACAU | AGUUGAAAGUCCUUUCAUG | | | | | | |
| | −20_4-4_bulge-symmetric_UGGU- | AAUACAUCCACGAUAAUAA | | | | | | |
| | UAGU | CUGGCUUUACUAGUCACUG | | | | | | |
| | −8_6-6_internal_loop- | UCGUACAUUGGCCACUCCCA | | | | | | |
| | symmetric_GAAUUC-AACUGG | GU | | | | | | |
| | −2_2-1_bulge-asymmetric_GC-A | | | | | | | |
| | 0_1-1_mismatch_A-C | | | | | | | |
| | 28_6-6_internal_loop- | | | | | | | |
| | symmetric_AGGCCA-CUAAAG | | | | | | | |
| | 40_4-4_bulge-symmetric_GAGU- | | | | | | | |
| | CAGA | | | | | | | |
| 359 | −10_6-6_internal_loop- | GCCACAACUCCCUCCUGAUA | 0.315 | 0.808 | 0.776 | 1.011 | 1.252 | 1.152 |
| | symmetric_AGGAAU-CAAGAA | UGUUGAAAGUCCUUUCCUG | | | | | | |
| | −6_2-1_bulge-asymmetric_AU-C | AAUACAUCCACGGCUACGA | | | | | | |
| | 0_1-1_mismatch_A-C | CAAGAAUUACACCACACUG | | | | | | |
| | 13_1-1_mismatch_U-C | UCGUCGAAUGGCCACUCCCA | | | | | | |
| | 28_6-6_internal_loop- | GU | | | | | | |
| | symmetric_AGGCCA-GAUAUG | | | | | | | |
| 360 | −33_4-4_bulge-symmetric_UUCG- | GCCACACAGACCUCCUGAUA | 0.205 | 0.897 | 0.893 | 1.062 | 1.621 | 1.619 |
| | ACAU | UGUUGAAAGUCCUUUCCUG | | | | | | |
| | −20_4-4_bulge-symmetric_UGGU- | AAUACAUCCACGGCUACGA | | | | | | |
| | UAGU | CAAGAAUUACUAGUCACUG | | | | | | |
| | −10_6-6_internal_loop- | UCGUACAUUGGCCACUCCCA | | | | | | |
| | symmetric_AGGAAU-CAAGAA | GU | | | | | | |
| | −6_2-1_bulge-asymmetric_AU-C | | | | | | | |
| | 0_1-1_mismatch_A-C | | | | | | | |
| | 13_1-1_mismatch_U-C | | | | | | | |
| | 28_6-6_internal_loop- | | | | | | | |
| | symmetric_AGGCCA-GAUAUG | | | | | | | |
| | 40_4-4_bulge-symmetric_GAGU- | | | | | | | |
| | CAGA | | | | | | | |
| 361 | −10_6-6_internal_loop- | GCCACAACUCCCUCCUACAA | 0.329 | 0.785 | 0.82 | 1.02 | 1.183 | 1.248 |
| | symmetric_AGGAAU-CGGAGA | ACUUGAAAGUCCUUUCAUG | | | | | | |
| | −6_0-1_bulge-asymmetric_-U | AAUACAACCACGGCUAAUU | | | | | | |
| | 0_1-1_mismatch_A-C | GACGGAGAUUACACCACAC | | | | | | |
| | 4_1-1_mismatch_A-A | UGUCGUCGAAUGGCCACUC | | | | | | |
| | 28_6-6_internal_loop- | CCAGU | | | | | | |
| | symmetric_AGGCCA-ACAAAC | | | | | | | |
| 362 | −33_4-4_bulge-symmetric_UUCG- | GCCACACAGACCUCCUACAA | 0.371 | 0.875 | 0.894 | 1.193 | 1.48 | 1.471 |
| | ACAU | ACUUGAAAGUCCUUUCAUG | | | | | | |
| | −20_4-4_bulge-symmetric_UGGU- | AAUACAACCACGGCUAAUU | | | | | | |
| | UAGU | GACGGAGAUUACUAGUCAC | | | | | | |
| | −10_6-6_internal_loop- | UGUCGUACAUUGGCCACUC | | | | | | |
| | symmetric_AGGAAU-CGGAGA | CCAGU | | | | | | |
| | −6_0-1_bulge-asymmetric_-U | | | | | | | |
| | 0_1-1_mismatch_A-C | | | | | | | |
| | 4_1-1_mismatch_A-A | | | | | | | |
| | 28_6-6_internal_loop- | | | | | | | |
| | symmetric_AGGCCA-ACAAAC | | | | | | | |
| | 40_4-4_bulge-symmetric_GAGU- | | | | | | | |
| | CAGA | | | | | | | |
| 363 | −3_1-1_mismatch_G-G | GCCACAACUCCCUCCUAAAA | 0.3 | 0.795 | 0.796 | 1.036 | 1.233 | 1.325 |
| | 0_1-1_mismatch_A-C | AGUUGAAAGUCCUUUCAUG | | | | | | |
| | 28_6-6_internal_loop- | AAUACAUCCACGGGUAAUG | | | | | | |
| | symmetric_AGGCCA-AAAAAG | AAUUCCUUUACACCACACU | | | | | | |
| | | GUCGUCGAAUGGCCACUCCC | | | | | | |
| | | AGU | | | | | | |
| 364 | −33_4-4_bulge-symmetric_UUCG- | GCCACACAGACCUCCUAAAA | 0.371 | 0.924 | 0.932 | 1.074 | 1.591 | 1.65 |
| | ACAU | AGUUGAAAGUCCUUUCAUG | | | | | | |
| | −20_4-4_bulge-symmetric_UGGU- | AAUACAUCCACGGGUAAUG | | | | | | |
| | UAGU | AAUUCCUUUACUAGUCACU | | | | | | |
| | −3_1-1_mismatch_G-G | GUCGUACAUUGGCCACUCCC | | | | | | |
| | 0_1-1_mismatch_A-C | AGU | | | | | | |
| | 28_6-6_internal_loop- | | | | | | | |
| | symmetric_AGGCCA-AAAAAG | | | | | | | |
| | 40_4-4_bulge-symmetric_GAGU- | | | | | | | |
| | CAGA | | | | | | | |
| 365 | −10_6-6_internal_loop- | GCCACAACUCCCUCCUGAAU | 0.333 | 0.794 | 0.771 | 1.081 | 1.228 | 1.21 |
| | symmetric_AGGAAU-UCUGAG | GAUUGAAAGUCCUUUCAUG | | | | | | |
| | −4_2-0_bulge-asymmetric_UA- | AAUACAUCCACGGCAUGAU | | | | | | |
| | 0_1-1_mismatch_A-C | CUGAGUUACACCACACUGU | | | | | | |
| | 28_6-6_internal_loop- | CGUCGAAUGGCCACUCCCAG | | | | | | |
| | symmetric_AGGCCA-GAAUGA | U | | | | | | |
| 366 | −16_6-6_internal_loop- | GCCACAACUCCCUCCUCAUG | 0.215 | 0.767 | 0.77 | 0.949 | 1.164 | 1.242 |
| | symmetric_GUGUAA-GGAAUG | AGUUGAAAGUCCUUUCAUG | | | | | | |
| | −3_4-3_bulge-asymmetric_UUAG- | AAUACAUCCACGGACUUGA | | | | | | |
| | ACU | AUUCCUGGAAUGCACACUG | | | | | | |
| | 0_1-1_mismatch_A-C | UCGUCGAAUGGCCACUCCCA | | | | | | |
| | 28_6-6_internal_loop- | GU | | | | | | |
| | symmetric_AGGCCA-CAUGAG | | | | | | | |
| 367 | −14_6-6_internal_loop- | GCCACAACUCCCUCCUUGUA | 0.29 | 0.757 | 0.781 | 0.879 | 1.187 | 1.213 |
| | symmetric_GUAAAG-GCGCUG | AGGAGAAAGUCCUUUCAUG | | | | | | |
| | −4_3-3_bulge-symmetric_UUA-GGC | AAUACAUCCACGGCGGCUG | | | | | | |
| | 0_1-1_mismatch_A-C | AAUUCGCGCUGACCACACU | | | | | | |
| | 26_6-6_internal_loop- | GUCGUCGAAUGGCCACUCCC | | | | | | |
| | symmetric_AAAGGC-UAAGGA | AGU | | | | | | |
| 368 | −33_4-4_bulge-symmetric_UUCG- | GCCACACAGACCUCCUUGUA | 0.242 | 0.839 | 0.863 | 1.042 | 1.541 | 1.477 |
| | ACAU | AGGAGAAAGUCCUUUCAUG | | | | | | |
| | −23_0-4_bulge-asymmetric_-UAGU | AAUACAUCCACGGCGGCUG | | | | | | |
| | −23_1-1_wobble_U-G | AAUUCGCGCUGUAGUCACU | | | | | | |
| | −22_1-1_wobble_G-U | GUCGUACAUUGGCCACUCCC | | | | | | |
| | −20_1-1_wobble_U-G | AGU | | | | | | |
| | −18_1-1_wobble_U-G | | | | | | | |
| | −14_4-0_bulge-asymmetric_AAAG- | | | | | | | |
| | −4_3-3_bulge-symmetric_UUA-GGC | | | | | | | |
| | 0_1-1_mismatch_A-C | | | | | | | |
| | 26_6-6_internal_loop- | | | | | | | |
| | symmetric_AAAGGC-UAAGGA | | | | | | | |
| | 40_4-4_bulge-symmetric_GAGU- | | | | | | | |
| | CAGA | | | | | | | |
| 369 | −4_6-6_internal_loop- | GCCACAACUCCCUCCUCAUU | 0.288 | 0.849 | 0.846 | 1.04 | 1.249 | 1.285 |
| | symmetric_UCAUUA-GGCGAU | GAUUGAAAGUCCUUUCAUG | | | | | | |
| | 0_1-1_mismatch_A-C | AAUACCCCACGGCGGCGAU | | | | | | |
| | 4_2-1_bulge-asymmetric_AU-C | AUUCCUUUACACCACACUG | | | | | | |
| | 28_6-6_internal_loop- | UCGUCGAAUGGCCACUCCCA | | | | | | |
| | symmetric_AGGCCA-CAUUGA | GU | | | | | | |
| 370 | −33_4-4_bulge-symmetric_UUCG- | GCCACACAGACCUCCUCAUU | 0.105 | 0.86 | 0.887 | 0.995 | 1.603 | 1.643 |
| | ACAU | GAUUGAAAGUCCUUUCAUG | | | | | | |
| | −20_4-4_bulge-symmetric_UGGU- | AAUACCCCACGGCGGCGAU | | | | | | |
| | UAGU | AUUCCUUUACUAGUCACUG | | | | | | |
| | −4_6-6_internal_loop- | UCGUACAUUGGCCACUCCCA | | | | | | |
| | symmetric_UCAUUA-GGCGAU | GU | | | | | | |
| | 0_1-1_mismatch_A-C | | | | | | | |
| | 4_2-1_bulge-asymmetric_AU-C | | | | | | | |
| | 28_6-6_internal_loop- | | | | | | | |
| | symmetric_AGGCCA-CAUUGA | | | | | | | |
| | 40_4-4_bulge-symmetric_GAGU- | | | | | | | |
| | CAGA | | | | | | | |
| 371 | −10_6-6_internal_loop- | GCCACAACUCCCUCCUUGAU | 0.329 | 0.801 | 0.734 | 0.906 | 1.227 | 0.988 |
| | symmetric_AGGAAU-CGAUAG | AAUCGAAAGUCCUUUCAUG | | | | | | |
| | −5_2-2_bulge-symmetric_UU-CU | AAUACAUCCACGGCUCUUG | | | | | | |
| | 0_1-1_mismatch_A-C | ACGAUAGUUACACCACACU | | | | | | |
| | 26_6-6_internal_loop- | GUCGUCGAAUGGCCACUCCC | | | | | | |
| | symmetric_AAAGGC-AUAAUC | AGU | | | | | | |
| 372 | −33_4-4_bulge-symmetric_UUCG- | GCCACACAGACCUCCUUGAU | 0.313 | 0.891 | 0.88 | 1.087 | 1.57 | 1.489 |
| | ACAU | AAUCGAAAGUCCUUUCAUG | | | | | | |
| | −20_4-4_bulge-symmetric_UGGU- | AAUACAUCCACGGCUCUUG | | | | | | |
| | UAGU | ACGAUAGUUACUAGUCACU | | | | | | |
| | −10_6-6_internal_loop- | GUCGUACAUUGGCCACUCCC | | | | | | |
| | symmetric_AGGAAU-CGAUAG | AGU | | | | | | |
| | −5_2-2_bulge-symmetric_UU-CU | | | | | | | |
| | 0_1-1_mismatch_A-C | | | | | | | |
| | 26_6-6_internal_loop- | | | | | | | |
| | symmetric_AAAGGC-AUAAUC | | | | | | | |
| | 40_4-4_bulge-symmetric_GAGU- | | | | | | | |
| | CAGA | | | | | | | |
| 373 | −20_6-6_internal_loop- | GCCACAACUCCCUCCUUGAU | 0.216 | 0.769 | 0.758 | 0.962 | 1.216 | 1.186 |
| | symmetric_UGUGGU-UGGAGU | GGCGGAAAGUCCCUUCAUG | | | | | | |
| | −5_4-4_bulge-symmetric_CAUU- | AAUACUAUCCACGGCUCUA | | | | | | |
| | CUAC | CAAUUCCUUUACUGGAGUC | | | | | | |
| | 0_1-1_mismatch_A-C | UGUCGUCGAAUGGCCACUC | | | | | | |
| | 5_0-1_bulge-asymmetric_-U | CCAGU | | | | | | |
| | 17_1-1_mismatch_A-C | | | | | | | |
| | 26_6-6_internal_loop- | | | | | | | |
| | symmetric_AAAGGC-AUGGCG | | | | | | | |
| 374 | −4_6-6_internal_loop- | GCCACAACUCCCUCCUCCAA | 0.254 | 0.798 | 0.77 | 0.965 | 1.25 | 1.219 |
| | symmetric_UCAUUA-GGUUAC | GAUUGAAAGUCCUUUCAAG | | | | | | |
| | 0_1-1_mismatch_A-C | AAUACAUCCACGGCGGUUA | | | | | | |
| | 12 1-1_mismatch_A-A | CAUUCCUUUACACCACACUG | | | | | | |
| | 28_6-6_internal_loop- | UCGUCGAAUGGCCACUCCCA | | | | | | |
| | symmetric_AGGCCA-CCAAGA | GU | | | | | | |
| 375 | −33_4-4_bulge-symmetric_UUCG- | GCCACACAGACCUCCUCCAA | 0.065 | 0.908 | 0.905 | 0.949 | 1.64 | 1.649 |
| | ACAU | GAUUGAAAGUCCUUUCAAG | | | | | | |
| | −20_4-4_bulge-symmetric_UGGU- | AAUACAUCCACGGCGGUUA | | | | | | |
| | UAGU | CAUUCCUUUACUAGUCACU | | | | | | |
| | −4_6-6_internal_loop- | GUCGUACAUUGGCCACUCCC | | | | | | |
| | symmetric_UCAUUA-GGUUAC | AGU | | | | | | |
| | 0_1-1_mismatch_A-C | | | | | | | |
| | 12_1-1_mismatch_A-A | | | | | | | |
| | 28_6-6_internal_loop- | | | | | | | |
| | symmetric_AGGCCA-CCAAGA | | | | | | | |
| | 40_4-4_bulge-symmetric_GAGU- | | | | | | | |
| | CAGA | | | | | | | |
| 376 | −4_6-6_internal_loop- | GCCACAACUCCCUCCUGACA | 0.377 | 0.84 | 0.821 | 1.029 | 1.255 | 1.35 |
| | symmetric_UCAUUA-GUGCAU | UAUUGAAAGUCCUUUCAUC | | | | | | |
| | 0_1-1_mismatch_A-C | AAUACAUCCACGGCGUGCA | | | | | | |
| | 11_1-1_mismatch_C-C | UAUUCCUUUACACCACACU | | | | | | |
| | 28_6-6_internal_loop- | GUCGUCGAAUGGCCACUCCC | | | | | | |
| | symmetric_AGGCCA-GACAUA | AGU | | | | | | |
| 377 | −33_4-4_bulge-symmetric_UUCG- | GCCACACAGACCUCCUGACA | 0.299 | 0.904 | 0.915 | 1.154 | 1.613 | 1.568 |
| | ACAU | UAUUGAAAGUCCUUUCAUC | | | | | | |
| | −20_4-4_bulge-symmetric_UGGU- | AAUACAUCCACGGCGUGCA | | | | | | |
| | UAGU | UAUUCCUUUACUAGUCACU | | | | | | |
| | −4_6-6_internal_loop- | GUCGUACAUUGGCCACUCCC | | | | | | |
| | symmetric_UCAUUA-GUGCAU | AGU | | | | | | |
| | 0_1-1_mismatch_A-C | | | | | | | |
| | 11_1-1_mismatch_C-C | | | | | | | |
| | 28_6-6_internal_loop- | | | | | | | |
| | symmetric_AGGCCA-GACAUA | | | | | | | |
| | 40_4-4_bulge-symmetric_GAGU- | | | | | | | |
| | CAGA | | | | | | | |
| 378 | −6_6-6_internal_loop- | GCCACAACUCCCUCCUGAUA | 0.316 | 0.815 | 0.761 | 1.047 | 1.302 | 1.283 |
| | symmetric_AUUCAU-UUCAUC | UGUUGAAAGUCCUUUCCUG | | | | | | |
| | 0_1-1_mismatch_A-C | AAUACAUUCACGGCUAUUC | | | | | | |
| | 3_1-1_wobble_G-U | AUCUCCUUUACACCACACUG | | | | | | |
| | 13 1-1_mismatch_U-C | UCGUCGAAUGGCCACUCCCA | | | | | | |
| | 28_6-6_internal_loop- | GU | | | | | | |
| | symmetric_AGGCCA-GAUAUG | | | | | | | |
| 379 | −33_4-4_bulge-symmetric_UUCG- | GCCACACAGACCUCCUGAUA | 0.09 | 0.894 | 0.886 | 0.952 | 1.644 | 1.628 |
| | ACAU | UGUUGAAAGUCCUUUCCUG | | | | | | |
| | −20_4-4_bulge-symmetric_UGGU- | AAUACAUUCACGGCUAUUC | | | | | | |
| | UAGU | AUCUCCUUUACUAGUCACU | | | | | | |
| | −6_6-6_internal_loop- | GUCGUACAUUGGCCACUCCC | | | | | | |
| | symmetric_AUUCAU-UUCAUC | AGU | | | | | | |
| | 0_1-1_mismatch_A-C | | | | | | | |
| | 3 1-1_wobble_G-U | | | | | | | |
| | 13_1-1_mismatch_U-C | | | | | | | |
| | 28_6-6_internal_loop- | | | | | | | |
| | symmetric_AGGCCA-GAUAUG | | | | | | | |
| | 40_4-4_bulge-symmetric_GAGU- | | | | | | | |
| | CAGA | | | | | | | |
| 380 | −8_6-6_internal_loop- | GCCACAACUCCCUCCUCUAU | 0.372 | 0.815 | 0.771 | 1.034 | 1.293 | 1.133 |
| | symmetric_GAAUUC-UGUUGG | UAUUGAAAGUCCUUUCAUU | | | | | | |
| | 0_1-1_mismatch_A-C | AAUACAUCCACGGCUAAUU | | | | | | |
| | 11_1-1_mismatch_C-U | GUUGGCUUUACACCACACU | | | | | | |
| | 28_6-6_internal_loop- | GUCGUCGAAUGGCCACUCCC | | | | | | |
| | symmetric_AGGCCA-CUAUUA | AGU | | | | | | |
| 381 | −33_4-4_bulge-symmetric_UUCG- | GCCACACAGACCUCCUCUAU | 0.292 | 0.912 | 0.909 | 1.128 | 1.642 | 1.666 |
| | ACAU | UAUUGAAAGUCCUUUCAUU | | | | | | |
| | −20_4-4_bulge-symmetric_UGGU- | AAUACAUCCACGGCUAAUU | | | | | | |
| | UAGU | GUUGGCUUUACUAGUCACU | | | | | | |
| | −8_6-6_internal_loop- | GUCGUACAUUGGCCACUCCC | | | | | | |
| | symmetric_GAAUUC-UGUUGG | AGU | | | | | | |
| | 0_1-1_mismatch_A-C | | | | | | | |
| | 11_1-1_mismatch_C-U | | | | | | | |
| | 28_6-6_internal loop- | | | | | | | |
| | symmetric_AGGCCA-CUAUUA | | | | | | | |
| | 40_4-4_bulge-symmetric_GAGU- | | | | | | | |
| | CAGA | | | | | | | |
| 382 | −14_6-6_internal_loop- | GCCACAACUCCCUCCUAACA | 0.261 | 0.769 | 0.725 | 1.023 | 1.16 | 1.289 |
| | symmetric_GUAAAG-GGUCCG | UCUUGAAAGUCCUUUCAUG | | | | | | |
| | −5_3-3_bulge-symmetric_AUU-UUG | AAUACAUCCACGGCUUUGG | | | | | | |
| | 0_1-1_mismatch_A-C | AAUUCGGUCCGACCACACU | | | | | | |
| | 28_6-6_internal_loop- | GUCGUCGAAUGGCCACUCCC | | | | | | |
| | symmetric_AGGCCA-AACAUC | AGU | | | | | | |
| 383 | −33_4-4_bulge-symmetric_UUCG- | GCCACACAGACCUCCUAACA | 0.29 | 0.894 | 0.883 | 1.126 | 1.564 | 1.568 |
| | ACAU | UCUUGAAAGUCCUUUCAUG | | | | | | |
| | −14_10−10_internal_loop- | AAUACAUCCACGGCUUUGG | | | | | | |
| | symmetric_UGGUGUAAAG- | AAUUCGGUCCGUAGUCACU | | | | | | |
| | GGUCCGUAGU | GUCGUACAUUGGCCACUCCC | | | | | | |
| | −5_3-3_bulge-symmetric_AUU-UUG | AGU | | | | | | |
| | 0_1-1_mismatch_A-C | | | | | | | |
| | 28_6-6_internal_loop- | | | | | | | |
| | symmetric_AGGCCA-AACAUC | | | | | | | |
| | 40_4-4_bulge-symmetric_GAGU- | | | | | | | |
| | CAGA | | | | | | | |
| 384 | −10_6-6_internal_loop- | GCCACAACUCCCUCCUCUCA | 0.317 | 0.785 | 0.701 | 1.123 | 1.242 | 1.188 |
| | symmetric_AGGAAU-UGCAUA | GGUUGAAAGUCCUUUCAUG | | | | | | |
| | −5_3-3_bulge-symmetric_AUU-UCC | GAUACAUCCACGGCUUCCG | | | | | | |
| | 0_1-1_mismatch_A-C | AUGCAUAUUACACCACACU | | | | | | |
| | 10_1-1_wobble_U-G | GUCGUCGAAUGGCCACUCCC | | | | | | |
| | 28_6-6_internal_loop- | AGU | | | | | | |
| | symmetric_AGGCCA-CUCAGG | | | | | | | |

### Example 5

### Selected Engineered Guide RNA Compositions Targeting the SNCA Codon 1 TIS

This example describes the top 48 engineered guide RNAs that target the SNCA Codon 1 transcription initiation site (TIS) of target SNCA mRNA. Self-annealing RNA structures, which comprised (i) the engineered guide RNAs shown in TABLE 3 and (ii) the RNA sequences of the SNCA TIS targeted by the engineered guide RNAs, were contacted with ADAR1 for 30 minutes under conditions that allowed for editing. The regions targeted by the engineered guide RNAs were subsequently assessed for editing using next generation sequencing (NGS). All polynucleotide sequences encoding for the engineered guide RNAs of TABLE 3, are encompassed herein, which are represented by each of the SEQ ID NOs shown in TABLE 3, with a T substituted for each U. For each sequence, the structural features formed in the double stranded RNA substrate upon hybridization of the guide RNA to the target SNCA RNA, are shown in the second column of TABLE 3. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, with reference to SEQ ID NO: 336, “−6_6-6_internal_loop-symmetric_AUUCAU-CCGCCC” is read as a structural feature formed in a guide-target RNA scaffold (target SNCA RNA sequence hybridized to an engineered guide RNA of SEQ ID NO: 336), where
a. the structural feature starts 6 nucleotides upstream (5′) (the −6 position) from the target A (0 position) of the target RNA sequence
b. six contiguous bases from the target RNA sequence and six contiguous bases from the engineered guide RNA form the structural feature
c. the structural feature is an internal symmetric loop
d. a sequence of AUUCAU from the target RNA side and a sequence of CCGCCC from the engineered guide RNA side participate in forming the internal symmetric loop.

TABLE 3
Top 48 engineered guide RNAs that target the SNCA TIS.

| SEQ ID NO | Structural Features (target/guide) |
|---|---|
| 336 | -6_6-6_internal_loop-symmetric_AUUCAU-CCGCCC; 0_1- |
| | 1_mismatch_A-C; 6_1-1_mismatch_G-G; 32_6-6_internal_loop- |
| | symmetric_CAAGGA-GAAGCC |
| 350 | -6_6-6_internal_loop-symmetric_AUUCAU-CGACAG; 0_1- |
| | 1_mismatch_A-C; 13_1-1_mismatch_U-C; 15_1-1_mismatch_A-C; 32_6- |
| | 6_internal_loop-symmetric_CAAGGA-AGGAUC |
| 293 | -8_6-6_internal_loop-symmetric_GAAUUC-UAUAAG; 0_1- |
| | 1_mismatch_A-C; 6_1-1_mismatch_G-G; 30_6-6_internal_loop- |
| | symmetric_GCCAAG-GAACGA |
| 303 | -18_6-6_internal_loop-symmetric_UGGUGU-UGGAGU; -6_3-3_bulge- |
| | symmetric_CAU-CAU; 0_1-1_mismatch_A-C; 6_1-1_mismatch_G-G; |
| | 10_1-1_mismatch_U-C; 30_6-6_internal_loop-symmetric_GCCAAG- |
| | GCGCAA |
| 306 | -10_6-6_internal_loop-symmetric_AGGAAU-CAGUAC; 0_1- |
| | 1_mismatch_A-C; 4_2-2_bulge-symmetric_AU-CA; 11_1-1_mismatch_C- |
| | C; 30_6-6_internal_loop-symmetric_GCCAAG-AGCCUA |
| 309 | -14_6-6_internal_loop-symmetric_GUAAAG-GAACCG; -5_4-4_bulge- |
| | symmetric_CAUU-CAAC; 0_1-1_mismatch_A-C; 4_1-1 mismatch_A-A; |
| | 30_6-6_internal_loop-symmetric_GCCAAG-GGACAA |
| 315 | -6_6-6_internal_loop-symmetric_AUUCAU-CUAGUG; 0_1- |
| | 1_mismatch_A-C; 5_2-2_bulge-symmetric_UG-GC; 30_6-6_internal_loop- |
| | symmetric_GCCAAG-GACUAA |
| 320 | -6_6-6_internal_loop-symmetric_AUUCAU-CGUCGC; 0_1- |
| | 1_mismatch_A-C; 11_1-1_mismatch_C-U; 19_1-1_mismatch_G-A; 30_6- |
| | 6_internal_loop-symmetric_GCCAAG-GCAUCA |
| 321 | -16_6-6_internal_loop-symmetric_GUGUAA-GAGAUG; -4_1-0_bulge- |
| | asymmetric_A-; 0_1-1_mismatch_A-C; 5_2-2_bulge-symmetric_UG-UC; |
| | 7_1-1_wobble_U-G; 30_6-6_internal_loop-symmetric_GCCAAG- |
| | AAAACA |
| 325 | -6_2-0_bulge-asymmetric_AU-; 0_1-1_mismatch_A-C; 5_1- |
| | 1_mismatch_U-C; 12_1-1_mismatch_A-G; 30_6-6_internal_loop- |
| | symmetric_GCCAAG-ACACUG |
| 338 | -14_6-6_internal_loop-symmetric_GUAAAG-GGAAUG; -5_2-0_bulge- |
| | asymmetric_UU-; 0_1-1_mismatch_A-C; 30_6-6_internal_loop- |
| | symmetric_GCCAAG-GACACG |
| 349 | -14_6-6_internal_loop-symmetric_GUAAAG-GACGCA; -6_1- |
| | 1_wobble_U-G; -3_2-0_bulge-asymmetric_AG-; 0_1-1_mismatch_A-C; |
| | 19_1-1_mismatch_G-A; 30_6-6_internal_loop-symmetric_GCCAAG- |
| | AGCCAG |
| 318 | -8_6-6_internal_loop-symmetric_GAAUUC-CGCCGA; 0_1- |
| | 1_mismatch_A-C; 2_1-1_wobble_G-U; 28_6-6_internal_loop- |
| | symmetric_AGGCCA-AUAGAG |
| 319 | -16_6-6_internal_loop-symmetric_GUGUAA-AAUGAA; -5_4-1_bulge- |
| | asymmetric_CAUU-U; 0_1-1_mismatch_A-C; 6_1-1_wobble_G-U; 28_6- |
| | 6_internal_loop-symmetric_AGGCCA-CUCAAG |
| 329 | -10_6-6_internal_loop-symmetric_AGGAAU-UCUGAG; 0_1- |
| | 1_mismatch_A-C; 5_2-2_bulge-symmetric_UG-AC; 11_1-1_mismatch_C- |
| | U; 28_6-6_internal_loop-symmetric_AGGCCA-AGUAGG |
| 334 | -16_6-6_internal_loop-symmetric_GUGUAA-AUUUUA; -4_2-0_bulge- |
| | asymmetric_UA-; 0_1-1_mismatch_A-C; 7_1-1_mismatch_U-C; 28_6- |
| | 6_internal_loop-symmetric_AGGCCA-ACAUUA |
| 347 | -10_6-6_internal_loop-symmetric_AGGAAU-CGCAGA; 0_1- |
| | 1_mismatch_A-C; 10_1-1_mismatch_U-C; 28_6-6_internal_loop- |
| | symmetric_AGGCCA-AUCAAC |
| 351 | 0_1-1_mismatch_A-C; 6_1-1_mismatch_G-G; 28_6-6_internal_loop- |
| | symmetric_AGGCCA-CUUUAC |
| 353 | -10_6-6_internal_loop-symmetric_AGGAAU-UAGAGC; -6_2-2_bulge- |
| | symmetric_AU-CG; 0_1-1_mismatch_A-C; 28_6-6_internal_loop- |
| | symmetric_AGGCCA-CUCGAG |
| 355 | -18_6-6_internal_loop-symmetric_UGGUGU-UGUAAU; -3_2-0_bulge- |
| | asymmetric_AG-; 0_1-1_mismatch_A-C; 18_0-2_bulge-asymmetric_-GA; |
| | 28_6-6_internal_loop-symmetric_AGGCCA-CUCAUC |
| 357 | -8_6-6_internal_loop-symmetric_GAAUUC-AACUGG; -2_2-1_bulge- |
| | asymmetric_GC-A; 0_1-1_mismatch_A-C; 28_6-6_internal_loop- |
| | symmetric_AGGCCA-CUAAAG |
| 359 | -10_6-6_internal_loop-symmetric_AGGAAU-CAAGAA; -6_2-1_bulge- |
| | asymmetric_AU-C; 0_1-1_mismatch_A-C; 13_1-1_mismatch_U-C; 28_6- |
| | 6_internal_loop-symmetric_AGGCCA-GAUAUG |
| 361 | -10_6-6_internal_loop-symmetric_AGGAAU-CGGAGA; -6_0-1_bulge- |
| | asymmetric_-U; 0_1-1_mismatch_A-C; 4_1-1_mismatch_A-A; 28_6- |
| | 6_internal_loop-symmetric_AGGCCA-ACAAAC |
| 363 | -3_1-1_mismatch_G-G; 0_1-1_mismatch_A-C; 28_6-6_internal_loop- |
| | symmetric_AGGCCA-AAAAAG |
| 365 | -10_6-6_internal_loop-symmetric_AGGAAU-UCUGAG; -4_2-0_bulge- |
| | asymmetric_UA-; 0_1-1_mismatch_A-C; 28_6-6_internal_loop- |
| | symmetric_AGGCCA-GAAUGA |
| 366 | -16_6-6_internal_loop-symmetric_GUGUAA-GGAAUG; -3_4-3_bulge- |
| | asymmetric_UUAG-ACU; 0_1-1_mismatch_A-C; 28_6-6_internal_loop- |
| | symmetric_AGGCCA-CAUGAG |
| 369 | -4_6-6_internal_loop-symmetric_UCAUUA-GGCGAU; 0_1- |
| | 1_mismatch_A-C; 4_2-1_bulge-asymmetric_AU-C; 28_6-6_internal_loop- |
| | symmetric_AGGCCA-CAUUGA |
| 374 | -4_6-6_internal_loop-symmetric_UCAUUA-GGUUAC; 0_1- |
| | 1_mismatch_A-C; 12_1-1_mismatch_A-A; 28_6-6_internal_loop- |
| | symmetric_AGGCCA-CCAAGA |
| 376 | -4_6-6_internal_loop-symmetric_UCAUUA-GUGCAU; 0_1- |
| | 1_mismatch_A-C; 11_1-1_mismatch_C-C; 28_6-6_internal_loop- |
| | symmetric_AGGCCA-GACAUA |
| 378 | -6_6-6_internal_loop-symmetric_AUUCAU-UUCAUC; 0_1- |
| | 1_mismatch_A-C; 3_1-1_wobble_G-U; 13_1-1_mismatch_U-C; 28_6- |
| | 6_internal_loop-symmetric_AGGCCA-GAUAUG |
| 380 | -8_6-6_internal_loop-symmetric_GAAUUC-UGUUGG; 0_1- |
| | 1_mismatch_A-C; 11_1-1_mismatch_C-U; 28_6-6_internal_loop- |
| | symmetric_AGGCCA-CUAUUA |
| 382 | -14_6-6_internal_loop-symmetric_GUAAAG-GGUCCG; -5_3-3_bulge- |
| | symmetric_AUU-UUG; 0_1-1_mismatch_A-C; 28_6-6_internal_loop- |
| | symmetric_AGGCCA-AACAUC |
| 384 | -10_6-6_internal_loop-symmetric_AGGAAU-UGCAUA; -5_3-3_bulge- |
| | symmetric_AUU-UCC; 0_1-1_mismatch A-C; 10_1-1_wobble_U-G; 28_6- |
| | 6_internal_loop-symmetric_AGGCCA-CUCAGG |
| 299 | -12_6-6_internal_loop-symmetric_AAAGGA-GGUUUG; -4_3-2_bulge- |
| | asymmetric_UUA-AC; 0_1-1_mismatch_A-C; 13_1-1_wobble_U-G; 26_6- |
| | 6_internal_loop-symmetric_AAAGGC-AAAGCA |
| 312 | -14_6-6_internal_loop-symmetric_GUAAAG-GAUCCA; -7_1- |
| | 1_mismatch_A-A; 0_1-1_mismatch_A-C; 26_6-6_internal_loop- |
| | symmetric_AAAGGC-AUGGCG |
| 323 | -12_6-6_internal_loop-symmetric_AAAGGA-AGUAAA; -2_2-1_bulge- |
| | asymmetric_GC-A; 0_1-1_mismatch_A-C; 26_6-6_internal_loop- |
| | symmetric_AAAGGC-AUAAUC |
| 327 | -8_6-6_internal_loop-symmetric_GAAUUC-AAAACA; -6_1-1_wobble_U- |
| | G; 0_1-1_mismatch_A-C; 9_1-1_mismatch_U-U; 26_6-6_internal_loop- |
| | symmetric_AAAGGC-AUAGUG |
| 341 | -16_6-6_internal_loop-symmetric_GUGUAA-CCUCUA; -7_0-1_bulge- |
| | asymmetric_-G; 0_1-1_mismatch_A-C; 11_1-1_mismatch_C-U; 26_6- |
| | 6_internal_loop-symmetric_AAAGGC-UAGGAG |
| 343 | -10_6-6_internal_loop-symmetric_AGGAAU-CGAAGA; -5_1- |
| | 1_mismatch_U-C; 0_1-1_mismatch_A-C; 26_6-6_internal_loop- |
| | symmetric_AAAGGC-UAACGC |
| 356 | -6_6-6_internal_loop-symmetric_AUUCAU-UCAUAC; 0_1- |
| | 1_mismatch_A-C; 5_2-2_bulge-symmetric_UG-AC; 26_6-6_internal_loop- |
| | symmetric_AAAGGC-AUAGGC |
| 367 | -14_6-6_internal_loop-symmetric_GUAAAG-GCGCUG; -4_3-3_bulge- |
| | symmetric_UUA-GGC; 0_1-1_mismatch_A-C; 26_6-6_internal_loop- |
| | symmetric_AAAGGC-UAAGGA |
| 371 | -10_6-6_internal_loop-symmetric_AGGAAU-CGAUAG; -5_2-2_bulge- |
| | symmetric_UU-CU; 0_1-1_mismatch_A-C; 26_6-6_internal_loop- |
| | symmetric_AAAGGC-AUAAUC |
| 373 | -20_6-6_internal_loop-symmetric_UGUGGU-UGGAGU; -5_4-4_bulge- |
| | symmetric_CAUU-CUAC; 0_1-1_mismatch_A-C; 5_0-1_bulge- |
| | asymmetric_-U; 17_1-1_mismatch_A-C; 26_6-6_internal_loop- |
| | symmetric_AAAGGC-AUGGCG |
| 295 | -6_6-6_internal_loop-symmetric_AUUCAU-UGCCUG; 0_1- |
| | 1_mismatch_A-C; 6_1-1_mismatch_G-G; 24_6-6_internal_loop- |
| | symmetric_UCAAAG-AAGUCC |
| 330 | -18_6-6_internal_loop-symmetric_UGGUGU-UGAAGU; -5_1- |
| | 1_mismatch_U-C; 0_1-1_mismatch_A-C; 24_6-6_internal_loop- |
| | symmetric_UCAAAG-AACGCU |
| 332 | -14_6-6_internal_loop-symmetric_GUAAAG-GACCUG; -5_1- |
| | 1_mismatch_U-C; 0_1-1_mismatch_A-C; 24_6-6_internal_loop- |
| | symmetric_UCAAAG-AACGCU |
| 340 | -8_6-6_internal_loop-symmetric_GAAUUC-CCUUGG; 0_1- |
| | 1_mismatch_A-C; 4_1-1_mismatch_A-C; 24_6-6_internal_loop- |
| | symmetric_UCAAAG-GGGGUU |
| 345 | -6_8-8_internal_loop-symmetric_GAAUUCAU-CGACAGGA; 0_1- |
| | 1_mismatch_A-C; 6_1-1_mismatch_G-A; 7_1-1_wobble_U-G; 24_6- |
| | 6_internal_loop-symmetric_UCAAAG-GCCAGU |

### Example 6

### Hardwired Mutations

In order to determine whether editing of an adenosine of an SNCA target RNA results in reduction of protein levels, SH-SY5Y A>G hardwired mutant cell lines were prepared expressing hardwired mutations in the TIS of Codon 1 and Codon 5, as well as hardwired mutations to the 3′UTR.

SH-SY5Y cells were obtained and subcultured. Hardwired SNCA A>G mutant cell lines were engineered using editing of DNA. Briefly, guide RNAs for each target locus and donor oligonucleotides containing the A>G mutation of interest were designed and delivered to SH-SY5Y cells via electroporation-based nucleofection. Following single cell clonal expansion and genotyping, clonal lines with the A>G mutation of interest were selected for further expansion and downstream analysis.

### Culturing and Maintenance of Experimental Cell Lines

Multiple clonal SH-SY5Y lines for each SNCA A>G mutation—three TIS codon1 A>G, three TIS codon5 A>G, four 3′UTR A>G clonal lines—were expanded and seeded on 6-well plates at 250,000 cells/well. Wild-type SH-SY5Y and primary cortical neurons from P1 humanized SNCA transgenic (hSNCA-Tg) or wild-type (WT) mice were cultured and included as controls. For undifferentiated experimental conditions, SH-SY5Y cell lines were maintained in proliferative culture media (DMEM+10% FBS, 1% GlutaMax, 1% Pen-Strep) until >80% confluent for collection for downstream transcript or protein analysis. For differentiated experimental conditions, SH-SY5Y cell lines were seeded in proliferative culture media. Following overnight incubation, culture media was exchanged for SH-SY5Y differentiation media (Neurobasal Plus+10% oN2, 2% B27, 1% GlutaMax, 1% Pen-Strep, 500 nM cAMP, SuM retinoic acid, 20 ng/uL GDNF). Differentiation media was replaced every 2 days until collection at 7 days post-differentiation.

### Total Human α-Synuclein Protein ELISA Assay

Cells were lysed in total protein lysis buffer (150 mM NaCl, 20 mM Tris pH7.5, 1 mM EDTA, 1 mM EGTA, 1% TritonX-100, 1× HaltTM protease/phosphatase inhibitor cocktail). Following lysis, total protein concentrations were measured using a protein assay kit. All protein samples were diluted to 200 ug/mL in total protein lysis buffer prior to testing with a human α-synuclein colorimetric ELISA kit. Samples were further diluted 1:20 in 2× ELISA reagent diluent, loaded in duplicate along with human α-synuclein kit standards and run per manufacturer's protocol. Based on the curve generated by the kit's human α-synuclein standards, absolute total α-synuclein protein levels (ug) were calculated and normalized to total protein (mg).

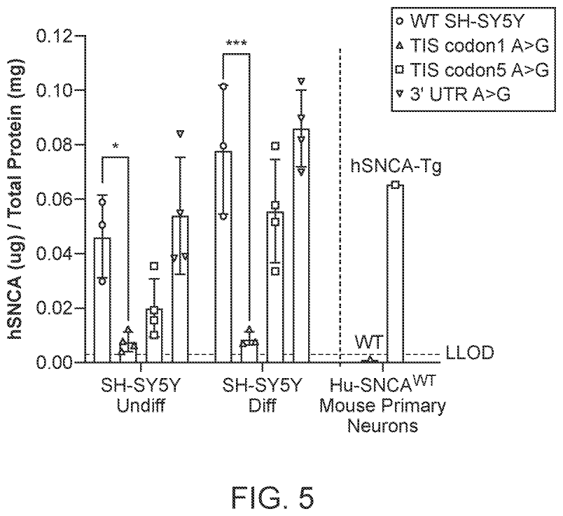

FIG. 5 depicts ELISA assessment of α-synuclein protein levels in SH-SY5Y A>G hardwired mutant cell lines. Total human α-synuclein protein levels were measured by ELISA in undifferentiated and differentiated SH-SY5Y wild-type (WT), TIS codon1 A>G mutant, TIS codon5 A>G mutant, and 3′UTR A>G mutant cell lines. Primary neurons from humanized SNCA (hSNCA-Tg) or wild-type (WT) mice were included as positive and negative total human α-synuclein protein controls, respectively. Codon1 TIS A>G hardwired mutation results in near-complete knockdown (>90%) of total α-synuclein protein. Codon5 TIS A>G hardwired mutation results in partial knockdown of total α-synuclein protein. 3′UTR A>G hardwired mutation does not impact total α-synuclein protein

### α-Synuclein Protein Immunoblot Assay

Cells were lysed in total protein lysis buffer (150 mM NaCl, 20 mM Tris pH7.5, 1 mM EDTA, 1 mM EGTA, 1% TritonX-100, 1× protease/phosphatase inhibitor cocktail). Following lysis, total protein concentrations were measured using a protein assay kit. 20 ug of total protein with sample buffer and reducing agent was incubated at 70° C. for 10 mins and loaded onto 4-12% Bis-Tris gels. Gels were run at 200V for 45 mins and transferred to nitrocellulose membrane blots using transfer stacks. Blots were blocked in blocking buffer for 10 mins at RT and incubated in rabbit monoclonal α-synuclein primary antibody [clone MJFR1, ab138501] at 1:2000 dilution overnight at 4° C. Following primary antibody incubation, blots were incubated in goat anti-rabbit IgG H&L HRP secondary antibody [ab205718] at 1:10000 dilution for 1 hr at RT. Substrate was added to blot for visualization and imaged on an imaging system. Blots were stripped in Western Blot stripping buffer, re-blocked in Pierce Fast blocking buffer for 10 mins at RT and re-probed with either mouse monoclonal GAPDH primary antibody [clone 6C5, ab8245] at 1:10000 dilution or mouse monoclonal β-actin primary antibody [clone 2F1-1] at 1:500 dilution for 2 hrs at RT and goat anti-mouse IgG H&L HRP secondary antibody [ab205719] at 1:10000 dilution for 1 hr at RT. Substrate was added to blot for visualization and imaged on an imaging system (ThermoFisher).

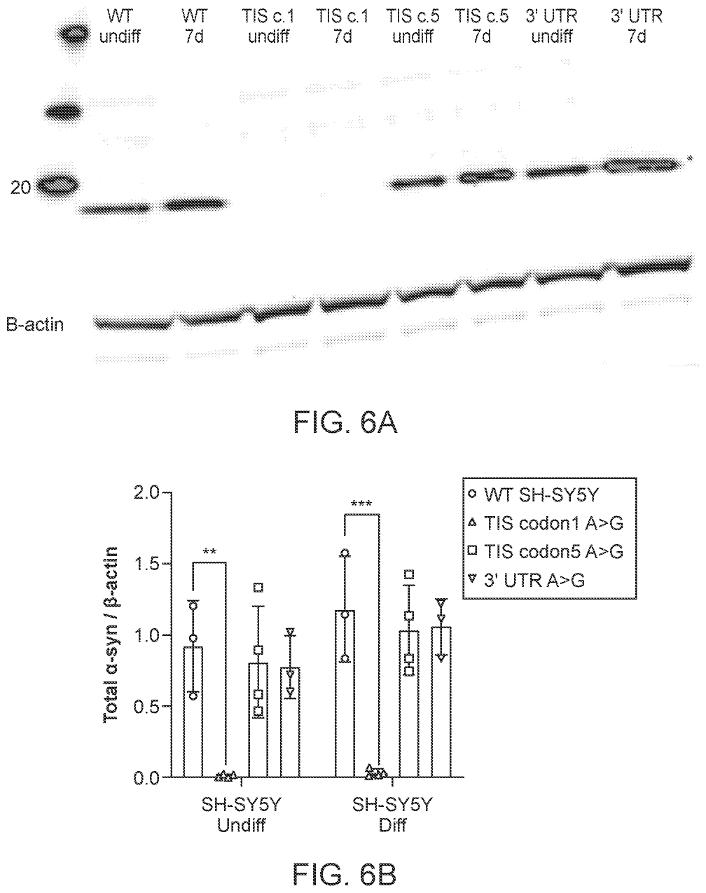

FIGS. 6A-6B show immunoblot assessment of α-synuclein protein levels in SH-SY5Y A>G hardwired mutant cell lines. Total human α-synuclein protein levels were measured by immunoblot in undifferentiated and differentiated SH-SY5Y wild-type (WT), TIS codon1 A>G mutant, TIS codon5 A>G mutant, and 3′UTR A>G mutant cell lines. Codon1 TIS A>G hardwired mutation results in complete knockdown of total α-synuclein protein. Codon5 TIS A>G and 3′UTR A>G hardwired mutations do not impact total α-synuclein protein. FIG. 6A shows representative immunoblot using α-synuclein specific antibody and beta-actin antibody as protein loading control. FIG. 6B shows quantitative densitometric analysis of immunoblot α-synuclein protein levels normalized to protein loading control.

SNCA mRNA Transcript Quantitative PCR

Cells were lysed in RLT buffer containing β-mercaptoethanol and total RNA extraction was performed according to manufacturer's protocol. cDNA synthesis from RNA samples was performed using a cDNA Reverse Transcription Kit with RNase inhibitor. For TaqMan-based detection of SNCA mRNA transcript levels, 2 uL cDNA template was added to 10 uL 2× TaqMan Fast Advanced Master Mix along with 1 uL 20× SNCA TaqMan assay (FAM; SNCA exon 2-3 or SNCA exon 3-4) and 1 uL 20× HPRT1 TaqMan assay in 20 uL total volume. All conditions were run in duplicate wells on the Real-Time PCR system. qPCR thermocycler settings were as follows: 50° C. for 2 mins, 95° C. for 20 sec, [40 cycles] 95° C. for 20 sec>60° C. for 30 sec. SNCA mRNA transcript levels were normalized to HPRT1 using the comparative CT method.

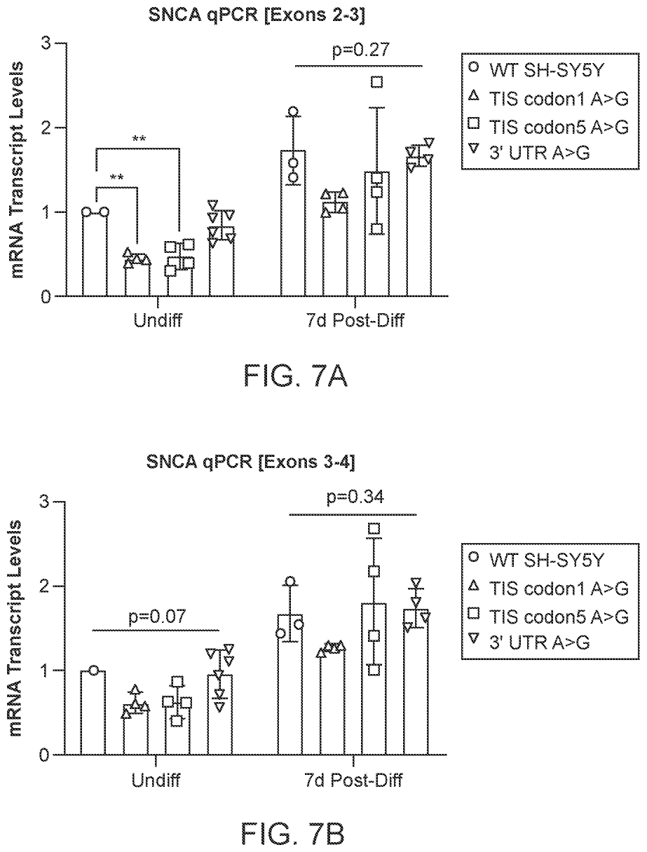

FIGS. 7A-7B show quantitative PCR assessment of SNCA mRNA transcript levels in SH-SY5Y A>G hardwired mutant cell lines. SNCA mRNA transcript levels were measured by quantitative PCR using TaqMan assays specific for either SNCA exon 2-3 junction (FIG. 7A) or SNCA exon 3-4 junction (FIG. 7B). qPCR analysis demonstrates a non-statistical trend towards decreased SNCA levels in undifferentiated TIS Codon1 and Codon5 A>G mutant SH-SY5Y lines and differentiated Codon1 TIS mutant SH-SY5Y line.

### Example 7

In Cell Editing Using Engineered gRNAs that Target the SNCA Codon 1 TIS

This example demonstrates in cell editing using the top 48 engineered guide RNA recited in TABLE 3 that target the SNCA Codon 1 TIS of Exon 2 in HEK293 cells expressing SNCA and ADAR1, as well as HEK293 cells in which ADAR2 was stably integrated via the Piggybac system.

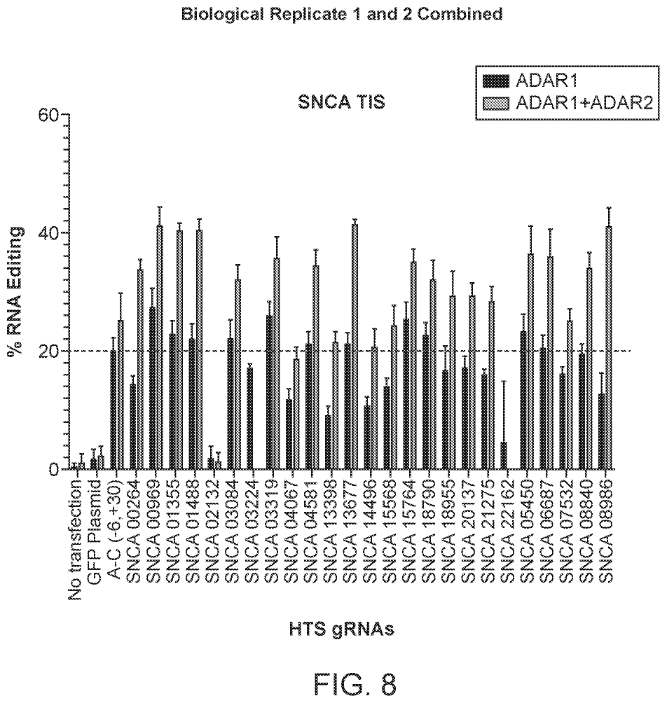

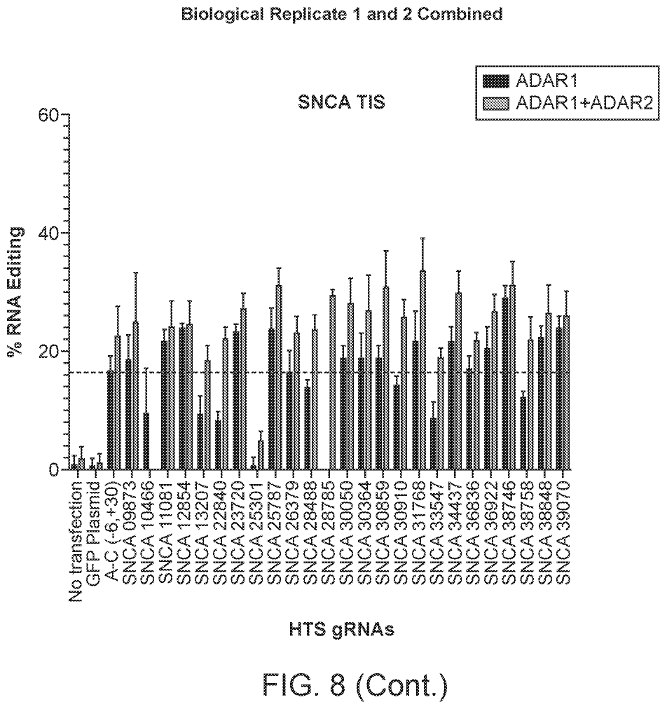

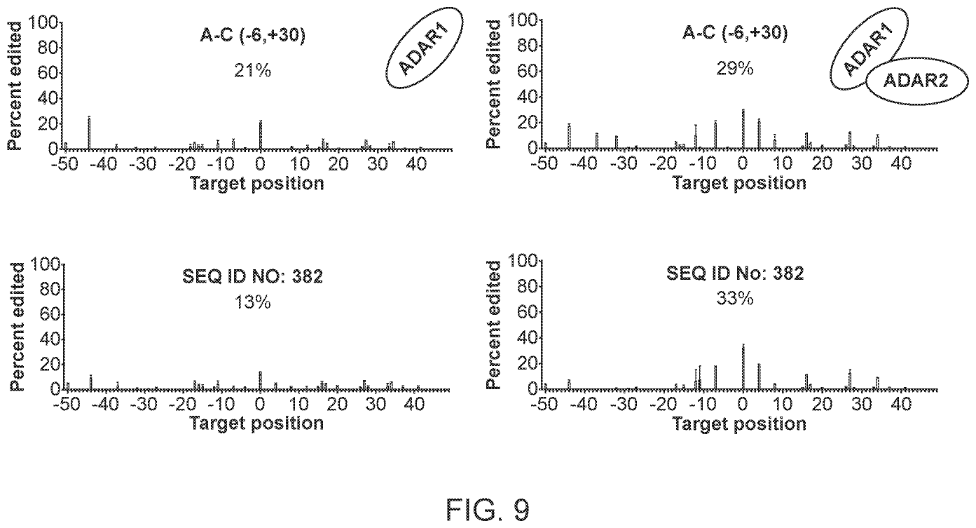

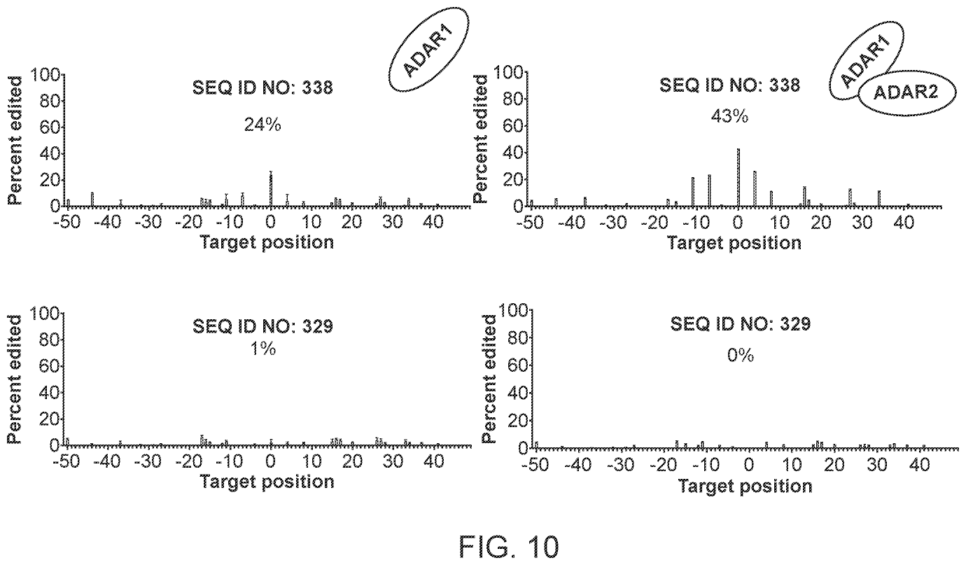

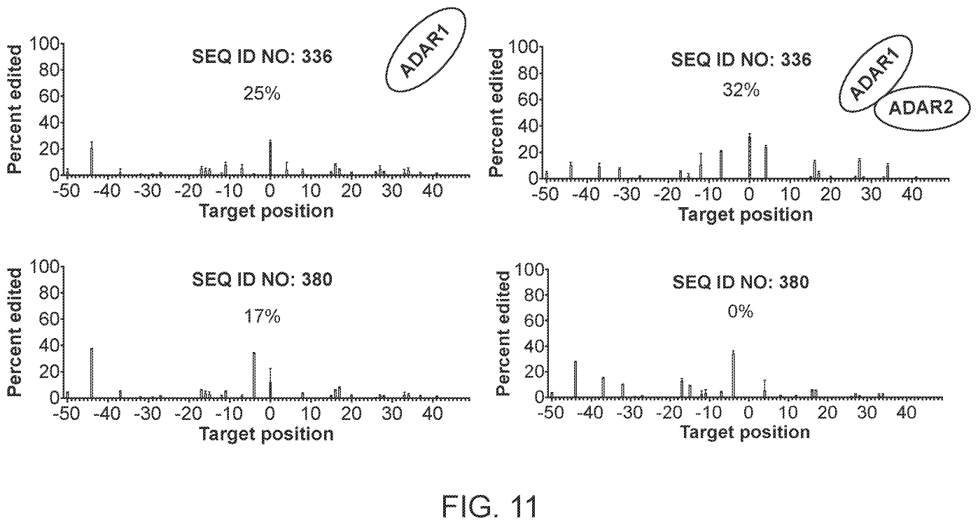

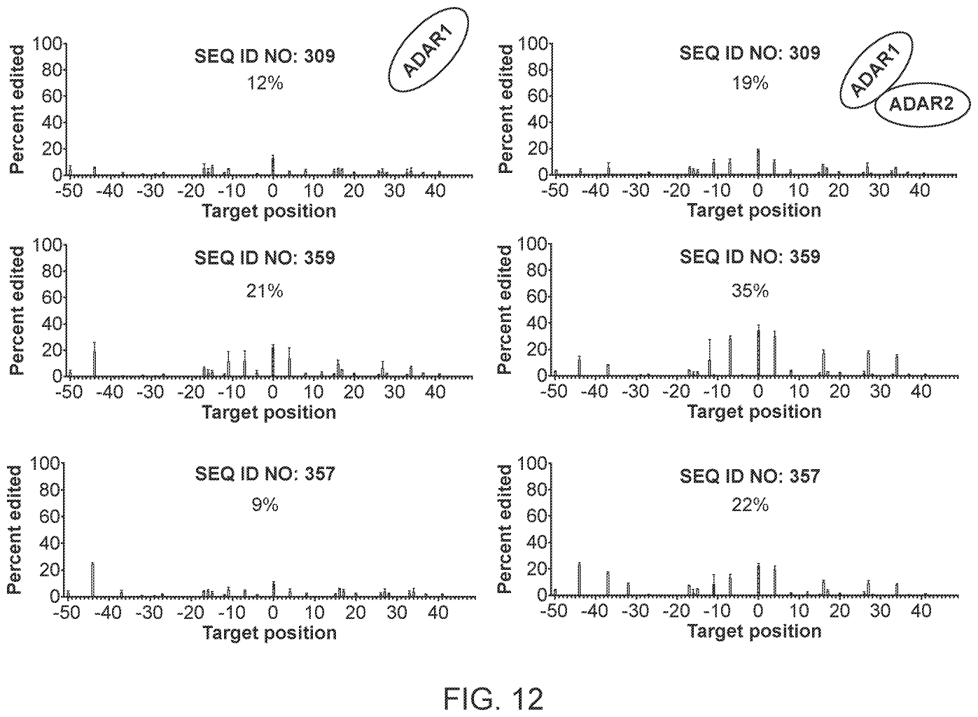

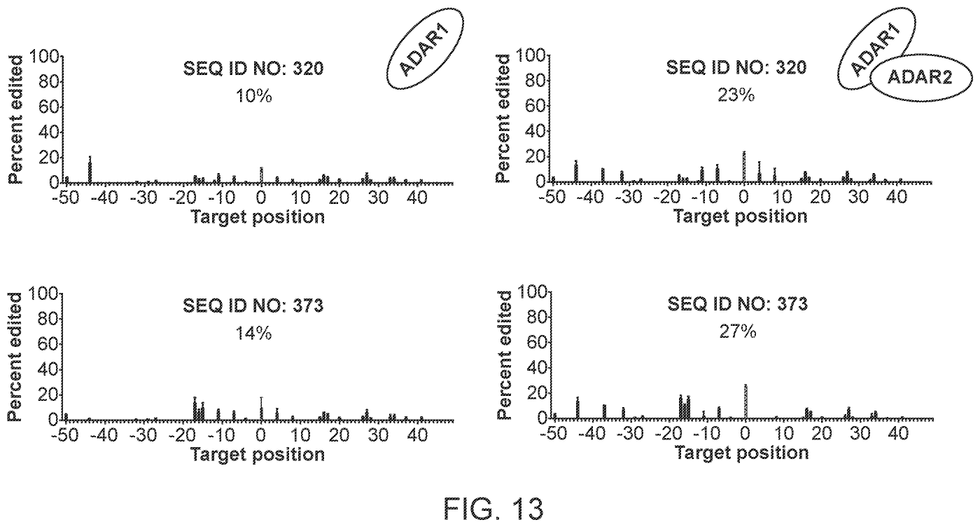

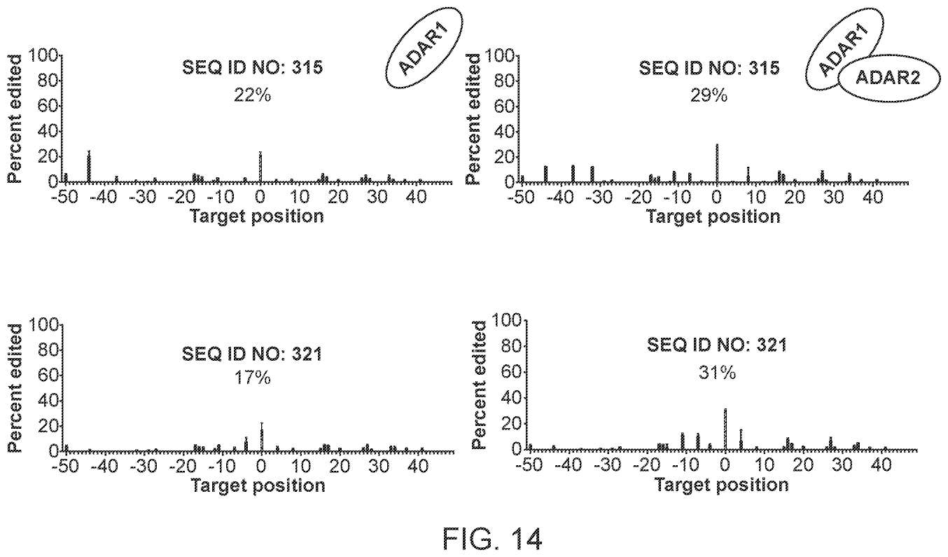

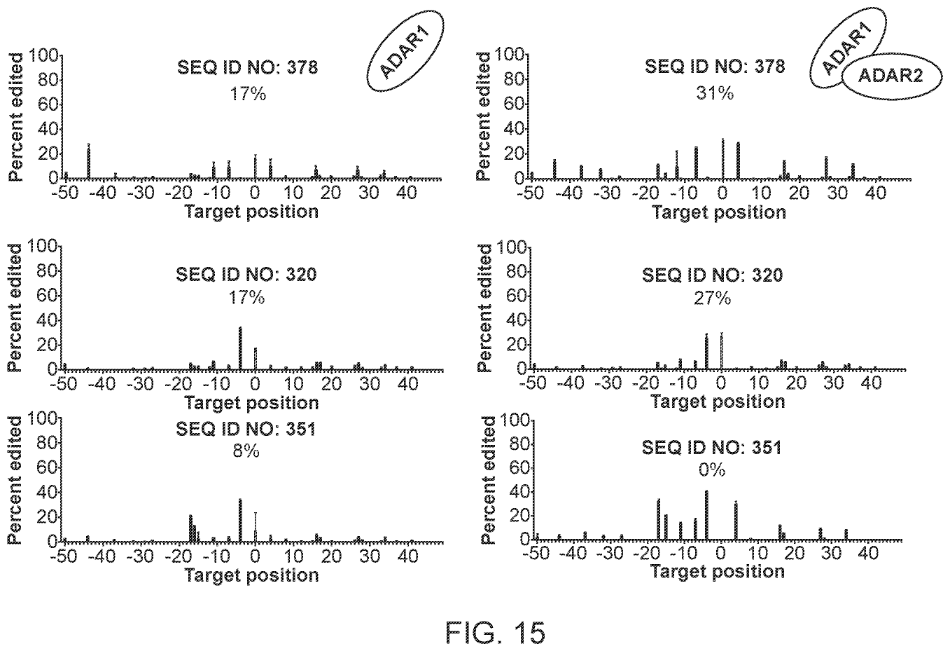

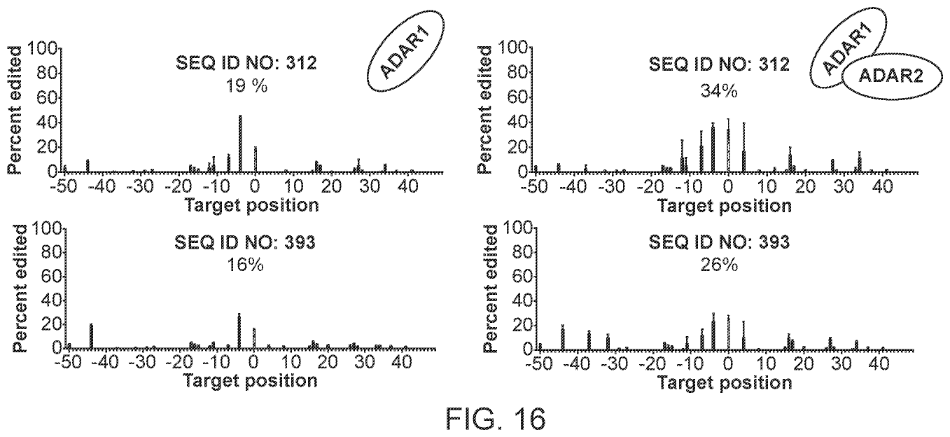

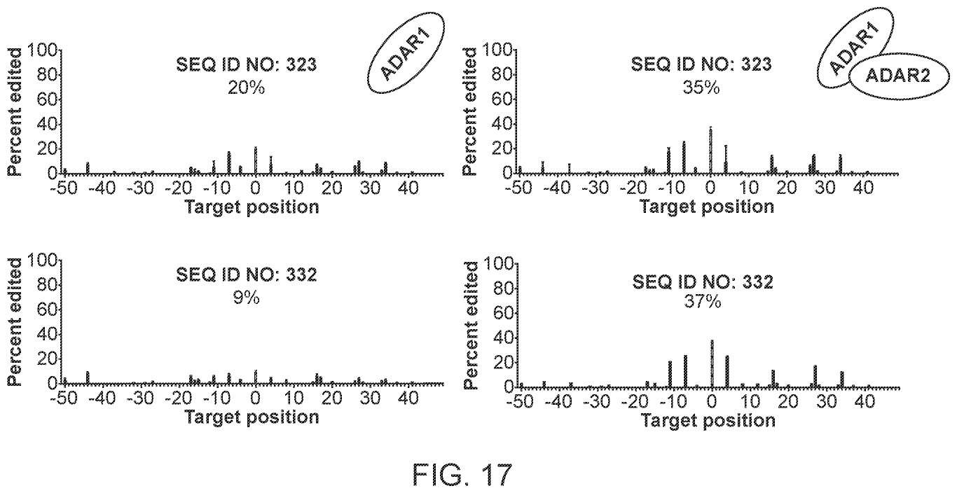

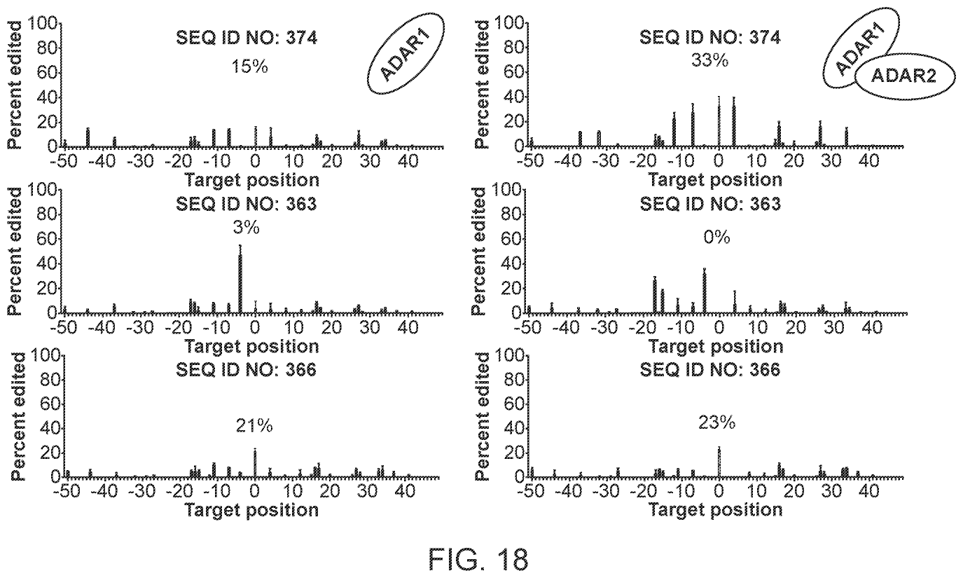

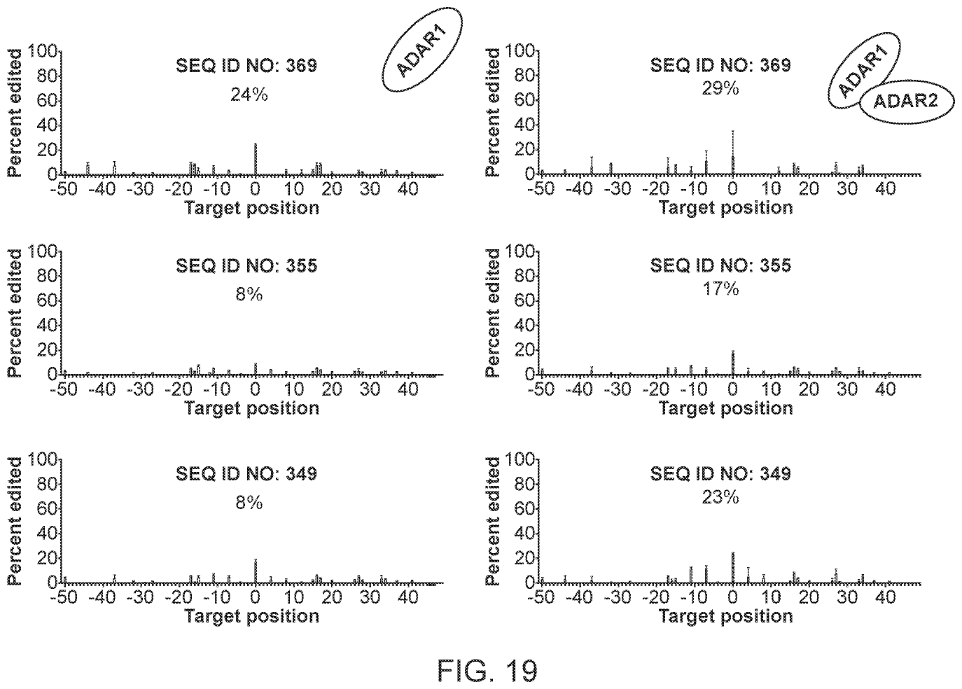

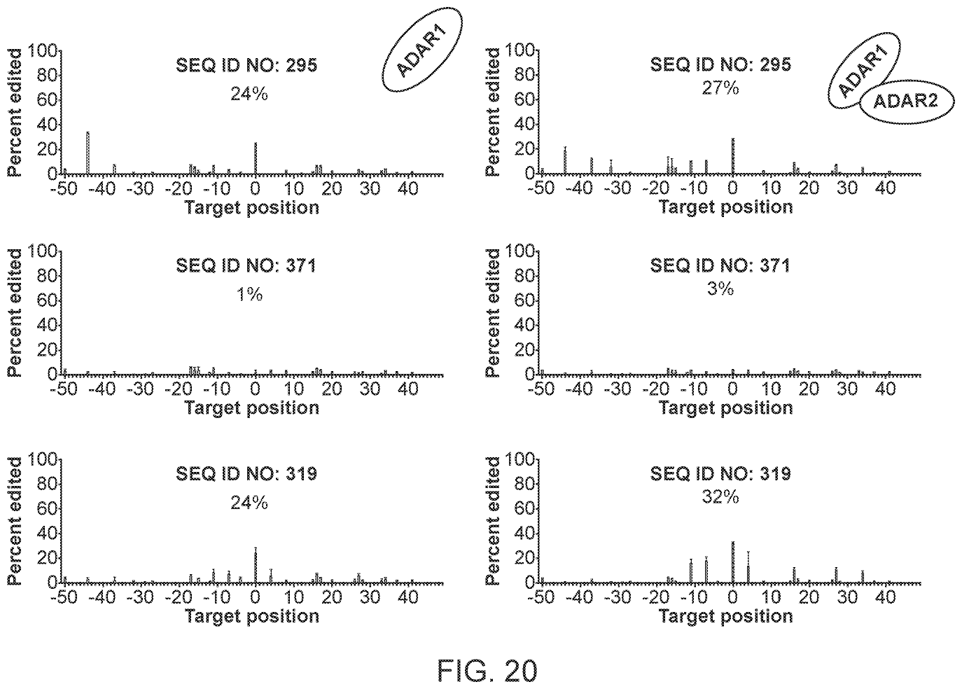

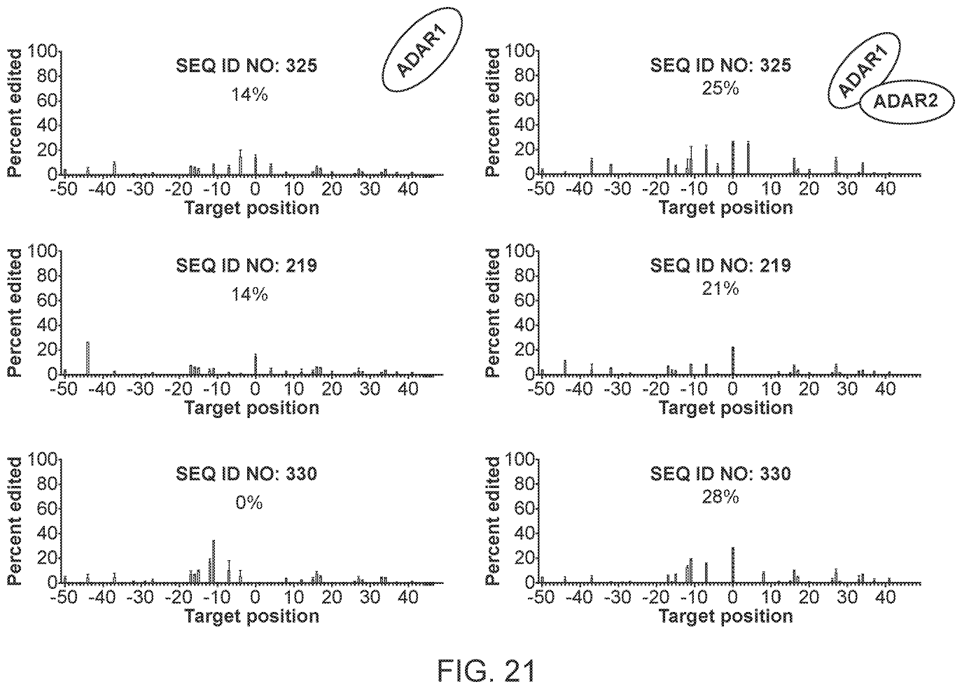

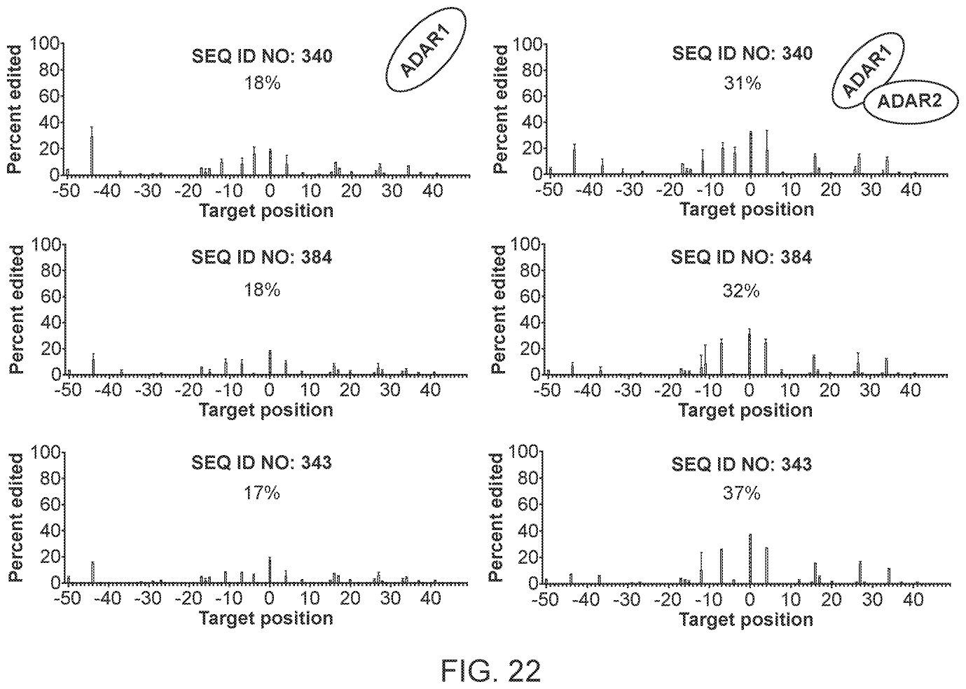

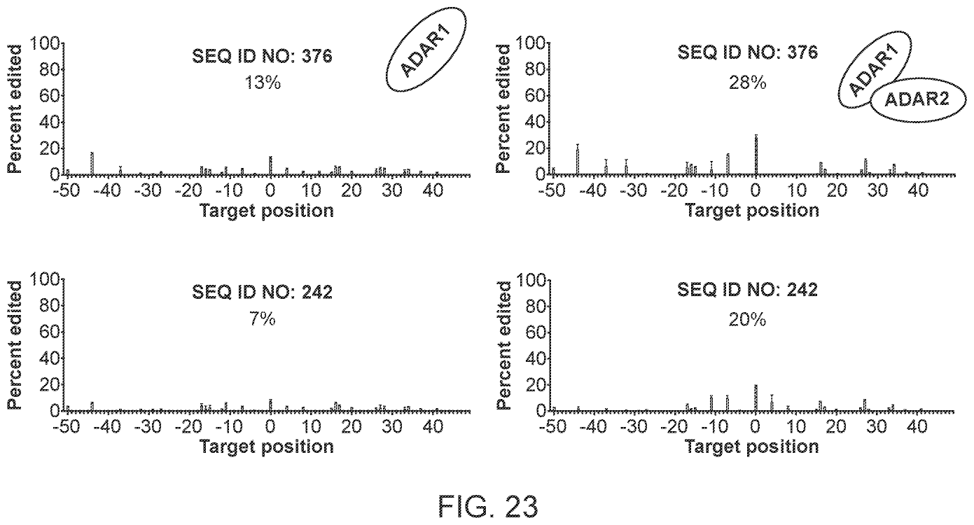

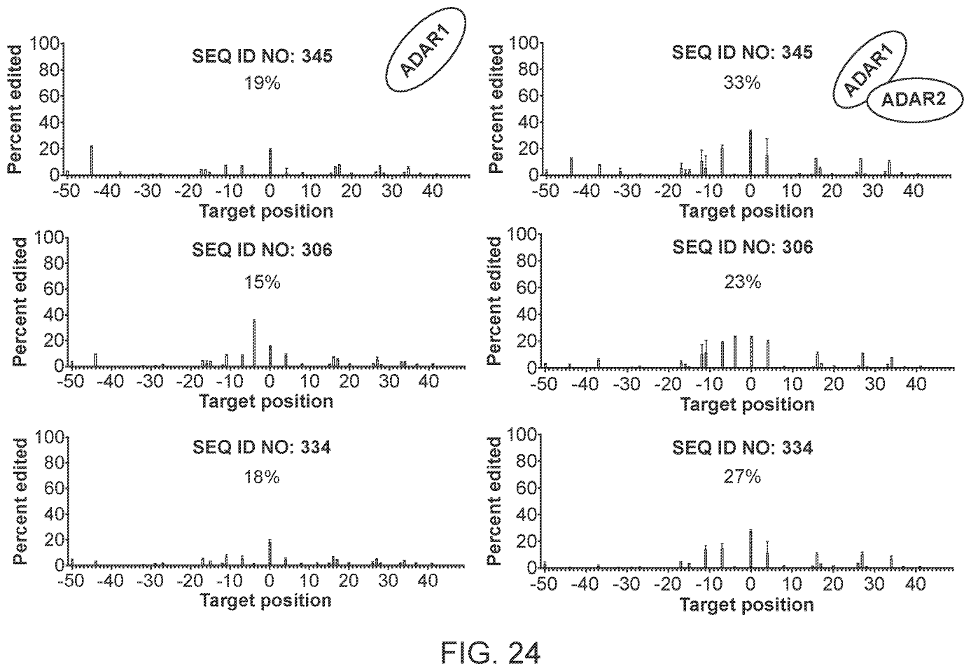

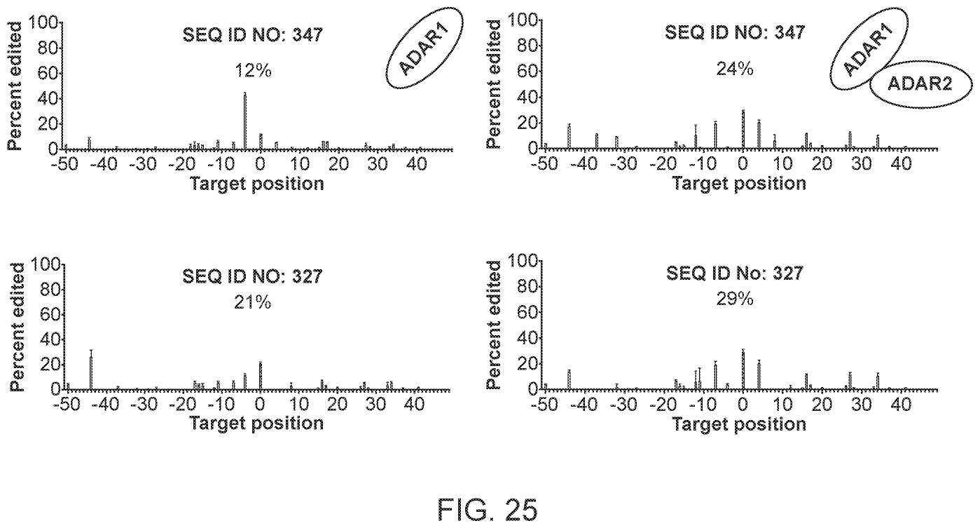

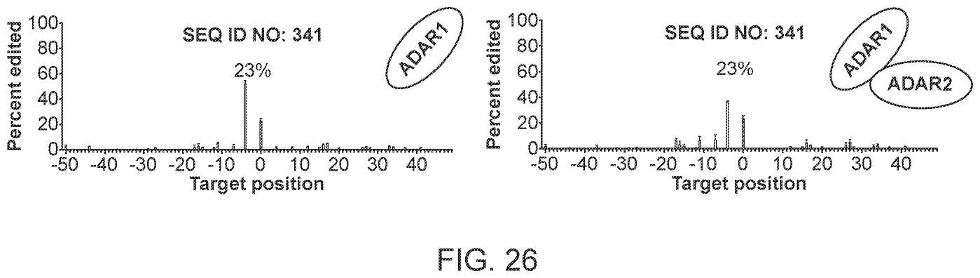

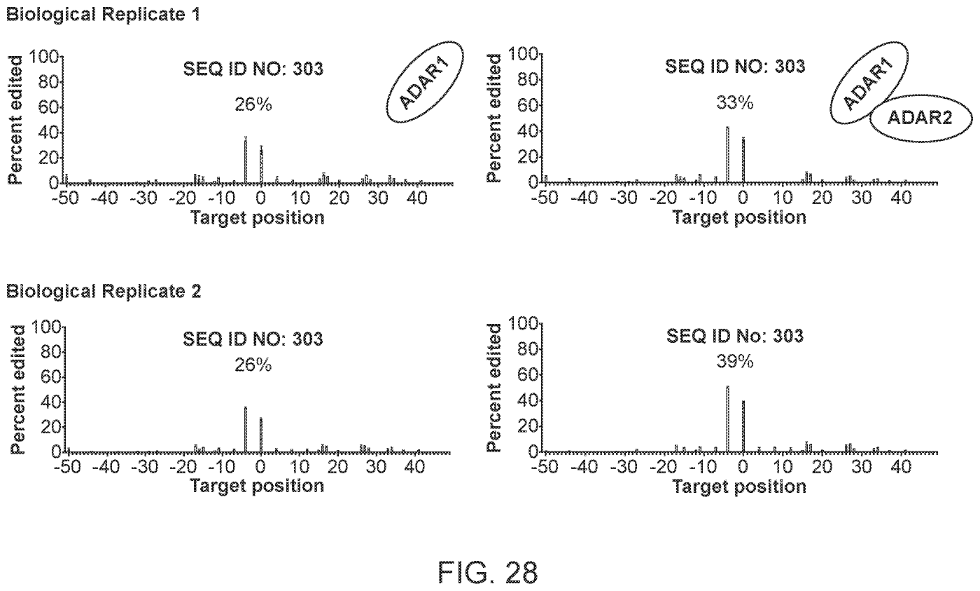

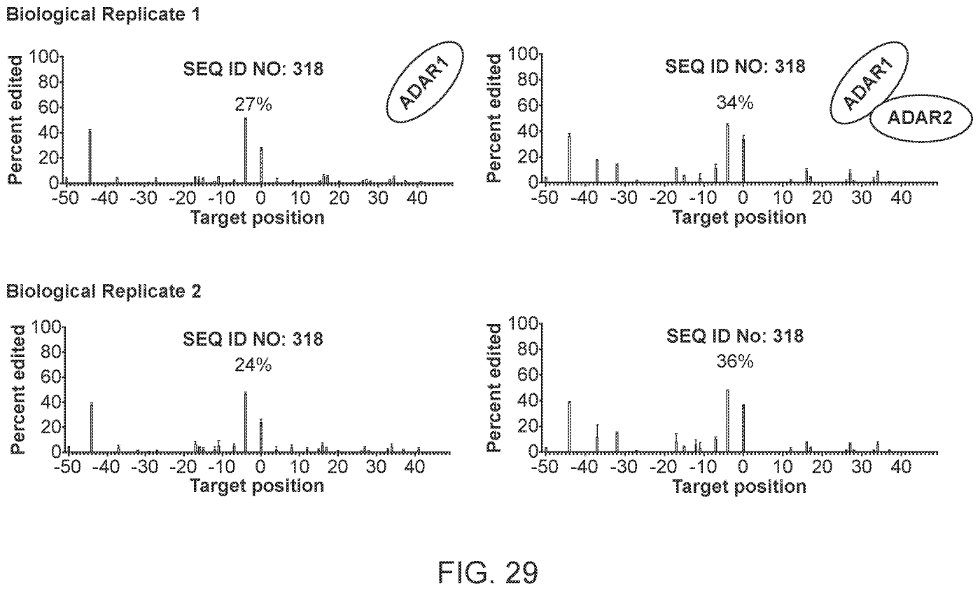

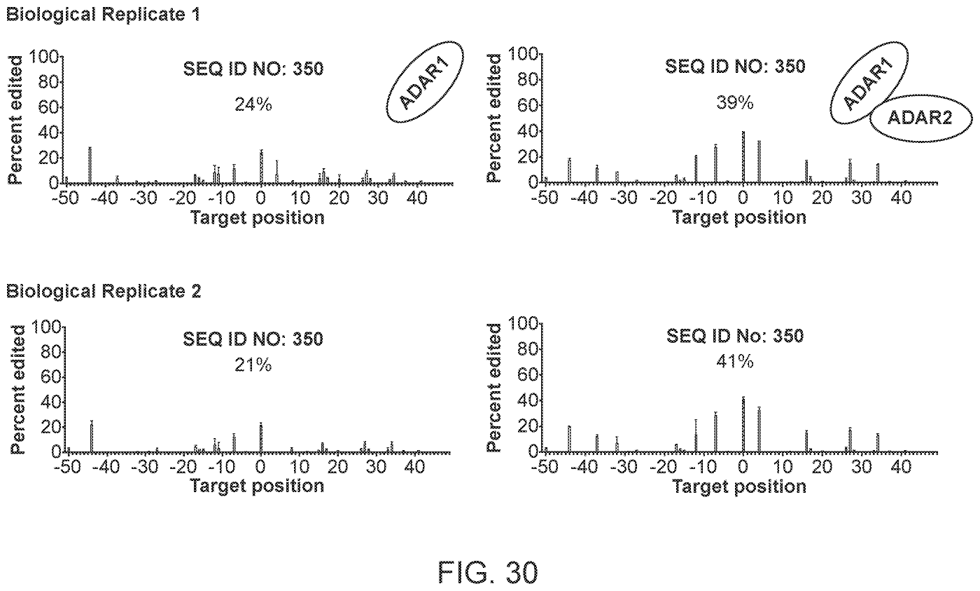

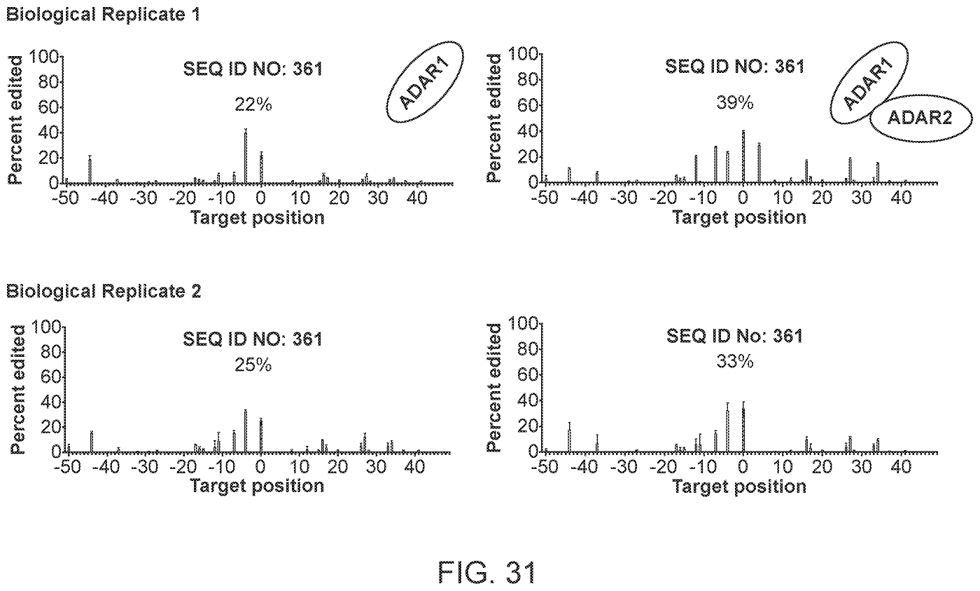

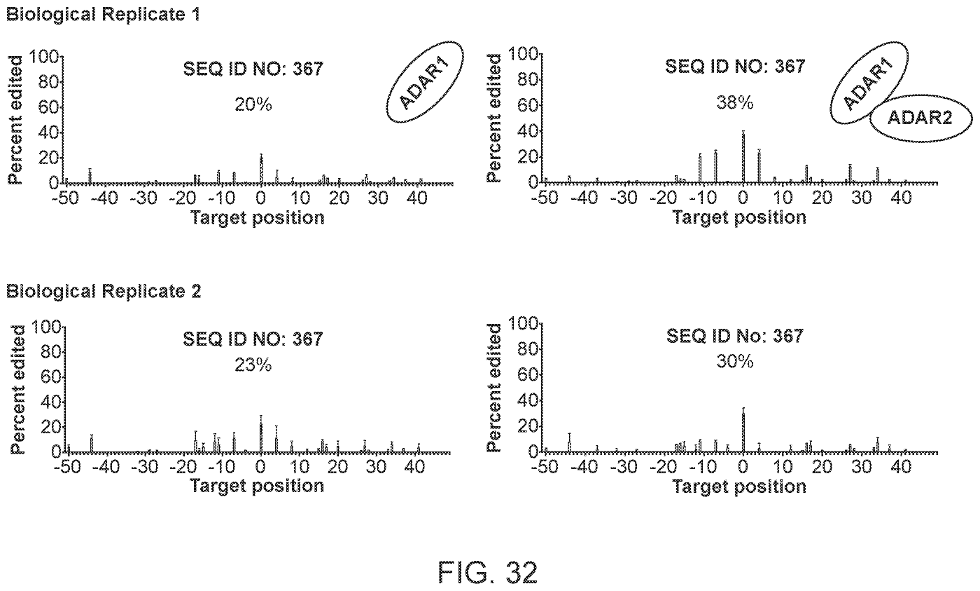

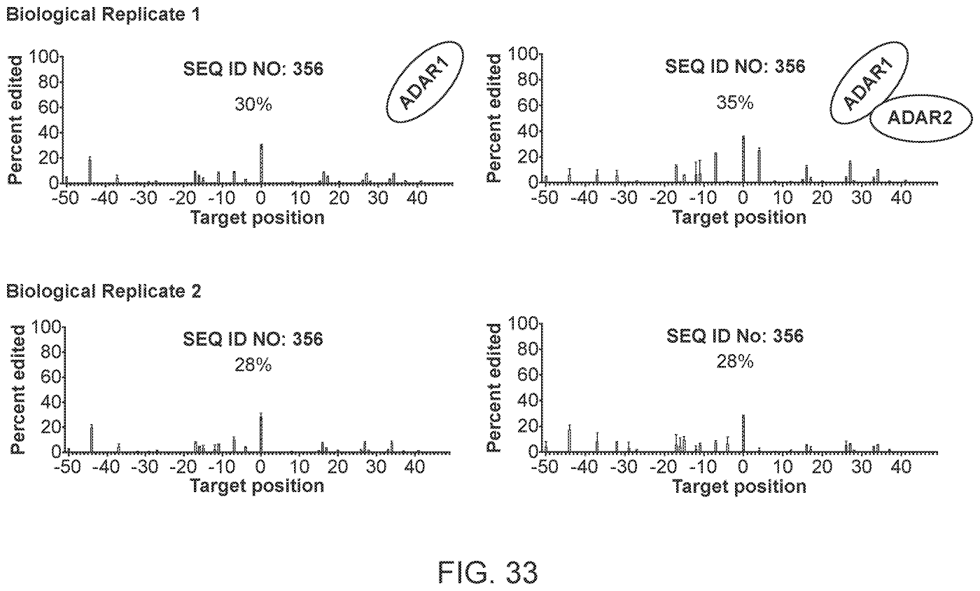

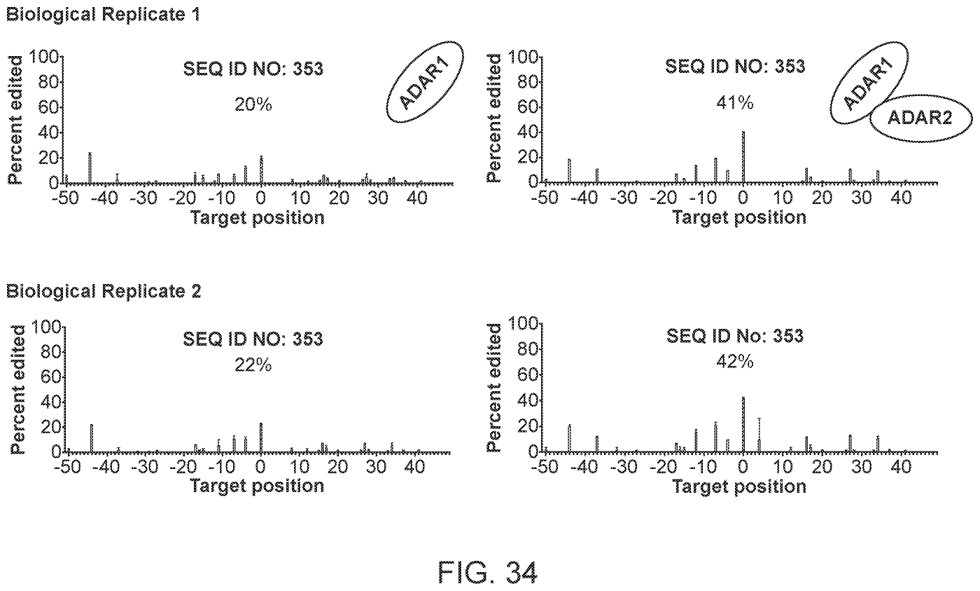

Each of the 48 guide RNAs recited in TABLE 3 were transfected into HEK293 and HEK293+ADAR2 cells. The cells were collected 48 hours post transfection, and RNA was collected, converted to DNA by reverse transcriptase and sequenced via Sanger sequencing. −48 hours after gRNA transfection, cells were harvested and analyzed for RNA by Sanger sequencing. FIG. 8 shows biological replicated of testing of the 48 gRNAs recited in TABLE 3. On target and off-target editing was determined for each guide RNA, which are recited in FIG. 9-FIG. 34.

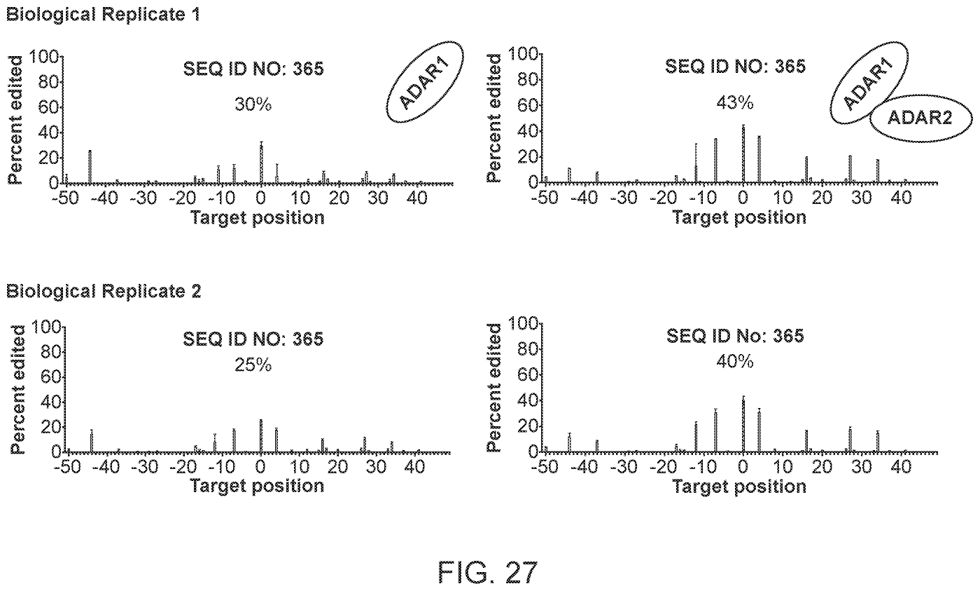

FIG. 27-FIG. 34 shows variants that produce the most on-target editing of SNCA Codon 1 TIS, corresponding to SEQ ID NO: 365, 303, 318, 350, 361, 367, 356, and 353 of TABLE 3.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

What is claimed is:

1. A composition comprising an engineered guide RNA, wherein:
a) the engineered guide RNA, upon hybridization to a sequence of a target SNCA RNA, forms a guide-target RNA scaffold with the sequence of the target SNCA RNA;
b) formation of the guide-target RNA scaffold substantially forms one or more structural features selected from the group consisting of: a bulge, an internal loop, and a hairpin;
c) the structural feature is not present within the engineered guide RNA prior to the hybridization of the engineered guide RNA to the SNCA target RNA; and
d) upon hybridization of the engineered guide RNA to the sequence of the target SNCA RNA, the engineered guide RNA facilitates RNA editing of one or more target adenosines in the sequence of the target SNCA RNA by an RNA editing entity.

2. The composition of claim 1, wherein the sequence of the target SNCA RNA is within the 3′ untranslated region (UTR).

3. The composition of claim 1, wherein the sequence of the target SNCA RNA is within the 5′ untranslated region (UTR).

4. The composition of claim 3, wherein the sequence of the target SNCA RNA in the 5′ UTR is a Kozak sequence.

5. The composition of claim 3, wherein the sequence of the target SNCA RNA in the 5′ UTR is an internal ribosomal entry site (IRES).

6. The composition of claim 3, wherein the sequence of the target SNCA RNA in the 5′ UTR is an iron response element (IRE).

7. The composition of claim 1, wherein the sequence of the target SNCA RNA comprises a translation initiation site.

8. The composition of claim 7, wherein the translation initiation site is the SNCA Codon 1 translation initiation site of Exon 2.

9. The composition of claim 7, wherein the translation initiation site is the SNCA Codon 1 translation initiation site of Exon 2 corresponding to position 226 of the SNCA transcript variant 1 of accession number NM_000345.4.

10. The composition of any one of claims 7-9, wherein the one or more structural features comprises: a first 6/6 symmetric internal loop at a position selected from the group consisting of: 32, 30, 28, 26, and 24, relative to the target adenosine at position 0.

11. The composition of claim 10, wherein the first 6/6 symmetric internal loop is at position 32, relative to the target adenosine at position 0.

12. The composition of claim 11, wherein the one or more structural features further comprises at least one structural feature selected from the group consisting of: a 6/6 symmetric internal loop at position −6 relative to position 0, an A/C mismatch at position 0, a U/C mismatch at position 13 relative to position 0, an A/C mismatch at position 15 relative to position 0, and any combination thereof.

13. The composition of claim 12, wherein the one or more structural features comprise further a 6/6 symmetric internal loop at position −6 relative to position 0, an A/C mismatch at position 0, a U/C mismatch at position 13 relative to position 0, and an A/C mismatch at position 15 relative to position 0.

14. The composition of claim 12 or claim 13, wherein the engineered guide RNA comprises at least about: 80%, 85%, 90%, 92%, 95%, 97%, or 99% sequence identity to SEQ ID NO: 350.

15. The composition of claim 14, wherein the engineered guide RNA comprises SEQ ID NO:
350.

16. The composition of claim 10, wherein the first 6/6 symmetric internal loop is at position 30, relative to the target adenosine at position 0.

17. The composition of claim 16, wherein the one or more structural features further comprises at least one structural feature selected from the group consisting of: a 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof.

18. The composition of claim 17, wherein the one or more structural features further comprise a 6/6 symmetric internal loop at position −18 relative to position 0, a 3/3 symmetric bulge at position −6 relative to position 0, an A/C mismatch at position 0, a G/G mismatch at position 6 relative to position 0, and a U/C mismatch at position 10 relative to position 0.

19. The composition of claim 17 or claim 18, wherein the engineered guide RNA comprises at least about: 80%, 85%, 90%, 92%, 95%, 97%, or 99% sequence identity to SEQ ID NO: 303.

20. The composition of claim 19, wherein the engineered guide RNA comprises SEQ ID NO:
303.

21. The composition of claim 10, wherein the first 6/6 symmetric internal loop is at position 28, relative to the target adenosine at position 0.

22. The composition of claim 21, wherein the one or more structural features further comprises at least one structural feature selected from the group consisting of: a 6/6 symmetric internal loop at position −8 relative to position 0, an A/C mismatch at position 0, a G/U Wobble at position 2 relative to position 0, and any combination thereof.

23. The composition of claim 22, wherein the one or more structural features further comprise a 6/6 symmetric internal loop at position −8 relative to position 0, an A/C mismatch at position 0, and a G/U Wobble at position 2 relative to position 0.

24. The composition of claim 22 or claim 23, wherein the engineered guide RNA comprises at least about: 80%, 85%, 90%, 92%, 95%, 97%, or 99% sequence identity to SEQ ID NO: 318.

25. The composition of claim 24, wherein the engineered guide RNA comprises SEQ ID NO: 318.

26. The composition of claim 21, wherein the one or more structural features further comprises at least one structural feature selected from the group consisting of: a 6/6 symmetric internal loop at position −10 relative to position 0, a 2/2 symmetric bulge at position −6 relative to position 0, an A/C mismatch at position 0, and any combination thereof.

27. The composition of claim 26, wherein the one or more structural features further comprise a 6/6 symmetric internal loop at position −10 relative to position 0, a 2/2 symmetric bulge at position −6 relative to position 0, and an A/C mismatch at position 0.

28. The composition of claim 26 or claim 27, wherein the engineered guide RNA comprises at least about: 80%, 85%, 90%, 92%, 95%, 97%, or 99% sequence identity to SEQ ID NO: 353.

29. The composition of claim 28, wherein the engineered guide RNA comprises SEQ ID NO: 353.

30. The composition of claim 21, wherein the one or more structural features further comprises at least one structural feature selected from the group consisting of: a 6/6 symmetric internal loop at position −10 relative to position 0, a 0/1 asymmetric bulge at position −6 relative to position 0, an A/C mismatch at position 0, a A/A mismatch at position 4 relative to position 0, and any combination thereof.

31. The composition of claim 30, wherein the one or more structural features further comprise a 6/6 symmetric internal loop at position −10 relative to position 0, a 0/1 asymmetric bulge at position −6 relative to position 0, an A/C mismatch at position 0, and a A/A mismatch at position 4 relative to position 0.

32. The composition of claim 30 or claim 31, wherein the engineered guide RNA comprises at least about: 80%, 85%, 90%, 92%, 95%, 97%, or 99% sequence identity to SEQ ID NO: 361.

33. The composition of claim 32, wherein the engineered guide RNA comprises SEQ ID NO: 361.

34. The composition of claim 21, wherein the one or more structural features further comprises at least one structural feature selected from the group consisting of: a 6/6 symmetric internal loop at position −10 relative to position 0, a 2/0 asymmetric bulge at position −4 relative to position 0, an A/C mismatch at position 0, and any combination thereof.

35. The composition of claim 34, wherein the one or more structural features further comprise a 6/6 symmetric internal loop at position −10 relative to position 0, a 2/0 asymmetric bulge at position −4 relative to position 0, and an A/C mismatch at position 0.

36. The composition of claim 34 or claim 35, wherein the engineered guide RNA comprises at least about: 80%, 85%, 90%, 92%, 95%, 97%, or 99% sequence identity to SEQ ID NO: 365.

37. The composition of claim 36, wherein the engineered guide RNA comprises SEQ ID NO: 365.

38. The composition of claim 10, wherein the first 6/6 symmetric internal loop is at position 26, relative to the target adenosine at position 0.

39. The composition of claim 38, wherein the one or more structural features further comprises at least one structural feature selected from the group consisting of: a 6/6 symmetric internal loop at position −6 relative to position 0, an A/C mismatch at position 0, a 2/2 symmetric bulge at position 5 relative to position 0, and any combination thereof.

40. The composition of claim 39, wherein the one or more structural features further comprise a 6/6 symmetric internal loop at position −6 relative to position 0, an A/C mismatch at position 0, and a 2/2 symmetric bulge at position 5 relative to position 0.

41. The composition of claim 39 or claim 40, wherein the engineered guide RNA comprises at least about: 80%, 85%, 90%, 92%, 95%, 97%, or 99% sequence identity to SEQ ID NO: 356.

42. The composition of claim 41, wherein the engineered guide RNA comprises SEQ ID NO: 356.

43. The composition of claim 38, wherein the one or more structural features further comprises at least one structural feature selected from the group consisting of: a 6/6 symmetric internal loop at position −14 relative to position 0, a 3/3 symmetric bulge at position −4 relative to position 0, an A/C mismatch at position 0, and any combination thereof.

44. The composition of claim 43, wherein the one or more structural features further comprise a 6/6 symmetric internal loop at position −14 relative to position 0, a 3/3 symmetric bulge at position −4 relative to position 0, and an A/C mismatch at position 0.

45. The composition of claim 43 or claim 44, wherein the engineered guide RNA comprises at least about: 80%, 85%, 90%, 92%, 95%, 97%, or 99% sequence identity to SEQ ID NO: 367.

46. The composition of claim 45, wherein the engineered guide RNA comprises SEQ ID NO: 367.

47. The composition of claim 10, wherein the first 6/6 symmetric internal loop is at position 24, relative to the target adenosine at position 0.

48. The composition of any one of claims 1-47, wherein the one or more structural features comprises at least a first 6/6 symmetric internal loop and at least a second 6/6 symmetric loop.

49. The composition of any one of claims 1-47, wherein the one or more structural features comprises the bulge, and wherein the bulge is a symmetric bulge.

50. The composition of any one of claims 1-47, wherein the one or more structural features comprises the bulge, and wherein the bulge is an asymmetric bulge.

51. The composition of any one of claims 1-50, wherein the one or more structural features comprises the internal loop, and wherein the internal loop is a symmetric internal loop.

52. The composition of any one of claims 1-50, wherein the one or more structural features comprises the internal loop, and wherein the internal loop is an asymmetric internal loop.

53. The composition of any one of claims 1-52, wherein the guide-target RNA scaffold comprises a Wobble base pair.

54. The composition of any one of claims 1-53, wherein the one or more structural features comprises the hairpin, and wherein the hairpin is a recruitment hairpin or a non-recruitment hairpin.

55. The composition of any one of claims 1-54, wherein upon hybridization of the engineered guide RNA to the sequence of the target SNCA RNA, the engineered guide RNA facilitates RNA editing of one or more adenosines in the sequence of the target SNCA RNA by an RNA editing entity.

56. The composition of claim 55, wherein the RNA editing entity comprises ADAR1, ADAR2, ADAR3, or any combination thereof.

57. The composition of any one of claims 1-56, wherein the engineered guide RNA comprises at least 80%, at least 85%, at least 90%, at least 95%, at least 99%, or 100% sequence identity to any one of SEQ ID NO: 2-SEQ ID NO: 11.

58. The composition of any one of claims 1-57, wherein the engineered guide RNA is encoded by an engineered polynucleotide.

59. The composition of claim 58, wherein the engineered polynucleotide is comprised in or on a vector.

60. The composition of claim 59, wherein the vector is a viral vector, and wherein the engineered polynucleotide is encapsidated in the viral vector.

61. The composition of claim 60, wherein the viral vector is an adeno-associated viral (AAV) vector, a derivative thereof.

62. The composition of claim 61, wherein the viral vector is an adeno-associated viral (AAV) and wherein the AAV vector is AAV1, AAV2, AAV3, AAV4, AAV5, AAV6, AAV7, AAV8, AAV9, AAV10, AAV11, or a derivative, a chimera, or a variant of any of these.

63. The composition of any one of claims 61-62, wherein the AAV vector is a recombinant AAV (rAAV) vector, a hybrid AAV vector, a chimeric AAV vector, a self-complementary AAV (scAAV) vector, or any combination thereof.

64. The composition of any one of claims 1-63, wherein the engineered guide RNA has at least 80%, 85%, 90%, 92%, 95%, 97%, or 99% sequence identity to any one of SEQ ID NO: 12-SEQ ID NO: 384.

65. The composition of any one of claims 1-63, wherein the engineered guide RNA has a sequence of any one of SEQ ID NO: 12-SEQ ID NO 384.

66. A pharmaceutical composition comprising:
a) the composition of any one of claims 1-65; and
b) a pharmaceutically acceptable: excipient, carrier, or diluent.

67. A method of treating a disease or a condition in a subject in need thereof, the method comprising administering to the subject a therapeutically effective amount of the composition of any one of claims 1-65 or the pharmaceutical composition of claim 66.

68. The method of claim 67, wherein the disease or condition comprises a synucleinopathy.

69. The method of claim 68, wherein the synucleinopathy comprises Parkinson's disease.

70. The method of any one of claims 67-69, wherein the subject is a human or a non-human animal.

71. The method of any one of claims 67-70, wherein the pharmaceutical composition or the composition is in unit dose form.

72. The method of any one of claims 67-71, wherein the administering is sufficient to treat one or more symptoms of the disease of condition.

73. The method of claim 72, wherein the disease or condition is a synucleinopathy.

74. The method of claim 73, wherein the one of more symptoms treated comprises muscle tone rigidity, bradykinesia, resting tremor, or any combination thereof.

75. The method of claim 73-74, wherein the administering is sufficient to reduce aggregation of alpha-synuclein protein, relative to:
(a) a level of aggregation prior to the administering;
(b) a level of accumulated aggregation in the subject in the absence of the administering; or
(c) both.

76. A method of treating Parkinson's disease in a subject in need thereof, the method comprising administering to the subject the composition of any one of claims 1-65 in an amount sufficient to treat the Parkinson's disease in the subject.

77. The method of claim 76, wherein the administering is sufficient to treat one or more symptoms of the Parkinson's disease in the subject, relative to prior to the administering.

78. The method of claim 77, wherein the one of more symptoms treated comprises muscle tone rigidity, bradykinesia, resting tremor, or any combination thereof.

79. The method of any one of claims 76-78, wherein the subject after the administering displays an increased Unified Parkinson's Disease Rating Scale (UPDRS) score, relative to a UPDRS score prior to the administering.

80. A method of editing an SNCA RNA, the method comprising contacting the SNCA RNA with the composition of any one of claims 1-65 and an RNA editing entity, thereby editing the SNCA RNA.

81. The method of claim 80, wherein the editing comprises editing one or more adenosines within the 3′ untranslated region (UTR) of the SNCA RNA.

82. The method of claim 80, wherein the editing comprises editing one or more adenosines within the 5′ untranslated region (UTR) of the SNCA RNA.

83. The method of claim 80, wherein the editing comprises editing one or more adenosines of a transcription initiation site (TIS) of the SNCA RNA.

84. The method of claim 83, wherein the translation initiation site is the SNCA Codon 1 translation initiation site of Exon 2, the Codon 5 translation initiate site of Exon 2, or both.

85. The method of any one of claims 80-84, wherein the SNCA RNA comprises a pre-mRNA transcript of SNCA.

86. The method of claim 85, wherein at least 40%, at least 50%, at least 60%, at least 70%, at least 80%, at least 85%, at least 90%, at least 95%, or at least 99% of the pre-mRNA transcripts of SNCA have at least one edit.

87. The method of claim 80, wherein the editing of SNCA RNA facilitates a protein knockdown.

88. The method of claim 87, wherein the protein knockdown comprises a reduction of at least 10%, relative to an amount of protein present prior to the contacting.

89. The method of claim 87, wherein the protein knockdown comprises a reduction of from about 10% to about 25%, relative to an amount of protein present prior to the contacting.

90. The method of claim 87, wherein the protein knockdown comprises a reduction of at least 50%, relative to an amount of protein present prior to the contacting.

91. The method of claim 87, wherein the protein knockdown comprises a knockdown of alpha-synuclein.

92. The method of any one of claims 87-91, wherein the knockdown is measured in an in vitro assay.

93. The method of any one of claims 87-91, wherein the knockdown is measured in an in vivo assay.

94. The method of any one of claims 87-91, wherein the knockdown is measured in a human subject.
